SEQUENCE LISTING (1175 sequences)

SEQ ID NO: 1 - RNA, 10 nt, Artificial Sequence
  source: /note=“Description of Artificial Sequence Synthetic oligonucleotide”
aagaccuuuu

SEQ ID NO: 2 - RNA, 25 nt, Artificial Sequence
  source: /note=“Description of Artificial Sequence Synthetic oligonucleotide”
auguguuuuugucaaaagaccuuuu

SEQ ID NO: 3 - RNA, 60 nt, Artificial Sequence
  source: /note=“Description of Artificial Sequence Synthetic oligonucleotide”
aggccagcuugccgguuuuuuagucgugcugcuucauguguuuuugucaaaagaccuuuu

SEQ ID NO: 4 - DNA, 10 bp, Artificial Sequence
  source: /note=“Description of Artificial Sequence Synthetic oligonucleotide”
aagacctttt

SEQ ID NO: 5 - DNA, 25 bp, Artificial Sequence
  source: /note=“Description of Artificial Sequence Synthetic oligonucleotide”
atgtgtttttgtcaaaagacctttt

SEQ ID NO: 6 - DNA, 60 bp, Artificial Sequence
  source: /note=“Description of Artificial Sequence Synthetic oligonucleotide”
aggccagcttgccggttttttagtcgtgctgcttcatgtgtttttgtcaaaagacctttt

SEQ ID NO: 7 - DNA, 20 bp, Artificial Sequence
  source: /note=“Description of Artificial Sequence Synthetic oligonucleotide”
tttttgtcaaaagacctttt

SEQ ID NO: 8 - DNA, 30 bp, Artificial Sequence
  source: /note=“Description of Artificial Sequence Synthetic oligonucleotide”
gcttcatgtgtttttgtcaaaagacctttt

SEQ ID NO: 9 - DNA, 50 bp, Artificial Sequence
  source: /note=“Description of Artificial Sequence Synthetic oligonucleotide”
gccggttttttagtcgtgctgcttcatgtgtttttgtcaaaagacctttt

SEQ ID NO: 10 - DNA, 40 bp, Artificial Sequence
  source: /note=“Description of Artificial Sequence Synthetic oligonucleotide”
tagtcgtgctgcttcatgtgtttttgtcaaaagacctttt

SEQ ID NO: 11 - DNA, 20 bp, Artificial Sequence
  source: /note=“Description of Artificial Sequence Synthetic oligonucleotide”
ccgaagttttcttcggtttt

SEQ ID NO: 12 - DNA, 25 bp, Artificial Sequence
  source: /note=“Description of Artificial Sequence Synthetic oligonucleotide”
tttttccgaagttttcttcggtttt

SEQ ID NO: 13 - DNA, 30 bp, Artificial Sequence
  source: /note=“Description of Artificial Sequence Synthetic oligonucleotide”
aacgctttttccgaagttttcttcggtttt

SEQ ID NO: 14 - DNA, 41 bp, Artificial Sequence
  source: /note=“Description of Artificial Sequence Synthetic oligonucleotide”
gcgttgttttcaacgctttttccgaagttttcttcggtttt

SEQ ID NO: 15 - DNA, 62 bp, Artificial Sequence
  source: /note=“Description of Artificial Sequence Synthetic oligonucleotide”
ggcttcttttgaagcctttttgcgttgttttcaacgctttttccgaagttttcttcggtttt

SEQ ID NO: 16 - DNA, 25 bp, Artificial Sequence
  source: /note=“Description of Artificial Sequence Synthetic oligonucleotide”
atgtgtttttgtcaaaagacctttt

SEQ ID NO: 17 - RNA, 25 nt, Artificial Sequence
  source: /note=“Description of Artificial Sequence Synthetic oligonucleotide”
aaaaaaaaaaaaaaaaaaaaaaaaa

SEQ ID NO: 18 - DNA, 25 bp, Artificial Sequence
  source: /note=“Description of Artificial Sequence Synthetic oligonucleotide”
ttttttttttttttttttttttttt

SEQ ID NO: 19 - RNA, 25 nt, Artificial Sequence
  source: /note=“Description of Artificial Sequence Synthetic oligonucleotide”
auguguuuuugucaaaagaccuuuu

SEQ ID NO: 20 - RNA, 25 nt, Artificial Sequence
  source: /note=“Description of Artificial Sequence Synthetic oligonucleotide”
aaaaaaaaaaaaaaaaaaaaaaaaa

SEQ ID NO: 21 - RNA, 25 nt, Artificial Sequence
  source: /note=“Description of Artificial Sequence Synthetic oligonucleotide”
uuuuuuuuuuuuuuuuuuuuuuuuu

SEQ ID NO: 22 - DNA, 20 bp, Artificial Sequence
  source: /note=“Description of Artificial Sequence Synthetic oligonucleotide”
tctgcagaaatgttccccgt

SEQ ID NO: 23 - RNA, 20 nt, Artificial Sequence
  source: /note=“Description of Artificial Sequence Synthetic oligonucleotide”
ucugcagaaauguuccccgu

SEQ ID NO: 24 - RNA, 20 nt, Artificial Sequence
  source: /note=“Description of Artificial Sequence Synthetic oligonucleotide”
uaauuucuacucuuguagau

SEQ ID NO: 25 - RNA, 40 nt, Artificial Sequence
  source: /note=“Description of Artificial Sequence Synthetic oligonucleotide”
uaauuucuacucuuguagauucugcagaaauguuccccgu

SEQ ID NO: 26 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauucugcagaaauguuccccgu

SEQ ID NO: 27 - DNA, 20 bp, Artificial Sequence
  source: /note=“Description of Artificial Sequence Synthetic oligonucleotide”
tctgcagaaatgttccccgt

SEQ ID NO: 28 - DNA, 20 bp, Artificial Sequence
  source: /note=“Description of Artificial Sequence Synthetic oligonucleotide”
tgcagagaaaggtggctcta

SEQ ID NO: 29 - DNA, 20 bp, Artificial Sequence
  source: /note=“Description of Artificial Sequence Synthetic oligonucleotide”
taatgctgacttggggtggc

SEQ ID NO: 30 - DNA, 20 bp, Artificial Sequence
  source: /note=“Description of Artificial Sequence Synthetic oligonucleotide”
taggacctccaggaagattc

SEQ ID NO: 31 - DNA, 20 bp, Artificial Sequence
  source: /note=“Description of Artificial Sequence Synthetic oligonucleotide”
tagtcaacgcgaccaccacg

SEQ ID NO: 32 - DNA, 20 bp, Artificial Sequence
  source: /note=“Description of Artificial Sequence Synthetic oligonucleotide”
tcctgaggtcaccttccaca

SEQ ID NO: 33 - DNA, 20 bp, Artificial Sequence
  source: /note=“Description of Artificial Sequence Synthetic oligonucleotide”
tattgtgcctgtcatcattc

SEQ ID NO: 34 - DNA, 21 bp, Artificial Sequence
  source: /note=“Description of Artificial Sequence Synthetic oligonucleotide”
tgacaggcacaatagaaacaa

SEQ ID NO: 35 - DNA, 21 bp, Artificial Sequence
  source: /note=“Description of Artificial Sequence Synthetic oligonucleotide”
gacaggcacaatagaaacaac

SEQ ID NO: 36 - DNA, 21 bp, Artificial Sequence
  source: /note=“Description of Artificial Sequence Synthetic oligonucleotide”
aaacaacggggaacatttctg

SEQ ID NO: 37 - DNA, 21 bp, Artificial Sequence
  source: /note=“Description of Artificial Sequence Synthetic oligonucleotide”
acaacggggaacatttctgca

SEQ ID NO: 38 - DNA, 21 bp, Artificial Sequence
  source: /note=“Description of Artificial Sequence Synthetic oligonucleotide”
tgatagagccacctttctctg

SEQ ID NO: 39 - DNA, 21 bp, Artificial Sequence
  source: /note=“Description of Artificial Sequence Synthetic oligonucleotide”
gggtcacttgtgccgtggtgg

SEQ ID NO: 40 - DNA, 21 bp, Artificial Sequence
  source: /note=“Description of Artificial Sequence Synthetic oligonucleotide”
ggcacaagtgacccaggtcaa

SEQ ID NO: 41 - DNA, 21 bp, Artificial Sequence
  source: /note=“Description of Artificial Sequence Synthetic oligonucleotide”
gtcctgctgctcccagttgac

SEQ ID NO: 42 - DNA, 21 bp, Artificial Sequence
  source: /note=“Description of Artificial Sequence Synthetic oligonucleotide”
tggccatttgtaatgctgact

SEQ ID NO: 43 - DNA, 21 bp, Artificial Sequence
  source: /note=“Description of Artificial Sequence Synthetic oligonucleotide”
tggcacatctccccatccttc

SEQ ID NO: 44 - DNA, 21 bp, Artificial Sequence
  source: /note=“Description of Artificial Sequence Synthetic oligonucleotide”
catctccccatccttcaagga

SEQ ID NO: 45 - DNA, 21 bp, Artificial Sequence
  source: /note=“Description of Artificial Sequence Synthetic oligonucleotide”
ccactcgatccttgaaggatg

SEQ ID NO: 46 - DNA, 21 bp, Artificial Sequence
  source: /note=“Description of Artificial Sequence Synthetic oligonucleotide”
ggccactcgatccttgaagga

SEQ ID NO: 47 - DNA, 21 bp, Artificial Sequence
  source: /note=“Description of Artificial Sequence Synthetic oligonucleotide”
cctggggccactcgatccttg

SEQ ID NO: 48 - DNA, 21 bp, Artificial Sequence
  source: /note=“Description of Artificial Sequence Synthetic oligonucleotide”
gactggagggtgaggcccagg

SEQ ID NO: 49 - DNA, 21 bp, Artificial Sequence
  source: /note=“Description of Artificial Sequence Synthetic oligonucleotide”
atcgttcacggtcagcgactg

SEQ ID NO: 50 - DNA, 21 bp, Artificial Sequence
  source: /note=“Description of Artificial Sequence Synthetic oligonucleotide”
gtcgctgaccgtgaacgatac

SEQ ID NO: 51 - DNA, 21 bp, Artificial Sequence
  source: /note=“Description of Artificial Sequence Synthetic oligonucleotide”
cgctgaccgtgaacgatacag

SEQ ID NO: 52 - DNA, 21 bp, Artificial Sequence
  source: /note=“Description of Artificial Sequence Synthetic oligonucleotide”
gcatctatcacacctaccctg

SEQ ID NO: 53 - DNA, 21 bp, Artificial Sequence
  source: /note=“Description of Artificial Sequence Synthetic oligonucleotide”
cctaccctgatgggacgtaca

SEQ ID NO: 54 - DNA, 21 bp, Artificial Sequence
  source: /note=“Description of Artificial Sequence Synthetic oligonucleotide”
taccctgatgggacgtacact

SEQ ID NO: 55 - DNA, 21 bp, Artificial Sequence
  source: /note=“Description of Artificial Sequence Synthetic oligonucleotide”
ccctgatgggacgtacactgg

SEQ ID NO: 56 - DNA, 21 bp, Artificial Sequence
  source: /note=“Description of Artificial Sequence Synthetic oligonucleotide”
ttctcccagtgtacgtcccat

SEQ ID NO: 57 - DNA, 21 bp, Artificial Sequence
  source: /note=“Description of Artificial Sequence Synthetic oligonucleotide”
ggagaatcttcctggaggtcc

SEQ ID NO: 58 - DNA, 21 bp, Artificial Sequence
  source: /note=“Description of Artificial Sequence Synthetic oligonucleotide”
catggctccaagcaatggaat

SEQ ID NO: 59 - DNA, 21 bp, Artificial Sequence
  source: /note=“Description of Artificial Sequence Synthetic oligonucleotide”
cgcggccatggctccaagcaa

SEQ ID NO: 60 - DNA, 21 bp, Artificial Sequence
  source: /note=“Description of Artificial Sequence Synthetic oligonucleotide”
tcgcggccatggctccaagca

SEQ ID NO: 61 - DNA, 21 bp, Artificial Sequence
  source: /note=“Description of Artificial Sequence Synthetic oligonucleotide”
catcgtggtggtcgcgttgac

SEQ ID NO: 62 - DNA, 21 bp, Artificial Sequence
  source: /note=“Description of Artificial Sequence Synthetic oligonucleotide”
aaagccctcagaatccattct

SEQ ID NO: 63 - DNA, 21 bp, Artificial Sequence
  source: /note=“Description of Artificial Sequence Synthetic oligonucleotide”
cattctgtggaaggtgacctc

SEQ ID NO: 64 - DNA, 21 bp, Artificial Sequence
  source: /note=“Description of Artificial Sequence Synthetic oligonucleotide”
ttctgtggaaggtgacctcag

SEQ ID NO: 65 - DNA, 21 bp, Artificial Sequence
  source: /note=“Description of Artificial Sequence Synthetic oligonucleotide”
cctgaggtcaccttccacaga

SEQ ID NO: 66 - DNA, 21 bp, Artificial Sequence
  source: /note=“Description of Artificial Sequence Synthetic oligonucleotide”
ttctcctgaggtcaccttcca

SEQ ID NO: 67 - DNA, 21 bp, Artificial Sequence
  source: /note=“Description of Artificial Sequence Synthetic oligonucleotide”
aggagaaaatcagctggacag

SEQ ID NO: 68 - DNA, 21 bp, Artificial Sequence
  source: /note=“Description of Artificial Sequence Synthetic oligonucleotide”
ggagaaaatcagctggacagg

SEQ ID NO: 69 - DNA, 21 bp, Artificial Sequence
  source: /note=“Description of Artificial Sequence Synthetic oligonucleotide”
gccccagtgctccctcacccc

SEQ ID NO: 70 - DNA, 21 bp, Artificial Sequence
  source: /note=“Description of Artificial Sequence Synthetic oligonucleotide”
tggacacagcttcctgggggt

SEQ ID NO: 71 - DNA, 21 bp, Artificial Sequence
  source: /note=“Description of Artificial Sequence Synthetic oligonucleotide”
tctgcctggacacagcttcct

SEQ ID NO: 72 - DNA, 21 bp, Artificial Sequence
  source: /note=“Description of Artificial Sequence Synthetic oligonucleotide”
agctgcacctgctgggctctg

SEQ ID NO: 73 - DNA, 21 bp, Artificial Sequence
  source: /note=“Description of Artificial Sequence Synthetic oligonucleotide”
gctgggctctgtggagagcag

SEQ ID NO: 74 - DNA, 21 bp, Artificial Sequence
  source: /note=“Description of Artificial Sequence Synthetic oligonucleotide”
tgggctctgtggagagcagcg

SEQ ID NO: 75 - DNA, 21 bp, Artificial Sequence
  source: /note=“Description of Artificial Sequence Synthetic oligonucleotide”
ctgcatgactacttcaatgtc

SEQ ID NO: 76 - DNA, 21 bp, Artificial Sequence
  source: /note=“Description of Artificial Sequence Synthetic oligonucleotide”
aatgtcctgagttacagaagc

SEQ ID NO: 77 - DNA, 21 bp, Artificial Sequence
  source: /note=“Description of Artificial Sequence Synthetic oligonucleotide”
tgggtaactgcagcttcttca

SEQ ID NO: 78 - DNA, 20 bp, Artificial Sequence
  source: /note=“Description of Artificial Sequence Synthetic oligonucleotide”
gacaggcacaatagaaacaa

SEQ ID NO: 79 - DNA, 20 bp, Artificial Sequence
  source: /note=“Description of Artificial Sequence Synthetic oligonucleotide”
acaggcacaatagaaacaac

SEQ ID NO: 80 - DNA, 20 bp, Artificial Sequence
  source: /note=“Description of Artificial Sequence Synthetic oligonucleotide”
caggcacaatagaaacaacg

SEQ ID NO: 81 - DNA, 20 bp, Artificial Sequence
  source: /note=“Description of Artificial Sequence Synthetic oligonucleotide”
gggaacatttctgcagagaa

SEQ ID NO: 82 - DNA, 20 bp, Artificial Sequence
  source: /note=“Description of Artificial Sequence Synthetic oligonucleotide”
aacatttctgcagagaaagg

SEQ ID NO: 83 - DNA, 20 bp, Artificial Sequence
  source: /note=“Description of Artificial Sequence Synthetic oligonucleotide”
atgtcacctctcctccacca

SEQ ID NO: 84 - DNA, 20 bp, Artificial Sequence
  source: /note=“Description of Artificial Sequence Synthetic oligonucleotide”
cttgtgccgtggtggaggag

SEQ ID NO: 85 - DNA, 20 bp, Artificial Sequence
  source: /note=“Description of Artificial Sequence Synthetic oligonucleotide”
ggtcacttgtgccgtggtgg

SEQ ID NO: 86 - DNA, 20 bp, Artificial Sequence
  source: /note=“Description of Artificial Sequence Synthetic oligonucleotide”
caccacggcacaagtgaccc

SEQ ID NO: 87 - DNA, 20 bp, Artificial Sequence
  source: /note=“Description of Artificial Sequence Synthetic oligonucleotide”
ctgggtcacttgtgccgtgg

SEQ ID NO: 88 - DNA, 20 bp, Artificial Sequence
  source: /note=“Description of Artificial Sequence Synthetic oligonucleotide”
gacctgggtcacttgtgccg

SEQ ID NO: 89 - DNA, 20 bp, Artificial Sequence
  source: /note=“Description of Artificial Sequence Synthetic oligonucleotide”
cacaagtgacccaggtcaac

SEQ ID NO: 90 - DNA, 20 bp, Artificial Sequence
  source: /note=“Description of Artificial Sequence Synthetic oligonucleotide”
acaagtgacccaggtcaact

SEQ ID NO: 91 - DNA, 20 bp, Artificial Sequence
  source: /note=“Description of Artificial Sequence Synthetic oligonucleotide”
ccaggtcaactgggagcagc

SEQ ID NO: 92 - DNA, 20 bp, Artificial Sequence
  source: /note=“Description of Artificial Sequence Synthetic oligonucleotide”
ctgctgctcccagttgacct

SEQ ID NO: 93 - DNA, 20 bp, Artificial Sequence
  source: /note=“Description of Artificial Sequence Synthetic oligonucleotide”
cctgctgctcccagttgacc

SEQ ID NO: 94 - DNA, 20 bp, Artificial Sequence
  source: /note=“Description of Artificial Sequence Synthetic oligonucleotide”
ggagcagcaggaccagcttc

SEQ ID NO: 95 - DNA, 20 bp, Artificial Sequence
  source: /note=“Description of Artificial Sequence Synthetic oligonucleotide”
cattacaaatggccagaagc

SEQ ID NO: 96 - DNA, 20 bp, Artificial Sequence
  source: /note=“Description of Artificial Sequence Synthetic oligonucleotide”
ggccatttgtaatgctgact

SEQ ID NO: 97 - DNA, 20 bp, Artificial Sequence
  source: /note=“Description of Artificial Sequence Synthetic oligonucleotide”
gccatttgtaatgctgactt

SEQ ID NO: 98 - DNA, 20 bp, Artificial Sequence
  source: /note=“Description of Artificial Sequence Synthetic oligonucleotide”
ccatttgtaatgctgacttg

SEQ ID NO: 99 - DNA, 20 bp, Artificial Sequence
  source: /note=“Description of Artificial Sequence Synthetic oligonucleotide”
tttgtaatgctgacttgggg

SEQ ID NO: 100 - DNA, 20 bp, Artificial Sequence
  source: /note=“Description of Artificial Sequence Synthetic oligonucleotide”
ccccaagtcagcattacaaa

SEQ ID NO: 101 - DNA, 20 bp, Artificial Sequence
  source: /note=“Description of Artificial Sequence Synthetic oligonucleotide”
gcacatctccccatccttca

SEQ ID NO: 102 - DNA, 20 bp, Artificial Sequence
  source: /note=“Description of Artificial Sequence Synthetic oligonucleotide”
cccatccttcaaggatcgag

SEQ ID NO: 103 - DNA, 20 bp, Artificial Sequence
  source: /note=“Description of Artificial Sequence Synthetic oligonucleotide”
cactcgatccttgaaggatg

SEQ ID NO: 104 - DNA, 20 bp, Artificial Sequence
  source: /note=“Description of Artificial Sequence Synthetic oligonucleotide”
ccactcgatccttgaaggat

SEQ ID NO: 105 - DNA, 20 bp, Artificial Sequence
  source: /note=“Description of Artificial Sequence Synthetic oligonucleotide”
gccactcgatccttgaagga

SEQ ID NO: 106 - DNA, 20 bp, Artificial Sequence
  source: /note=“Description of Artificial Sequence Synthetic oligonucleotide”
ttcaaggatcgagtggcccc

SEQ ID NO: 107 - DNA, 20 bp, Artificial Sequence
  source: /note=“Description of Artificial Sequence Synthetic oligonucleotide”
tggggccactcgatccttga

SEQ ID NO: 108 - DNA, 20 bp, Artificial Sequence
  source: /note=“Description of Artificial Sequence Synthetic oligonucleotide”
gatcgagtggccccaggtcc

SEQ ID NO: 109 - DNA, 20 bp, Artificial Sequence
  source: /note=“Description of Artificial Sequence Synthetic oligonucleotide”
agtggccccaggtcccggcc

SEQ ID NO: 110 - DNA, 20 bp, Artificial Sequence
  source: /note=“Description of Artificial Sequence Synthetic oligonucleotide”
gtggccccaggtcccggcct

SEQ ID NO: 111 - DNA, 20 bp, Artificial Sequence
  source: /note=“Description of Artificial Sequence Synthetic oligonucleotide”
gaggcccaggccgggacctg

SEQ ID NO: 112 - DNA, 20 bp, Artificial Sequence
  source: /note=“Description of Artificial Sequence Synthetic oligonucleotide”
tgaggcccaggccgggacct

SEQ ID NO: 113 - DNA, 20 bp, Artificial Sequence
  source: /note=“Description of Artificial Sequence Synthetic oligonucleotide”
gtgaggcccaggccgggacc

SEQ ID NO: 114 - DNA, 20 bp, Artificial Sequence
  source: /note=“Description of Artificial Sequence Synthetic oligonucleotide”
tggagggtgaggcccaggcc

SEQ ID NO: 115 - DNA, 20 bp, Artificial Sequence
  source: /note=“Description of Artificial Sequence Synthetic oligonucleotide”
ctggagggtgaggcccaggc

SEQ ID NO: 116 - DNA, 20 bp, Artificial Sequence
  source: /note=“Description of Artificial Sequence Synthetic oligonucleotide”
gcgactggagggtgaggccc

SEQ ID NO: 117 - DNA, 20 bp, Artificial Sequence
  source: /note=“Description of Artificial Sequence Synthetic oligonucleotide”
cggtcagcgactggagggtg

SEQ ID NO: 118 - DNA, 20 bp, Artificial Sequence
  source: /note=“Description of Artificial Sequence Synthetic oligonucleotide”
gttcacggtcagcgactgga

SEQ ID NO: 119 - DNA, 20 bp, Artificial Sequence
  source: /note=“Description of Artificial Sequence Synthetic oligonucleotide”
cgttcacggtcagcgactgg

SEQ ID NO: 120 - DNA, 20 bp, Artificial Sequence
  source: /note=“Description of Artificial Sequence Synthetic oligonucleotide”
tatcgttcacggtcagcgac

SEQ ID NO: 121 - DNA, 20 bp, Artificial Sequence
  source: /note=“Description of Artificial Sequence Synthetic oligonucleotide”
tcgctgaccgtgaacgatac

SEQ ID NO: 122 - DNA, 20 bp, Artificial Sequence
  source: /note=“Description of Artificial Sequence Synthetic oligonucleotide”
cgctgaccgtgaacgataca

SEQ ID NO: 123 - DNA, 20 bp, Artificial Sequence
  source: /note=“Description of Artificial Sequence Synthetic oligonucleotide”
gctgaccgtgaacgatacag

SEQ ID NO: 124 - DNA, 20 bp, Artificial Sequence
  source: /note=“Description of Artificial Sequence Synthetic oligonucleotide”
gtactcccctgtatcgttca

SEQ ID NO: 125 - DNA, 20 bp, Artificial Sequence
  source: /note=“Description of Artificial Sequence Synthetic oligonucleotide”
atctatcacacctaccctga

SEQ ID NO: 126 - DNA, 20 bp, Artificial Sequence
  source: /note=“Description of Artificial Sequence Synthetic oligonucleotide”
tctatcacacctaccctgat

SEQ ID NO: 127 - DNA, 20 bp, Artificial Sequence
  source: /note=“Description of Artificial Sequence Synthetic oligonucleotide”
taccctgatgggacgtacac

SEQ ID NO: 128 - DNA, 20 bp, Artificial Sequence
  source: /note=“Description of Artificial Sequence Synthetic oligonucleotide”
accctgatgggacgtacact

SEQ ID NO: 129 - DNA, 20 bp, Artificial Sequence
  source: /note=“Description of Artificial Sequence Synthetic oligonucleotide”
agtgtacgtcccatcagggt

SEQ ID NO: 130 - DNA, 20 bp, Artificial Sequence
  source: /note=“Description of Artificial Sequence Synthetic oligonucleotide”
tcccagtgtacgtcccatca

SEQ ID NO: 131 - DNA, 20 bp, Artificial Sequence
  source: /note=“Description of Artificial Sequence Synthetic oligonucleotide”
ctcccagtgtacgtcccatc

SEQ ID NO: 132 - DNA, 20 bp, Artificial Sequence
  source: /note=“Description of Artificial Sequence Synthetic oligonucleotide”
gtacactgggagaatcttcc

SEQ ID NO: 133 - DNA, 20 bp, Artificial Sequence
  source: /note=“Description of Artificial Sequence Synthetic oligonucleotide”
cactgggagaatcttcctgg

SEQ ID NO: 134 - DNA, 20 bp, Artificial Sequence
  source: /note=“Description of Artificial Sequence Synthetic oligonucleotide”
ctgagctttctaggacctcc

SEQ ID NO: 135 - DNA, 20 bp, Artificial Sequence
  source: /note=“Description of Artificial Sequence Synthetic oligonucleotide”
aggttccagattccattgct

SEQ ID NO: 136 - DNA, 20 bp, Artificial Sequence
  source: /note=“Description of Artificial Sequence Synthetic oligonucleotide”
aagcaatggaatctggaacc

SEQ ID NO: 137 - DNA, 20 bp, Artificial Sequence
  source: /note=“Description of Artificial Sequence Synthetic oligonucleotide”
gattccattgcttggagcca

SEQ ID NO: 138 - DNA, 20 bp, Artificial Sequence
  source: /note=“Description of Artificial Sequence Synthetic oligonucleotide”
tggctccaagcaatggaatc

SEQ ID NO: 139 - DNA, 20 bp, Artificial Sequence
  source: /note=“Description of Artificial Sequence Synthetic oligonucleotide”
gcggccatggctccaagcaa

SEQ ID NO: 140 - DNA, 20 bp, Artificial Sequence
  source: /note=“Description of Artificial Sequence Synthetic oligonucleotide”
tggagccatggccgcgacgc

SEQ ID NO: 141 - DNA, 20 bp, Artificial Sequence
  source: /note=“Description of Artificial Sequence Synthetic oligonucleotide”
agccatggccgcgacgctgg

SEQ ID NO: 142 - DNA, 20 bp, Artificial Sequence
  source: /note=“Description of Artificial Sequence Synthetic oligonucleotide”
gaccaccagcgtcgcggcca

SEQ ID NO: 143 - DNA, 20 bp, Artificial Sequence
  source: /note=“Description of Artificial Sequence Synthetic oligonucleotide”
gcagatgaccaccagcgtcg

SEQ ID NO: 144 - DNA, 20 bp, Artificial Sequence
  source: /note=“Description of Artificial Sequence Synthetic oligonucleotide”
catctgcacagcagtcatcg

SEQ ID NO: 145 - DNA, 20 bp, Artificial Sequence
  source: /note=“Description of Artificial Sequence Synthetic oligonucleotide”
ctgcacagcagtcatcgtgg

SEQ ID NO: 146 - DNA, 20 bp, Artificial Sequence
  source: /note=“Description of Artificial Sequence Synthetic oligonucleotide”
agccctcagaatccattctg

SEQ ID NO: 147 - DNA, 20 bp, Artificial Sequence
  source: /note=“Description of Artificial Sequence Synthetic oligonucleotide”
ctcagaatccattctgtgga

SEQ ID NO: 148 - DNA, 20 bp, Artificial Sequence
  source: /note=“Description of Artificial Sequence Synthetic oligonucleotide”
ttccacagaatggattctga

SEQ ID NO: 149 - DNA, 20 bp, Artificial Sequence
  source: /note=“Description of Artificial Sequence Synthetic oligonucleotide”
cttccacagaatggattctg

SEQ ID NO: 150 - DNA, 20 bp, Artificial Sequence
  source: /note=“Description of Artificial Sequence Synthetic oligonucleotide”
attctgtggaaggtgacctc

SEQ ID NO: 151 - DNA, 20 bp, Artificial Sequence
  source: /note=“Description of Artificial Sequence Synthetic oligonucleotide”
tgaggtcaccttccacagaa

SEQ ID NO: 152 - DNA, 20 bp, Artificial Sequence
  source: /note=“Description of Artificial Sequence Synthetic oligonucleotide”
gacctcaggagaaaatcagc

SEQ ID NO: 153 - DNA, 20 bp, Artificial Sequence
  source: /note=“Description of Artificial Sequence Synthetic oligonucleotide”
caggagaaaatcagctggac

SEQ ID NO: 154 - DNA, 20 bp, Artificial Sequence
  source: /note=“Description of Artificial Sequence Synthetic oligonucleotide”
gtccagctgattttctcctg

SEQ ID NO: 155 - DNA, 20 bp, Artificial Sequence
  source: /note=“Description of Artificial Sequence Synthetic oligonucleotide”
gagaaaatcagctggacagg

SEQ ID NO: 156 - DNA, 20 bp, Artificial Sequence
  source: /note=“Description of Artificial Sequence Synthetic oligonucleotide”
aatcagctggacaggaggaa

SEQ ID NO: 157 - DNA, 20 bp, Artificial Sequence
  source: /note=“Description of Artificial Sequence Synthetic oligonucleotide”
cccagtgctccctcaccccc

SEQ ID NO: 158 - DNA, 20 bp, Artificial Sequence
  source: /note=“Description of Artificial Sequence Synthetic oligonucleotide”
ctgggggtgagggagcactg

SEQ ID NO: 159 - DNA, 20 bp, Artificial Sequence
  source: /note=“Description of Artificial Sequence Synthetic oligonucleotide”
cctgggggtgagggagcact

SEQ ID NO: 160 - DNA, 20 bp, Artificial Sequence
  source: /note=“Description of Artificial Sequence Synthetic oligonucleotide”
tcctgggggtgagggagcac

SEQ ID NO: 161 - DNA, 20 bp, Artificial Sequence
  source: /note=“Description of Artificial Sequence Synthetic oligonucleotide”
acacagcttcctgggggtga

SEQ ID NO: 162 - DNA, 20 bp, Artificial Sequence
  source: /note=“Description of Artificial Sequence Synthetic oligonucleotide”
gacacagcttcctgggggtg

SEQ ID NO: 163 - DNA, 20 bp, Artificial Sequence
  source: /note=“Description of Artificial Sequence Synthetic oligonucleotide”
acccccaggaagctgtgtcc

SEQ ID NO: 164 - DNA, 20 bp, Artificial Sequence
  source: /note=“Description of Artificial Sequence Synthetic oligonucleotide”
gcctggacacagcttcctgg

SEQ ID NO: 165 - DNA, 20 bp, Artificial Sequence
  source: /note=“Description of Artificial Sequence Synthetic oligonucleotide”
tgcctggacacagcttcctg

SEQ ID NO: 166 - DNA, 20 bp, Artificial Sequence
  source: /note=“Description of Artificial Sequence Synthetic oligonucleotide”
ctgcctggacacagcttcct

SEQ ID NO: 167 - DNA, 20 bp, Artificial Sequence
  source: /note=“Description of Artificial Sequence Synthetic oligonucleotide”
tctgcctggacacagcttcc

SEQ ID NO: 168 - DNA, 20 bp, Artificial Sequence
  source: /note=“Description of Artificial Sequence Synthetic oligonucleotide”
caggcagaagctgcacctgc

SEQ ID NO: 169 - DNA, 20 bp, Artificial Sequence
  source: /note=“Description of Artificial Sequence Synthetic oligonucleotide”
aggcagaagctgcacctgct

SEQ ID NO: 170 - DNA, 20 bp, Artificial Sequence
  source: /note=“Description of Artificial Sequence Synthetic oligonucleotide”
cagcaggtgcagcttctgcc

SEQ ID NO: 171 - DNA, 20 bp, Artificial Sequence
  source: /note=“Description of Artificial Sequence Synthetic oligonucleotide”
gctgcacctgctgggctctg

SEQ ID NO: 172 - DNA, 20 bp, Artificial Sequence
  source: /note=“Description of Artificial Sequence Synthetic oligonucleotide”
tgctctccacagagcccagc

SEQ ID NO: 173 - DNA, 20 bp, Artificial Sequence
  source: /note=“Description of Artificial Sequence Synthetic oligonucleotide”
ctgggctctgtggagagcag

SEQ ID NO: 174 - DNA, 20 bp, Artificial Sequence
  source: /note=“Description of Artificial Sequence Synthetic oligonucleotide”
tgggctctgtggagagcagc

SEQ ID NO: 175 - DNA, 20 bp, Artificial Sequence
  source: /note=“Description of Artificial Sequence Synthetic oligonucleotide”
gggctctgtggagagcagcg

SEQ ID NO: 176 - DNA, 20 bp, Artificial Sequence
  source: /note=“Description of Artificial Sequence Synthetic oligonucleotide”
ctgtggagagcagcggggag

SEQ ID NO: 177 - DNA, 20 bp, Artificial Sequence
  source: /note=“Description of Artificial Sequence Synthetic oligonucleotide”
attgaagtagtcatgcagct

SEQ ID NO: 178 - DNA, 20 bp, Artificial Sequence
  source: /note=“Description of Artificial Sequence Synthetic oligonucleotide”
tgtcctgagttacagaagcc

SEQ ID NO: 179 - DNA, 20 bp, Artificial Sequence
  source: /note=“Description of Artificial Sequence Synthetic oligonucleotide”
gtcctgagttacagaagcct

SEQ ID NO: 180 - DNA, 20 bp, Artificial Sequence
  source: /note=“Description of Artificial Sequence Synthetic oligonucleotide”
tacccaggcttctgtaactc

SEQ ID NO: 181 - DNA, 20 bp, Artificial Sequence
  source: /note=“Description of Artificial Sequence Synthetic oligonucleotide”
tgaagaagctgcagttaccc

SEQ ID NO: 182 - DNA, 20 bp, Artificial Sequence
  source: /note=“Description of Artificial Sequence Synthetic oligonucleotide”
tgcagcttcttcacagagac

SEQ ID NO: 183 - DNA, 20 bp, Artificial Sequence
  source: /note=“Description of Artificial Sequence Synthetic oligonucleotide”
gttgtttctattgtgcctgt

SEQ ID NO: 184 - DNA, 20 bp, Artificial Sequence
  source: /note=“Description of Artificial Sequence Synthetic oligonucleotide”
cgttgtttctattgtgcctg

SEQ ID NO: 185 - DNA, 20 bp, Artificial Sequence
  source: /note=“Description of Artificial Sequence Synthetic oligonucleotide”
ccgttgtttctattgtgcct

SEQ ID NO: 186 - DNA, 20 bp, Artificial Sequence
  source: /note=“Description of Artificial Sequence Synthetic oligonucleotide”
ccacggcacaagtgacccag

SEQ ID NO: 187 - DNA, 20 bp, Artificial Sequence
  source: /note=“Description of Artificial Sequence Synthetic oligonucleotide”
agttgacctgggtcacttgt

SEQ ID NO: 188 - DNA, 20 bp, Artificial Sequence
  source: /note=“Description of Artificial Sequence Synthetic oligonucleotide”
aagtcagcattacaaatggc

SEQ ID NO: 189 - DNA, 20 bp, Artificial Sequence
  source: /note=“Description of Artificial Sequence Synthetic oligonucleotide”
catccttcaaggatcgagtg

SEQ ID NO: 190 - DNA, 20 bp, Artificial Sequence
  source: /note=“Description of Artificial Sequence Synthetic oligonucleotide”
atccttcaaggatcgagtgg

SEQ ID NO: 191 - DNA, 20 bp, Artificial Sequence
  source: /note=“Description of Artificial Sequence Synthetic oligonucleotide”
aggatcgagtggccccaggt

SEQ ID NO: 192 - DNA, 20 bp, Artificial Sequence
  source: /note=“Description of Artificial Sequence Synthetic oligonucleotide”
aggtcccggcctgggcctca

SEQ ID NO: 193 - DNA, 20 bp, Artificial Sequence
  source: /note=“Description of Artificial Sequence Synthetic oligonucleotide”
ggcctgggcctcaccctcca

SEQ ID NO: 194 - DNA, 20 bp, Artificial Sequence
  source: /note=“Description of Artificial Sequence Synthetic oligonucleotide”
cggtcagcgactggagggtg

SEQ ID NO: 195 - DNA, 20 bp, Artificial Sequence
  source: /note=“Description of Artificial Sequence Synthetic oligonucleotide”
gtcgctgaccgtgaacgata

SEQ ID NO: 196 - DNA, 20 bp, Artificial Sequence
  source: /note=“Description of Artificial Sequence Synthetic oligonucleotide”
tgtatcgttcacggtcagcg

SEQ ID NO: 197 - DNA, 20 bp, Artificial Sequence
  source: /note=“Description of Artificial Sequence Synthetic oligonucleotide”
ctgtatcgttcacggtcagc

SEQ ID NO: 198 - DNA, 20 bp, Artificial Sequence
  source: /note=“Description of Artificial Sequence Synthetic oligonucleotide”
atcagggtaggtgtgataga

SEQ ID NO: 199 - DNA, 20 bp, Artificial Sequence
  source: /note=“Description of Artificial Sequence Synthetic oligonucleotide”
agtgtacgtcccatcagggt

SEQ ID NO: 200 - DNA, 20 bp, Artificial Sequence
  source: /note=“Description of Artificial Sequence Synthetic oligonucleotide”
ggaagattctcccagtgtac

SEQ ID NO: 201 - DNA, 20 bp, Artificial Sequence
  source: /note=“Description of Artificial Sequence Synthetic oligonucleotide”
tggaggtcctagaaagctca

SEQ ID NO: 202 - DNA, 20 bp, Artificial Sequence
  source: /note=“Description of Artificial Sequence Synthetic oligonucleotide”
agcaatggaatctggaacct

SEQ ID NO: 203 - DNA, 20 bp, Artificial Sequence
  source: /note=“Description of Artificial Sequence Synthetic oligonucleotide”
agattccattgcttggagcc

SEQ ID NO: 204 - DNA, 20 bp, Artificial Sequence
  source: /note=“Description of Artificial Sequence Synthetic oligonucleotide”
gattccattgcttggagcca

SEQ ID NO: 205 - DNA, 20 bp, Artificial Sequence
  source: /note=“Description of Artificial Sequence Synthetic oligonucleotide”
attgcttggagccatggccg

SEQ ID NO: 206 - DNA, 20 bp, Artificial Sequence
  source: /note=“Description of Artificial Sequence Synthetic oligonucleotide”
ttgcttggagccatggccgc

SEQ ID NO: 207 - DNA, 20 bp, Artificial Sequence
  source: /note=“Description of Artificial Sequence Synthetic oligonucleotide”
cagaatggattctgagggct

SEQ ID NO: 208 - DNA, 20 bp, Artificial Sequence
  source: /note=“Description of Artificial Sequence Synthetic oligonucleotide”
acagaatggattctgagggc

SEQ ID NO: 209 - DNA, 20 bp, Artificial Sequence
  source: /note=“Description of Artificial Sequence Synthetic oligonucleotide”
ttctgtggaaggtgacctca

SEQ ID NO: 210 - DNA, 20 bp, Artificial Sequence
  source: /note=“Description of Artificial Sequence Synthetic oligonucleotide”
gctgattttctcctgaggtc

SEQ ID NO: 211 - DNA, 20 bp, Artificial Sequence
  source: /note=“Description of Artificial Sequence Synthetic oligonucleotide”
tcctgtccagctgattttct

SEQ ID NO: 212 - DNA, 20 bp, Artificial Sequence
  source: /note=“Description of Artificial Sequence Synthetic oligonucleotide”
ttcctcctgtccagctgatt

SEQ ID NO: 213 - DNA, 20 bp, Artificial Sequence
  source: /note=“Description of Artificial Sequence Synthetic oligonucleotide”
tgggggtgagggagcactgg

SEQ ID NO: 214 - DNA, 20 bp, Artificial Sequence
  source: /note=“Description of Artificial Sequence Synthetic oligonucleotide”
agtgctccctcacccccagg

SEQ ID NO: 215 - DNA, 20 bp, Artificial Sequence
  source: /note=“Description of Artificial Sequence Synthetic oligonucleotide”
tcacccccaggaagctgtgt

SEQ ID NO: 216 - DNA, 20 bp, Artificial Sequence
  source: /note=“Description of Artificial Sequence Synthetic oligonucleotide”
caggaagctgtgtccaggca

SEQ ID NO: 217 - DNA, 20 bp, Artificial Sequence
  source: /note=“Description of Artificial Sequence Synthetic oligonucleotide”
aggaagctgtgtccaggcag

SEQ ID NO: 218 - DNA, 20 bp, Artificial Sequence
  source: /note=“Description of Artificial Sequence Synthetic oligonucleotide”
ggcagaagctgcacctgctg

SEQ ID NO: 219 - DNA, 20 bp, Artificial Sequence
  source: /note=“Description of Artificial Sequence Synthetic oligonucleotide”
cagagcccagcaggtgcagc

SEQ ID NO: 220 - DNA, 20 bp, Artificial Sequence
  source: /note=“Description of Artificial Sequence Synthetic oligonucleotide”
gctgctctccacagagccca

SEQ ID NO: 221 - DNA, 20 bp, Artificial Sequence
  source: /note=“Description of Artificial Sequence Synthetic oligonucleotide”
cgctgctctccacagagccc

SEQ ID NO: 222 - DNA, 20 bp, Artificial Sequence
  source: /note=“Description of Artificial Sequence Synthetic oligonucleotide”
atgtcctgagttacagaagc

SEQ ID NO: 223 - DNA, 20 bp, Artificial Sequence
  source: /note=“Description of Artificial Sequence Synthetic oligonucleotide”
gagcacacccactgcgatgt

SEQ ID NO: 224 - DNA, 20 bp, Artificial Sequence
  source: /note=“Description of Artificial Sequence Synthetic oligonucleotide”
gatggccaggagactgaaga

SEQ ID NO: 225 - DNA, 20 bp, Artificial Sequence
  source: /note=“Description of Artificial Sequence Synthetic oligonucleotide”
ctgctcaccggagcgggatg

SEQ ID NO: 226 - DNA, 20 bp, Artificial Sequence
  source: /note=“Description of Artificial Sequence Synthetic oligonucleotide”
gtctgtggccatgcccatca

SEQ ID NO: 227 - DNA, 20 bp, Artificial Sequence
  source: /note=“Description of Artificial Sequence Synthetic oligonucleotide”
tcaccggagcgggatgcgga

SEQ ID NO: 228 - DNA, 20 bp, Artificial Sequence
  source: /note=“Description of Artificial Sequence Synthetic oligonucleotide”
gtggcaggcagcgcagaacc

SEQ ID NO: 229 - DNA, 20 bp, Artificial Sequence
  source: /note=“Description of Artificial Sequence Synthetic oligonucleotide”
agcacaccagcacattgccc

SEQ ID NO: 230 - DNA, 20 bp, Artificial Sequence
  source: /note=“Description of Artificial Sequence Synthetic oligonucleotide”
caggttgctgttgagccaca

SEQ ID NO: 231 - DNA, 20 bp, Artificial Sequence
  source: /note=“Description of Artificial Sequence Synthetic oligonucleotide”
cttcattgcctgcttcgtcc

SEQ ID NO: 232 - DNA, 20 bp, Artificial Sequence
  source: /note=“Description of Artificial Sequence Synthetic oligonucleotide”
gtacaccgaggagcccatga

SEQ ID NO: 233 - DNA, 20 bp, Artificial Sequence
  source: /note=“Description of Artificial Sequence Synthetic oligonucleotide”
gatggcaatgtagcggtcaa

SEQ ID NO: 234 - DNA, 20 bp, Artificial Sequence
  source: /note=“Description of Artificial Sequence Synthetic oligonucleotide”
ctcctcggtgtacatcacgg

SEQ ID NO: 235 - DNA, 20 bp, Artificial Sequence
  source: /note=“Description of Artificial Sequence Synthetic oligonucleotide”
cgaggagcccatgatgggca

SEQ ID NO: 236 - DNA, 20 bp, Artificial Sequence
  source: /note=“Description of Artificial Sequence Synthetic oligonucleotide”
gggctcctcggtgtacatca

SEQ ID NO: 237 - DNA, 20 bp, Artificial Sequence
  source: /note=“Description of Artificial Sequence Synthetic oligonucleotide”
ctttgtggtgtcactggcgg

SEQ ID NO: 238 - DNA, 20 bp, Artificial Sequence
  source: /note=“Description of Artificial Sequence Synthetic oligonucleotide”
ccgctccggtgagcagggcc

SEQ ID NO: 239 - DNA, 20 bp, Artificial Sequence
  source: /note=“Description of Artificial Sequence Synthetic oligonucleotide”
gggttctgcgctgcctgcca

SEQ ID NO: 240 - DNA, 20 bp, Artificial Sequence
  source: /note=“Description of Artificial Sequence Synthetic oligonucleotide”
ggacgaagcaggcaatgaag

SEQ ID NO: 241 - DNA, 20 bp, Artificial Sequence
  source: /note=“Description of Artificial Sequence Synthetic oligonucleotide”
gtgctgatggtgatggcaaa

SEQ ID NO: 242 - DNA, 20 bp, Artificial Sequence
  source: /note=“Description of Artificial Sequence Synthetic oligonucleotide”
agcgcagaacccggtgctga

SEQ ID NO: 243 - DNA, 20 bp, Artificial Sequence
  source: /note=“Description of Artificial Sequence Synthetic oligonucleotide”
gagctccatcttcagtctcc

SEQ ID NO: 244 - DNA, 20 bp, Artificial Sequence
  source: /note=“Description of Artificial Sequence Synthetic oligonucleotide”
tgctgatggtgatggcaaag

SEQ ID NO: 245 - DNA, 20 bp, Artificial Sequence
  source: /note=“Description of Artificial Sequence Synthetic oligonucleotide”
ggcggcggccgacatcgcag

SEQ ID NO: 246 - DNA, 20 bp, Artificial Sequence
  source: /note=“Description of Artificial Sequence Synthetic oligonucleotide”
aatgaagaggcagccgtggc

SEQ ID NO: 247 - DNA, 20 bp, Artificial Sequence
  source: /note=“Description of Artificial Sequence Synthetic oligonucleotide”
gggcaatgtgctggtgtgct

SEQ ID NO: 248 - DNA, 20 bp, Artificial Sequence
  source: /note=“Description of Artificial Sequence Synthetic oligonucleotide”
catgcccatcatgggctcct

SEQ ID NO: 249 - DNA, 20 bp, Artificial Sequence
  source: /note=“Description of Artificial Sequence Synthetic oligonucleotide”
aatgtagcggtcaatggcga

SEQ ID NO: 250 - DNA, 20 bp, Artificial Sequence
  source: /note=“Description of Artificial Sequence Synthetic oligonucleotide”
agtagttggtgacgttctgc

SEQ ID NO: 251 - DNA, 20 bp, Artificial Sequence
  source: /note=“Description of Artificial Sequence Synthetic oligonucleotide”
agcggtcaatggcgatggcc

SEQ ID NO: 252 - DNA, 20 bp, Artificial Sequence
  source: /note=“Description of Artificial Sequence Synthetic oligonucleotide”
cgcatcccgctccggtgagc

SEQ ID NO: 253 - DNA, 20 bp, Artificial Sequence
  source: /note=“Description of Artificial Sequence Synthetic oligonucleotide”
gcatcccgctccggtgagca

SEQ ID NO: 254 - DNA, 20 bp, Artificial Sequence
  source: /note=“Description of Artificial Sequence Synthetic oligonucleotide”
tgggcaatgtgctggtgtgc

SEQ ID NO: 255 - DNA, 20 bp, Artificial Sequence
  source: /note=“Description of Artificial Sequence Synthetic oligonucleotide”
caactactttgtggtgtcac

SEQ ID NO: 256 - DNA, 20 bp, Artificial Sequence
  source: /note=“Description of Artificial Sequence Synthetic oligonucleotide”
cgctccggtgagcagggccg

SEQ ID NO: 257 - DNA, 20 bp, Artificial Sequence
  source: /note=“Description of Artificial Sequence Synthetic oligonucleotide”
gatggtgatggcaaagggga

SEQ ID NO: 258 - DNA, 20 bp, Artificial Sequence
  source: /note=“Description of Artificial Sequence Synthetic oligonucleotide”
ggtgtacatcacggtggagc

SEQ ID NO: 259 - DNA, 20 bp, Artificial Sequence
  source: /note=“Description of Artificial Sequence Synthetic oligonucleotide”
gaacgtcaccaactactttg

SEQ ID NO: 260 - DNA, 20 bp, Artificial Sequence
  source: /note=“Description of Artificial Sequence Synthetic oligonucleotide”
cagtgacaccacaaagtagt

SEQ ID NO: 261 - DNA, 20 bp, Artificial Sequence
  source: /note=“Description of Artificial Sequence Synthetic oligonucleotide”
ggccatcctgggcaatgtgc

SEQ ID NO: 262 - DNA, 20 bp, Artificial Sequence
  source: /note=“Description of Artificial Sequence Synthetic oligonucleotide”
cccggccctgctcaccggag

SEQ ID NO: 263 - DNA, 20 bp, Artificial Sequence
  source: /note=“Description of Artificial Sequence Synthetic oligonucleotide”
caccagcacattgcccagga

SEQ ID NO: 264 - DNA, 20 bp, Artificial Sequence
  source: /note=“Description of Artificial Sequence Synthetic oligonucleotide”
tttgccatcaccatcagcac

SEQ ID NO: 265 - DNA, 20 bp, Artificial Sequence
  source: /note=“Description of Artificial Sequence Synthetic oligonucleotide”
ctccaccgtgatgtacaccg

SEQ ID NO: 266 - DNA, 20 bp, Artificial Sequence
  source: /note=“Description of Artificial Sequence Synthetic oligonucleotide”
ggagctggccattgctgtgc

SEQ ID NO: 267 - DNA, 20 bp, Artificial Sequence
  source: /note=“Description of Artificial Sequence Synthetic oligonucleotide”
caggatggccagcacagcaa

SEQ ID NO: 268 - DNA, 20 bp, Artificial Sequence
  source: /note=“Description of Artificial Sequence Synthetic oligonucleotide”
gaacccggtgctgatggtga

SEQ ID NO: 269 - DNA, 20 bp, Artificial Sequence
  source: /note=“Description of Artificial Sequence Synthetic oligonucleotide”
tggagctctgcgtgaggacc

SEQ ID NO: 270 - DNA, 20 bp, Artificial Sequence
  source: /note=“Description of Artificial Sequence Synthetic oligonucleotide”
cccgctccggtgagcagggc

SEQ ID NO: 271 - DNA, 20 bp, Artificial Sequence
  source: /note=“Description of Artificial Sequence Synthetic oligonucleotide”
aggcaatgaagaggcagccg

SEQ ID NO: 272 - DNA, 20 bp, Artificial Sequence
  source: /note=“Description of Artificial Sequence Synthetic oligonucleotide”
ccggccctgctcaccggagc

SEQ ID NO: 273 - DNA, 20 bp, Artificial Sequence
  source: /note=“Description of Artificial Sequence Synthetic oligonucleotide”
gcggcggccgacatcgcagt

SEQ ID NO: 274 - DNA, 20 bp, Artificial Sequence
  source: /note=“Description of Artificial Sequence Synthetic oligonucleotide”
ggtgctgatggtgatggcaa

SEQ ID NO: 275 - DNA, 20 bp, Artificial Sequence
  source: /note=“Description of Artificial Sequence Synthetic oligonucleotide”
ctactttgtggtgtcactgg

SEQ ID NO: 276 - DNA, 20 bp, Artificial Sequence
  source: /note=“Description of Artificial Sequence Synthetic oligonucleotide”
tacaccgaggagcccatgat

SEQ ID NO: 277 - DNA, 20 bp, Artificial Sequence
  source: /note=“Description of Artificial Sequence Synthetic oligonucleotide”
tctgtggccatgcccatcat

SEQ ID NO: 278 - DNA, 20 bp, Artificial Sequence
  source: /note=“Description of Artificial Sequence Synthetic oligonucleotide”
attgctgtgctggccatcct

SEQ ID NO: 279 - DNA, 20 bp, Artificial Sequence
  source: /note=“Description of Artificial Sequence Synthetic oligonucleotide”
cgtgaggaccaggacgaagc

SEQ ID NO: 280 - DNA, 20 bp, Artificial Sequence
  source: /note=“Description of Artificial Sequence Synthetic oligonucleotide”
ttgccatcaccatcagcacc

SEQ ID NO: 281 - DNA, 20 bp, Artificial Sequence
  source: /note=“Description of Artificial Sequence Synthetic oligonucleotide”
ggatgcggatggcaatgtag

SEQ ID NO: 282 - DNA, 20 bp, Artificial Sequence
  source: /note=“Description of Artificial Sequence Synthetic oligonucleotide”
ttgccatccgcatcccgctc

SEQ ID NO: 283 - DNA, 20 bp, Artificial Sequence
  source: /note=“Description of Artificial Sequence Synthetic oligonucleotide”
tgaagatggagctctgcgtg

SEQ ID NO: 284 - DNA, 20 bp, Artificial Sequence
  source: /note=“Description of Artificial Sequence Synthetic oligonucleotide”
cattgctgtgctggccatcc

SEQ ID NO: 285 - DNA, 20 bp, Artificial Sequence
  source: /note=“Description of Artificial Sequence Synthetic oligonucleotide”
tgctggtgtgctgggccgtg

SEQ ID NO: 286 - DNA, 21 bp, Artificial Sequence
  source: /note=“Description of Artificial Sequence Synthetic oligonucleotide”
ggctcctcggtgtacatcacg

SEQ ID NO: 287 - DNA, 21 bp, Artificial Sequence
  source: /note=“Description of Artificial Sequence Synthetic oligonucleotide”
gagctctgcgtgaggaccagg

SEQ ID NO: 288 - DNA, 21 bp, Artificial Sequence
  source: /note=“Description of Artificial Sequence Synthetic oligonucleotide”
gatggagctctgcgtgaggac

SEQ ID NO: 289 - DNA, 21 bp, Artificial Sequence
  source: /note=“Description of Artificial Sequence Synthetic oligonucleotide”
ccagcacaccagcacattgcc

SEQ ID NO: 290 - DNA, 21 bp, Artificial Sequence
  source: /note=“Description of Artificial Sequence Synthetic oligonucleotide”
aggaccaggacgaagcaggca

SEQ ID NO: 291 - DNA, 21 bp, Artificial Sequence
  source: /note=“Description of Artificial Sequence Synthetic oligonucleotide”
tgccatccgcatcccgctccg

SEQ ID NO: 292 - DNA, 21 bp, Artificial Sequence
  source: /note=“Description of Artificial Sequence Synthetic oligonucleotide”
gtgtggctcaacagcaacctg

SEQ ID NO: 293 - DNA, 21 bp, Artificial Sequence
  source: /note=“Description of Artificial Sequence Synthetic oligonucleotide”
agctccaccgtgatgtacacc

SEQ ID NO: 294 - DNA, 21 bp, Artificial Sequence
  source: /note=“Description of Artificial Sequence Synthetic oligonucleotide”
gtagcggtcaatggcgatggc

SEQ ID NO: 295 - DNA, 21 bp, Artificial Sequence
  source: /note=“Description of Artificial Sequence Synthetic oligonucleotide”
cggtgctgatggtgatggcaa

SEQ ID NO: 296 - DNA, 21 bp, Artificial Sequence
  source: /note=“Description of Artificial Sequence Synthetic oligonucleotide”
ccctgctcaccggagcgggat

SEQ ID NO: 297 - DNA, 21 bp, Artificial Sequence
  source: /note=“Description of Artificial Sequence Synthetic oligonucleotide”
gtgacgttctgcaggttgctg

SEQ ID NO: 298 - DNA, 21 bp, Artificial Sequence
  source: /note=“Description of Artificial Sequence Synthetic oligonucleotide”
gctccaccgtgatgtacaccg

SEQ ID NO: 299 - DNA, 21 bp, Artificial Sequence
  source: /note=“Description of Artificial Sequence Synthetic oligonucleotide”
actgaagatggagctctgcgt

SEQ ID NO: 300 - DNA, 21 bp, Artificial Sequence
  source: /note=“Description of Artificial Sequence Synthetic oligonucleotide”
ccagctccaccgtgatgtaca

SEQ ID NO: 301 - DNA, 21 bp, Artificial Sequence
  source: /note=“Description of Artificial Sequence Synthetic oligonucleotide”
cctttgccatcaccatcagca

SEQ ID NO: 302 - DNA, 21 bp, Artificial Sequence
  source: /note=“Description of Artificial Sequence Synthetic oligonucleotide”
ccggtgctgatggtgatggca

SEQ ID NO: 303 - DNA, 21 bp, Artificial Sequence
  source: /note=“Description of Artificial Sequence Synthetic oligonucleotide”
cctgggcaatgtgctggtgtg

SEQ ID NO: 304 - DNA, 21 bp, Artificial Sequence
  source: /note=“Description of Artificial Sequence Synthetic oligonucleotide”
aggcagccgtggcaggcagcg

SEQ ID NO: 305 - DNA, 21 bp, Artificial Sequence
  source: /note=“Description of Artificial Sequence Synthetic oligonucleotide”
gcgatggccaggagactgaag

SEQ ID NO: 306 - DNA, 21 bp, Artificial Sequence
  source: /note=“Description of Artificial Sequence Synthetic oligonucleotide”
cgatggccaggagactgaaga

SEQ ID NO: 307 - DNA, 21 bp, Artificial Sequence
  source: /note=“Description of Artificial Sequence Synthetic oligonucleotide”
tcccgctccggtgagcagggc

SEQ ID NO: 308 - DNA, 21 bp, Artificial Sequence
  source: /note=“Description of Artificial Sequence Synthetic oligonucleotide”
tgcttcgtcctggtcctcacg

SEQ ID NO: 309 - DNA, 21 bp, Artificial Sequence
  source: /note=“Description of Artificial Sequence Synthetic oligonucleotide”
accaggacgaagcaggcaatg

SEQ ID NO: 310 - DNA, 21 bp, Artificial Sequence
  source: /note=“Description of Artificial Sequence Synthetic oligonucleotide”
atgtacaccgaggagcccatg

SEQ ID NO: 311 - DNA, 21 bp, Artificial Sequence
  source: /note=“Description of Artificial Sequence Synthetic oligonucleotide”
tcgtctgtggccatgcccatc

SEQ ID NO: 312 - DNA, 21 bp, Artificial Sequence
  source: /note=“Description of Artificial Sequence Synthetic oligonucleotide”
tcaatggcgatggccaggaga

SEQ ID NO: 313 - DNA, 21 bp, Artificial Sequence
  source: /note=“Description of Artificial Sequence Synthetic oligonucleotide”
ggtgctgatggtgatggcaaa

SEQ ID NO: 314 - DNA, 21 bp, Artificial Sequence
  source: /note=“Description of Artificial Sequence Synthetic oligonucleotide”
tagcggtcaatggcgatggcc

SEQ ID NO: 315 - DNA, 21 bp, Artificial Sequence
  source: /note=“Description of Artificial Sequence Synthetic oligonucleotide”
tccgcatcccgctccggtgag

SEQ ID NO: 316 - DNA, 21 bp, Artificial Sequence
  source: /note=“Description of Artificial Sequence Synthetic oligonucleotide”
ctggcggcggccgacatcgca

SEQ ID NO: 317 - DNA, 21 bp, Artificial Sequence
  source: /note=“Description of Artificial Sequence Synthetic oligonucleotide”
gccattgctgtgctggccatc

SEQ ID NO: 318 - DNA, 21 bp, Artificial Sequence
  source: /note=“Description of Artificial Sequence Synthetic oligonucleotide”
atcccgctccggtgagcaggg

SEQ ID NO: 319 - DNA, 21 bp, Artificial Sequence
  source: /note=“Description of Artificial Sequence Synthetic oligonucleotide”
agactgaagatggagctctgc

SEQ ID NO: 320 - DNA, 21 bp, Artificial Sequence
  source: /note=“Description of Artificial Sequence Synthetic oligonucleotide”
ccccggccctgctcaccggag

SEQ ID NO: 321 - DNA, 21 bp, Artificial Sequence
  source: /note=“Description of Artificial Sequence Synthetic oligonucleotide”
atggtgatggcaaaggggatg

SEQ ID NO: 322 - DNA, 21 bp, Artificial Sequence
  source: /note=“Description of Artificial Sequence Synthetic oligonucleotide”
gctcctcggtgtacatcacgg

SEQ ID NO: 323 - DNA, 20 bp, Artificial Sequence
  source: /note=“Description of Artificial Sequence Synthetic oligonucleotide”
tgtcgatggcaatagccaag

SEQ ID NO: 324 - DNA, 20 bp, Artificial Sequence
  source: /note=“Description of Artificial Sequence Synthetic oligonucleotide”
agaagttggtgacgttctgc

SEQ ID NO: 325 - DNA, 20 bp, Artificial Sequence
  source: /note=“Description of Artificial Sequence Synthetic oligonucleotide”
ttcgccatcaccatcagcac

SEQ ID NO: 326 - DNA, 20 bp, Artificial Sequence
  source: /note=“Description of Artificial Sequence Synthetic oligonucleotide”
gaagaagaggcagccatggc

SEQ ID NO: 327 - DNA, 20 bp, Artificial Sequence
  source: /note=“Description of Artificial Sequence Synthetic oligonucleotide”
cacaagcacgttacccagga

SEQ ID NO: 328 - DNA, 20 bp, Artificial Sequence
  source: /note=“Description of Artificial Sequence Synthetic oligonucleotide”
caacttcttcgtggtatctc

SEQ ID NO: 329 - DNA, 20 bp, Artificial Sequence
  source: /note=“Description of Artificial Sequence Synthetic oligonucleotide”
caggatggccagcacagcaa

SEQ ID NO: 330 - DNA, 20 bp, Artificial Sequence
  source: /note=“Description of Artificial Sequence Synthetic oligonucleotide”
aattccactccggtgagcca

SEQ ID NO: 331 - DNA, 20 bp, Artificial Sequence
  source: /note=“Description of Artificial Sequence Synthetic oligonucleotide”
agcgcagaagccagtgctga

SEQ ID NO: 332 - DNA, 20 bp, Artificial Sequence
  source: /note=“Description of Artificial Sequence Synthetic oligonucleotide”
gtgctgatggtgatggcgaa

SEQ ID NO: 333 - DNA, 20 bp, Artificial Sequence
  source: /note=“Description of Artificial Sequence Synthetic oligonucleotide”
ggagctggccattgctgtgc

SEQ ID NO: 334 - DNA, 20 bp, Artificial Sequence
  source: /note=“Description of Artificial Sequence Synthetic oligonucleotide”
aatagccaagaggctgaaga

SEQ ID NO: 335 - DNA, 20 bp, Artificial Sequence
  source: /note=“Description of Artificial Sequence Synthetic oligonucleotide”
ctcctcggtgtacatcatgg

SEQ ID NO: 336 - DNA, 20 bp, Artificial Sequence
  source: /note=“Description of Artificial Sequence Synthetic oligonucleotide”
ggacaaagcaggcgaagaag

SEQ ID NO: 337 - DNA, 20 bp, Artificial Sequence
  source: /note=“Description of Artificial Sequence Synthetic oligonucleotide”
tctggcggcggctgacatcg

SEQ ID NO: 338 - DNA, 20 bp, Artificial Sequence
  source: /note=“Description of Artificial Sequence Synthetic oligonucleotide”
tgggtaacgtgcttgtgtgc

SEQ ID NO: 339 - DNA, 20 bp, Artificial Sequence
  source: /note=“Description of Artificial Sequence Synthetic oligonucleotide”
gatgtacaccgaggagccca

SEQ ID NO: 340 - DNA, 20 bp, Artificial Sequence
  source: /note=“Description of Artificial Sequence Synthetic oligonucleotide”
taacccctggctcaccggag

SEQ ID NO: 341 - DNA, 20 bp, Artificial Sequence
  source: /note=“Description of Artificial Sequence Synthetic oligonucleotide”
tcaccggagtggaattcgga

SEQ ID NO: 342 - DNA, 20 bp, Artificial Sequence
  source: /note=“Description of Artificial Sequence Synthetic oligonucleotide”
gcggcggctgacatcgcggt

SEQ ID NO: 343 - DNA, 20 bp, Artificial Sequence
  source: /note=“Description of Artificial Sequence Synthetic oligonucleotide”
gatggtgatggcgaatggga

SEQ ID NO: 344 - DNA, 20 bp, Artificial Sequence
  source: /note=“Description of Artificial Sequence Synthetic oligonucleotide”
ggcttctgcgctgcctgcca

SEQ ID NO: 345 - DNA, 20 bp, Artificial Sequence
  source: /note=“Description of Artificial Sequence Synthetic oligonucleotide”
attccactccggtgagccag

SEQ ID NO: 346 - DNA, 20 bp, Artificial Sequence
  source: /note=“Description of Artificial Sequence Synthetic oligonucleotide”
ggtgtacatcatggtggagc

SEQ ID NO: 347 - DNA, 20 bp, Artificial Sequence
  source: /note=“Description of Artificial Sequence Synthetic oligonucleotide”
attgctgtgctggccatcct

SEQ ID NO: 348 - DNA, 20 bp, Artificial Sequence
  source: /note=“Description of Artificial Sequence Synthetic oligonucleotide”
ctccaccatgatgtacaccg

SEQ ID NO: 349 - DNA, 20 bp, Artificial Sequence
  source: /note=“Description of Artificial Sequence Synthetic oligonucleotide”
ggcggcggctgacatcgcgg

SEQ ID NO: 350 - DNA, 20 bp, Artificial Sequence
  source: /note=“Description of Artificial Sequence Synthetic oligonucleotide”
tacaccgaggagcccatggc

SEQ ID NO: 351 - DNA, 20 bp, Artificial Sequence
  source: /note=“Description of Artificial Sequence Synthetic oligonucleotide”
gggtaacgtgcttgtgtgct

SEQ ID NO: 352 - DNA, 20 bp, Artificial Sequence
  source: /note=“Description of Artificial Sequence Synthetic oligonucleotide”
caggttgctgttgatccaca

SEQ ID NO: 353 - DNA, 20 bp, Artificial Sequence
  source: /note=“Description of Artificial Sequence Synthetic oligonucleotide”
tgaagatggaactctgcgtg

SEQ ID NO: 354 - DNA, 20 bp, Artificial Sequence
  source: /note=“Description of Artificial Sequence Synthetic oligonucleotide”
gatggcgatgtatctgtcga

SEQ ID NO: 355 - DNA, 20 bp, Artificial Sequence
  source: /note=“Description of Artificial Sequence Synthetic oligonucleotide”
cttcttcgcctgctttgtcc

SEQ ID NO: 356 - DNA, 20 bp, Artificial Sequence
  source: /note=“Description of Artificial Sequence Synthetic oligonucleotide”
aggcgaagaagaggcagcca

SEQ ID NO: 357 - DNA, 20 bp, Artificial Sequence
  source: /note=“Description of Artificial Sequence Synthetic oligonucleotide”
tgcttgtgtgctgggccgtg

SEQ ID NO: 358 - DNA, 20 bp, Artificial Sequence
  source: /note=“Description of Artificial Sequence Synthetic oligonucleotide”
gaagccagtgctgatggtga

SEQ ID NO: 359 - DNA, 20 bp, Artificial Sequence
  source: /note=“Description of Artificial Sequence Synthetic oligonucleotide”
cgtgaggaccaggacaaagc

SEQ ID NO: 360 - DNA, 20 bp, Artificial Sequence
  source: /note=“Description of Artificial Sequence Synthetic oligonucleotide”
tggaactctgcgtgaggacc

SEQ ID NO: 361 - DNA, 20 bp, Artificial Sequence
  source: /note=“Description of Artificial Sequence Synthetic oligonucleotide”
cattgctgtgctggccatcc

SEQ ID NO: 362 - DNA, 20 bp, Artificial Sequence
  source: /note=“Description of Artificial Sequence Synthetic oligonucleotide”
ttctcccgccatgggctcct

SEQ ID NO: 363 - DNA, 20 bp, Artificial Sequence
  source: /note=“Description of Artificial Sequence Synthetic oligonucleotide”
tggctcaccggagtggaatt

SEQ ID NO: 364 - DNA, 20 bp, Artificial Sequence
  source: /note=“Description of Artificial Sequence Synthetic oligonucleotide”
tgctgatggtgatggcgaat

SEQ ID NO: 365 - DNA, 20 bp, Artificial Sequence
  source: /note=“Description of Artificial Sequence Synthetic oligonucleotide”
cttcgtggtatctctggcgg

SEQ ID NO: 366 - DNA, 20 bp, Artificial Sequence
  source: /note=“Description of Artificial Sequence Synthetic oligonucleotide”
agcacacaagcacgttaccc

SEQ ID NO: 367 - DNA, 20 bp, Artificial Sequence
  source: /note=“Description of Artificial Sequence Synthetic oligonucleotide”
gggctcctcggtgtacatca

SEQ ID NO: 368 - DNA, 20 bp, Artificial Sequence
  source: /note=“Description of Artificial Sequence Synthetic oligonucleotide”
gtacaccgaggagcccatgg

SEQ ID NO: 369 - DNA, 20 bp, Artificial Sequence
  source: /note=“Description of Artificial Sequence Synthetic oligonucleotide”
gaacgtcaccaacttcttcg

SEQ ID NO: 370 - DNA, 20 bp, Artificial Sequence
  source: /note=“Description of Artificial Sequence Synthetic oligonucleotide”
tcgccatccgaattccactc

SEQ ID NO: 371 - DNA, 20 bp, Artificial Sequence
  source: /note=“Description of Artificial Sequence Synthetic oligonucleotide”
gagttccatcttcagcctct

SEQ ID NO: 372 - DNA, 20 bp, Artificial Sequence
  source: /note=“Description of Artificial Sequence Synthetic oligonucleotide”
gaattccactccggtgagcc

SEQ ID NO: 373 - DNA, 20 bp, Artificial Sequence
  source: /note=“Description of Artificial Sequence Synthetic oligonucleotide”
cagagataccacgaagaagt

SEQ ID NO: 374 - DNA, 20 bp, Artificial Sequence
  source: /note=“Description of Artificial Sequence Synthetic oligonucleotide”
cttcttcgtggtatctctgg

SEQ ID NO: 375 - DNA, 21 bp, Artificial Sequence
  source: /note=“Description of Artificial Sequence Synthetic oligonucleotide”
cagtgctgatggtgatggcga

SEQ ID NO: 376 - DNA, 21 bp, Artificial Sequence
  source: /note=“Description of Artificial Sequence Synthetic oligonucleotide”
cgaattccactccggtgagcc

SEQ ID NO: 377 - DNA, 21 bp, Artificial Sequence
  source: /note=“Description of Artificial Sequence Synthetic oligonucleotide”
ccgaattccactccggtgagc

SEQ ID NO: 378 - DNA, 21 bp, Artificial Sequence
  source: /note=“Description of Artificial Sequence Synthetic oligonucleotide”
gctgaagatggaactctgcgt

SEQ ID NO: 379 - DNA, 21 bp, Artificial Sequence
  source: /note=“Description of Artificial Sequence Synthetic oligonucleotide”
cgtgcttgtgtgctgggccgt

SEQ ID NO: 380 - DNA, 21 bp, Artificial Sequence
  source: /note=“Description of Artificial Sequence Synthetic oligonucleotide”
gtgaggaccaggacaaagcag

SEQ ID NO: 381 - DNA, 21 bp, Artificial Sequence
  source: /note=“Description of Artificial Sequence Synthetic oligonucleotide”
tcgatggcaatagccaagagg

SEQ ID NO: 382 - DNA, 21 bp, Artificial Sequence
  source: /note=“Description of Artificial Sequence Synthetic oligonucleotide”
catcgacagatacatcgccat

SEQ ID NO: 383 - DNA, 21 bp, Artificial Sequence
  source: /note=“Description of Artificial Sequence Synthetic oligonucleotide”
gtacaccgaggagcccatggc

SEQ ID NO: 384 - DNA, 21 bp, Artificial Sequence
  source: /note=“Description of Artificial Sequence Synthetic oligonucleotide”
gctccaccatgatgtacaccg

SEQ ID NO: 385 - DNA, 21 bp, Artificial Sequence
  source: /note=“Description of Artificial Sequence Synthetic oligonucleotide”
aagccagtgctgatggtgatg

SEQ ID NO: 386 - DNA, 21 bp, Artificial Sequence
  source: /note=“Description of Artificial Sequence Synthetic oligonucleotide”
caccgcgatgtcagccgccgc

SEQ ID NO: 387 - DNA, 21 bp, Artificial Sequence
  source: /note=“Description of Artificial Sequence Synthetic oligonucleotide”
aggctgaagatggaactctgc

SEQ ID NO: 388 - DNA, 21 bp, Artificial Sequence
  source: /note=“Description of Artificial Sequence Synthetic oligonucleotide”
gccgccgccagagataccacg

SEQ ID NO: 389 - DNA, 21 bp, Artificial Sequence
  source: /note=“Description of Artificial Sequence Synthetic oligonucleotide”
agctccaccatgatgtacacc

SEQ ID NO: 390 - DNA, 21 bp, Artificial Sequence
  source: /note=“Description of Artificial Sequence Synthetic oligonucleotide”
aggcagccatggcaggcagcg

SEQ ID NO: 391 - DNA, 21 bp, Artificial Sequence
  source: /note=“Description of Artificial Sequence Synthetic oligonucleotide”
cctggctcaccggagtggaat

SEQ ID NO: 392 - DNA, 21 bp, Artificial Sequence
  source: /note=“Description of Artificial Sequence Synthetic oligonucleotide”
ccagctccaccatgatgtaca

SEQ ID NO: 393 - DNA, 21 bp, Artificial Sequence
  source: /note=“Description of Artificial Sequence Synthetic oligonucleotide”
accaggacaaagcaggcgaag

SEQ ID NO: 394 - DNA, 21 bp, Artificial Sequence
  source: /note=“Description of Artificial Sequence Synthetic oligonucleotide”
cctgggtaacgtgcttgtgtg

SEQ ID NO: 395 - DNA, 21 bp, Artificial Sequence
  source: /note=“Description of Artificial Sequence Synthetic oligonucleotide”
aggaccaggacaaagcaggcg

SEQ ID NO: 396 - DNA, 21 bp, Artificial Sequence
  source: /note=“Description of Artificial Sequence Synthetic oligonucleotide”
tcagccgccgccagagatacc

SEQ ID NO: 397 - DNA, 21 bp, Artificial Sequence
  source: /note=“Description of Artificial Sequence Synthetic oligonucleotide”
ggctcctcggtgtacatcatg

SEQ ID NO: 398 - DNA, 21 bp, Artificial Sequence
  source: /note=“Description of Artificial Sequence Synthetic oligonucleotide”
ctggcggcggctgacatcgcg

SEQ ID NO: 399 - DNA, 21 bp, Artificial Sequence
  source: /note=“Description of Artificial Sequence Synthetic oligonucleotide”
gatggaactctgcgtgaggac

SEQ ID NO: 400 - DNA, 21 bp, Artificial Sequence
  source: /note=“Description of Artificial Sequence Synthetic oligonucleotide”
gctcctcggtgtacatcatgg

SEQ ID NO: 401 - DNA, 21 bp, Artificial Sequence
  source: /note=“Description of Artificial Sequence Synthetic oligonucleotide”
tgtacaccgaggagcccatgg

SEQ ID NO: 402 - DNA, 21 bp, Artificial Sequence
  source: /note=“Description of Artificial Sequence Synthetic oligonucleotide”
gccattgctgtgctggccatc

SEQ ID NO: 403 - DNA, 21 bp, Artificial Sequence
  source: /note=“Description of Artificial Sequence Synthetic oligonucleotide”
caatagccaagaggctgaaga

SEQ ID NO: 404 - DNA, 21 bp, Artificial Sequence
  source: /note=“Description of Artificial Sequence Synthetic oligonucleotide”
atggtgatggcgaatgggatg

SEQ ID NO: 405 - DNA, 21 bp, Artificial Sequence
  source: /note=“Description of Artificial Sequence Synthetic oligonucleotide”
atgtacaccgaggagcccatg

SEQ ID NO: 406 - DNA, 21 bp, Artificial Sequence
  source: /note=“Description of Artificial Sequence Synthetic oligonucleotide”
gtgtggatcaacagcaacctg

SEQ ID NO: 407 - DNA, 21 bp, Artificial Sequence
  source: /note=“Description of Artificial Sequence Synthetic oligonucleotide”
tgctttgtcctggtcctcacg

SEQ ID NO: 408 - DNA, 21 bp, Artificial Sequence
  source: /note=“Description of Artificial Sequence Synthetic oligonucleotide”
gtaacccctggctcaccggag

SEQ ID NO: 409 - DNA, 21 bp, Artificial Sequence
  source: /note=“Description of Artificial Sequence Synthetic oligonucleotide”
ccagcacacaagcacgttacc

SEQ ID NO: 410 - DNA, 21 bp, Artificial Sequence
  source: /note=“Description of Artificial Sequence Synthetic oligonucleotide”
tatctgtcgatggcaatagcc

SEQ ID NO: 411 - DNA, 21 bp, Artificial Sequence
  source: /note=“Description of Artificial Sequence Synthetic oligonucleotide”
gcaatagccaagaggctgaag

SEQ ID NO: 412 - DNA, 21 bp, Artificial Sequence
  source: /note=“Description of Artificial Sequence Synthetic oligonucleotide”
agtgctgatggtgatggcgaa

SEQ ID NO: 413 - DNA, 21 bp, Artificial Sequence
  source: /note=“Description of Artificial Sequence Synthetic oligonucleotide”
acaccgaggagcccatggcgg

SEQ ID NO: 414 - DNA, 21 bp, Artificial Sequence
  source: /note=“Description of Artificial Sequence Synthetic oligonucleotide”
cgccatccgaattccactccg

SEQ ID NO: 415 - DNA, 20 bp, Artificial Sequence
  source: /note=“Description of Artificial Sequence Synthetic oligonucleotide”
tggtgtcactggcggcggcc

SEQ ID NO: 416 - DNA, 20 bp, Artificial Sequence
  source: /note=“Description of Artificial Sequence Synthetic oligonucleotide”
ccatcaccatcagcaccggg

SEQ ID NO: 417 - DNA, 20 bp, Artificial Sequence
  source: /note=“Description of Artificial Sequence Synthetic oligonucleotide”
ccatcggcctgactcccatg

SEQ ID NO: 418 - DNA, 20 bp, Artificial Sequence
  source: /note=“Description of Artificial Sequence Synthetic oligonucleotide”
gctgaccgcagttgttccaa

SEQ ID NO: 419 - DNA, 20 bp, Artificial Sequence
  source: /note=“Description of Artificial Sequence Synthetic oligonucleotide”
aggatgtggtccccatgaac

SEQ ID NO: 420 - DNA, 20 bp, Artificial Sequence
  source: /note=“Description of Artificial Sequence Synthetic oligonucleotide”
cctgtgtgctggtgcccctg

SEQ ID NO: 421 - DNA, 20 bp, Artificial Sequence
  source: /note=“Description of Artificial Sequence Synthetic oligonucleotide”
cggatcttcctggcggcgcg

SEQ ID NO: 422 - DNA, 20 bp, Artificial Sequence
  source: /note=“Description of Artificial Sequence Synthetic oligonucleotide”
ccctctgctggctgccccta

SEQ ID NO: 423 - DNA, 20 bp, Artificial Sequence
  source: /note=“Description of Artificial Sequence Synthetic oligonucleotide”
ttctgccccgactgcagcca

SEQ ID NO: 424 - DNA, 20 bp, Artificial Sequence
  source: /note=“Description of Artificial Sequence Synthetic oligonucleotide”
aaggcagctggcaccagtgc

SEQ ID NO: 425 - DNA, 21 bp, Artificial Sequence
  source: /note=“Description of Artificial Sequence Synthetic oligonucleotide”
taagggcatcattgccatctg

SEQ ID NO: 426 - DNA, 21 bp, Artificial Sequence
  source: /note=“Description of Artificial Sequence Synthetic oligonucleotide”
cggcctgactcccatgctagg

SEQ ID NO: 427 - DNA, 21 bp, Artificial Sequence
  source: /note=“Description of Artificial Sequence Synthetic oligonucleotide”
gcagttgttccaacctagcat

SEQ ID NO: 428 - DNA, 21 bp, Artificial Sequence
  source: /note=“Description of Artificial Sequence Synthetic oligonucleotide”
ccgcagttgttccaacctagc

SEQ ID NO: 429 - DNA, 21 bp, Artificial Sequence
  source: /note=“Description of Artificial Sequence Synthetic oligonucleotide”
caagaaccactcccagggctg

SEQ ID NO: 430 - DNA, 21 bp, Artificial Sequence
  source: /note=“Description of Artificial Sequence Synthetic oligonucleotide”
cttggccctccccgcagccct

SEQ ID NO: 431 - DNA, 21 bp, Artificial Sequence
  source: /note=“Description of Artificial Sequence Synthetic oligonucleotide”
cacttggccctccccgcagcc

SEQ ID NO: 432 - DNA, 21 bp, Artificial Sequence
  source: /note=“Description of Artificial Sequence Synthetic oligonucleotide”
ggccaagtggcctgtctcttt

SEQ ID NO: 433 - DNA, 21 bp, Artificial Sequence
  source: /note=“Description of Artificial Sequence Synthetic oligonucleotide”
ttcatggggaccacatcctca

SEQ ID NO: 434 - DNA, 21 bp, Artificial Sequence
  source: /note=“Description of Artificial Sequence Synthetic oligonucleotide”
tgaagtacaccatgtagttca

SEQ ID NO: 435 - DNA, 21 bp, Artificial Sequence
  source: /note=“Description of Artificial Sequence Synthetic oligonucleotide”
ctggtgcccctgctgctcatg

SEQ ID NO: 436 - DNA, 21 bp, Artificial Sequence
  source: /note=“Description of Artificial Sequence Synthetic oligonucleotide”
gctcatgctgggtgtctattt

SEQ ID NO: 437 - DNA, 21 bp, Artificial Sequence
  source: /note=“Description of Artificial Sequence Synthetic oligonucleotide”
cttcagctgtcgtcgcgccgc

SEQ ID NO: 438 - DNA, 21 bp, Artificial Sequence
  source: /note=“Description of Artificial Sequence Synthetic oligonucleotide”
cgcgacgacagctgaagcaga

SEQ ID NO: 439 - DNA, 21 bp, Artificial Sequence
  source: /note=“Description of Artificial Sequence Synthetic oligonucleotide”
gatggagagccagcctctgcc

SEQ ID NO: 440 - DNA, 21 bp, Artificial Sequence
  source: /note=“Description of Artificial Sequence Synthetic oligonucleotide”
gcgtggctgcagtcggggcag

SEQ ID NO: 441 - DNA, 21 bp, Artificial Sequence
  source: /note=“Description of Artificial Sequence Synthetic oligonucleotide”
acgatggccaggtacatgagc

SEQ ID NO: 442 - DNA, 21 bp, Artificial Sequence
  source: /note=“Description of Artificial Sequence Synthetic oligonucleotide”
ctctcccacaccaattcggtt

SEQ ID NO: 443 - DNA, 21 bp, Artificial Sequence
  source: /note=“Description of Artificial Sequence Synthetic oligonucleotide”
gattcacaaccgaattggtgt

SEQ ID NO: 444 - DNA, 21 bp, Artificial Sequence
  source: /note=“Description of Artificial Sequence Synthetic oligonucleotide”
gggattcacaaccgaattggt

SEQ ID NO: 445 - DNA, 21 bp, Artificial Sequence
  source: /note=“Description of Artificial Sequence Synthetic oligonucleotide”
cgtagatgaagggattcacaa

SEQ ID NO: 446 - DNA, 21 bp, Artificial Sequence
  source: /note=“Description of Artificial Sequence Synthetic oligonucleotide”
ggatacggtaggcgtagatga

SEQ ID NO: 447 - DNA, 21 bp, Artificial Sequence
  source: /note=“Description of Artificial Sequence Synthetic oligonucleotide”
tcatctacgcctaccgtatcc

SEQ ID NO: 448 - DNA, 21 bp, Artificial Sequence
  source: /note=“Description of Artificial Sequence Synthetic oligonucleotide”
cggatacggtaggcgtagatg

SEQ ID NO: 449 - DNA, 21 bp, Artificial Sequence
  source: /note=“Description of Artificial Sequence Synthetic oligonucleotide”
gcggaaggtctggcggaactc

SEQ ID NO: 450 - DNA, 21 bp, Artificial Sequence
  source: /note=“Description of Artificial Sequence Synthetic oligonucleotide”
aatgatcttgcggaaggtctg

SEQ ID NO: 451 - DNA, 21 bp, Artificial Sequence
  source: /note=“Description of Artificial Sequence Synthetic oligonucleotide”
gacgtggctgcgaatgatctt

SEQ ID NO: 452 - DNA, 21 bp, Artificial Sequence
  source: /note=“Description of Artificial Sequence Synthetic oligonucleotide”
ttgctgcctcaggacgtggct

SEQ ID NO: 453 - DNA, 21 bp, Artificial Sequence
  source: /note=“Description of Artificial Sequence Synthetic oligonucleotide”
caaggcagctggcaccagtgc

SEQ ID NO: 454 - DNA, 21 bp, Artificial Sequence
  source: /note=“Description of Artificial Sequence Synthetic oligonucleotide”
cgggcactggtgccagctgcc

SEQ ID NO: 455 - DNA, 21 bp, Artificial Sequence
  source: /note=“Description of Artificial Sequence Synthetic oligonucleotide”
cttggcagctcatggcagtga

SEQ ID NO: 456 - DNA, 21 bp, Artificial Sequence
  source: /note=“Description of Artificial Sequence Synthetic oligonucleotide”
ccgtctcaacggccacccgcc

SEQ ID NO: 457 - DNA, 21 bp, Artificial Sequence
  source: /note=“Description of Artificial Sequence Synthetic oligonucleotide”
cacactcctggcgggtggccg

SEQ ID NO: 458 - DNA, 21 bp, Artificial Sequence
  source: /note=“Description of Artificial Sequence Synthetic oligonucleotide”
tgccgttggcccacactcctg

SEQ ID NO: 459 - DNA, 21 bp, Artificial Sequence
  source: /note=“Description of Artificial Sequence Synthetic oligonucleotide”
ccattgggcctccgctcaggg

SEQ ID NO: 460 - DNA, 21 bp, Artificial Sequence
  source: /note=“Description of Artificial Sequence Synthetic oligonucleotide”
catagccattgggcctccgct

SEQ ID NO: 461 - DNA, 21 bp, Artificial Sequence
  source: /note=“Description of Artificial Sequence Synthetic oligonucleotide”
aatggctatgccctggggctg

SEQ ID NO: 462 - DNA, 21 bp, Artificial Sequence
  source: /note=“Description of Artificial Sequence Synthetic oligonucleotide”
atgccctggggctggtgagtg

SEQ ID NO: 463 - DNA, 21 bp, Artificial Sequence
  source: /note=“Description of Artificial Sequence Synthetic oligonucleotide”
gccctggggctggtgagtgga

SEQ ID NO: 464 - DNA, 21 bp, Artificial Sequence
  source: /note=“Description of Artificial Sequence Synthetic oligonucleotide”
tggtgagtggagggagtgccc

SEQ ID NO: 465 - DNA, 21 bp, Artificial Sequence
  source: /note=“Description of Artificial Sequence Synthetic oligonucleotide”
gagggagtgcccaagagtccc

SEQ ID NO: 466 - DNA, 21 bp, Artificial Sequence
  source: /note=“Description of Artificial Sequence Synthetic oligonucleotide”
agggagtgcccaagagtccca

SEQ ID NO: 467 - DNA, 21 bp, Artificial Sequence
  source: /note=“Description of Artificial Sequence Synthetic oligonucleotide”
gtctgggaggcccgtgttccc

SEQ ID NO: 468 - DNA, 21 bp, Artificial Sequence
  source: /note=“Description of Artificial Sequence Synthetic oligonucleotide”
catggctaaggagctccacgt

SEQ ID NO: 469 - DNA, 21 bp, Artificial Sequence
  source: /note=“Description of Artificial Sequence Synthetic oligonucleotide”
gagctccttagccatgagctc

SEQ ID NO: 470 - DNA, 21 bp, Artificial Sequence
  source: /note=“Description of Artificial Sequence Synthetic oligonucleotide”
gctccttagccatgagctcaa

SEQ ID NO: 471 - DNA, 21 bp, Artificial Sequence
  source: /note=“Description of Artificial Sequence Synthetic oligonucleotide”
ggcctagatgaccccctggcc

SEQ ID NO: 472 - DNA, 21 bp, Artificial Sequence
  source: /note=“Description of Artificial Sequence Synthetic oligonucleotide”
ccccctggcccaggatggagc

SEQ ID NO: 473 - DNA, 21 bp, Artificial Sequence
  source: /note=“Description of Artificial Sequence Synthetic oligonucleotide”
ctcctgctccatcctgggcca

SEQ ID NO: 474 - DNA, 20 bp, Artificial Sequence
  source: /note=“Description of Artificial Sequence Synthetic oligonucleotide”
ccgtgatgtacaccgaggag

SEQ ID NO: 475 - DNA, 20 bp, Artificial Sequence
  source: /note=“Description of Artificial Sequence Synthetic oligonucleotide”
ctttgccatcaccatcagca

SEQ ID NO: 476 - DNA, 20 bp, Artificial Sequence
  source: /note=“Description of Artificial Sequence Synthetic oligonucleotide”
tttgccatcaccatcagcac

SEQ ID NO: 477 - DNA, 20 bp, Artificial Sequence
  source: /note=“Description of Artificial Sequence Synthetic oligonucleotide”
ttgcctgcttcgtcctggtc

SEQ ID NO: 478 - DNA, 20 bp, Artificial Sequence
  source: /note=“Description of Artificial Sequence Synthetic oligonucleotide”
tcctggtcctcacgcagagc

SEQ ID NO: 479 - DNA, 20 bp, Artificial Sequence
  source: /note=“Description of Artificial Sequence Synthetic oligonucleotide”
tcttcagtctcctggccatc

SEQ ID NO: 480 - DNA, 20 bp, Artificial Sequence
  source: /note=“Description of Artificial Sequence Synthetic oligonucleotide”
gtctcctggccatcgccatt

SEQ ID NO: 481 - DNA, 20 bp, Artificial Sequence
  source: /note=“Description of Artificial Sequence Synthetic oligonucleotide”
acctagcatgggagtcaggc

SEQ ID NO: 482 - DNA, 20 bp, Artificial Sequence
  source: /note=“Description of Artificial Sequence Synthetic oligonucleotide”
aacctagcatgggagtcagg

SEQ ID NO: 483 - DNA, 20 bp, Artificial Sequence
  source: /note=“Description of Artificial Sequence Synthetic oligonucleotide”
atgctaggttggaacaactg

SEQ ID NO: 484 - DNA, 20 bp, Artificial Sequence
  source: /note=“Description of Artificial Sequence Synthetic oligonucleotide”
gcagccctgggagtggttct

SEQ ID NO: 485 - DNA, 20 bp, Artificial Sequence
  source: /note=“Description of Artificial Sequence Synthetic oligonucleotide”
cgcagccctgggagtggttc

SEQ ID NO: 486 - DNA, 20 bp, Artificial Sequence
  source: /note=“Description of Artificial Sequence Synthetic oligonucleotide”
agggctgcggggagggccaa

SEQ ID NO: 487 - DNA, 20 bp, Artificial Sequence
  source: /note=“Description of Artificial Sequence Synthetic oligonucleotide”
tggggaccacatcctcaaag

SEQ ID NO: 488 - DNA, 20 bp, Artificial Sequence
  source: /note=“Description of Artificial Sequence Synthetic oligonucleotide”
catgaactacatggtgtact

SEQ ID NO: 489 - DNA, 20 bp, Artificial Sequence
  source: /note=“Description of Artificial Sequence Synthetic oligonucleotide”
atgaactacatggtgtactt

SEQ ID NO: 490 - DNA, 20 bp, Artificial Sequence
  source: /note=“Description of Artificial Sequence Synthetic oligonucleotide”
acttctttgcctgtgtgctg

SEQ ID NO: 491 - DNA, 20 bp, Artificial Sequence
  source: /note=“Description of Artificial Sequence Synthetic oligonucleotide”
tgctgctcatgctgggtgtc

SEQ ID NO: 492 - DNA, 20 bp, Artificial Sequence
  source: /note=“Description of Artificial Sequence Synthetic oligonucleotide”
caaatagacacccagcatga

SEQ ID NO: 493 - DNA, 20 bp, Artificial Sequence
  source: /note=“Description of Artificial Sequence Synthetic oligonucleotide”
gctgtcgtcgcgccgccagg

SEQ ID NO: 494 - DNA, 20 bp, Artificial Sequence
  source: /note=“Description of Artificial Sequence Synthetic oligonucleotide”
tggcggcgcgacgacagctg

SEQ ID NO: 495 - DNA, 20 bp, Artificial Sequence
  source: /note=“Description of Artificial Sequence Synthetic oligonucleotide”
tctgcttcagctgtcgtcgc

SEQ ID NO: 496 - DNA, 20 bp, Artificial Sequence
  source: /note=“Description of Artificial Sequence Synthetic oligonucleotide”
ggcagaggctggctctccat

SEQ ID NO: 497 - DNA, 20 bp, Artificial Sequence
  source: /note=“Description of Artificial Sequence Synthetic oligonucleotide”
cggcagaggctggctctcca

SEQ ID NO: 498 - DNA, 20 bp, Artificial Sequence
  source: /note=“Description of Artificial Sequence Synthetic oligonucleotide”
ccggcagaggctggctctcc

SEQ ID NO: 499 - DNA, 20 bp, Artificial Sequence
  source: /note=“Description of Artificial Sequence Synthetic oligonucleotide”
cactgcagaaggaggtccat

SEQ ID NO: 500 - DNA, 20 bp, Artificial Sequence
  source: /note=“Description of Artificial Sequence Synthetic oligonucleotide”
tgctgccaagtcactggcca

SEQ ID NO: 501 - DNA, 20 bp, Artificial Sequence
  source: /note=“Description of Artificial Sequence Synthetic oligonucleotide”
acaatgatggccagtgactt

SEQ ID NO: 502 - DNA, 20 bp, Artificial Sequence
  source: /note=“Description of Artificial Sequence Synthetic oligonucleotide”
tacacatcatcaactgcttc

SEQ ID NO: 503 - DNA, 20 bp, Artificial Sequence
  source: /note=“Description of Artificial Sequence Synthetic oligonucleotide”
ctttcttctgccccgactgc

SEQ ID NO: 504 - DNA, 20 bp, Artificial Sequence
  source: /note=“Description of Artificial Sequence Synthetic oligonucleotide”
gactgcagccacgcccctct

SEQ ID NO: 505 - DNA, 20 bp, Artificial Sequence
  source: /note=“Description of Artificial Sequence Synthetic oligonucleotide”
tctctggctcatgtacctgg

SEQ ID NO: 506 - DNA, 20 bp, Artificial Sequence
  source: /note=“Description of Artificial Sequence Synthetic oligonucleotide”
caaccgaattggtgtgggag

SEQ ID NO: 507 - DNA, 20 bp, Artificial Sequence
  source: /note=“Description of Artificial Sequence Synthetic oligonucleotide”
acaccaattcggttgtgaat

SEQ ID NO: 508 - DNA, 20 bp, Artificial Sequence
  source: /note=“Description of Artificial Sequence Synthetic oligonucleotide”
gttgtgaatcccttcatcta

SEQ ID NO: 509 - DNA, 20 bp, Artificial Sequence
  source: /note=“Description of Artificial Sequence Synthetic oligonucleotide”
ttcatctacgcctaccgtat

SEQ ID NO: 510 - DNA, 20 bp, Artificial Sequence
  source: /note=“Description of Artificial Sequence Synthetic oligonucleotide”
tctacgcctaccgtatccgc

SEQ ID NO: 511 - DNA, 20 bp, Artificial Sequence
  source: /note=“Description of Artificial Sequence Synthetic oligonucleotide”
cgagttccgccagaccttcc

SEQ ID NO: 512 - DNA, 20 bp, Artificial Sequence
  source: /note=“Description of Artificial Sequence Synthetic oligonucleotide”
gccagaccttccgcaagatc

SEQ ID NO: 513 - DNA, 20 bp, Artificial Sequence
  source: /note=“Description of Artificial Sequence Synthetic oligonucleotide”
ccagaccttccgcaagatca

SEQ ID NO: 514 - DNA, 20 bp, Artificial Sequence
  source: /note=“Description of Artificial Sequence Synthetic oligonucleotide”
gcaagatcattcgcagccac

SEQ ID NO: 515 - DNA, 20 bp, Artificial Sequence
  source: /note=“Description of Artificial Sequence Synthetic oligonucleotide”
caagatcattcgcagccacg

SEQ ID NO: 516 - DNA, 20 bp, Artificial Sequence
  source: /note=“Description of Artificial Sequence Synthetic oligonucleotide”
cagccacgtcctgaggcagc

SEQ ID NO: 517 - DNA, 20 bp, Artificial Sequence
  source: /note=“Description of Artificial Sequence Synthetic oligonucleotide”
aggcagctggcaccagtgcc

SEQ ID NO: 518 - DNA, 20 bp, Artificial Sequence
  source: /note=“Description of Artificial Sequence Synthetic oligonucleotide”
tcactgccatgagctgccaa

SEQ ID NO: 519 - DNA, 20 bp, Artificial Sequence
  source: /note=“Description of Artificial Sequence Synthetic oligonucleotide”
tctcaacggccacccgccag

SEQ ID NO: 520 - DNA, 20 bp, Artificial Sequence
  source: /note=“Description of Artificial Sequence Synthetic oligonucleotide”
ctcagggtggggagcactgc

SEQ ID NO: 521 - DNA, 20 bp, Artificial Sequence
  source: /note=“Description of Artificial Sequence Synthetic oligonucleotide”
caccctgagcggaggcccaa

SEQ ID NO: 522 - DNA, 20 bp, Artificial Sequence
  source: /note=“Description of Artificial Sequence Synthetic oligonucleotide”
accctgagcggaggcccaat

SEQ ID NO: 523 - DNA, 20 bp, Artificial Sequence
  source: /note=“Description of Artificial Sequence Synthetic oligonucleotide”
agggcatagccattgggcct

SEQ ID NO: 524 - DNA, 20 bp, Artificial Sequence
  source: /note=“Description of Artificial Sequence Synthetic oligonucleotide”
ctcaccagccccagggcata

SEQ ID NO: 525 - DNA, 20 bp, Artificial Sequence
  source: /note=“Description of Artificial Sequence Synthetic oligonucleotide”
tccactcaccagccccaggg

SEQ ID NO: 526 - DNA, 20 bp, Artificial Sequence
  source: /note=“Description of Artificial Sequence Synthetic oligonucleotide”
tgggactcttgggcactccc

SEQ ID NO: 527 - DNA, 20 bp, Artificial Sequence
  source: /note=“Description of Artificial Sequence Synthetic oligonucleotide”
ctgggactcttgggcactcc

SEQ ID NO: 528 - DNA, 20 bp, Artificial Sequence
  source: /note=“Description of Artificial Sequence Synthetic oligonucleotide”
cctgggactcttgggcactc

SEQ ID NO: 529 - DNA, 20 bp, Artificial Sequence
  source: /note=“Description of Artificial Sequence Synthetic oligonucleotide”
aggggaacacgggcctccca

SEQ ID NO: 530 - DNA, 20 bp, Artificial Sequence
  source: /note=“Description of Artificial Sequence Synthetic oligonucleotide”
cgtctgggaggcccgtgttc

SEQ ID NO: 531 - DNA, 20 bp, Artificial Sequence
  source: /note=“Description of Artificial Sequence Synthetic oligonucleotide”
agacgtggagctccttagcc

SEQ ID NO: 532 - DNA, 20 bp, Artificial Sequence
  source: /note=“Description of Artificial Sequence Synthetic oligonucleotide”
ttgagctcatggctaaggag

SEQ ID NO: 533 - DNA, 20 bp, Artificial Sequence
  source: /note=“Description of Artificial Sequence Synthetic oligonucleotide”
ctggcctagatgaccccctg

SEQ ID NO: 534 - DNA, 20 bp, Artificial Sequence
  source: /note=“Description of Artificial Sequence Synthetic oligonucleotide”
tggcctagatgaccccctgg

SEQ ID NO: 535 - DNA, 20 bp, Artificial Sequence
  source: /note=“Description of Artificial Sequence Synthetic oligonucleotide”
tcctgggccagggggtcatc

SEQ ID NO: 536 - DNA, 20 bp, Artificial Sequence
  source: /note=“Description of Artificial Sequence Synthetic oligonucleotide”
ctggcccaggatggagcagg

SEQ ID NO: 537 - DNA, 20 bp, Artificial Sequence
  source: /note=“Description of Artificial Sequence Synthetic oligonucleotide”
tggcccaggatggagcagga

SEQ ID NO: 538 - DNA, 20 bp, Artificial Sequence
  source: /note=“Description of Artificial Sequence Synthetic oligonucleotide”
cgcgagttccgccagacctt

SEQ ID NO: 539 - DNA, 20 bp, Artificial Sequence
  source: /note=“Description of Artificial Sequence Synthetic oligonucleotide”
ccctggggctggtgagtgga

SEQ ID NO: 540 - DNA, 20 bp, Artificial Sequence
  source: /note=“Description of Artificial Sequence Synthetic oligonucleotide”
caggacgatgtgcagcggcc

SEQ ID NO: 541 - DNA, 20 bp, Artificial Sequence
  source: /note=“Description of Artificial Sequence Synthetic oligonucleotide”
accgcacgttcagaagtcgg

SEQ ID NO: 542 - DNA, 20 bp, Artificial Sequence
  source: /note=“Description of Artificial Sequence Synthetic oligonucleotide”
acaactgtgtaaattttgtg

SEQ ID NO: 543 - DNA, 20 bp, Artificial Sequence
  source: /note=“Description of Artificial Sequence Synthetic oligonucleotide”
caactgtgtaaattttgtga

SEQ ID NO: 544 - DNA, 20 bp, Artificial Sequence
  source: /note=“Description of Artificial Sequence Synthetic oligonucleotide”
acctgtgacaaccagaaatc

SEQ ID NO: 545 - DNA, 20 bp, Artificial Sequence
  source: /note=“Description of Artificial Sequence Synthetic oligonucleotide”
cctgtgacaaccagaaatcc

SEQ ID NO: 546 - DNA, 20 bp, Artificial Sequence
  source: /note=“Description of Artificial Sequence Synthetic oligonucleotide”
tgtggcttctcacagatgga

SEQ ID NO: 547 - DNA, 20 bp, Artificial Sequence
  source: /note=“Description of Artificial Sequence Synthetic oligonucleotide”
tctgtgagaagccacaggaa

SEQ ID NO: 548 - DNA, 20 bp, Artificial Sequence
  source: /note=“Description of Artificial Sequence Synthetic oligonucleotide”
aagctcccctaccatgactt

SEQ ID NO: 549 - DNA, 20 bp, Artificial Sequence
  source: /note=“Description of Artificial Sequence Synthetic oligonucleotide”
gaataaagtcatggtagggg

SEQ ID NO: 550 - DNA, 20 bp, Artificial Sequence
  source: /note=“Description of Artificial Sequence Synthetic oligonucleotide”
agaataaagtcatggtaggg

SEQ ID NO: 551 - DNA, 20 bp, Artificial Sequence
  source: /note=“Description of Artificial Sequence Synthetic oligonucleotide”
ctaccatgactttattctgg

SEQ ID NO: 552 - DNA, 20 bp, Artificial Sequence
  source: /note=“Description of Artificial Sequence S ynthetic oligonucleotide”
taccatgactttattctgga

SEQ ID NO: 553 - DNA, 20 bp, Artificial Sequence
  source: /note=“Description of Artificial Sequence Synthetic oligonucleotide”
taatgcactttggagaagca

SEQ ID NO: 554 - DNA, 20 bp, Artificial Sequence
  source: /note=“Description of Artificial Sequence Synthetic oligonucleotide”
ttcataatgcactttggaga

SEQ ID NO: 555 - DNA, 20 bp, Artificial Sequence
  source: /note=“Description of Artificial Sequence Synthetic oligonucleotide”
aagtgcattatgaaggaaaa

SEQ ID NO: 556 - DNA, 20 bp, Artificial Sequence
  source: /note=“Description of Artificial Sequence Synthetic oligonucleotide”
tgtgttcctgtagctctgat

SEQ ID NO: 557 - DNA, 20 bp, Artificial Sequence
  source: /note=“Description of Artificial Sequence Synthetic oligonucleotide”
tgtagctctgatgagtgcaa

SEQ ID NO: 558 - DNA, 20 bp, Artificial Sequence
  source: /note=“Description of Artificial Sequence Synthetic oligonucleotide”
agtgacaggcatcagcctcc

SEQ ID NO: 559 - DNA, 20 bp, Artificial Sequence
  source: /note=“Description of Artificial Sequence Synthetic oligonucleotide”
agtggtggcaggaggctgat

SEQ ID NO: 560 - DNA, 20 bp, Artificial Sequence
  source: /note=“Description of Artificial Sequence Synthetic oligonucleotide”
aggttgaactcagcttctgc

SEQ ID NO: 561 - DNA, 20 bp, Artificial Sequence
  source: /note=“Description of Artificial Sequence Synthetic oligonucleotide”
caggttgaactcagcttctg

SEQ ID NO: 562 - DNA, 20 bp, Artificial Sequence
  source: /note=“Description of Artificial Sequence Synthetic oligonucleotide”
acctgggaaaccggcaagac

SEQ ID NO: 563 - DNA, 20 bp, Artificial Sequence
  source: /note=“Description of Artificial Sequence Synthetic oligonucleotide”
cgtcttgccggtttcccagg

SEQ ID NO: 564 - DNA, 20 bp, Artificial Sequence
  source: /note=“Description of Artificial Sequence Synthetic oligonucleotide”
gcgtcttgccggtttcccag

SEQ ID NO: 565 - DNA, 20 bp, Artificial Sequence
  source: /note=“Description of Artificial Sequence Synthetic oligonucleotide”
tgagcttccgcgtcttgccg

SEQ ID NO: 566 - DNA, 20 bp, Artificial Sequence
  source: /note=“Description of Artificial Sequence Synthetic oligonucleotide”
gcgagcactgtgccatcatc

SEQ ID NO: 567 - DNA, 20 bp, Artificial Sequence
  source: /note=“Description of Artificial Sequence Synthetic oligonucleotide”
ggatgatggcacagtgctcg

SEQ ID NO: 568 - DNA, 20 bp, Artificial Sequence
  source: /note=“Description of Artificial Sequence Synthetic oligonucleotide”
aggatgatggcacagtgctc

SEQ ID NO: 569 - DNA, 20 bp, Artificial Sequence
  source: /note=“Description of Artificial Sequence Synthetic oligonucleotide”
cgtgtgccaacaacatcaac

SEQ ID NO: 570 - DNA, 20 bp, Artificial Sequence
  source: /note=“Description of Artificial Sequence Synthetic oligonucleotide”
gctcaatgggcagcagctct

SEQ ID NO: 571 - DNA, 20 bp, Artificial Sequence
  source: /note=“Description of Artificial Sequence Synthetic oligonucleotide”
accagggtgtccagctcaat

SEQ ID NO: 572 - DNA, 20 bp, Artificial Sequence
  source: /note=“Description of Artificial Sequence Synthetic oligonucleotide”
caccagggtgtccagctcaa

SEQ ID NO: 573 - DNA, 20 bp, Artificial Sequence
  source: /note=“Description of Artificial Sequence Synthetic oligonucleotide”
ccaccagggtgtccagctca

SEQ ID NO: 574 - DNA, 20 bp, Artificial Sequence
  source: /note=“Description of Artificial Sequence Synthetic oligonucleotide”
gcttggccttatagacctca

SEQ ID NO: 575 - DNA, 20 bp, Artificial Sequence
  source: /note=“Description of Artificial Sequence Synthetic oligonucleotide”
gagcagtttgagacagtggc

SEQ ID NO: 576 - DNA, 20 bp, Artificial Sequence
  source: /note=“Description of Artificial Sequence Synthetic oligonucleotide”
agaggcatactcctcatagg

SEQ ID NO: 577 - DNA, 20 bp, Artificial Sequence
  source: /note=“Description of Artificial Sequence Synthetic oligonucleotide”
ctatgaggagtatgcctctt

SEQ ID NO: 578 - DNA, 20 bp, Artificial Sequence
  source: /note=“Description of Artificial Sequence Synthetic oligonucleotide”
aagaggcatactcctcatag

SEQ ID NO: 579 - DNA, 20 bp, Artificial Sequence
  source: /note=“Description of Artificial Sequence Synthetic oligonucleotide”
tatgaggagtatgcctcttg

SEQ ID NO: 580 - DNA, 20 bp, Artificial Sequence
  source: /note=“Description of Artificial Sequence Synthetic oligonucleotide”
gattgatgtctgagaagatg

SEQ ID NO: 581 - DNA, 20 bp, Artificial Sequence
  source: /note=“Description of Artificial Sequence Synthetic oligonucleotide”
ctcctcagccgtcaggaact

SEQ ID NO: 582 - DNA, 20 bp, Artificial Sequence
  source: /note=“Description of Artificial Sequence Synthetic oligonucleotide”
gttcctgacggctgaggagc

SEQ ID NO: 583 - DNA, 20 bp, Artificial Sequence
  source: /note=“Description of Artificial Sequence Synthetic oligonucleotide”
gctcctcagccgtcaggaac

SEQ ID NO: 584 - DNA, 20 bp, Artificial Sequence
  source: /note=“Description of Artificial Sequence Synthetic oligonucleotide”
tgacggctgaggagcggaag

SEQ ID NO: 585 - DNA, 20 bp, Artificial Sequence
  source: /note=“Description of Artificial Sequence Synthetic oligonucleotide”
tcttccgctcctcagccgtc

SEQ ID NO: 586 - DNA, 20 bp, Artificial Sequence
  source: /note=“Description of Artificial Sequence Synthetic oligonucleotide”
aactccgtcttccgctcctc

SEQ ID NO: 587 - DNA, 20 bp, Artificial Sequence
  source: /note=“Description of Artificial Sequence Synthetic oligonucleotide”
caactccgtcttccgctcct

SEQ ID NO: 588 - DNA, 20 bp, Artificial Sequence
  source: /note=“Description of Artificial Sequence Synthetic oligonucleotide”
ccaactccgtcttccgctcc

SEQ ID NO: 589 - DNA, 20 bp, Artificial Sequence
  source: /note=“Description of Artificial Sequence Synthetic oligonucleotide”
acgccaagggcaacctacag

SEQ ID NO: 590 - DNA, 20 bp, Artificial Sequence
  source: /note=“Description of Artificial Sequence Synthetic oligonucleotide”
cgccaagggcaacctacagg

SEQ ID NO: 591 - DNA, 20 bp, Artificial Sequence
  source: /note=“Description of Artificial Sequence Synthetic oligonucleotide”
agctgatgacatgccgcgtc

SEQ ID NO: 592 - DNA, 20 bp, Artificial Sequence
  source: /note=“Description of Artificial Sequence Synthetic oligonucleotide”
gggcgagggagctgcccagc

SEQ ID NO: 593 - DNA, 20 bp, Artificial Sequence
  source: /note=“Description of Artificial Sequence Synthetic oligonucleotide”
cgggcgagggagctgcccag

SEQ ID NO: 594 - DNA, 20 bp, Artificial Sequence
  source: /note=“Description of Artificial Sequence Synthetic oligonucleotide”
ccgggcgagggagctgccca

SEQ ID NO: 595 - DNA, 20 bp, Artificial Sequence
  source: /note=“Description of Artificial Sequence Synthetic oligonucleotide”
tcgcccgggggattgctcac

SEQ ID NO: 596 - DNA, 20 bp, Artificial Sequence
  source: /note=“Description of Artificial Sequence Synthetic oligonucleotide”
acatggagtgtgatcactgt

SEQ ID NO: 597 - DNA, 20 bp, Artificial Sequence
  source: /note=“Description of Artificial Sequence Synthetic oligonucleotide”
cagtgatcacactccatgtg

SEQ ID NO: 598 - DNA, 20 bp, Artificial Sequence
  source: /note=“Description of Artificial Sequence Synthetic oligonucleotide”
tgtgggaggcccaagatgcc

SEQ ID NO: 599 - DNA, 20 bp, Artificial Sequence
  source: /note=“Description of Artificial Sequence Synthetic oligonucleotide”
tgtgcacgatgggcatcttg

SEQ ID NO: 600 - DNA, 20 bp, Artificial Sequence
  source: /note=“Description of Artificial Sequence Synthetic oligonucleotide”
cgaggatattggagctcttg

SEQ ID NO: 601 - DNA, 20 bp, Artificial Sequence
  source: /note=“Description of Artificial Sequence Synthetic oligonucleotide”
atatcctcgtgaagaacgac

SEQ ID NO: 602 - DNA, 20 bp, Artificial Sequence
  source: /note=“Description of Artificial Sequence Synthetic oligonucleotide”
gacgcagggaaagcccaaag

SEQ ID NO: 603 - DNA, 20 bp, Artificial Sequence
  source: /note=“Description of Artificial Sequence Synthetic oligonucleotide”
ctgcgtctggaccctactct

SEQ ID NO: 604 - DNA, 20 bp, Artificial Sequence
  source: /note=“Description of Artificial Sequence Synthetic oligonucleotide”
tgcgtctggaccctactctg

SEQ ID NO: 605 - DNA, 20 bp, Artificial Sequence
  source: /note=“Description of Artificial Sequence Synthetic oligonucleotide”
cagacagagtagggtccaga

SEQ ID NO: 606 - DNA, 20 bp, Artificial Sequence
  source: /note=“Description of Artificial Sequence Synthetic oligonucleotide”
gccagcacgatcccaccgca

SEQ ID NO: 607 - DNA, 20 bp, Artificial Sequence
  source: /note=“Description of Artificial Sequence Synthetic oligonucleotide”
aaggaaaaaaaaaagcctgg

SEQ ID NO: 608 - DNA, 20 bp, Artificial Sequence
  source: /note=“Description of Artificial Sequence Synthetic oligonucleotide”
acaccagcaatcctgacttg

SEQ ID NO: 609 - DNA, 20 bp, Artificial Sequence
  source: /note=“Description of Artificial Sequence Synthetic oligonucleotide”
actagcaacaagtcaggatt

SEQ ID NO: 610 - DNA, 20 bp, Artificial Sequence
  source: /note=“Description of Artificial Sequence Synthetic oligonucleotide”
gcaactcccagtggtggcag

SEQ ID NO: 611 - DNA, 20 bp, Artificial Sequence
  source: /note=“Description of Artificial Sequence Synthetic oligonucleotide”
tgtcatcatcatcttctact

SEQ ID NO: 612 - DNA, 20 bp, Artificial Sequence
  source: /note=“Description of Artificial Sequence Synthetic oligonucleotide”
gacctcagcaaagcgacctt

SEQ ID NO: 613 - DNA, 20 bp, Artificial Sequence
  source: /note=“Description of Artificial Sequence Synthetic oligonucleotide”
aggccaagctgaagcagaac

SEQ ID NO: 614 - DNA, 20 bp, Artificial Sequence
  source: /note=“Description of Artificial Sequence Synthetic oligonucleotide”
aggagtatgcctcttggaag

SEQ ID NO: 615 - DNA, 20 bp, Artificial Sequence
  source: /note=“Description of Artificial Sequence Synthetic oligonucleotide”
cctcttggaagacagagaag

SEQ ID NO: 616 - DNA, 20 bp, Artificial Sequence
  source: /note=“Description of Artificial Sequence Synthetic oligonucleotide”
ttctcatgcttcagattgat

SEQ ID NO: 617 - DNA, 20 bp, Artificial Sequence
  source: /note=“Description of Artificial Sequence Synthetic oligonucleotide”
ctcgtgaagaacgacctaac

SEQ ID NO: 618 - DNA, 20 bp, Artificial Sequence
  source: /note=“Description of Artificial Sequence Synthetic oligonucleotide”
ggccgctgcacatcgtcctg

SEQ ID NO: 619 - DNA, 20 bp, Artificial Sequence
  source: /note=“Description of Artificial Sequence Synthetic oligonucleotide”
gcggggtctgccatgggtcg

SEQ ID NO: 620 - DNA, 20 bp, Artificial Sequence
  source: /note=“Description of Artificial Sequence Synthetic oligonucleotide”
agttgctcatgcaggatttc

SEQ ID NO: 621 - DNA, 20 bp, Artificial Sequence
  source: /note=“Description of Artificial Sequence Synthetic oligonucleotide”
ccagaataaagtcatggtag

SEQ ID NO: 622 - DNA, 20 bp, Artificial Sequence
  source: /note=“Description of Artificial Sequence Synthetic oligonucleotide”
cccctaccatgactttattc

SEQ ID NO: 623 - DNA, 20 bp, Artificial Sequence
  source: /note=“Description of Artificial Sequence Synthetic oligonucleotide”
aagtcatggtaggggagctt

SEQ ID NO: 624 - DNA, 20 bp, Artificial Sequence
  source: /note=“Description of Artificial Sequence Synthetic oligonucleotide”
agtcatggtaggggagcttg

SEQ ID NO: 625 - DNA, 20 bp, Artificial Sequence
  source: /note=“Description of Artificial Sequence Synthetic oligonucleotide”
attgcactcatcagagctac

SEQ ID NO: 626 - DNA, 20 bp, Artificial Sequence
  source: /note=“Description of Artificial Sequence Synthetic oligonucleotide”
cctagagtgaagagattcat

SEQ ID NO: 627 - DNA, 20 bp, Artificial Sequence
  source: /note=“Description of Artificial Sequence Synthetic oligonucleotide”
ccaatgaatctcttcactct

SEQ ID NO: 628 - DNA, 20 bp, Artificial Sequence
  source: /note=“Description of Artificial Sequence Synthetic oligonucleotide”
aaagtcatggtaggggagct

SEQ ID NO: 629 - DNA, 20 bp, Artificial Sequence
  source: /note=“Description of Artificial Sequence Synthetic oligonucleotide”
gtgagcaatcccccgggcga

SEQ ID NO: 630 - DNA, 20 bp, Artificial Sequence
  source: /note=“Description of Artificial Sequence Synthetic oligonucleotide”
gtcgttcttcacgaggatat

SEQ ID NO: 631 - DNA, 20 bp, Artificial Sequence
  source: /note=“Description of Artificial Sequence Synthetic oligonucleotide”
gccgcgtcaggtactcctgt

SEQ ID NO: 632 - DNA, 20 bp, Artificial Sequence
  source: /note=“Description of Artificial Sequence Synthetic oligonucleotide”
gacgcggcatgtcatcagct

SEQ ID NO: 633 - DNA, 20 bp, Artificial Sequence
  source: /note=“Description of Artificial Sequence Synthetic oligonucleotide”
gcttctgctgccggttaacg

SEQ ID NO: 634 - DNA, 20 bp, Artificial Sequence
  source: /note=“Description of Artificial Sequence Synthetic oligonucleotide”
gtggatgacctggctaacag

SEQ ID NO: 635 - DNA, 20 bp, Artificial Sequence
  source: /note=“Description of Artificial Sequence Synthetic oligonucleotide”
gtgatcacactccatgtggg

SEQ ID NO: 636 - DNA, 20 bp, Artificial Sequence
  source: /note=“Description of Artificial Sequence Synthetic oligonucleotide”
gcccattgagctggacaccc

SEQ ID NO: 637 - DNA, 20 bp, Artificial Sequence
  source: /note=“Description of Artificial Sequence Synthetic oligonucleotide”
gcggtcatcttccaggatga

SEQ ID NO: 638 - DNA, 20 bp, Artificial Sequence
  source: /note=“Description of Artificial Sequence Synthetic oligonucleotide”
gggagctgcccagcttgcgc

SEQ ID NO: 639 - DNA, 20 bp, Artificial Sequence
  source: /note=“Description of Artificial Sequence Synthetic oligonucleotide”
gttgatgttgttggcacacg

SEQ ID NO: 640 - DNA, 20 bp, Artificial Sequence
  source: /note=“Description of Artificial Sequence Synthetic oligonucleotide”
ggcatcttgggcctcccaca

SEQ ID NO: 641 - DNA, 20 bp, Artificial Sequence
  source: /note=“Description of Artificial Sequence Synthetic oligonucleotide”
gcggcatgtcatcagctggg

SEQ ID NO: 642 - DNA, 20 bp, Artificial Sequence
  source: /note=“Description of Artificial Sequence Synthetic oligonucleotide”
gctcctcagccgtcaggaac

SEQ ID NO: 643 - DNA, 20 bp, Artificial Sequence
  source: /note=“Description of Artificial Sequence Synthetic oligonucleotide”
gctggtgttatattctgatg

SEQ ID NO: 644 - DNA, 20 bp, Artificial Sequence
  source: /note=“Description of Artificial Sequence Synthetic oligonucleotide”
ccgacttctgaacgtgcggt

SEQ ID NO: 645 - DNA, 20 bp, Artificial Sequence
  source: /note=“Description of Artificial Sequence Synthetic oligonucleotide”
tgctggcgatacgcgtccac

SEQ ID NO: 646 - DNA, 20 bp, Artificial Sequence
  source: /note=“Description of Artificial Sequence Synthetic oligonucleotide”
cccgacttctgaacgtgcgg

SEQ ID NO: 647 - DNA, 20 bp, Artificial Sequence
  source: /note=“Description of Artificial Sequence Synthetic oligonucleotide”
ccaccgcacgttcagaagtc

SEQ ID NO: 648 - DNA, 20 bp, Artificial Sequence
  source: /note=“Description of Artificial Sequence Synthetic oligonucleotide”
tcacccgacttctgaacgtg

SEQ ID NO: 649 - DNA, 20 bp, Artificial Sequence
  source: /note=“Description of Artificial Sequence Synthetic oligonucleotide”
cccaccgcacgttcagaagt

SEQ ID NO: 650 - DNA, 20 bp, Artificial Sequence
  source: /note=“Description of Artificial Sequence Synthetic oligonucleotide”
cgagcagcggggtctgccat

SEQ ID NO: 651 - DNA, 20 bp, Artificial Sequence
  source: /note=“Description of Artificial Sequence Synthetic oligonucleotide”
acgagcagcggggtctgcca

SEQ ID NO: 652 - DNA, 20 bp, Artificial Sequence
  source: /note=“Description of Artificial Sequence Synthetic oligonucleotide”
agcggggtctgccatgggtc

SEQ ID NO: 653 - DNA, 20 bp, Artificial Sequence
  source: /note=“Description of Artificial Sequence Synthetic oligonucleotide”
cctgagcagcccccgaccca

SEQ ID NO: 654 - DNA, 20 bp, Artificial Sequence
  source: /note=“Description of Artificial Sequence Synthetic oligonucleotide”
ccatgggtcgggggctgctc

SEQ ID NO: 655 - DNA, 20 bp, Artificial Sequence
  source: /note=“Description of Artificial Sequence Synthetic oligonucleotide”
aacgtgcggtgggatcgtgc

SEQ ID NO: 656 - DNA, 20 bp, Artificial Sequence
  source: /note=“Description of Artificial Sequence Synthetic oligonucleotide”
ggacgatgtgcagcggccac

SEQ ID NO: 657 - DNA, 20 bp, Artificial Sequence
  source: /note=“Description of Artificial Sequence Synthetic oligonucleotide”
gtccacaggacgatgtgcag

SEQ ID NO: 658 - DNA, 20 bp, Artificial Sequence
  source: /note=“Description of Artificial Sequence Synthetic oligonucleotide”
catgggtcgggggctgctca

SEQ ID NO: 659 - DNA, 20 bp, Artificial Sequence
  source: /note=“Description of Artificial Sequence Synthetic oligonucleotide”
cagcggggtctgccatgggt

SEQ ID NO: 660 - DNA, 20 bp, Artificial Sequence
  source: /note=“Description of Artificial Sequence Synthetic oligonucleotide”
atgggtcgggggctgctcag

SEQ ID NO: 661 - DNA, 20 bp, Artificial Sequence
  source: /note=“Description of Artificial Sequence Synthetic oligonucleotide”
cggggtctgccatgggtcgg

SEQ ID NO: 662 - DNA, 20 bp, Artificial Sequence
  source: /note=“Description of Artificial Sequence Synthetic oligonucleotide”
aggaagtctgtgtggctgta

SEQ ID NO: 663 - DNA, 20 bp, Artificial Sequence
  source: /note=“Description of Artificial Sequence Synthetic oligonucleotide”
ctccatctgtgagaagccac

SEQ ID NO: 664 - DNA, 20 bp, Artificial Sequence
  source: /note=“Description of Artificial Sequence Synthetic oligonucleotide”
atgatagtcactgacaacaa

SEQ ID NO: 665 - DNA, 20 bp, Artificial Sequence
  source: /note=“Description of Artificial Sequence Synthetic oligonucleotide”
gatgctgcagttgctcatgc

SEQ ID NO: 666 - DNA, 20 bp, Artificial Sequence
  source: /note=“Description of Artificial Sequence Synthetic oligonucleotide”
acagccacacagacttcctg

SEQ ID NO: 667 - DNA, 20 bp, Artificial Sequence
  source: /note=“Description of Artificial Sequence Synthetic oligonucleotide”
gaagccacaggaagtctgtg

SEQ ID NO: 668 - DNA, 20 bp, Artificial Sequence
  source: /note=“Description of Artificial Sequence Synthetic oligonucleotide”
ttcctgtggcttctcacaga

SEQ ID NO: 669 - DNA, 20 bp, Artificial Sequence
  source: /note=“Description of Artificial Sequence Synthetic oligonucleotide”
ctgtggcttctcacagatgg

SEQ ID NO: 670 - DNA, 20 bp, Artificial Sequence
  source: /note=“Description of Artificial Sequence Synthetic oligonucleotide”
tcacaaaatttacacagttg

SEQ ID NO: 671 - DNA, 20 bp, Artificial Sequence
  source: /note=“Description of Artificial Sequence Synthetic oligonucleotide”
gacaacatcatcttctcaga

SEQ ID NO: 672 - DNA, 20 bp, Artificial Sequence
  source: /note=“Description of Artificial Sequence Synthetic oligonucleotide”
tccagaataaagtcatggta

SEQ ID NO: 673 - DNA, 20 bp, Artificial Sequence
  source: /note=“Description of Artificial Sequence Synthetic oligonucleotide”
ggtaggggagcttggggtca

SEQ ID NO: 674 - DNA, 20 bp, Artificial Sequence
  source: /note=“Description of Artificial Sequence Synthetic oligonucleotide”
ttctccaaagtgcattatga

SEQ ID NO: 675 - DNA, 20 bp, Artificial Sequence
  source: /note=“Description of Artificial Sequence Synthetic oligonucleotide”
catcttccagaataaagtca

SEQ ID NO: 676 - DNA, 20 bp, Artificial Sequence
  source: /note=“Description of Artificial Sequence Synthetic oligonucleotide”
cacatgaagaaagtctcacc

SEQ ID NO: 677 - DNA, 20 bp, Artificial Sequence
  source: /note=“Description of Artificial Sequence Synthetic oligonucleotide”
ttccagaataaagtcatggt

SEQ ID NO: 678 - DNA, 20 bp, Artificial Sequence
  source: /note=“Description of Artificial Sequence Synthetic oligonucleotide”
ttttccttcataatgcactt

SEQ ID NO: 679 - DNA, 20 bp, Artificial Sequence
  source: /note=“Description of Artificial Sequence Synthetic oligonucleotide”
cacagttgtggaaacttgac

SEQ ID NO: 680 - DNA, 20 bp, Artificial Sequence
  source: /note=“Description of Artificial Sequence Synthetic oligonucleotide”
cccaactccgtcttccgctc

SEQ ID NO: 681 - DNA, 20 bp, Artificial Sequence
  source: /note=“Description of Artificial Sequence Synthetic oligonucleotide”
ggctttccctgcgtctggac

SEQ ID NO: 682 - DNA, 20 bp, Artificial Sequence
  source: /note=“Description of Artificial Sequence Synthetic oligonucleotide”
ctgaggtctataaggccaag

SEQ ID NO: 683 - DNA, 20 bp, Artificial Sequence
  source: /note=“Description of Artificial Sequence Synthetic oligonucleotide”
tgatgtgagattttccacct

SEQ ID NO: 684 - DNA, 20 bp, Artificial Sequence
  source: /note=“Description of Artificial Sequence Synthetic oligonucleotide”
cctatgaggagtatgcctct

SEQ ID NO: 685 - DNA, 20 bp, Artificial Sequence
  source: /note=“Description of Artificial Sequence Synthetic oligonucleotide”
aagtgacaggcatcagcctc

SEQ ID NO: 686 - DNA, 20 bp, Artificial Sequence
  source: /note=“Description of Artificial Sequence Synthetic oligonucleotide”
ccatgaccccaagctcccct

SEQ ID NO: 687 - DNA, 20 bp, Artificial Sequence
  source: /note=“Description of Artificial Sequence Synthetic oligonucleotide”
cttcataatgcactttggag

SEQ ID NO: 688 - DNA, 20 bp, Artificial Sequence
  source: /note=“Description of Artificial Sequence Synthetic oligonucleotide”
ttcatgtgttcctgtagctc

SEQ ID NO: 689 - DNA, 20 bp, Artificial Sequence
  source: /note=“Description of Artificial Sequence Synthetic oligonucleotide”
ttctggaagatgctgcttct

SEQ ID NO: 690 - DNA, 20 bp, Artificial Sequence
  source: /note=“Description of Artificial Sequence Synthetic oligonucleotide”
cccaccagggtgtccagctc

SEQ ID NO: 691 - DNA, 20 bp, Artificial Sequence
  source: /note=“Description of Artificial Sequence Synthetic oligonucleotide”
agacagtggcagtcaagatc

SEQ ID NO: 692 - DNA, 20 bp, Artificial Sequence
  source: /note=“Description of Artificial Sequence Synthetic oligonucleotide”
cctgcgtctggaccctactc

SEQ ID NO: 693 - DNA, 20 bp, Artificial Sequence
  source: /note=“Description of Artificial Sequence Synthetic oligonucleotide”
cacaactgtgtaaattttgt

SEQ ID NO: 694 - DNA, 20 bp, Artificial Sequence
  source: /note=“Description of Artificial Sequence Synthetic oligonucleotide”
gagaagcagcatcttccaga

SEQ ID NO: 695 - DNA, 20 bp, Artificial Sequence
  source: /note=“Description of Artificial Sequence Synthetic oligonucleotide”
tggttgtcacaggtggaaaa

SEQ ID NO: 696 - DNA, 20 bp, Artificial Sequence
  source: /note=“Description of Artificial Sequence Synthetic oligonucleotide”
ccaggttgaactcagcttct

SEQ ID NO: 697 - DNA, 21 bp, Artificial Sequence
  source: /note=“Description of Artificial Sequence Synthetic oligonucleotide”
atcacaaaatttacacagttg

SEQ ID NO: 698 - DNA, 21 bp, Artificial Sequence
  source: /note=“Description of Artificial Sequence Synthetic oligonucleotide”
ggcatcagcctcctgccacca

SEQ ID NO: 699 - DNA, 21 bp, Artificial Sequence
  source: /note=“Description of Artificial Sequence Synthetic oligonucleotide”
gttagccaggtcatccacaga

SEQ ID NO: 700 - DNA, 21 bp, Artificial Sequence
  source: /note=“Description of Artificial Sequence Synthetic oligonucleotide”
gctgggcagctccctcgcccg

SEQ ID NO: 701 - DNA, 21 bp, Artificial Sequence
  source: /note=“Description of Artificial Sequence Synthetic oligonucleotide”
caggaggctgatgcctgtcac

SEQ ID NO: 702 - DNA, 21 bp, Artificial Sequence
  source: /note=“Description of Artificial Sequence Synthetic oligonucleotide”
gaggagcggaagacggagttg

SEQ ID NO: 703 - DNA, 21 bp, Artificial Sequence
  source: /note=“Description of Artificial Sequence Synthetic oligonucleotide”
cgtctggaccctactctgtct

SEQ ID NO: 704 - DNA, 21 bp, Artificial Sequence
  source: /note=“Description of Artificial Sequence Synthetic oligonucleotide”
tttttccttcataatgcactt

SEQ ID NO: 705 - DNA, 21 bp, Artificial Sequence
  source: /note=“Description of Artificial Sequence Synthetic oligonucleotide”
ccattgagctggacaccctgg

SEQ ID NO: 706 - DNA, 21 bp, Artificial Sequence
  source: /note=“Description of Artificial Sequence Synthetic oligonucleotide”
cttctccaaagtgcattatga

SEQ ID NO: 707 - DNA, 21 bp, Artificial Sequence
  source: /note=“Description of Artificial Sequence Synthetic oligonucleotide”
gcccaagatgcccatcgtgca

SEQ ID NO: 708 - DNA, 21 bp, Artificial Sequence
  source: /note=“Description of Artificial Sequence Synthetic oligonucleotide”
tcatgtgttcctgtagctctg

SEQ ID NO: 709 - DNA, 21 bp, Artificial Sequence
  source: /note=“Description of Artificial Sequence Synthetic oligonucleotide”
gtgatgctgcagttgctcatg

SEQ ID NO: 710 - DNA, 21 bp, Artificial Sequence
  source: /note=“Description of Artificial Sequence Synthetic oligonucleotide”
tctcatgcttcagattgatgt

SEQ ID NO: 711 - DNA, 21 bp, Artificial Sequence
  source: /note=“Description of Artificial Sequence Synthetic oligonucleotide”
tccctatgaggagtatgcctc

SEQ ID NO: 712 - DNA, 21 bp, Artificial Sequence
  source: /note=“Description of Artificial Sequence Synthetic oligonucleotide”
catcacaaaatttacacagtt

SEQ ID NO: 713 - DNA, 21 bp, Artificial Sequence
  source: /note=“Description of Artificial Sequence Synthetic oligonucleotide”
attgagctggacaccctggtg

SEQ ID NO: 714 - DNA, 21 bp, Artificial Sequence
  source: /note=“Description of Artificial Sequence Synthetic oligonucleotide”
cagtcaagatctttccctatg

SEQ ID NO: 715 - DNA, 21 bp, Artificial Sequence
  source: /note=“Description of Artificial Sequence Synthetic oligonucleotide”
aggatttctggttgtcacagg

SEQ ID NO: 716 - DNA, 21 bp, Artificial Sequence
  source: /note=“Description of Artificial Sequence Synthetic oligonucleotide”
tccacagtgatcacactccat

SEQ ID NO: 717 - DNA, 21 bp, Artificial Sequence
  source: /note=“Description of Artificial Sequence Synthetic oligonucleotide”
agcagaacacttcagagcagt

SEQ ID NO: 718 - DNA, 21 bp, Artificial Sequence
  source: /note=“Description of Artificial Sequence Synthetic oligonucleotide”
ccggcaagacgcggaagctca

SEQ ID NO: 719 - DNA, 21 bp, Artificial Sequence
  source: /note=“Description of Artificial Sequence Synthetic oligonucleotide”
gatgtcagagcggtcatcttc

SEQ ID NO: 720 - DNA, 21 bp, Artificial Sequence
  source: /note=“Description of Artificial Sequence Synthetic oligonucleotide”
tcattgcactcatcagagcta

SEQ ID NO: 721 - DNA, 21 bp, Artificial Sequence
  source: /note=“Description of Artificial Sequence Synthetic oligonucleotide”
cttccagaataaagtcatggt

SEQ ID NO: 722 - DNA, 21 bp, Artificial Sequence
  source: /note=“Description of Artificial Sequence Synthetic oligonucleotide”
agattttccacctgtgacaac

SEQ ID NO: 723 - DNA, 21 bp, Artificial Sequence
  source: /note=“Description of Artificial Sequence Synthetic oligonucleotide”
actgcagcatcacctccatct

SEQ ID NO: 724 - DNA, 21 bp, Artificial Sequence
  source: /note=“Description of Artificial Sequence Synthetic oligonucleotide”
agctgggcagctccctcgccc

SEQ ID NO: 725 - DNA, 21 bp, Artificial Sequence
  source: /note=“Description of Artificial Sequence Synthetic oligonucleotide”
tgacggctgaggagcggaaga

SEQ ID NO: 726 - DNA, 21 bp, Artificial Sequence
  source: /note=“Description of Artificial Sequence Synthetic oligonucleotide”
cattgagctggacaccctggt

SEQ ID NO: 727 - DNA, 21 bp, Artificial Sequence
  source: /note=“Description of Artificial Sequence Synthetic oligonucleotide”
agcaaagcgacctttccccac

SEQ ID NO: 728 - DNA, 21 bp, Artificial Sequence
  source: /note=“Description of Artificial Sequence Synthetic oligonucleotide”
cgcgttaaccggcagcagaag

SEQ ID NO: 729 - DNA, 21 bp, Artificial Sequence
  source: /note=“Description of Artificial Sequence Synthetic oligonucleotide”
gaaatatgactagcaacaagt

SEQ ID NO: 730 - DNA, 21 bp, Artificial Sequence
  source: /note=“Description of Artificial Sequence Synthetic oligonucleotide”
agacagagtagggtccagacg

SEQ ID NO: 731 - DNA, 21 bp, Artificial Sequence
  source: /note=“Description of Artificial Sequence Synthetic oligonucleotide”
caggatttctggttgtcacag

SEQ ID NO: 732 - DNA, 21 bp, Artificial Sequence
  source: /note=“Description of Artificial Sequence Synthetic oligonucleotide”
ctcctgtaggttgcccttggc

SEQ ID NO: 733 - DNA, 21 bp, Artificial Sequence
  source: /note=“Description of Artificial Sequence Synthetic oligonucleotide”
acagagtagggtccagacgca

SEQ ID NO: 734 - DNA, 21 bp, Artificial Sequence
  source: /note=“Description of Artificial Sequence Synthetic oligonucleotide”
gcttctccaaagtgcattatg

SEQ ID NO: 735 - DNA, 21 bp, Artificial Sequence
  source: /note=“Description of Artificial Sequence Synthetic oligonucleotide”
gcagcagaagctgagttcaac

SEQ ID NO: 736 - DNA, 21 bp, Artificial Sequence
  source: /note=“Description of Artificial Sequence Synthetic oligonucleotide”
tgaggagcggaagacggagtt

SEQ ID NO: 737 - DNA, 21 bp, Artificial Sequence
  source: /note=“Description of Artificial Sequence Synthetic oligonucleotide”
ctttggagaagcagcatcttc

SEQ ID NO: 738 - DNA, 21 bp, Artificial Sequence
  source: /note=“Description of Artificial Sequence Synthetic oligonucleotide”
ctcccctaccatgactttatt

SEQ ID NO: 739 - DNA, 21 bp, Artificial Sequence
  source: /note=“Description of Artificial Sequence Synthetic oligonucleotide”
gacagagtagggtccagacgc

SEQ ID NO: 740 - DNA, 21 bp, Artificial Sequence
  source: /note=“Description of Artificial Sequence Synthetic oligonucleotide”
ctgaggagcggaagacggagt

SEQ ID NO: 741 - DNA, 21 bp, Artificial Sequence
  source: /note=“Description of Artificial Sequence Synthetic oligonucleotide”
gggcatcttgggcctcccaca

SEQ ID NO: 742 - DNA, 21 bp, Artificial Sequence
  source: /note=“Description of Artificial Sequence Synthetic oligonucleotide”
ccaagaggcatactcctcata

SEQ ID NO: 743 - DNA, 21 bp, Artificial Sequence
  source: /note=“Description of Artificial Sequence Synthetic oligonucleotide”
agaatgacgagaacataacac

SEQ ID NO: 744 - DNA, 21 bp, Artificial Sequence
  source: /note=“Description of Artificial Sequence Synthetic oligonucleotide”
cctgacgcggcatgtcatcag

SEQ ID NO: 745 - DNA, 21 bp, Artificial Sequence
  source: /note=“Description of Artificial Sequence Synthetic oligonucleotide”
agcgagcactgtgccatcatc

SEQ ID NO: 746 - DNA, 21 bp, Artificial Sequence
  source: /note=“Description of Artificial Sequence Synthetic oligonucleotide”
gcaggttaggtcgttcttcac

SEQ ID NO: 747 - DNA, 21 bp, Artificial Sequence
  source: /note=“Description of Artificial Sequence Synthetic oligonucleotide”
acctccatctgtgagaagcca

SEQ ID NO: 748 - DNA, 21 bp, Artificial Sequence
  source: /note=“Description of Artificial Sequence Synthetic oligonucleotide”
taaagtcatggtaggggagct

SEQ ID NO: 749 - DNA, 21 bp, Artificial Sequence
  source: /note=“Description of Artificial Sequence Synthetic oligonucleotide”
tcagagctacaggaacacatg

SEQ ID NO: 750 - DNA, 21 bp, Artificial Sequence
  source: /note=“Description of Artificial Sequence Synthetic oligonucleotide”
tctcagacatcaatctgaagc

SEQ ID NO: 751 - DNA, 21 bp, Artificial Sequence
  source: /note=“Description of Artificial Sequence Synthetic oligonucleotide”
catcagcctcctgccaccact

SEQ ID NO: 752 - DNA, 21 bp, Artificial Sequence
  source: /note=“Description of Artificial Sequence Synthetic oligonucleotide”
cgctcctcagccgtcaggaac

SEQ ID NO: 753 - DNA, 21 bp, Artificial Sequence
  source: /note=“Description of Artificial Sequence Synthetic oligonucleotide”
aacctgggaaaccggcaagac

SEQ ID NO: 754 - DNA, 21 bp, Artificial Sequence
  source: /note=“Description of Artificial Sequence Synthetic oligonucleotide”
tccacgccaagggcaacctac

SEQ ID NO: 755 - DNA, 21 bp, Artificial Sequence
  source: /note=“Description of Artificial Sequence Synthetic oligonucleotide”
gaggtgagcaatcccccgggc

SEQ ID NO: 756 - DNA, 21 bp, Artificial Sequence
  source: /note=“Description of Artificial Sequence Synthetic oligonucleotide”
cagcagaagctgagttcaacc

SEQ ID NO: 757 - DNA, 21 bp, Artificial Sequence
  source: /note=“Description of Artificial Sequence Synthetic oligonucleotide”
tccaagaggcatactcctcat

SEQ ID NO: 758 - DNA, 21 bp, Artificial Sequence
  source: /note=“Description of Artificial Sequence Synthetic oligonucleotide”
agcagaagctgagttcaacct

SEQ ID NO: 759 - DNA, 21 bp, Artificial Sequence
  source: /note=“Description of Artificial Sequence Synthetic oligonucleotide”
ccagttcctgacggctgagga

SEQ ID NO: 760 - DNA, 21 bp, Artificial Sequence
  source: /note=“Description of Artificial Sequence Synthetic oligonucleotide”
aggagtatgcctcttggaaga

SEQ ID NO: 761 - DNA, 21 bp, Artificial Sequence
  source: /note=“Description of Artificial Sequence Synthetic oligonucleotide”
ttccaagaggcatactcctca

SEQ ID NO: 762 - DNA, 21 bp, Artificial Sequence
  source: /note=“Description of Artificial Sequence Synthetic oligonucleotide”
caactgtgtaaattttgtgat

SEQ ID NO: 763 - DNA, 21 bp, Artificial Sequence
  source: /note=“Description of Artificial Sequence Synthetic oligonucleotide”
tgaaggaaaaaaaaaagcctg

SEQ ID NO: 764 - DNA, 21 bp, Artificial Sequence
  source: /note=“Description of Artificial Sequence Synthetic oligonucleotide”
cgtcttccgctcctcagccgt

SEQ ID NO: 765 - DNA, 21 bp, Artificial Sequence
  source: /note=“Description of Artificial Sequence Synthetic oligonucleotide”
ccaggtcatccacagacagag

SEQ ID NO: 766 - DNA, 21 bp, Artificial Sequence
  source: /note=“Description of Artificial Sequence Synthetic oligonucleotide”
gcctagagtgaagagattcat

SEQ ID NO: 767 - DNA, 21 bp, Artificial Sequence
  source: /note=“Description of Artificial Sequence Synthetic oligonucleotide”
gttctccaaagtgcattatga

SEQ ID NO: 768 - DNA, 21 bp, Artificial Sequence
  source: /note=“Description of Artificial Sequence Synthetic oligonucleotide”
gcatcttccagaataaagtca

SEQ ID NO: 769 - DNA, 20 bp, Artificial Sequence
  source: /note=“Description of Artificial Sequence Synthetic oligonucleotide”
caaccgtctggtggccgacg

SEQ ID NO: 770 - DNA, 20 bp, Artificial Sequence
  source: /note=“Description of Artificial Sequence Synthetic oligonucleotide”
caggatcggggctgtcgctt

SEQ ID NO: 771 - DNA, 20 bp, Artificial Sequence
  source: /note=“Description of Artificial Sequence Synthetic oligonucleotide”
tcgggcctcgctggccgtaa

SEQ ID NO: 772 - DNA, 20 bp, Artificial Sequence
  source: /note=“Description of Artificial Sequence Synthetic oligonucleotide”
gaggtagtcggccatgcgcc

SEQ ID NO: 773 - DNA, 20 bp, Artificial Sequence
  source: /note=“Description of Artificial Sequence Synthetic oligonucleotide”
caggtgttgtcgggcctcgc

SEQ ID NO: 774 - DNA, 20 bp, Artificial Sequence
  source: /note=“Description of Artificial Sequence Synthetic oligonucleotide”
ggaggtagtcggccatgcgc

SEQ ID NO: 775 - DNA, 20 bp, Artificial Sequence
  source: /note=“Description of Artificial Sequence Synthetic oligonucleotide”
ggcatactcaatgcgtacat

SEQ ID NO: 776 - DNA, 20 bp, Artificial Sequence
  source: /note=“Description of Artificial Sequence Synthetic oligonucleotide”
ccgccttgtcatcaaccgtc

SEQ ID NO: 777 - DNA, 20 bp, Artificial Sequence
  source: /note=“Description of Artificial Sequence Synthetic oligonucleotide”
aggatcggggctgtcgcttc

SEQ ID NO: 778 - DNA, 20 bp, Artificial Sequence
  source: /note=“Description of Artificial Sequence Synthetic oligonucleotide”
ccttgtcatcaaccgtctgg

SEQ ID NO: 779 - DNA, 20 bp, Artificial Sequence
  source: /note=“Description of Artificial Sequence Synthetic oligonucleotide”
tactcaatgcgtacattggt

SEQ ID NO: 780 - DNA, 20 bp, Artificial Sequence
  source: /note=“Description of Artificial Sequence Synthetic oligonucleotide”
gggttccattacggccagcg

SEQ ID NO: 781 - DNA, 20 bp, Artificial Sequence
  source: /note=“Description of Artificial Sequence Synthetic oligonucleotide”
ggcactgcttctgcgtacaa

SEQ ID NO: 782 - DNA, 20 bp, Artificial Sequence
  source: /note=“Description of Artificial Sequence Synthetic oligonucleotide”
ggttgatgacaaggcggcac

SEQ ID NO: 783 - DNA, 20 bp, Artificial Sequence
  source: /note=“Description of Artificial Sequence Synthetic oligonucleotide”
tgctggggccttcctcgagg

SEQ ID NO: 784 - DNA, 20 bp, Artificial Sequence
  source: /note=“Description of Artificial Sequence Synthetic oligonucleotide”
ttgctggctgtggagcggac

SEQ ID NO: 785 - DNA, 20 bp, Artificial Sequence
  source: /note=“Description of Artificial Sequence Synthetic oligonucleotide”
ttctcctaccttcgggaatc

SEQ ID NO: 786 - DNA, 20 bp, Artificial Sequence
  source: /note=“Description of Artificial Sequence Synthetic oligonucleotide”
gactggcttgggcagttcca

SEQ ID NO: 787 - DNA, 20 bp, Artificial Sequence
  source: /note=“Description of Artificial Sequence Synthetic oligonucleotide”
catgcagcccttgcctgctg

SEQ ID NO: 788 - DNA, 20 bp, Artificial Sequence
  source: /note=“Description of Artificial Sequence Synthetic oligonucleotide”
agcaaaggacgaggtctaga

SEQ ID NO: 789 - DNA, 20 bp, Artificial Sequence
  source: /note=“Description of Artificial Sequence Synthetic oligonucleotide”
gcctgctggggccttcctcg

SEQ ID NO: 790 - DNA, 20 bp, Artificial Sequence
  source: /note=“Description of Artificial Sequence Synthetic oligonucleotide”
cagactcaccagattcccga

SEQ ID NO: 791 - DNA, 20 bp, Artificial Sequence
  source: /note=“Description of Artificial Sequence Synthetic oligonucleotide”
acctcgtcctttgctggctg

SEQ ID NO: 792 - DNA, 20 bp, Artificial Sequence
  source: /note=“Description of Artificial Sequence Synthetic oligonucleotide”
ctcaccagattcccgaaggt

SEQ ID NO: 793 - DNA, 20 bp, Artificial Sequence
  source: /note=“Description of Artificial Sequence Synthetic oligonucleotide”
tacgcagaagcagtgcccgc

SEQ ID NO: 794 - DNA, 20 bp, Artificial Sequence
  source: /note=“Description of Artificial Sequence Synthetic oligonucleotide”
aggtgtacagcagtggctgg

SEQ ID NO: 795 - DNA, 20 bp, Artificial Sequence
  source: /note=“Description of Artificial Sequence Synthetic oligonucleotide”
ggtgtacagcagtggctggt

SEQ ID NO: 796 - DNA, 20 bp, Artificial Sequence
  source: /note=“Description of Artificial Sequence Synthetic oligonucleotide”
cggatgtggtcagccttgtg

SEQ ID NO: 797 - DNA, 20 bp, Artificial Sequence
  source: /note=“Description of Artificial Sequence Synthetic oligonucleotide”
cactgacagcgtgaacaggt

SEQ ID NO: 798 - DNA, 20 bp, Artificial Sequence
  source: /note=“Description of Artificial Sequence Synthetic oligonucleotide”
actgacagcgtgaacaggta

SEQ ID NO: 799 - DNA, 20 bp, Artificial Sequence
  source: /note=“Description of Artificial Sequence Synthetic oligonucleotide”
gctcactctctgtctgggct

SEQ ID NO: 800 - DNA, 20 bp, Artificial Sequence
  source: /note=“Description of Artificial Sequence Synthetic oligonucleotide”
ctggctgtggagcggactgg

SEQ ID NO: 801 - DNA, 20 bp, Artificial Sequence
  source: /note=“Description of Artificial Sequence Synthetic oligonucleotide”
gctctgactgtacggggcaa

SEQ ID NO: 802 - DNA, 20 bp, Artificial Sequence
  source: /note=“Description of Artificial Sequence Synthetic oligonucleotide”
agctctgactgtacggggca

SEQ ID NO: 803 - DNA, 20 bp, Artificial Sequence
  source: /note=“Description of Artificial Sequence Synthetic oligonucleotide”
acagtaccccttccagctct

SEQ ID NO: 804 - DNA, 20 bp, Artificial Sequence
  source: /note=“Description of Artificial Sequence Synthetic oligonucleotide”
cgtcggccaccagacggttg

SEQ ID NO: 805 - DNA, 20 bp, Artificial Sequence
  source: /note=“Description of Artificial Sequence Synthetic oligonucleotide”
ccagccactgctgtacacct

SEQ ID NO: 806 - DNA, 20 bp, Artificial Sequence
  source: /note=“Description of Artificial Sequence Synthetic oligonucleotide”
accccggccctgcctatgcc

SEQ ID NO: 807 - DNA, 20 bp, Artificial Sequence
  source: /note=“Description of Artificial Sequence Synthetic oligonucleotide”
ggtatcagcagtgcaggagg

SEQ ID NO: 808 - DNA, 20 bp, Artificial Sequence
  source: /note=“Description of Artificial Sequence Synthetic oligonucleotide”
gatgtggtcagccttgtgca

SEQ ID NO: 809 - DNA, 20 bp, Artificial Sequence
  source: /note=“Description of Artificial Sequence Synthetic oligonucleotide”
ggatgtggtcagccttgtgc

SEQ ID NO: 810 - DNA, 20 bp, Artificial Sequence
  source: /note=“Description of Artificial Sequence Synthetic oligonucleotide”
ggccacgcatcctggccttt

SEQ ID NO: 811 - DNA, 20 bp, Artificial Sequence
  source: /note=“Description of Artificial Sequence Synthetic oligonucleotide”
gaaaggccaggatgcgtggc

SEQ ID NO: 812 - DNA, 20 bp, Artificial Sequence
  source: /note=“Description of Artificial Sequence Synthetic oligonucleotide”
actgcttgtccaggccacgc

SEQ ID NO: 813 - DNA, 20 bp, Artificial Sequence
  source: /note=“Description of Artificial Sequence Synthetic oligonucleotide”
tctggactccaactgcttgt

SEQ ID NO: 814 - DNA, 20 bp, Artificial Sequence
  source: /note=“Description of Artificial Sequence Synthetic oligonucleotide”
gtctggactccaactgcttg

SEQ ID NO: 815 - DNA, 20 bp, Artificial Sequence
  source: /note=“Description of Artificial Sequence Synthetic oligonucleotide”
gcttccgtctggactccaac

SEQ ID NO: 816 - DNA, 20 bp, Artificial Sequence
  source: /note=“Description of Artificial Sequence Synthetic oligonucleotide”
gacggaagctggagtcggca

SEQ ID NO: 817 - DNA, 20 bp, Artificial Sequence
  source: /note=“Description of Artificial Sequence Synthetic oligonucleotide”
cgctgtcagtgaaaaccact

SEQ ID NO: 818 - DNA, 20 bp, Artificial Sequence
  source: /note=“Description of Artificial Sequence Synthetic oligonucleotide”
ctgacagcgtgaacaggtag

SEQ ID NO: 819 - DNA, 20 bp, Artificial Sequence
  source: /note=“Description of Artificial Sequence Synthetic oligonucleotide”
ttacggccagcgaggcccga

SEQ ID NO: 820 - DNA, 20 bp, Artificial Sequence
  source: /note=“Description of Artificial Sequence Synthetic oligonucleotide”
attacggccagcgaggcccg

SEQ ID NO: 821 - DNA, 20 bp, Artificial Sequence
  source: /note=“Description of Artificial Sequence Synthetic oligonucleotide”
ggaatctggtgagtctgagg

SEQ ID NO: 822 - DNA, 20 bp, Artificial Sequence
  source: /note=“Description of Artificial Sequence Synthetic oligonucleotide”
ccctcagactcaccagattc

SEQ ID NO: 823 - DNA, 20 bp, Artificial Sequence
  source: /note=“Description of Artificial Sequence Synthetic oligonucleotide”
cgaaggtaggagaaggtctt

SEQ ID NO: 824 - DNA, 20 bp, Artificial Sequence
  source: /note=“Description of Artificial Sequence Synthetic oligonucleotide”
gaaggtaggagaaggtcttg

SEQ ID NO: 825 - DNA, 20 bp, Artificial Sequence
  source: /note=“Description of Artificial Sequence Synthetic oligonucleotide”
gcacctttggctcactctct

SEQ ID NO: 826 - DNA, 20 bp, Artificial Sequence
  source: /note=“Description of Artificial Sequence Synthetic oligonucleotide”
tcgaggaggtggcagagggt

SEQ ID NO: 827 - DNA, 20 bp, Artificial Sequence
  source: /note=“Description of Artificial Sequence Synthetic oligonucleotide”
tggaactgcccaagccagtc

SEQ ID NO: 828 - DNA, 20 bp, Artificial Sequence
  source: /note=“Description of Artificial Sequence Synthetic oligonucleotide”
agggacggggcccacagggg

SEQ ID NO: 829 - DNA, 20 bp, Artificial Sequence
  source: /note=“Description of Artificial Sequence Synthetic oligonucleotide”
gggacggggcccacaggggc

SEQ ID NO: 830 - DNA, 20 bp, Artificial Sequence
  source: /note=“Description of Artificial Sequence Synthetic oligonucleotide”
ctccacagccagcaaaggac

SEQ ID NO: 831 - DNA, 20 bp, Artificial Sequence
  source: /note=“Description of Artificial Sequence Synthetic oligonucleotide”
cagccagcaaaggacgaggt

SEQ ID NO: 832 - DNA, 20 bp, Artificial Sequence
  source: /note=“Description of Artificial Sequence Synthetic oligonucleotide”
ctgccttctagacctcgtcc

SEQ ID NO: 833 - DNA, 20 bp, Artificial Sequence
  source: /note=“Description of Artificial Sequence Synthetic oligonucleotide”
cctaaggaggatgcgcctag

SEQ ID NO: 834 - DNA, 20 bp, Artificial Sequence
  source: /note=“Description of Artificial Sequence Synthetic oligonucleotide”
tggcctcctgcactgctgat

SEQ ID NO: 835 - DNA, 20 bp, Artificial Sequence
  source: /note=“Description of Artificial Sequence Synthetic oligonucleotide”
agcagtgcaggaggccacat

SEQ ID NO: 836 - DNA, 20 bp, Artificial Sequence
  source: /note=“Description of Artificial Sequence Synthetic oligonucleotide”
ccgactccagcttccgtctg

SEQ ID NO: 837 - DNA, 20 bp, Artificial Sequence
  source: /note=“Description of Artificial Sequence Synthetic oligonucleotide”
ggggttccattacggccagc

SEQ ID NO: 838 - DNA, 20 bp, Artificial Sequence
  source: /note=“Description of Artificial Sequence Synthetic oligonucleotide”
cacagcagatcctcctctgg

SEQ ID NO: 839 - DNA, 20 bp, Artificial Sequence
  source: /note=“Description of Artificial Sequence Synthetic oligonucleotide”
attgccccgtacagtcagag

SEQ ID NO: 840 - DNA, 20 bp, Artificial Sequence
  source: /note=“Description of Artificial Sequence Synthetic oligonucleotide”
cccgtacagtcagagctgga

SEQ ID NO: 841 - DNA, 20 bp, Artificial Sequence
  source: /note=“Description of Artificial Sequence Synthetic oligonucleotide”
tggtggaggagcaggcagtg

SEQ ID NO: 842 - DNA, 20 bp, Artificial Sequence
  source: /note=“Description of Artificial Sequence Synthetic oligonucleotide”
tccttaggcataggcagggc

SEQ ID NO: 843 - DNA, 20 bp, Artificial Sequence
  source: /note=“Description of Artificial Sequence Synthetic oligonucleotide”
cggccctgcctatgcctaag

SEQ ID NO: 844 - DNA, 20 bp, Artificial Sequence
  source: /note=“Description of Artificial Sequence Synthetic oligonucleotide”
taggcataggcagggccggg

SEQ ID NO: 845 - DNA, 20 bp, Artificial Sequence
  source: /note=“Description of Artificial Sequence Synthetic oligonucleotide”
aggcagggccggggtgggag

SEQ ID NO: 846 - DNA, 20 bp, Artificial Sequence
  source: /note=“Description of Artificial Sequence Synthetic oligonucleotide”
gcaggatcggggctgtcgct

SEQ ID NO: 847 - DNA, 20 bp, Artificial Sequence
  source: /note=“Description of Artificial Sequence Synthetic oligonucleotide”
ctgcacaaggctgaccacat

SEQ ID NO: 848 - DNA, 20 bp, Artificial Sequence
  source: /note=“Description of Artificial Sequence Synthetic oligonucleotide”
tgcacaaggctgaccacatc

SEQ ID NO: 849 - DNA, 20 bp, Artificial Sequence
  source: /note=“Description of Artificial Sequence Synthetic oligonucleotide”
ctgaccacatccggaaaggc

SEQ ID NO: 850 - DNA, 20 bp, Artificial Sequence
  source: /note=“Description of Artificial Sequence Synthetic oligonucleotide”
ggccacgcatcctggccttt

SEQ ID NO: 851 - DNA, 20 bp, Artificial Sequence
  source: /note=“Description of Artificial Sequence Synthetic oligonucleotide”
gcgtggcctggacaagcagt

SEQ ID NO: 852 - DNA, 20 bp, Artificial Sequence
  source: /note=“Description of Artificial Sequence Synthetic oligonucleotide”
gacaagcagttggagtccag

SEQ ID NO: 853 - DNA, 20 bp, Artificial Sequence
  source: /note=“Description of Artificial Sequence Synthetic oligonucleotide”
gttggagtccagacggaagc

SEQ ID NO: 854 - DNA, 20 bp, Artificial Sequence
  source: /note=“Description of Artificial Sequence Synthetic oligonucleotide”
atgcgtacattggtggggcc

SEQ ID NO: 855 - DNA, 20 bp, Artificial Sequence
  source: /note=“Description of Artificial Sequence Synthetic oligonucleotide”
tggccccaccaatgtacgca

SEQ ID NO: 856 - DNA, 20 bp, Artificial Sequence
  source: /note=“Description of Artificial Sequence Synthetic oligonucleotide”
gctacctgttcacgctgtca

SEQ ID NO: 857 - DNA, 20 bp, Artificial Sequence
  source: /note=“Description of Artificial Sequence Synthetic oligonucleotide”
tgacagcgtgaacaggtagc

SEQ ID NO: 858 - DNA, 20 bp, Artificial Sequence
  source: /note=“Description of Artificial Sequence Synthetic oligonucleotide”
gtcgggcctcgctggccgta

SEQ ID NO: 859 - DNA, 20 bp, Artificial Sequence
  source: /note=“Description of Artificial Sequence Synthetic oligonucleotide”
gcacttgcctaggctggtat

SEQ ID NO: 860 - DNA, 20 bp, Artificial Sequence
  source: /note=“Description of Artificial Sequence Synthetic oligonucleotide”
gggaatctggtgagtctgag

SEQ ID NO: 861 - DNA, 20 bp, Artificial Sequence
  source: /note=“Description of Artificial Sequence Synthetic oligonucleotide”
ctcaccagattcccgaaggt

SEQ ID NO: 862 - DNA, 20 bp, Artificial Sequence
  source: /note=“Description of Artificial Sequence Synthetic oligonucleotide”
ctcctaccttcgggaatctg

SEQ ID NO: 863 - DNA, 20 bp, Artificial Sequence
  source: /note=“Description of Artificial Sequence Synthetic oligonucleotide”
caagaccttctcctaccttc

SEQ ID NO: 864 - DNA, 20 bp, Artificial Sequence
  source: /note=“Description of Artificial Sequence Synthetic oligonucleotide”
ccaagaccttctcctacctt

SEQ ID NO: 865 - DNA, 20 bp, Artificial Sequence
  source: /note=“Description of Artificial Sequence Synthetic oligonucleotide”
gccaagaccttctcctacct

SEQ ID NO: 866 - DNA, 20 bp, Artificial Sequence
  source: /note=“Description of Artificial Sequence Synthetic oligonucleotide”
tatgcacagcagatcctcct

SEQ ID NO: 867 - DNA, 20 bp, Artificial Sequence
  source: /note=“Description of Artificial Sequence Synthetic oligonucleotide”
caaaggtgctggacccagag

SEQ ID NO: 868 - DNA, 20 bp, Artificial Sequence
  source: /note=“Description of Artificial Sequence Synthetic oligonucleotide”
ggctcactctctgtctgggc

SEQ ID NO: 869 - DNA, 20 bp, Artificial Sequence
  source: /note=“Description of Artificial Sequence Synthetic oligonucleotide”
agggtaccccagcccagaca

SEQ ID NO: 870 - DNA, 20 bp, Artificial Sequence
  source: /note=“Description of Artificial Sequence Synthetic oligonucleotide”
agagggtaccccagcccaga

SEQ ID NO: 871 - DNA, 20 bp, Artificial Sequence
  source: /note=“Description of Artificial Sequence Synthetic oligonucleotide”
gtaccctctgccacctcctc

SEQ ID NO: 872 - DNA, 20 bp, Artificial Sequence
  source: /note=“Description of Artificial Sequence Synthetic oligonucleotide”
ccttcctcgaggaggtggca

SEQ ID NO: 873 - DNA, 20 bp, Artificial Sequence
  source: /note=“Description of Artificial Sequence Synthetic oligonucleotide”
atgactggcttgggcagttc

SEQ ID NO: 874 - DNA, 20 bp, Artificial Sequence
  source: /note=“Description of Artificial Sequence Synthetic oligonucleotide”
ggcccctgtgggccccgtcc

SEQ ID NO: 875 - DNA, 20 bp, Artificial Sequence
  source: /note=“Description of Artificial Sequence Synthetic oligonucleotide”
aggacgaggtctagaaggca

SEQ ID NO: 876 - DNA, 20 bp, Artificial Sequence
  source: /note=“Description of Artificial Sequence Synthetic oligonucleotide”
tataagtggaggcgtcgcgc

SEQ ID NO: 877 - DNA, 20 bp, Artificial Sequence
  source: /note=“Description of Artificial Sequence Synthetic oligonucleotide”
gggcacgcgtttaatataag

SEQ ID NO: 878 - DNA, 20 bp, Artificial Sequence
  source: /note=“Description of Artificial Sequence Synthetic oligonucleotide”
actcacgctggatagcctcc

SEQ ID NO: 879 - DNA, 20 bp, Artificial Sequence
  source: /note=“Description of Artificial Sequence Synthetic oligonucleotide”
ggccgagatgtctcgctccg

SEQ ID NO: 880 - DNA, 20 bp, Artificial Sequence
  source: /note=“Description of Artificial Sequence Synthetic oligonucleotide”
cacgcgtttaatataagtgg

SEQ ID NO: 881 - DNA, 20 bp, Artificial Sequence
  source: /note=“Description of Artificial Sequence Synthetic oligonucleotide”
aagtggaggcgtcgcgctgg

SEQ ID NO: 882 - DNA, 20 bp, Artificial Sequence
  source: /note=“Description of Artificial Sequence Synthetic oligonucleotide”
gagtagcgcgagcacagcta

SEQ ID NO: 883 - DNA, 20 bp, Artificial Sequence
  source: /note=“Description of Artificial Sequence Synthetic oligonucleotide”
agtggaggcgtcgcgctggc

SEQ ID NO: 884 - DNA, 20 bp, Artificial Sequence
  source: /note=“Description of Artificial Sequence Synthetic oligonucleotide”
gcccgaatgctgtcagcttc

SEQ ID NO: 885 - DNA, 20 bp, Artificial Sequence
  source: /note=“Description of Artificial Sequence Synthetic oligonucleotide”
cgcgagcacagctaaggcca

SEQ ID NO: 886 - DNA, 20 bp, Artificial Sequence
  source: /note=“Description of Artificial Sequence Synthetic oligonucleotide”
ctcgcgctactctctctttc

SEQ ID NO: 887 - DNA, 20 bp, Artificial Sequence
  source: /note=“Description of Artificial Sequence Synthetic oligonucleotide”
ggccacggagcgagacatct

SEQ ID NO: 888 - DNA, 20 bp, Artificial Sequence
  source: /note=“Description of Artificial Sequence Synthetic oligonucleotide”
cgtgagtaaacctgaatctt

SEQ ID NO: 889 - DNA, 20 bp, Artificial Sequence
  source: /note=“Description of Artificial Sequence Synthetic oligonucleotide”
agtcacatggttcacacggc

SEQ ID NO: 890 - DNA, 20 bp, Artificial Sequence
  source: /note=“Description of Artificial Sequence Synthetic oligonucleotide”
aagtcaacttcaatgtcgga

SEQ ID NO: 891 - DNA, 20 bp, Artificial Sequence
  source: /note=“Description of Artificial Sequence Synthetic oligonucleotide”
cagtaagtcaacttcaatgt

SEQ ID NO: 892 - DNA, 20 bp, Artificial Sequence
  source: /note=“Description of Artificial Sequence Synthetic oligonucleotide”
acccagacacatagcaattc

SEQ ID NO: 893 - DNA, 20 bp, Artificial Sequence
  source: /note=“Description of Artificial Sequence Synthetic oligonucleotide”
gcatactcatctttttcagt

SEQ ID NO: 894 - DNA, 20 bp, Artificial Sequence
  source: /note=“Description of Artificial Sequence Synthetic oligonucleotide”
acagcccaagatagttaagt

SEQ ID NO: 895 - DNA, 20 bp, Artificial Sequence
  source: /note=“Description of Artificial Sequence Synthetic oligonucleotide”
ggcatactcatctttttcag

SEQ ID NO: 896 - DNA, 20 bp, Artificial Sequence
  source: /note=“Description of Artificial Sequence Synthetic oligonucleotide”
ttcctgaagctgacagcatt

SEQ ID NO: 897 - DNA, 20 bp, Artificial Sequence
  source: /note=“Description of Artificial Sequence Synthetic oligonucleotide”
tcacgtcatccagcagagaa

SEQ ID NO: 898 - DNA, 20 bp, Artificial Sequence
  source: /note=“Description of Artificial Sequence Synthetic oligonucleotide”
cagcccaagatagttaagtg

SEQ ID NO: 899 - RNA, 18 nt, Artificial Sequence
  source: /note=“Description of Artificial Sequence Synthetic oligonucleotide”
aauucucucuccauucuu

SEQ ID NO: 900 - RNA, 19 nt, Artificial Sequence
  source: /note=“Description of Artificial Sequence Synthetic oligonucleotide”
aauucucucuccauucuuc

SEQ ID NO: 901 - RNA, 20 nt, Artificial Sequence
  source: /note=“Description of Artificial Sequence Synthetic oligonucleotide”
aauucucucuccauucuuca

SEQ ID NO: 902 - RNA, 21 nt, Artificial Sequence
  source: /note=“Description of Artificial Sequence Synthetic oligonucleotide”
aauucucucuccauucuucag

SEQ ID NO: 903 - RNA, 22 nt, Artificial Sequence
  source: /note=“Description of Artificial Sequence Synthetic oligonucleotide”
aauucucucuccauucuucagu

SEQ ID NO: 904 - RNA, 23 nt, Artificial Sequence
  source: /note=“Description of Artificial Sequence Synthetic oligonucleotide”
aauucucucuccauucuucagua

SEQ ID NO: 905 - RNA, 24 nt, Artificial Sequence
  source: /note=“Description of Artificial Sequence Synthetic oligonucleotide”
aauucucucuccauucuucaguaa

SEQ ID NO: 906 - RNA, 18 nt, Artificial Sequence
  source: /note=“Description of Artificial Sequence Synthetic oligonucleotide”
acuuuccauucucugcug

SEQ ID NO: 907 - RNA, 19 nt, Artificial Sequence
  source: /note=“Description of Artificial Sequence Synthetic oligonucleotide”
acuuuccauucucugcugg

SEQ ID NO: 908 - RNA, 20 nt, Artificial Sequence
  source: /note=“Description of Artificial Sequence Synthetic oligonucleotide”
acuuuccauucucugcugga

SEQ ID NO: 909 - RNA, 21 nt, Artificial Sequence
  source: /note=“Description of Artificial Sequence Synthetic oligonucleotide”
acuuuccauucucugcuggau

SEQ ID NO: 910 - RNA, 22 nt, Artificial Sequence
  source: /note=“Description of Artificial Sequence Synthetic oligonucleotide”
acuuuccauucucugcuggaug

SEQ ID NO: 911 - RNA, 23 nt, Artificial Sequence
  source: /note=“Description of Artificial Sequence Synthetic oligonucleotide”
acuuuccauucucugcuggauga

SEQ ID NO: 912 - RNA, 24 nt, Artificial Sequence
  source: /note=“Description of Artificial Sequence Synthetic oligonucleotide”
acuuuccauucucugcuggaugac

SEQ ID NO: 913 - RNA, 18 nt, Artificial Sequence
  source: /note=“Description of Artificial Sequence Synthetic oligonucleotide”
agcaaggacuggucuuuc

SEQ ID NO: 914 - RNA, 19 nt, Artificial Sequence
  source: /note=“Description of Artificial Sequence Synthetic oligonucleotide”
agcaaggacuggucuuucu

SEQ ID NO: 915 - RNA, 20 nt, Artificial Sequence
  source: /note=“Description of Artificial Sequence Synthetic oligonucleotide”
agcaaggacuggucuuucua

SEQ ID NO: 916 - RNA, 21 nt, Artificial Sequence
  source: /note=“Description of Artificial Sequence Synthetic oligonucleotide”
agcaaggacuggucuuucuau

SEQ ID NO: 917 - RNA, 22 nt, Artificial Sequence
  source: /note=“Description of Artificial Sequence Synthetic oligonucleotide”
agcaaggacuggucuuucuauc

SEQ ID NO: 918 - RNA, 23 nt, Artificial Sequence
  source: /note=“Description of Artificial Sequence Synthetic oligonucleotide”
agcaaggacuggucuuucuaucu

SEQ ID NO: 919 - RNA, 24 nt, Artificial Sequence
  source: /note=“Description of Artificial Sequence Synthetic oligonucleotide”
agcaaggacuggucuuucuaucuc

SEQ ID NO: 920 - RNA, 18 nt, Artificial Sequence
  source: /note=“Description of Artificial Sequence Synthetic oligonucleotide”
agugggggugaauucagu

SEQ ID NO: 921 - RNA, 19 nt, Artificial Sequence
  source: /note=“Description of Artificial Sequence Synthetic oligonucleotide”
agugggggugaauucagug

SEQ ID NO: 922 - RNA, 20 nt, Artificial Sequence
  source: /note=“Description of Artificial Sequence Synthetic oligonucleotide”
agugggggugaauucagugu

SEQ ID NO: 923 - RNA, 21 nt, Artificial Sequence
  source: /note=“Description of Artificial Sequence Synthetic oligonucleotide”
agugggggugaauucagugua

SEQ ID NO: 924 - RNA, 22 nt, Artificial Sequence
  source: /note=“Description of Artificial Sequence Synthetic oligonucleotide”
agugggggugaauucaguguag

SEQ ID NO: 925 - RNA, 23 nt, Artificial Sequence
  source: /note=“Description of Artificial Sequence Synthetic oligonucleotide”
agugggggugaauucaguguagu

SEQ ID NO: 926 - RNA, 24 nt, Artificial Sequence
  source: /note=“Description of Artificial Sequence Synthetic oligonucleotide”
agugggggugaauucaguguagua

SEQ ID NO: 927 - RNA, 18 nt, Artificial Sequence
  source: /note=“Description of Artificial Sequence Synthetic oligonucleotide”
auccauccgacauugaag

SEQ ID NO: 928 - RNA, 19 nt, Artificial Sequence
  source: /note=“Description of Artificial Sequence Synthetic oligonucleotide”
auccauccgacauugaagu

SEQ ID NO: 929 - RNA, 20 nt, Artificial Sequence
  source: /note=“Description of Artificial Sequence Synthetic oligonucleotide”
auccauccgacauugaaguu

SEQ ID NO: 930 - RNA, 21 nt, Artificial Sequence
  source: /note=“Description of Artificial Sequence Synthetic oligonucleotide”
auccauccgacauugaaguug

SEQ ID NO: 931 - RNA, 22 nt, Artificial Sequence
  source: /note=“Description of Artificial Sequence Synthetic oligonucleotide”
auccauccgacauugaaguuga

SEQ ID NO: 932 - RNA, 23 nt, Artificial Sequence
  source: /note=“Description of Artificial Sequence Synthetic oligonucleotide”
auccauccgacauugaaguugac

SEQ ID NO: 933 - RNA, 24 nt, Artificial Sequence
  source: /note=“Description of Artificial Sequence Synthetic oligonucleotide”
auccauccgacauugaaguugacu

SEQ ID NO: 934 - RNA, 18 nt, Artificial Sequence
  source: /note=“Description of Artificial Sequence Synthetic oligonucleotide”
caauucucucuccauucu

SEQ ID NO: 935 - RNA, 19 nt, Artificial Sequence
  source: /note=“Description of Artificial Sequence Synthetic oligonucleotide”
caauucucucuccauucuu

SEQ ID NO: 936 - RNA, 20 nt, Artificial Sequence
  source: /note=“Description of Artificial Sequence Synthetic oligonucleotide”
caauucucucuccauucuuc

SEQ ID NO: 937 - RNA, 21 nt, Artificial Sequence
  source: /note=“Description of Artificial Sequence Synthetic oligonucleotide”
caauucucucuccauucuuca

SEQ ID NO: 938 - RNA, 22 nt, Artificial Sequence
  source: /note=“Description of Artificial Sequence Synthetic oligonucleotide”
caauucucucuccauucuucag

SEQ ID NO: 939 - RNA, 23 nt, Artificial Sequence
  source: /note=“Description of Artificial Sequence Synthetic oligonucleotide”
caauucucucuccauucuucagu

SEQ ID NO: 940 - RNA, 24 nt, Artificial Sequence
  source: /note=“Description of Artificial Sequence Synthetic oligonucleotide”
caauucucucuccauucuucagua

SEQ ID NO: 941 - RNA, 18 nt, Artificial Sequence
  source: /note=“Description of Artificial Sequence Synthetic oligonucleotide”
cagugggggugaauucag

SEQ ID NO: 942 - RNA, 19 nt, Artificial Sequence
  source: /note=“Description of Artificial Sequence Synthetic oligonucleotide”
cagugggggugaauucagu

SEQ ID NO: 943 - RNA, 20 nt, Artificial Sequence
  source: /note=“Description of Artificial Sequence Synthetic oligonucleotide”
cagugggggugaauucagug

SEQ ID NO: 944 - RNA, 21 nt, Artificial Sequence
  source: /note=“Description of Artificial Sequence Synthetic oligonucleotide”
cagugggggugaauucagugu

SEQ ID NO: 945 - RNA, 22 nt, Artificial Sequence
  source: /note=“Description of Artificial Sequence Synthetic oligonucleotide”
cagugggggugaauucagugua

SEQ ID NO: 946 - RNA, 23 nt, Artificial Sequence
  source: /note=“Description of Artificial Sequence Synthetic oligonucleotide”
cagugggggugaauucaguguag

SEQ ID NO: 947 - RNA, 24 nt, Artificial Sequence
  source: /note=“Description of Artificial Sequence Synthetic oligonucleotide”
cagugggggugaauucaguguagu

SEQ ID NO: 948 - RNA, 18 nt, Artificial Sequence
  source: /note=“Description of Artificial Sequence Synthetic oligonucleotide”
cauucucugcuggaugac

SEQ ID NO: 949 - RNA, 19 nt, Artificial Sequence
  source: /note=“Description of Artificial Sequence Synthetic oligonucleotide”
cauucucugcuggaugacg

SEQ ID NO: 950 - RNA, 20 nt, Artificial Sequence
  source: /note=“Description of Artificial Sequence Synthetic oligonucleotide”
cauucucugcuggaugacgu

SEQ ID NO: 951 - RNA, 21 nt, Artificial Sequence
  source: /note=“Description of Artificial Sequence Synthetic oligonucleotide”
cauucucugcuggaugacgug

SEQ ID NO: 952 - RNA, 22 nt, Artificial Sequence
  source: /note=“Description of Artificial Sequence Synthetic oligonucleotide”
cauucucugcuggaugacguga

SEQ ID NO: 953 - RNA, 23 nt, Artificial Sequence
  source: /note=“Description of Artificial Sequence Synthetic oligonucleotide”
cauucucugcuggaugacgugag

SEQ ID NO: 954 - RNA, 24 nt, Artificial Sequence
  source: /note=“Description of Artificial Sequence Synthetic oligonucleotide”
cauucucugcuggaugacgugagu

SEQ ID NO: 955 - RNA, 18 nt, Artificial Sequence
  source: /note=“Description of Artificial Sequence Synthetic oligonucleotide”
cccgauauuccucaggua

SEQ ID NO: 956 - RNA, 19 nt, Artificial Sequence
  source: /note=“Description of Artificial Sequence Synthetic oligonucleotide”
cccgauauuccucagguac

SEQ ID NO: 957 - RNA, 20 nt, Artificial Sequence
  source: /note=“Description of Artificial Sequence Synthetic oligonucleotide”
cccgauauuccucagguacu

SEQ ID NO: 958 - RNA, 21 nt, Artificial Sequence
  source: /note=“Description of Artificial Sequence Synthetic oligonucleotide”
cccgauauuccucagguacuc

SEQ ID NO: 959 - RNA, 22 nt, Artificial Sequence
  source: /note=“Description of Artificial Sequence Synthetic oligonucleotide”
cccgauauuccucagguacucc

SEQ ID NO: 960 - RNA, 23 nt, Artificial Sequence
  source: /note=“Description of Artificial Sequence Synthetic oligonucleotide”
cccgauauuccucagguacucca

SEQ ID NO: 961 - RNA, 24 nt, Artificial Sequence
  source: /note=“Description of Artificial Sequence Synthetic oligonucleotide”
cccgauauuccucagguacuccaa

SEQ ID NO: 962 - RNA, 18 nt, Artificial Sequence
  source: /note=“Description of Artificial Sequence Synthetic oligonucleotide”
ccgauauuccucagguac

SEQ ID NO: 963 - RNA, 19 nt, Artificial Sequence
  source: /note=“Description of Artificial Sequence Synthetic oligonucleotide”
ccgauauuccucagguacu

SEQ ID NO: 964 - RNA, 20 nt, Artificial Sequence
  source: /note=“Description of Artificial Sequence Synthetic oligonucleotide”
ccgauauuccucagguacuc

SEQ ID NO: 965 - RNA, 21 nt, Artificial Sequence
  source: /note=“Description of Artificial Sequence Synthetic oligonucleotide”
ccgauauuccucagguacucc

SEQ ID NO: 966 - RNA, 22 nt, Artificial Sequence
  source: /note=“Description of Artificial Sequence Synthetic oligonucleotide”
ccgauauuccucagguacucca

SEQ ID NO: 967 - RNA, 23 nt, Artificial Sequence
  source: /note=“Description of Artificial Sequence Synthetic oligonucleotide”
ccgauauuccucagguacuccaa

SEQ ID NO: 968 - RNA, 24 nt, Artificial Sequence
  source: /note=“Description of Artificial Sequence Synthetic oligonucleotide”
ccgauauuccucagguacuccaaa

SEQ ID NO: 969 - RNA, 18 nt, Artificial Sequence
  source: /note=“Description of Artificial Sequence Synthetic oligonucleotide”
cucacgucauccagcaga

SEQ ID NO: 970 - RNA, 19 nt, Artificial Sequence
  source: /note=“Description of Artificial Sequence Synthetic oligonucleotide”
cucacgucauccagcagag

SEQ ID NO: 971 - RNA, 20 nt, Artificial Sequence
  source: /note=“Description of Artificial Sequence Synthetic oligonucleotide”
cucacgucauccagcagaga

SEQ ID NO: 972 - RNA, 21 nt, Artificial Sequence
  source: /note=“Description of Artificial Sequence Synthetic oligonucleotide”
cucacgucauccagcagagaa

SEQ ID NO: 973 - RNA, 22 nt, Artificial Sequence
  source: /note=“Description of Artificial Sequence Synthetic oligonucleotide”
cucacgucauccagcagagaau

SEQ ID NO: 974 - RNA, 23 nt, Artificial Sequence
  source: /note=“Description of Artificial Sequence Synthetic oligonucleotide”
cucacgucauccagcagagaaug

SEQ ID NO: 975 - RNA, 24 nt, Artificial Sequence
  source: /note=“Description of Artificial Sequence Synthetic oligonucleotide”
cucacgucauccagcagagaaugg

SEQ ID NO: 976 - RNA, 18 nt, Artificial Sequence
  source: /note=“Description of Artificial Sequence Synthetic oligonucleotide”
cugaauugcuaugugucu

SEQ ID NO: 977 - RNA, 19 nt, Artificial Sequence
  source: /note=“Description of Artificial Sequence Synthetic oligonucleotide”
cugaauugcuaugugucug

SEQ ID NO: 978 - RNA, 20 nt, Artificial Sequence
  source: /note=“Description of Artificial Sequence Synthetic oligonucleotide”
cugaauugcuaugugucugg

SEQ ID NO: 979 - RNA, 21 nt, Artificial Sequence
  source: /note=“Description of Artificial Sequence Synthetic oligonucleotide”
cugaauugcuaugugucuggg

SEQ ID NO: 980 - RNA, 22 nt, Artificial Sequence
  source: /note=“Description of Artificial Sequence Synthetic oligonucleotide”
cugaauugcuaugugucugggu

SEQ ID NO: 981 - RNA, 23 nt, Artificial Sequence
  source: /note=“Description of Artificial Sequence Synthetic oligonucleotide”
cugaauugcuaugugucuggguu

SEQ ID NO: 982 - RNA, 24 nt, Artificial Sequence
  source: /note=“Description of Artificial Sequence Synthetic oligonucleotide”
cugaauugcuaugugucuggguuu

SEQ ID NO: 983 - RNA, 18 nt, Artificial Sequence
  source: /note=“Description of Artificial Sequence Synthetic oligonucleotide”
gaguaccugaggaauauc

SEQ ID NO: 984 - RNA, 19 nt, Artificial Sequence
  source: /note=“Description of Artificial Sequence Synthetic oligonucleotide”
gaguaccugaggaauaucg

SEQ ID NO: 985 - RNA, 20 nt, Artificial Sequence
  source: /note=“Description of Artificial Sequence Synthetic oligonucleotide”
gaguaccugaggaauaucgg

SEQ ID NO: 986 - RNA, 21 nt, Artificial Sequence
  source: /note=“Description of Artificial Sequence Synthetic oligonucleotide”
gaguaccugaggaauaucggg

SEQ ID NO: 987 - RNA, 22 nt, Artificial Sequence
  source: /note=“Description of Artificial Sequence Synthetic oligonucleotide”
gaguaccugaggaauaucggga

SEQ ID NO: 988 - RNA, 23 nt, Artificial Sequence
  source: /note=“Description of Artificial Sequence Synthetic oligonucleotide”
gaguaccugaggaauaucgggaa

SEQ ID NO: 989 - RNA, 24 nt, Artificial Sequence
  source: /note=“Description of Artificial Sequence Synthetic oligonucleotide”
gaguaccugaggaauaucgggaaa

SEQ ID NO: 990 - RNA, 18 nt, Artificial Sequence
  source: /note=“Description of Artificial Sequence Synthetic oligonucleotide”
uaucucuuguacuacacu

SEQ ID NO: 991 - RNA, 19 nt, Artificial Sequence
  source: /note=“Description of Artificial Sequence Synthetic oligonucleotide”
uaucucuuguacuacacug

SEQ ID NO: 992 - RNA, 20 nt, Artificial Sequence
  source: /note=“Description of Artificial Sequence Synthetic oligonucleotide”
uaucucuuguacuacacuga

SEQ ID NO: 993 - RNA, 21 nt, Artificial Sequence
  source: /note=“Description of Artificial Sequence Synthetic oligonucleotide”
uaucucuuguacuacacugaa

SEQ ID NO: 994 - RNA, 22 nt, Artificial Sequence
  source: /note=“Description of Artificial Sequence Synthetic oligonucleotide”
uaucucuuguacuacacugaau

SEQ ID NO: 995 - RNA, 23 nt, Artificial Sequence
  source: /note=“Description of Artificial Sequence Synthetic oligonucleotide”
uaucucuuguacuacacugaauu

SEQ ID NO: 996 - RNA, 24 nt, Artificial Sequence
  source: /note=“Description of Artificial Sequence Synthetic oligonucleotide”
uaucucuuguacuacacugaauuc

SEQ ID NO: 997 - RNA, 18 nt, Artificial Sequence
  source: /note=“Description of Artificial Sequence Synthetic oligonucleotide”
ucaauucucucuccauuc

SEQ ID NO: 998 - RNA, 19 nt, Artificial Sequence
  source: /note=“Description of Artificial Sequence Synthetic oligonucleotide”
ucaauucucucuccauucu

SEQ ID NO: 999 - RNA, 20 nt, Artificial Sequence
  source: /note=“Description of Artificial Sequence Synthetic oligonucleotide”
ucaauucucucuccauucuu

SEQ ID NO: 1000 - RNA, 21 nt, Artificial Sequence
  source: /note=“Description of Artificial Sequence Synthetic oligonucleotide”
ucaauucucucuccauucuuc

SEQ ID NO: 1001 - RNA, 22 nt, Artificial Sequence
  source: /note=“Description of Artificial Sequence Synthetic oligonucleotide”
ucaauucucucuccauucuuca

SEQ ID NO: 1002 - RNA, 23 nt, Artificial Sequence
  source: /note=“Description of Artificial Sequence Synthetic oligonucleotide”
ucaauucucucuccauucuucag

SEQ ID NO: 1003 - RNA, 24 nt, Artificial Sequence
  source: /note=“Description of Artificial Sequence Synthetic oligonucleotide”
ucaauucucucuccauucuucagu

SEQ ID NO: 1004 - RNA, 18 nt, Artificial Sequence
  source: /note=“Description of Artificial Sequence Synthetic oligonucleotide”
ucacagcccaagauaguu

SEQ ID NO: 1005 - RNA, 19 nt, Artificial Sequence
  source: /note=“Description of Artificial Sequence Synthetic oligonucleotide”
ucacagcccaagauaguua

SEQ ID NO: 1006 - RNA, 20 nt, Artificial Sequence
  source: /note=“Description of Artificial Sequence Synthetic oligonucleotide”
ucacagcccaagauaguuaa

SEQ ID NO: 1007 - RNA, 21 nt, Artificial Sequence
  source: /note=“Description of Artificial Sequence Synthetic oligonucleotide”
ucacagcccaagauaguuaag

SEQ ID NO: 1008 - RNA, 22 nt, Artificial Sequence
  source: /note=“Description of Artificial Sequence Synthetic oligonucleotide”
ucacagcccaagauaguuaagu

SEQ ID NO: 1009 - RNA, 23 nt, Artificial Sequence
  source: /note=“Description of Artificial Sequence Synthetic oligonucleotide”
ucacagcccaagauaguuaagug

SEQ ID NO: 1010 - RNA, 24 nt, Artificial Sequence
  source: /note=“Description of Artificial Sequence Synthetic oligonucleotide”
ucacagcccaagauaguuaagugg

SEQ ID NO: 1011 - RNA, 18 nt, Artificial Sequence
  source: /note=“Description of Artificial Sequence Synthetic oligonucleotide”
ucagugggggugaauuca

SEQ ID NO: 1012 - RNA, 19 nt, Artificial Sequence
  source: /note=“Description of Artificial Sequence Synthetic oligonucleotide”
ucagugggggugaauucag

SEQ ID NO: 1013 - RNA, 20 nt, Artificial Sequence
  source: /note=“Description of Artificial Sequence Synthetic oligonucleotide”
ucagugggggugaauucagu

SEQ ID NO: 1014 - RNA, 21 nt, Artificial Sequence
  source: /note=“Description of Artificial Sequence Synthetic oligonucleotide”
ucagugggggugaauucagug

SEQ ID NO: 1015 - RNA, 22 nt, Artificial Sequence
  source: /note=“Description of Artificial Sequence Synthetic oligonucleotide”
ucagugggggugaauucagugu

SEQ ID NO: 1016 - RNA, 23 nt, Artificial Sequence
  source: /note=“Description of Artificial Sequence Synthetic oligonucleotide”
ucagugggggugaauucagugua

SEQ ID NO: 1017 - RNA, 24 nt, Artificial Sequence
  source: /note=“Description of Artificial Sequence Synthetic oligonucleotide”
ucagugggggugaauucaguguag

SEQ ID NO: 1018 - RNA, 18 nt, Artificial Sequence
  source: /note=“Description of Artificial Sequence Synthetic oligonucleotide”
uggccuggaggcuaucca

SEQ ID NO: 1019 - RNA, 19 nt, Artificial Sequence
  source: /note=“Description of Artificial Sequence Synthetic oligonucleotide”
uggccuggaggcuauccag

SEQ ID NO: 1020 - RNA, 20 nt, Artificial Sequence
  source: /note=“Description of Artificial Sequence Synthetic oligonucleotide”
uggccuggaggcuauccagc

SEQ ID NO: 1021 - RNA, 21 nt, Artificial Sequence
  source: /note=“Description of Artificial Sequence Synthetic oligonucleotide”
uggccuggaggcuauccagcg

SEQ ID NO: 1022 - RNA, 22 nt, Artificial Sequence
  source: /note=“Description of Artificial Sequence Synthetic oligonucleotide”
uggccuggaggcuauccagcgu

SEQ ID NO: 1023 - RNA, 23 nt, Artificial Sequence
  source: /note=“Description of Artificial Sequence Synthetic oligonucleotide”
uggccuggaggcuauccagcgug

SEQ ID NO: 1024 - RNA, 24 nt, Artificial Sequence
  source: /note=“Description of Artificial Sequence Synthetic oligonucleotide”
uggccuggaggcuauccagcguga

SEQ ID NO: 1025 - RNA, 18 nt, Artificial Sequence
  source: /note=“Description of Artificial Sequence Synthetic oligonucleotide”
auagaucgagacauguaa

SEQ ID NO: 1026 - RNA, 19 nt, Artificial Sequence
  source: /note=“Description of Artificial Sequence Synthetic oligonucleotide”
auagaucgagacauguaag

SEQ ID NO: 1027 - RNA, 20 nt, Artificial Sequence
  source: /note=“Description of Artificial Sequence Synthetic oligonucleotide”
auagaucgagacauguaagc

SEQ ID NO: 1028 - RNA, 21 nt, Artificial Sequence
  source: /note=“Description of Artificial Sequence Synthetic oligonucleotide”
auagaucgagacauguaagca

SEQ ID NO: 1029 - RNA, 22 nt, Artificial Sequence
  source: /note=“Description of Artificial Sequence Synthetic oligonucleotide”
auagaucgagacauguaagcag

SEQ ID NO: 1030 - RNA, 23 nt, Artificial Sequence
  source: /note=“Description of Artificial Sequence Synthetic oligonucleotide”
auagaucgagacauguaagcagc

SEQ ID NO: 1031 - RNA, 24 nt, Artificial Sequence
  source: /note=“Description of Artificial Sequence Synthetic oligonucleotide”
auagaucgagacauguaagcagca

SEQ ID NO: 1032 - RNA, 18 nt, Artificial Sequence
  source: /note=“Description of Artificial Sequence Synthetic oligonucleotide”
cauagaucgagacaugua

SEQ ID NO: 1033 - RNA, 19 nt, Artificial Sequence
  source: /note=“Description of Artificial Sequence Synthetic oligonucleotide”
cauagaucgagacauguaa

SEQ ID NO: 1034 - RNA, 20 nt, Artificial Sequence
  source: /note=“Description of Artificial Sequence Synthetic oligonucleotide”
cauagaucgagacauguaag

SEQ ID NO: 1035 - RNA, 21 nt, Artificial Sequence
  source: /note=“Description of Artificial Sequence Synthetic oligonucleotide”
cauagaucgagacauguaagc

SEQ ID NO: 1036 - RNA, 22 nt, Artificial Sequence
  source: /note=“Description of Artificial Sequence Synthetic oligonucleotide”
cauagaucgagacauguaagca

SEQ ID NO: 1037 - RNA, 23 nt, Artificial Sequence
  source: /note=“Description of Artificial Sequence Synthetic oligonucleotide”
cauagaucgagacauguaagcag

SEQ ID NO: 1038 - RNA, 24 nt, Artificial Sequence
  source: /note=“Description of Artificial Sequence Synthetic oligonucleotide”
cauagaucgagacauguaagcagc

SEQ ID NO: 1039 - RNA, 18 nt, Artificial Sequence
  source: /note=“Description of Artificial Sequence Synthetic oligonucleotide”
cuccacugucuuuuucau

SEQ ID NO: 1040 - RNA, 19 nt, Artificial Sequence
  source: /note=“Description of Artificial Sequence Synthetic oligonucleotide”
cuccacugucuuuuucaua

SEQ ID NO: 1041 - RNA, 20 nt, Artificial Sequence
  source: /note=“Description of Artificial Sequence Synthetic oligonucleotide”
cuccacugucuuuuucauag

SEQ ID NO: 1042 - RNA, 21 nt, Artificial Sequence
  source: /note=“Description of Artificial Sequence Synthetic oligonucleotide”
cuccacugucuuuuucauaga

SEQ ID NO: 1043 - RNA, 22 nt, Artificial Sequence
  source: /note=“Description of Artificial Sequence Synthetic oligonucleotide”
cuccacugucuuuuucauagau

SEQ ID NO: 1044 - RNA, 23 nt, Artificial Sequence
  source: /note=“Description of Artificial Sequence Synthetic oligonucleotide”
cuccacugucuuuuucauagauc

SEQ ID NO: 1045 - RNA, 24 nt, Artificial Sequence
  source: /note=“Description of Artificial Sequence Synthetic oligonucleotide”
cuccacugucuuuuucauagaucg

SEQ ID NO: 1046 - RNA, 18 nt, Artificial Sequence
  source: /note=“Description of Artificial Sequence Synthetic oligonucleotide”
ucauagaucgagacaugu

SEQ ID NO: 1047 - RNA, 19 nt, Artificial Sequence
  source: /note=“Description of Artificial Sequence Synthetic oligonucleotide”
ucauagaucgagacaugua

SEQ ID NO: 1048 - RNA, 20 nt, Artificial Sequence
  source: /note=“Description of Artificial Sequence Synthetic oligonucleotide”
ucauagaucgagacauguaa

SEQ ID NO: 1049 - RNA, 21 nt, Artificial Sequence
  source: /note=“Description of Artificial Sequence Synthetic oligonucleotide”
ucauagaucgagacauguaag

SEQ ID NO: 1050 - RNA, 22 nt, Artificial Sequence
  source: /note=“Description of Artificial Sequence Synthetic oligonucleotide”
ucauagaucgagacauguaagc

SEQ ID NO: 1051 - RNA, 23 nt, Artificial Sequence
  source: /note=“Description of Artificial Sequence Synthetic oligonucleotide”
ucauagaucgagacauguaagca

SEQ ID NO: 1052 - RNA, 24 nt, Artificial Sequence
  source: /note=“Description of Artificial Sequence Synthetic oligonucleotide”
ucauagaucgagacauguaagcag

SEQ ID NO: 1053 - RNA, 18 nt, Artificial Sequence
  source: /note=“Description of Artificial Sequence Synthetic oligonucleotide”
uccacugucuuuuucaua

SEQ ID NO: 1054 - RNA, 19 nt, Artificial Sequence
  source: /note=“Description of Artificial Sequence Synthetic oligonucleotide”
uccacugucuuuuucauag

SEQ ID NO: 1055 - RNA, 20 nt, Artificial Sequence
  source: /note=“Description of Artificial Sequence Synthetic oligonucleotide”
uccacugucuuuuucauaga

SEQ ID NO: 1056 - RNA, 21 nt, Artificial Sequence
  source: /note=“Description of Artificial Sequence Synthetic oligonucleotide”
uccacugucuuuuucauagau

SEQ ID NO: 1057 - RNA, 22 nt, Artificial Sequence
  source: /note=“Description of Artificial Sequence Synthetic oligonucleotide”
uccacugucuuuuucauagauc

SEQ ID NO: 1058 - RNA, 23 nt, Artificial Sequence
  source: /note=“Description of Artificial Sequence Synthetic oligonucleotide”
uccacugucuuuuucauagaucg

SEQ ID NO: 1059 - RNA, 24 nt, Artificial Sequence
  source: /note=“Description of Artificial Sequence Synthetic oligonucleotide”
uccacugucuuuuucauagaucga

SEQ ID NO: 1060 - RNA, 18 nt, Artificial Sequence
  source: /note=“Description of Artificial Sequence Synthetic oligonucleotide”
ucuccacugucuuuuuca

SEQ ID NO: 1061 - RNA, 19 nt, Artificial Sequence
  source: /note=“Description of Artificial Sequence Synthetic oligonucleotide”
ucuccacugucuuuuucau

SEQ ID NO: 1062 - RNA, 20 nt, Artificial Sequence
  source: /note=“Description of Artificial Sequence Synthetic oligonucleotide”
ucuccacugucuuuuucaua

SEQ ID NO: 1063 - RNA, 21 nt, Artificial Sequence
  source: /note=“Description of Artificial Sequence Synthetic oligonucleotide”
ucuccacugucuuuuucauag

SEQ ID NO: 1064 - RNA, 22 nt, Artificial Sequence
  source: /note=“Description of Artificial Sequence Synthetic oligonucleotide”
ucuccacugucuuuuucauaga

SEQ ID NO: 1065 - RNA, 23 nt, Artificial Sequence
  source: /note=“Description of Artificial Sequence Synthetic oligonucleotide”
ucuccacugucuuuuucauagau

SEQ ID NO: 1066 - RNA, 24 nt, Artificial Sequence
  source: /note=“Description of Artificial Sequence Synthetic oligonucleotide”
ucuccacugucuuuuucauagauc

SEQ ID NO: 1067 - RNA, 18 nt, Artificial Sequence
  source: /note=“Description of Artificial Sequence Synthetic oligonucleotide”
uucuccacugucuuuuuc

SEQ ID NO: 1068 - RNA, 19 nt, Artificial Sequence
  source: /note=“Description of Artificial Sequence Synthetic oligonucleotide”
uucuccacugucuuuuuca

SEQ ID NO: 1069 - RNA, 20 nt, Artificial Sequence
  source: /note=“Description of Artificial Sequence Synthetic oligonucleotide”
uucuccacugucuuuuucau

SEQ ID NO: 1070 - RNA, 21 nt, Artificial Sequence
  source: /note=“Description of Artificial Sequence Synthetic oligonucleotide”
uucuccacugucuuuuucaua

SEQ ID NO: 1071 - RNA, 22 nt, Artificial Sequence
  source: /note=“Description of Artificial Sequence Synthetic oligonucleotide”
uucuccacugucuuuuucauag

SEQ ID NO: 1072 - RNA, 23 nt, Artificial Sequence
  source: /note=“Description of Artificial Sequence Synthetic oligonucleotide”
uucuccacugucuuuuucauaga

SEQ ID NO: 1073 - RNA, 24 nt, Artificial Sequence
  source: /note=“Description of Artificial Sequence Synthetic oligonucleotide”
uucuccacugucuuuuucauagau

SEQ ID NO: 1074 - RNA, 18 nt, Artificial Sequence
  source: /note=“Description of Artificial Sequence Synthetic oligonucleotide”
uuucuccacugucuuuuu

SEQ ID NO: 1075 - RNA, 19 nt, Artificial Sequence
  source: /note=“Description of Artificial Sequence Synthetic oligonucleotide”
uuucuccacugucuuuuuc

SEQ ID NO: 1076 - RNA, 20 nt, Artificial Sequence
  source: /note=“Description of Artificial Sequence Synthetic oligonucleotide”
uuucuccacugucuuuuuca

SEQ ID NO: 1077 - RNA, 21 nt, Artificial Sequence
  source: /note=“Description of Artificial Sequence Synthetic oligonucleotide”
uuucuccacugucuuuuucau

SEQ ID NO: 1078 - RNA, 22 nt, Artificial Sequence
  source: /note=“Description of Artificial Sequence Synthetic oligonucleotide”
uuucuccacugucuuuuucaua

SEQ ID NO: 1079 - RNA, 23 nt, Artificial Sequence
  source: /note=“Description of Artificial Sequence Synthetic oligonucleotide”
uuucuccacugucuuuuucauag

SEQ ID NO: 1080 - RNA, 24 nt, Artificial Sequence
  source: /note=“Description of Artificial Sequence Synthetic oligonucleotide”
uuucuccacugucuuuuucauaga

SEQ ID NO: 1081 - RNA, 18 nt, Artificial Sequence
  source: /note=“Description of Artificial Sequence Synthetic oligonucleotide”
uuuucuccacugucuuuu

SEQ ID NO: 1082 - RNA, 19 nt, Artificial Sequence
  source: /note=“Description of Artificial Sequence Synthetic oligonucleotide”
uuuucuccacugucuuuuu

SEQ ID NO: 1083 - RNA, 20 nt, Artificial Sequence
  source: /note=“Description of Artificial Sequence Synthetic oligonucleotide”
uuuucuccacugucuuuuuc

SEQ ID NO: 1084 - RNA, 21 nt, Artificial Sequence
  source: /note=“Description of Artificial Sequence Synthetic oligonucleotide”
uuuucuccacugucuuuuuca

SEQ ID NO: 1085 - RNA, 22 nt, Artificial Sequence
  source: /note=“Description of Artificial Sequence Synthetic oligonucleotide”
uuuucuccacugucuuuuucau

SEQ ID NO: 1086 - RNA, 23 nt, Artificial Sequence
  source: /note=“Description of Artificial Sequence Synthetic oligonucleotide”
uuuucuccacugucuuuuucaua

SEQ ID NO: 1087 - RNA, 24 nt, Artificial Sequence
  source: /note=“Description of Artificial Sequence Synthetic oligonucleotide”
uuuucuccacugucuuuuucauag

SEQ ID NO: 1088 - DNA, 21 bp, Artificial Sequence
  source: /note=“Description of Artificial Sequence Synthetic oligonucleotide”
gaggtaaagcgtttgcatttg

SEQ ID NO: 1089 - DNA, 21 bp, Artificial Sequence
  source: /note=“Description of Artificial Sequence Synthetic oligonucleotide”
cctctaaagcttatgcttaca

SEQ ID NO: 1090 - DNA, 21 bp, Artificial Sequence
  source: /note=“Description of Artificial Sequence Synthetic oligonucleotide”
agtcgatttacttgtagcact

SEQ ID NO: 1091 - DNA, 21 bp, Artificial Sequence
  source: /note=“Description of Artificial Sequence Synthetic oligonucleotide”
cttgtagcactgcacagttaa

SEQ ID NO: 1092 - DNA, 21 bp, Artificial Sequence
  source: /note=“Description of Artificial Sequence Synthetic oligonucleotide”
tccattacaggataaaagact

SEQ ID NO: 1093 - DNA, 21 bp, Artificial Sequence
  source: /note=“Description of Artificial Sequence Synthetic oligonucleotide”
ctccattacaggataaaagac

SEQ ID NO: 1094 - DNA, 21 bp, Artificial Sequence
  source: /note=“Description of Artificial Sequence Synthetic oligonucleotide”
tctccattacaggataaaaga

SEQ ID NO: 1095 - DNA, 21 bp, Artificial Sequence
  source: /note=“Description of Artificial Sequence Synthetic oligonucleotide”
atcctgtaatggagaaaaatc

SEQ ID NO: 1096 - DNA, 21 bp, Artificial Sequence
  source: /note=“Description of Artificial Sequence Synthetic oligonucleotide”
tcctgtaatggagaaaaatcc

SEQ ID NO: 1097 - DNA, 21 bp, Artificial Sequence
  source: /note=“Description of Artificial Sequence Synthetic oligonucleotide”
aaacatgagtaagttgttttg

SEQ ID NO: 1098 - DNA, 21 bp, Artificial Sequence
  source: /note=“Description of Artificial Sequence Synthetic oligonucleotide”
gctttcaaacatgagtaagtt

SEQ ID NO: 1099 - DNA, 21 bp, Artificial Sequence
  source: /note=“Description of Artificial Sequence Synthetic oligonucleotide”
aaagccaaaccattcattgtc

SEQ ID NO: 1100 - DNA, 21 bp, Artificial Sequence
  source: /note=“Description of Artificial Sequence Synthetic oligonucleotide”
gtaacagcagtcatcatccat

SEQ ID NO: 1101 - DNA, 21 bp, Artificial Sequence
  source: /note=“Description of Artificial Sequence Synthetic oligonucleotide”
accatcctcatggattggtgt

SEQ ID NO: 1102 - DNA, 21 bp, Artificial Sequence
  source: /note=“Description of Artificial Sequence Synthetic oligonucleotide”
tgtccatcatttcaccatcct

SEQ ID NO: 1103 - DNA, 21 bp, Artificial Sequence
  source: /note=“Description of Artificial Sequence Synthetic oligonucleotide”
gaaatttctgtccatcatttc

SEQ ID NO: 1104 - DNA, 21 bp, Artificial Sequence
  source: /note=“Description of Artificial Sequence Synthetic oligonucleotide”
agaaatttctgtccatcattt

SEQ ID NO: 1105 - DNA, 21 bp, Artificial Sequence
  source: /note=“Description of Artificial Sequence Synthetic oligonucleotide”
ttttagaaatttctgtccatc

SEQ ID NO: 1106 - DNA, 21 bp, Artificial Sequence
  source: /note=“Description of Artificial Sequence Synthetic oligonucleotide”
cttttagaaatttctgtccat

SEQ ID NO: 1107 - DNA, 21 bp, Artificial Sequence
  source: /note=“Description of Artificial Sequence Synthetic oligonucleotide”
ttttcttttagaaatttctgt

SEQ ID NO: 1108 - DNA, 21 bp, Artificial Sequence
  source: /note=“Description of Artificial Sequence Synthetic oligonucleotide”
taaaagaaaagaaagaatttt

SEQ ID NO: 1109 - DNA, 21 bp, Artificial Sequence
  source: /note=“Description of Artificial Sequence Synthetic oligonucleotide”
aaacatttacatcttaccatt

SEQ ID NO: 1110 - DNA, 21 bp, Artificial Sequence
  source: /note=“Description of Artificial Sequence Synthetic oligonucleotide”
catcttaccatttcttcttca

SEQ ID NO: 1111 - DNA, 21 bp, Artificial Sequence
  source: /note=“Description of Artificial Sequence Synthetic oligonucleotide”
tatagataatgaagaagaaat

SEQ ID NO: 1112 - DNA, 21 bp, Artificial Sequence
  source: /note=“Description of Artificial Sequence Synthetic oligonucleotide”
ttcttcattatctatagaaag

SEQ ID NO: 1113 - DNA, 21 bp, Artificial Sequence
  source: /note=“Description of Artificial Sequence Synthetic oligonucleotide”
ctggcctgtacttcgaagaac

SEQ ID NO: 1114 - DNA, 21 bp, Artificial Sequence
  source: /note=“Description of Artificial Sequence Synthetic oligonucleotide”
cttaccaatgtagtaacaact

SEQ ID NO: 1115 - DNA, 21 bp, Artificial Sequence
  source: /note=“Description of Artificial Sequence Synthetic oligonucleotide”
gcacgtcattgtggccattgt

SEQ ID NO: 1116 - DNA, 21 bp, Artificial Sequence
  source: /note=“Description of Artificial Sequence Synthetic oligonucleotide”
tttagcacgtcattgtggcca

SEQ ID NO: 1117 - DNA, 21 bp, Artificial Sequence
  source: /note=“Description of Artificial Sequence Synthetic oligonucleotide”
ccatcagctccagagaagctc

SEQ ID NO: 1118 - DNA, 21 bp, Artificial Sequence
  source: /note=“Description of Artificial Sequence Synthetic oligonucleotide”
tctccctgcagatttaccatc

SEQ ID NO: 1119 - DNA, 21 bp, Artificial Sequence
  source: /note=“Description of Artificial Sequence Synthetic oligonucleotide”
aaatgctttacctttgcagtg

SEQ ID NO: 1120 - DNA, 21 bp, Artificial Sequence
  source: /note=“Description of Artificial Sequence Synthetic oligonucleotide”
aatgctttacctttgcagtga

SEQ ID NO: 1121 - DNA, 21 bp, Artificial Sequence
  source: /note=“Description of Artificial Sequence Synthetic oligonucleotide”
cctttgcagtgataggttttg

SEQ ID NO: 1122 - DNA, 21 bp, Artificial Sequence
  source: /note=“Description of Artificial Sequence Synthetic oligonucleotide”
cagtgataggttttgtcattc

SEQ ID NO: 1123 - DNA, 21 bp, Artificial Sequence
  source: /note=“Description of Artificial Sequence Synthetic oligonucleotide”
aagggaatgacaaaacctatc

SEQ ID NO: 1124 - DNA, 21 bp, Artificial Sequence
  source: /note=“Description of Artificial Sequence Synthetic oligonucleotide”
caagggaatgacaaaacctat

SEQ ID NO: 1125 - DNA, 21 bp, Artificial Sequence
  source: /note=“Description of Artificial Sequence Synthetic oligonucleotide”
gtcattcccttgaaaatcctg

SEQ ID NO: 1126 - DNA, 21 bp, Artificial Sequence
  source: /note=“Description of Artificial Sequence Synthetic oligonucleotide”
tcattcccttgaaaatcctga

SEQ ID NO: 1127 - DNA, 21 bp, Artificial Sequence
  source: /note=“Description of Artificial Sequence Synthetic oligonucleotide”
tgaaggtttaattccgcatag

SEQ ID NO: 1128 - DNA, 21 bp, Artificial Sequence
  source: /note=“Description of Artificial Sequence Synthetic oligonucleotide”
gaaggtttaattccgcatagg

SEQ ID NO: 1129 - DNA, 21 bp, Artificial Sequence
  source: /note=“Description of Artificial Sequence Synthetic oligonucleotide”
aaggtttaattccgcataggt

SEQ ID NO: 1130 - DNA, 21 bp, Artificial Sequence
  source: /note=“Description of Artificial Sequence Synthetic oligonucleotide”
attccgcataggttatttcct

SEQ ID NO: 1131 - DNA, 21 bp, Artificial Sequence
  source: /note=“Description of Artificial Sequence Synthetic oligonucleotide”
gcaactgaacaggaaataacc

SEQ ID NO: 1132 - DNA, 21 bp, Artificial Sequence
  source: /note=“Description of Artificial Sequence Synthetic oligonucleotide”
agcaactgaacaggaaataac

SEQ ID NO: 1133 - DNA, 21 bp, Artificial Sequence
  source: /note=“Description of Artificial Sequence Synthetic oligonucleotide”
ctgttcagttgctaaaatgga

SEQ ID NO: 1134 - DNA, 21 bp, Artificial Sequence
  source: /note=“Description of Artificial Sequence Synthetic oligonucleotide”
tattgcctttaggttttcgtt

SEQ ID NO: 1135 - DNA, 21 bp, Artificial Sequence
  source: /note=“Description of Artificial Sequence Synthetic oligonucleotide”
attgcctttaggttttcgttg

SEQ ID NO: 1136 - DNA, 21 bp, Artificial Sequence
  source: /note=“Description of Artificial Sequence Synthetic oligonucleotide”
ttgcctttaggttttcgttgc

SEQ ID NO: 1137 - DNA, 21 bp, Artificial Sequence
  source: /note=“Description of Artificial Sequence Synthetic oligonucleotide”
ggttttcgttgctgcctcttt

SEQ ID NO: 1138 - DNA, 21 bp, Artificial Sequence
  source: /note=“Description of Artificial Sequence Synthetic oligonucleotide”
cgttgctgcctctttgggttt

SEQ ID NO: 1139 - DNA, 21 bp, Artificial Sequence
  source: /note=“Description of Artificial Sequence Synthetic oligonucleotide”
gttgctgcctctttgggtttg

SEQ ID NO: 1140 - DNA, 21 bp, Artificial Sequence
  source: /note=“Description of Artificial Sequence Synthetic oligonucleotide”
ggtttgggggcagattcaggt

SEQ ID NO: 1141 - DNA, 21 bp, Artificial Sequence
  source: /note=“Description of Artificial Sequence Synthetic oligonucleotide”
ggggcagattcaggtctgagt

SEQ ID NO: 1142 - protein, 1334 aa, Artificial Sequence
  source: /note=“Description of Artificial Sequence Synthetic polypeptide”
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1143 - protein, 1363 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1144 - protein, 1363 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1145 - protein, 1363 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1146 - protein, 1331 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1147 - protein, 1331 aa, Artificial Sequence
  source: /note=“Description of Artificial Sequence Synthetic polypeptide”
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1148 - protein, 1363 aa, Artificial Sequence; identical to SEQ ID NO: 1143
  source: /note=“Description of Artificial Sequence Synthetic polypeptide”

SEQ ID NO: 1149 - protein, 1361 aa, Artificial Sequence
  source: /note=“Description of Artificial Sequence Synthetic polypeptide”
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 1150 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1151 - RNA, 20 nt, Artificial Sequence
  source: /note=“Description of Artificial Sequence Synthetic oligonucleotide”
uagauguucucaucuuuaau

SEQ ID NO: 1152 - DNA, 25 bp, Artificial Sequence
  source: /note=“Description of Artificial Sequence Synthetic oligonucleotide”
atgtgtttttgtcaaaagacctttt

SEQ ID NO: 1153 - DNA, 20 bp, Artificial Sequence
  source: /note=“Description of Artificial Sequence Synthetic oligonucleotide”
ggtgtacagcagtggctggt

SEQ ID NO: 1154 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagaugguguacagcaguggcuggu

SEQ ID NO: 1155 - DNA, 20 bp, Artificial Sequence
  source: /note=“Description of Artificial Sequence Synthetic oligonucleotide”
tgatgtgagattttccacct

SEQ ID NO: 1156 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauugaugugagauuuuccaccu

SEQ ID NO: 1157 - DNA, 20 bp, Artificial Sequence
  source: /note=“Description of Artificial Sequence Synthetic oligonucleotide”
tgcagagaaaggtggctcta

SEQ ID NO: 1158 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauugcagagaaagguggcucua

SEQ ID NO: 1159 - DNA, 21 bp, Artificial Sequence
  source: /note=“Description of Artificial Sequence Synthetic oligonucleotide”
gcaactgaacaggaaataacc

SEQ ID NO: 1160 - RNA, 41 nt, Artificial Sequence
  source: /note=“Description of Artificial Sequence Synthetic oligonucleotide”
uaauuucuacucuuguagaugcaacugaacaggaaauaacc

SEQ ID NO: 1161 - DNA, 20 bp, Artificial Sequence
  source: /note=“Description of Artificial Sequence Synthetic oligonucleotide”
cctgtgtgctggtgcccctg

SEQ ID NO: 1162 - DNA, 65 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauccugugugcuggugccccug

SEQ ID NO: 1163 - DNA, 21 bp, Artificial Sequence
  source: /note=“Description of Artificial Sequence Synthetic oligonucleotide”
actgacagcgtgaacaggtag

SEQ ID NO: 1164 - DNA, 66 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauacugacagcgugaacagguag

SEQ ID NO: 1165 - DNA, 20 bp, Artificial Sequence
  source: /note=“Description of Artificial Sequence Synthetic oligonucleotide”
tgatgtgagattttccacct

SEQ ID NO: 1166 - DNA, 65 bp, Artificial Sequence; identical to SEQ ID NO: 1156
  source: /note=“Description of Artificial Sequence Synthetic oligonucleotide”
  source: /note=“Description of Combined DNA/RNA Molecule Synthetic oligonucleotide”

SEQ ID NO: 1167 - DNA, 20 bp, Artificial Sequence
  source: /note=“Description of Artificial Sequence Synthetic oligonucleotide”
tgcagagaaaggtggctcta

SEQ ID NO: 1168 - DNA, 65 bp, Artificial Sequence; identical to SEQ ID NO: 1158
  source: /note=“Description of Artificial Sequence Synthetic oligonucleotide”
  source: /note=“Description of Combined DNA/RNA Molecule Synthetic oligonucleotide”

SEQ ID NO: 1169 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
acugacagcgugaacagguag

SEQ ID NO: 1170 - DNA, 66 bp, Artificial Sequence; identical to SEQ ID NO: 1164
  source: /note=“Description of Artificial Sequence Synthetic oligonucleotide”
  source: /note=“Description of Combined DNA/RNA Molecule Synthetic oligonucleotide”

SEQ ID NO: 1171 - DNA, 21 bp, Artificial Sequence
  source: /note=“Description of Artificial Sequence Synthetic oligonucleotide”
  source: /note=“Description of Combined DNA/RNA Molecule Synthetic oligonucleotide”
ugaugugagauuuuccaccug

SEQ ID NO: 1172 - DNA, 66 bp, Artificial Sequence
  source: /note=“Description of Artificial Sequence Synthetic oligonucleotide”
  source: /note=“Description of Combined DNA/RNA Molecule Synthetic oligonucleotide”
atgtgtttttgtcaaaagaccttttuaauuucuacucuuguagauugaugugagauuuuccaccug

SEQ ID NO: 1173 - DNA, 21 bp, Artificial Sequence
  source: /note=“Description of Artificial Sequence Synthetic oligonucleotide”
tgatgtgagattttccacctg

SEQ ID NO: 1174 - DNA, 21 bp, Artificial Sequence
  source: /note=“Description of Artificial Sequence Synthetic oligonucleotide”
actgacagcgtgaacaggtag

SEQ ID NO: 1175 - DNA, 3993 bp, Artificial Sequence
  source: /note=“Description of Artificial Sequence Synthetic oligonucleotide”
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

(19) United States
(12) Patent Application Publication
(10) Pub. No.: US 2025/0262301 A1
(43) Pub. Date: Aug. 21, 2025

(54) METHODS OF INDUCING ANTIBODY-DEPENDENT CELLULAR CYTOTOXICITY (ADCC) USING MODIFIED NATURAL KILLER (NK) CELLS

(71) Applicant: Shoreline Biosciences, Inc., San Diego, CA (US)

(72) Inventors: Christopher BORGES, Reading, MA (US); Karrie Ka Wai WONG, Somerville, MA (US)

(73) Assignee: Shoreline Biosciences, Inc., San Diego, CA (US)

(21) Appl. No.: 18/250,422

(22) PCT Filed: Oct. 26, 2021
(86) PCT No.: PCT/US2021/056554

§ 371 Date: Apr. 25, 2023

Related U.S. Application Data
(60) Provisional application No. 63/165,786, filed on Mar. 25, 2021.
(60) Provisional application No. 63/115,112, filed on Nov. 18, 2020.
(60) Provisional application No. 63/105,464, filed on Oct. 26, 2020.

(51) Int. Cl. A61K 40/15; A61K 39/395; A61K 40/42; A61P 35/00

(52) U.S. Cl. CPC A61K 40/15; A61K 39/39558; A61K 40/4224; A61K 40/4229; A61K 40/4249; A61P 35/00

(57) ABSTRACT

The present disclosure is directed to the use of modified NK cells for immunotherapy in combination with an antibody, or antigen-binding fragment thereof, to induce an enhanced antibody-dependent cellular cytotoxicity (ADCC) effect.

### CROSS-REFERENCE TO RELATED APPLICATIONS

This application is a U.S. National Stage Application under 35 U.S.C. § 371 of International Patent Application No. PCT/US2021/056554, filed Oct. 26, 2021, which claims the benefit of U.S. Provisional Application No. 63/105,464, filed on Oct. 26, 2020; U.S. Provisional Application No. 63/115,112, filed on Nov. 18, 2020; and U.S. Provisional Application No. 63/165,786, filed on Mar. 25, 2021, the entire contents of each of which are expressly incorporated herein by reference.

### SEQUENCE LISTING

This application incorporates by reference in its entirety the Computer Readable Form (CRF) of a Substitute Sequence Listing in ASCII text format submitted via Patent Center. The Substitute Sequence Listing text file submitted via Patent Center is entitled “14735-027-999_SUB_SEQ_LISTING.txt,” was created on Nov. 16, 2023, and is 415,811 bytes in size.

### BACKGROUND

NK cells are useful for immunotherapy approaches, for example, in the context of immunooncology. NK cells are a type of cytotoxic innate lymphocyte. NK cells play an important role in tumor immunity, and the cytotoxic activity of NK cells is tightly regulated by a network of activating and inhibitory pathways (see, e.g., Bald, T., Krummel, M. F., Smyth, M. J. et al. (2020) Nat Immunol 21, 835-847; and Huntington, N. D., Cursons, J. & Rautela, J. (2020) Nat Rev Cancer 20, 437-454; incorporated in their entireties herein by reference).

The use of naturally occurring or modified NK cells in immunotherapy approaches, e.g., via autologous or allogeneic NK cell transfer, has been reported, and while some success has been achieved, such approaches are typically characterized by a suboptimal NK cell response. In the context of immune-oncology, it is believed that this suboptimal response is, at least in part, to tumors harnessing NK cell inhibitory pathways to suppress cytotoxic NK cell activity, limit NK cell invasion, and/or inhibit NK cell proliferation and survival. Thus, application of NK cells in the therapy of solid tumors has seen limited success to date.

Initial work has been performed in trying to focus NK cell response on specific cells, e.g., by expressing a chimeric antigen receptor in NK cells that targets the NK cells to tumor cells, or by modulating activating or inhibitory NK cell pathways to achieve a stronger and/or more sustained NK cell response. See, e.g., Liu et al. (2020) New England J. Medicine 382(6):545-553; incorporated in its entirety herein by reference.

In pursuit of an off-the shelf allogeneic NK cell therapy, an induced pluripotent stem cell line has been developed in which cells express an enhanced version of CD16 (hnCD16), and NK cells have been derived from this iPSC line. See, e.g., Li et al., Cell Stem Cell. 2018 Aug. 2; 23(2):181-192.e5; incorporated in its entirety herein by reference.

However, to date all of these approaches have seen limited success. Therefore, there remains a need for the development of better therapeutic approaches for immunotherapy.

### SUMMARY

The present disclosure provides modified NK cells (or other lymphocytes) that are useful in NK cell therapy, e.g., in the context of immunotherapeutic approaches, particularly in combination with a therapeutic antibody, or antigen-binding portion thereof, to generate striking antibody-dependent cellular cytotoxicity (ADCC) effects, thereby surprisingly increasing the effectiveness of the modified NK cells in killing target cells, e.g. cancer cells. ADCC is a mechanism of cell-mediated immune defense, where an immune effector cell actively lyses a target cell after its membrane-surface antigens have been bound by specific antibodies. To participate in ADCC, the immune effector cells must express Fc-gamma receptors (FcγR) to be able to recognize the Fc region of the antibodies that bind to the target cells. Most immune effector cells have both activating and inhibitory FcγR. An advantage of using NK cells to target cancer cells via ADCC is that, unlike other effector cells, NK cells only have activating FcγRs (e.g., FcγR IIIa, also known as CD16a, and FcγR IIc, also known as CD32c) and are believed to be the most important effectors of ADCC in humans. Thus, the use of the modified NK cells disclosed herein and antibodies targeting cancer cell-specific antigens to elicit ADCC provides novel and surprisingly effective immunotherapies.

In some embodiments, the modified NK cells provided herein can serve as an off-the-shelf clinical solution for patients having, or having been diagnosed with, a hyperproliferative disease, such as, for example, a cancer. In some embodiments, the modified NK cells exhibit an enhanced survival, proliferation, NK cell response level, NK cell response duration, resistance against reduction of NK cell functional persistence, and/or target recognition as compared to non-modified NK cells. For example, the modified NK cells provided herein may comprise genomic edits that result in a loss-of-function in TGF beta receptor 2 (TGFbetaR2) and/or a loss-of-function of CISH. In some embodiments, the modified NK cells comprise genomic edits that result in a loss-of-function of TGFbetaR2. In some embodiments, the modified NK cells comprise genomic edits that result in a loss-of-function of CISH. In some embodiments, the modified NK cells comprise genomic edits that result in a loss-of-function of TGFbetaR2 and a loss-of-function of CISH. In some embodiments, the modified NK cells consist of genomic edits that result in a loss-of-function of TGFbetaR2. In some embodiments, the modified NK cells consist of genomic edits that result in a loss-of-function of CISH. In some embodiments, the modified NK cells consist of genomic edits that result in a loss-of-function of TGFbetaR2 and a loss-of-function of CISH. Other modified NK cells that may be useful in the methods described herein are described in WO2020/168300, published on 17 Sep. 2020, the entire contents of which are expressly incorporated by reference herein.

In some embodiments, the modified NK cells provided herein may comprise genomic edits that result in: expression of a chimeric antigen receptor (CAR) of interest, e.g., a CAR targeting mesothelin, EGFR, HER2 and/or MICA/B; expression of a CD16 variant, e.g., a non-naturally occurring CD16 variant such as, for example, hnCD16 (see, e.g., Zhu et al., Blood 2017, 130:4452, the contents of which are incorporated herein in their entirety by reference); expression of an IL15/IL15RA fusion; a loss-of-function in TGF beta receptor 2 (TGFbetaR2); and/or expression of a dominant-negative variant of TGFbetaR2; a loss-of-function of ADORA2A; a loss-of-function of B2M; expression of HLA-G: a loss-of-function of a CIITA; a loss-of-function of a PD1; a loss-of-function of TIGIT; and/or a loss-of-function of CISH; or any combination of two or more thereof in the modified NK cell. In one embodiment, the modified NK cell comprises genomic edits that result in a loss-of-function of TGFbetaR2 and a loss-of-function of CISH. In one embodiment, the modified NK cell comprises genomic edits that result in a loss-of-function of TGFbetaR2 and a loss-of-function of TIGIT. In one embodiment, the modified NK cell comprises genomic edits that result in a loss-of-function of TGFbetaR2 and a loss-of-function of ADORA2A. In one embodiment, the modified NK cell comprises genomic edits that result in a loss-of-function of TGFbetaR2 and a loss-of-function of NKG2A. In one embodiment, the modified NK cell comprises genomic edits that result in a loss-of-function of CISH and a loss-of-function of TIGIT. In one embodiment, the modified NK cell comprises genomic edits that result in a loss-of-function of CISH and a loss-of-function of ADORA2A. In one embodiment, the modified NK cell comprises genomic edits that result in a loss-of-function of CISH and a loss-of-function of NKG2A. In one embodiment, the modified NK cell comprises genomic edits that result in a loss-of-function of TIGIT and a loss-of-function of ADORA2A. In one embodiment, the modified NK cell comprises genomic edits that result in a loss-of-function of TIGIT and a loss-of-function of NKG2A. In one embodiment, the modified NK cell comprises genomic edits that result in a loss-of-function of ADORA2A and a loss-of-function of NKG2A. In one embodiment, the modified NK cell comprises genomic edits that result in a loss-of-function of TGFbetaR2, a loss-of-function of CISH, and a loss-of-function of TIGIT. In one embodiment, the modified NK cell comprises genomic edits that result in a loss-of-function of TGFbetaR2, a loss-of-function of CISH, and a loss-of-function of ADORA2A. In one embodiment, the modified NK cell comprises genomic edits that result in a loss-of-function of TGFbetaR2, a loss-of-function of CISH, and a loss-of-function of NKG2A. In one embodiment, the modified NK cell comprises genomic edits that result in a loss-of-function of TGFbetaR2, a loss-of-function of TIGIT, and a loss-of-function of ADORA2A. In one embodiment, the modified NK cell comprises genomic edits that result in a loss-of-function of TGFbetaR2, a loss-of-function of TIGIT, and a loss-of-function of NKG2A. In one embodiment, the modified NK cell comprises genomic edits that result in a loss-of-function of TGFbetaR2, a loss-of-function of ADORA2A, and a loss-of-function of NKG2A. In one embodiment, the modified NK cell comprises genomic edits that result in a loss-of-function of CISH, a loss-of-function of TIGIT, and a loss-of-function of ADORA2A. In one embodiment, the modified NK cell comprises genomic edits that result in a loss-of-function of CISH, a loss-of-function of TIGIT, and a loss-of-function of NKG2A. In one embodiment, the modified NK cell comprises genomic edits that result in a loss-of-function of CISH, a loss-of-function of ADORA2A, and a loss-of-function of NKG2A. In one embodiment, the modified NK cell comprises genomic edits that result in a loss-of-function of TIGIT, a loss-of-function of ADORA2A, and a loss-of-function of NKG2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of a soluble MICA and/or MICB, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of a soluble MICA and/or MICB, expression of an exogenous IL-12, expression of an exogenous IL-18, a loss of function in TGFbetaR2, a loss of function in B2M, a loss of function of PD1, a loss of function of TIGIT, and/or a loss of function of ADORA2A.

In some embodiments, the modified NK cells provided herein may comprise genomic edits that result in: expression of an exogenous a CD16 variant, e.g., hnCD16, expression of an exogenous IL15/IL15RA fusion, expression of an exogenous HLA-G, expression of an exogenous DN-TGFbetaR2, expression of an exogenous IL-12, expression of an exogenous IL-18, a loss of function in TGFbetaR2, a loss of function in B2M, a loss of function of PD1, a loss of function of TIGIT, and/or a loss of function of ADORA2A.

In some embodiments, the disclosure features a modified NK cell, wherein the modified NK cell does not express endogenous CD3, CD4, and/or CD8; and expresses at least one endogenous gene encoding: (i) CD56 (NCAM), CD49, and/or CD45; (ii) NK cell receptor (cluster of differentiation 16 (CD16)); (iii) natural killer group-2 member D (NKG2D); (iv) CD69; (v) a natural cytotoxicity receptor; or any combination of two or more thereof, wherein the modified NK cell further: (1) comprises at least one exogenous nucleic acid construct encoding: (i) a chimeric antigen receptor (CAR); (ii) a non-naturally occurring variant of immunoglobulin gamma Fc region receptor III (FcγRIII, CD16); (iii) interleukin 15 (IL-15); (iv) IL-15 receptor (IL-15R), or a variant thereof; (v) interleukin 12 (IL-12); (vi) interleukin-12 receptor (IL-12R), or a variant thereof, (vii) human leukocyte antigen G (HLA-G); (viii) human leukocyte antigen E (HLA-E); (ix) a nucleic acid sequence encoding leukocyte surface antigen cluster of differentiation CD47 (CD47); or any combination of two or more thereof; and/or (2) exhibits a loss of function of at least one of: (i) transforming growth factor beta receptor 2 (TGFβR2); (ii) adenosine A2a receptor (ADORA2A); (iii) T cell immunoreceptor with Ig and ITIM domains (TIGIT); (iv) β-2 microgobulin (B2M); (v) programmed cell death protein 1 (PD-1); (vi) cytokine inducible SH2 containing protein (CISH); (vii) class II, major histocompatibility complex, transactivator (CIITA); (viii) natural killer cell receptor NKG2A (natural killer group 2A); (ix) two or more HLA class II histocompatibility antigen alpha chain genes, and/or two or more HLA class II histocompatibility antigen beta chain genes; (x) cluster of differentiation 32B (CD32B, FCGR2B); (xi) T cell receptor alpha constant (TRAC); or any combination of two or more thereof. In one embodiment, the modified NK cell exhibits a loss of function of TGFβR2 and a loss-of-function of CISH. In one embodiment, the modified NK cell exhibits a loss-of-function of TGFbetaR2 and a loss-of-function of TIGIT. In one embodiment, the modified NK cell exhibits a loss-of-function of TGFbetaR2 and a loss-of-function of ADORA2A. In one embodiment, the modified NK cell exhibits a loss-of-function of TGFbetaR2 and a loss-of-function of NKG2A. In one embodiment, the modified NK cell exhibits a loss-of-function of CISH and a loss-of-function of TIGIT. In one embodiment, the modified NK cell exhibits a loss-of-function of CISH and a loss-of-function of ADORA2A. In one embodiment, the modified NK cell exhibits a loss-of-function of CISH and a loss-of-function of NKG2A. In one embodiment, the modified NK cell exhibits a loss-of-function of TIGIT and a loss-of-function of ADORA2A. In one embodiment, the modified NK cell exhibits a loss-of-function of TIGIT and a loss-of-function of NKG2A. In one embodiment, the modified NK cell exhibits a loss-of-function of ADORA2A and a loss-of-function of NKG2A. In one embodiment, the modified NK cell exhibits a loss-of-function of TGFbetaR2, a loss-of-function of CISH, and a loss-of-function of TIGIT. In one embodiment, the modified NK cell exhibits a loss-of-function of TGFbetaR2, a loss-of-function of CISH, and a loss-of-function of ADORA2A. In one embodiment, the modified NK cell exhibits a loss-of-function of TGFbetaR2, a loss-of-function of CISH, and a loss-of-function of NKG2A. In one embodiment, the modified NK cell exhibits a loss-of-function of TGFbetaR2, a loss-of-function of TIGIT, and a loss-of-function of ADORA2A. In one embodiment, the modified NK cell exhibits a loss-of-function of TGFbetaR2, a loss-of-function of TIGIT, and a loss-of-function of NKG2A. In one embodiment, the modified NK cell exhibits a loss-of-function of TGFbetaR2, a loss-of-function of ADORA2A, and a loss-of-function of NKG2A. In one embodiment, the modified NK cell exhibits a loss-of-function of CISH, a loss-of-function of TIGIT, and a loss-of-function of ADORA2A. In one embodiment, the modified NK cell exhibits a loss-of-function of CISH, a loss-of-function of TIGIT, and a loss-of-function of NKG2A. In one embodiment, the modified NK cell exhibits a loss-of-function of CISH, a loss-of-function of ADORA2A, and a loss-of-function of NKG2A. In one embodiment, the modified NK cell exhibits a loss-of-function of TIGIT, a loss-of-function of ADORA2A, and a loss-of-function of NKG2A.

In one embodiment, the modified NK cell does not express endogenous CD3, CD4, and/or CD8; and expresses at least one endogenous gene encoding: (i) CD56 (NCAM), CD49, and/or CD45; (ii) NK cell receptor (cluster of differentiation 16 (CD16)); (iii) natural killer group-2 member D (NKG2D); (iv) CD69; (v) a natural cytotoxicity receptor; or any combination of two or more thereof; wherein the modified NK cell further: (1) comprises at least one exogenous nucleic acid construct encoding: (i) a chimeric antigen receptor (CAR); (ii) a non-naturally occurring variant of immunoglobulin gamma Fc region receptor III (FcγRIII, CD16); (iii) interleukin 15 (IL-15); (iv) IL-15 receptor (IL-15R), or a variant thereof; (v) interleukin 12 (IL-12); (vi) interleukin-12 receptor (IL-12R), or a variant thereof; (vii) human leukocyte antigen G (HLA-G); (viii) human leukocyte antigen E (HLA-E); (ix) a nucleic acid sequence encoding leukocyte surface antigen cluster of differentiation CD47 (CD47); or any combination of two or more thereof; and/or (2) exhibits a loss of function of transforming growth factor beta receptor 2 (TGFβR2), cytokine inducible SH2 containing protein (CISH), or a combination thereof.

In some embodiments, the modified NK cells comprise genomic edits that result in: expression of a CD16 variant, e.g., a non-naturally occurring CD16 variant such as, for example, hnCD16 (see, e.g., Zhu et al., Blood 2017, 130:4452, the contents of which are incorporated herein in their entirety by reference); expression of an IL15/IL15RA fusion; a loss-of-function in TGF beta receptor 2 (TGFbetaR2); and a loss-of-function of CISH.

In another aspect, disclosed herein is a method of treating cancer in a subject, the method comprising administering to the subject a modified natural killer (NK) cell and a molecule comprising an Fc domain that binds cancer cells, e.g., an antibody, or an antigen-binding portion thereof, wherein the modified NK cell exhibits a loss of function of transforming growth factor beta receptor 2 (TGFβR2) and cytokine inducible SH2 containing protein (CISH), wherein the administering induces ADCC of a cancer cell in the subject, thereby treating the cancer in the subject.

In one aspect, disclosed herein is a method of inducing antibody-dependent cell-mediated cytotoxicity (ADCC) of a cancer cell, the method comprising contacting the cancer cell with a modified natural killer (NK) cell and a molecule comprising an Fc domain that binds cancer cells, e.g., an antibody, or antigen-binding portion thereof, wherein the modified NK cell exhibits a loss of function of transforming growth factor beta receptor 2 (TGFβR2) and cytokine inducible SH2 containing protein (CISH), thereby inducing ADCC of the cancer cell. In one embodiment, the contact is in vivo in a subject.

In one embodiment, the administration increases ADCC or enhances ADCC. In one embodiment, the administration increases ADCC by at least about 10%, at least about 15%, 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 2-fold, at least about 3-fold, at least about 4-fold, at least about 5-fold, at least about 6-fold, at least about 7-fold, at least about 8-fold, at least about 9-fold, or at least about 10-fold as compared to ADCC of a cancer cell using an unmodified NK cell and the antibody.

In another embodiment, the administering decreases tumor volume in the subject by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% after administering. In one embodiment, the administration decreases tumor volume in the subject at the values listed above at least about 5 days, 7 days, 10 days, 14 days, 21 days, 30 days, 1 month, 40 days, two months, three months, four months, five months, six months, seven months, eight months, nine months, ten months, eleven months, one year after administering.

In one embodiment, the administering increases the survival time of the subject. In one embodiment, the survival time of the subject is increased by at least about two-fold, about three-fold, about four-fold, or about five-fold as compared to a subject, e.g., comparator subject, who has not be administered the modified NK cell and the antibody; by at least about two-fold, about three-fold, about four-fold, or about five-fold as compared to a subject, e.g., comparator subject, who has been administered the antibody alone; and/or by at least about 50% about 75%, about 100%, about 150%, about two-fold, about three-fold, about four-fold, or about five-fold as compared to a subject, e.g., comparator subject, who has been administered the modified NK cell alone. In one embodiment, the comparator subject is a subject with the same type of cancer cell as the subject. In one embodiment, the comparator subject is a subject with the same type of cancer cell as the subject and a comparable tumor burden as the subject. In one embodiment, the survival time of the comparator subject is an average survival time calculated from a population of subjects having the same type of cancer cell, and/or the same stage of cancer, and/or the same amount of tumor burden as the subject.

In one embodiment, the contacting is in vitro. In one embodiment, the contacting is in a subject.

In one embodiment, the administration increases a level of TNFα by at least about two fold, at least about three-fold, at least about four-fold, or at least about five-fold as compared to a control level expression of TNFα. In one embodiment, the control level of TNFα is a level of TNFα produced by an unmodified NK cell under the same conditions. In another embodiment, the control level of TNFα is a reference level of TNFα. In one embodiment, the modified NK cell comprises an increase in level of TNFα by at least about two fold as compared to a control level expression of TNFα, wherein the control level of TNFα is a level of TNFα produced by an unmodified NK cell under the same conditions. In one embodiment, the modified NK cell comprises an increase in level of TNFα by at least about three fold as compared to the control level expression of TNFα.

In one embodiment, the administration increases a level of IFNγ by at least about two fold, at least about three-fold, at least about four-fold, or at least about five-fold as compared to a control level expression of IFNγ. In one embodiment, the control level of IFNγ is a level of IFNγ produced by an unmodified NK cell under the same conditions. In another embodiment, the control level of IFNγ is a reference level of IFNγ. In one embodiment, the modified NK cell comprises an increase in level of IFNγ by at least about two fold as compared to a control level expression of IFNγ, wherein the control level of IFNγ is a level of IFNγ produced by an unmodified NK cell under the same conditions. In one embodiment, the modified NK cell comprises an increase in level of IFNγ by at least about three fold as compared to the control level expression of IFNγ.

In one embodiment, the administration decreases normalized total integrated red object intensity in a tumor spheroid assay by at least about 20%, at least about 25%, at least about 30%, at least about 35%, at least about 40%, at least about 45%, at least about 50%, at least about 55%, at least about 60%, at least about 65%, at least bout 70%, at least about 75%, at least about 80%, at least about 85%, at least about 90%, at least about 95% or 100% as compared to a control level of normalized total integrated red object intensity, wherein the control level of normalized total integrated red object intensity is a level of normalized total integrated red object intensity produced using an unmodified NK cell under the same conditions. In one embodiment, the modified NK cell comprises a decrease in normalized total integrated red object intensity in a tumor spheroid assay by at least about 20% as compared to a control level of normalized total integrated red object intensity. In one embodiment, the control level of normalized total integrated red object intensity is a level of normalized total integrated red object intensity produced using an unmodified NK cell under the same conditions. In one embodiment, the modified NK cell comprises a decrease in normalized total integrated red object intensity in the tumor speroid assay by at least about 25%, at least about 30%, at least about 40%, at least about 50%, at least about 60%, at least about 75%, or about 100% as compared to the control level of normalized total integrated red object intensity.

In one embodiment, the administration increases a level of a cytolytic granule produced by the modified NK cell by at least about two fold, at least about three fold, at least about four fold, at least about five fold, at least about ten fold, or at least about twenty fold as compared to a control level expression of the cytolytic granule. In one embodiment, the control level of cytolytic granule is a level of cytolytic granule produced by an unmodified NK cell under the same conditions. In another embodiment, the control level of a cytolytic granule is a reference level of cytolytic granule. In one embodiment, the cytolytic granule is selected from the group consisting of GZMB, GZMA and GZMH. In one embodiment, the modified NK cell comprises an increase in level of a cytolytic granule by at least about two fold as compared to a control level expression of the cytolytic granule. In one embodiment, the cytolytic granule is selected from the group consisting of GZMB, GZMA and GZMH. In one embodiment, the control level of cytolytic granule is a level of cytolytic granule produced by an unmodified NK cell under the same conditions. In one embodiment, the modified NK cell comprises an increase in level of the cytolytic granule by at least about three fold as compared to the control level expression of the cytolytic granule.

In one embodiment, the administration increases a level of a cytolytic granule produced by the modified NK cell by at least about one hour, at least about two hours, at least about three hours, at least about four hours, or at least about five hours earlier as compared to a control level expression of the cytolytic granule. In one embodiment, the control level of cytolytic granule is a level of cytolytic granule produced by an unmodified NK cell under the same conditions. For example, the administration increases the level of the cytolytic granule produced by the modified NK cell by at least about one hour, at least about two hours, at least about three hours, at least about four hours, or at least about five hours earlier as compared to an observed increase in the level of the cytolytic granule produced by the unmodified NK cell under the same conditions. In another embodiment, the control level of a cytolytic granule is a reference level of cytolytic granule. In one embodiment, the cytolytic granule is selected from the group consisting of GZMB, GZMA and GZMH.

In one embodiment, the administration increases a production rate of a cytolytic granule by the modified NK cell by at least about two fold, at least about three fold, at least about four fold, or at least about five fold as compared to a control production rate of the cytolytic granule. In one embodiment, the control production rate of cytolytic granule is a production rate of cytolytic granule by an unmodified NK cell under the same conditions. In another embodiment, the control production rate of a cytolytic granule is a reference production rate of cytolytic granule. In one embodiment, the cytolytic granule is selected from the group consisting of GZMB, GZMA and GZMH. In one embodiment, the modified NK cell comprises an increase in production rate of a cytolytic granule by at least about two fold as compared to a control production rate of the cytolytic granule. In one embodiment, the cytolytic granule is selected from the group consisting of GZMB, GZMA and GZMH. In one embodiment, the control production rate of cytolytic granule is a production rate of cytolytic granule by an unmodified NK cell under the same conditions. In one embodiment, the modified NK cell comprises an increase in production rate of the cytolytic granule by at least about three fold as compared to the control production rate of the cytolytic granule.

In one embodiment, the administration increases a level of CD107a in the modified NK cells by at least about two fold, at least about three fold, at least about four fold, or at least about five fold as compared to a control level expression of CD107a. In one embodiment, the control level of CD107a is a level of CD107a in an unmodified NK cell under the same conditions. In another embodiment, the control level of CD107a is a reference level of CD107a. In one embodiment, the modified NK cell comprises an increase in level of CD107a by at least about two fold as compared to a control level expression of CD107a. In one embodiment, the control level of CD107a is a level of CD107a in an unmodified NK cell under the same conditions. In one embodiment, the modified NK cell comprises an increase in level of CD107a by at least about three fold as compared to the control level expression of CD107a.

In one embodiment, the cytotoxicity activity of the modified NK cell under a nutrient-depriving condition is at least about 20%, at least about 25%, at least about 30%, at least about 35%, at least about 40%, at least about 45%, at least about 50%, at least about 55%, at least about 60%, at least about 65%, at least bout 70%, at least about 75%, at least about 80%, at least about 85%, at least about 90%, at least about 95% or 100% higher as compared to a control level of cytotoxicity activity, wherein the control level of cytotoxicity activity is a cytotoxicity level of an unmodified NK cell under the same conditions. In one embodiment, the modified NK cell comprises an increase in cytotoxicity activity under a nutrient-depriving condition by at least about 20% as compared to a control level of cytotoxicity activity. In one embodiment, the control level of cytotoxicity activity is a cytotoxicity level of an unmodified NK cell under the same conditions. In one embodiment, the modified NK cell comprises an increase in cytotoxicity activity under the nutrient-depriving condition by at least about 25%, at least about 30%, at least about 40%, at least about 50%, at least about 60%, at least about 75% or about 100% as compared to the control level of cytotoxicity activity.

In one embodiment, the spare respiratory capacity of the modified NK cell is at least 20%, at least about 25%, at least about 30%, at least about 35%, at least about 40%, at least about 45%, at least about 50%, at least about 55%, at least about 60%, at least about 65%, at least bout 70%, at least about 75%, at least about 80%, at least about 85%, at least about 90%, at least about 95% or 100% higher as compared to a control level of spare respiratory capacity, wherein the control level of spare respiratory capacity is a level of spare respiratory capacity of an unmodified NK cell under the same conditions. In one embodiment, the modified NK cell comprises an increase in spare respiratory capacity by at least 20% as compared to a control level of spare respiratory capacity. In one embodiment, the control level of spare respiratory capacity is a level of spare respiratory capacity of an unmodified NK cell under the same conditions. In one embodiment, the modified NK cell comprises an increase in spare respiratory capacity by at least about 25%, at least about 30%, at least about 40%, at least about 50%, at least about 60%, at least about 75% or about 100% as compared to the control level of spare respiratory capacity.

In one embodiment, the molecule comprising an Fc domain that binds cancer cells, e.g., antibody, or antigen-binding portion thereof, binds epidermal growth factor receptor (EGFR), HER2, or CD20. In one embodiment, the antibody is cetuximab, trastuzumab, or rituximab, or an antigen-binding portion thereof.

In one embodiment, the modified NK cell is administered concurrently with the antibody. In one embodiment, the antibody is administered prior to the modified NK cell. In one embodiment, the antibody is administered 1 day, 2 days, 3 days, 4 days, 5 days, 6 days, 7 days, 8 days, 9 days, 10 days, 11 days, 12 days, 13 days, or 2 weeks prior to the modified NK cell. In one embodiment, the modified NK cell is administered prior to the antibody. In one embodiment, the modified NK cell is administered 1 day, 2 days, 3 days, 4 days, 5 days, 6 days, 7 days, 8 days, 9 days, 10 days, 11 days, 12 days, 13 days, or 2 weeks prior to the antibody. In another embodiment, the modified NK cell is administered once, and the antibody is administered at least two, three, four, or five times. In another embodiment, the modified NK cell is administered at least one, two, three, four or five times, and the antibody is administered at least one, two, three, four or five times, either concurrently or sequentially.

In one embodiment, the cancer cell is a head and neck cancer cell, breast cancer cell, colorectal cancer cell, gastric cancer cell, renal cell carcinoma (RCC) cell, or non-small cell lung cancer (NSCLC) cell, solid tumor cell, bladder cancer cell, hepatocellular carcinoma cell, prostate cancer cell, ovarian/uterine cancer cell, pancreatic cancer cell, mesothelioma cell, melanoma cell, glioblastoma cell, cervical cancer cell, oral cavity cancer cell, cancer of the pharynx, thyroid cancer cell, gallbladder cancer cell, soft tissue sarcoma, or a hematological cancer cell. In one embodiment, the cancer cell is a head and neck cancer cell.

In one embodiment, the modified NK cell has been modified using CRISPR prior to the administering. In one embodiment, the modified NK cell has been modified using a RNA guided nuclease and at least one guide RNA (gRNA). In one embodiment, the RNA guided nuclease comprises a sequence of SEQ ID NO: 1142, SEQ ID NO:1143, SEQ ID NO: 1144, SEQ ID NO: 1145, SEQ ID NO: 1146, SEQ ID NO:1147, SEQ ID NO: 1148, SEQ ID NO: 1149, or SEQ ID NO: 1150. In one embodiment, the RNA guided nuclease comprises a sequence of SEQ ID NO: 1146. In one embodiment, the gRNA targets a DNA sequence of any one of SEQ ID NOs:769-875 or 1174. In one embodiment the gRNA targets a DNA sequence of any one of SEQ ID NOs:540-768 or 1173. In one embodiment, the gRNA comprises a sequence of SEQ ID NO: 1164 or SEQ ID NO: 1170, and/or SEQ ID NO: 1166 or SEQ ID NO: 1172. In one embodiment, the modified NK cell was generated from a NK cell, e.g., a mature NK, or a stem cell. In one embodiment, the stem cell is an induced pluripotent stem cell (iPS) cell, a hematopoietic stem cell (HSC), or an embryonic stem cell. In one embodiment, the NK cell is an iNK cell.

### BRIEF DESCRIPTION OF THE DRAWINGS

FIGS. 1A and 1B depict that robust single and double-gene editing of TGFBR2 and CISH was achieved in NK cells. 72 hours after CRISPR-EngCas12a editing for each KO combination, editing at CISH and TGFBR2 were assessed by NGS in FIG. 1A, and viability was assessed by AO/PI staining in FIG. 1B. Data were obtained from three unique NK cell donors, representative of a minimum of five independent experiments.

FIGS. 2A and 2B depict that knockout (KO) of CISH and TGFBR2 by CRISPR-EngCas12a increased phosphorylation of STAT5 (pSTAT5) upon IL-15 stimulation and reduced phosphorylation of SMAD2/3 (pSMAD2/3) upon TGF-β stimulation. NK cells were cytokine-starved for 18 hours, 72 hours after CRISPR-EngCas12a editing, followed by re-stimulation for 120 min with IL-15 (FIG. 2A) or IL-15 and TGF-β (FIG. 2B), and analyzed by phosphoflow cytometry assay. Data are representative of four unique NK cell donors in two independent experiments. Statistical difference is the result of 1-way ANOVA analysis (*p<0.05; **p<0.01; ***p<0.001, ****p<0.0001).

FIGS. 3A, 3B, 3C, and 3D depict that double KO (DKO) of CISH/TGFBR2 in NK cells by CRISPR-EngCas12a editing increased inflammatory cytokine production after co-culturing with spheroids of ovarian cancer cell line SK-OV-3 (FIGS. 3A and 3B) and prostate cancer cell line PC-3 (FIGS. 3C and 3D) in comparison to unedited control NK cells. Supernatants were harvested at the conclusion of the spheroid assay (120 hrs) and analyzed for TNF-α and IFN-γ by AlphaLISA (+TGF-β conditions). Statistical difference is the result of 2-way ANOVA analysis (*p<0.05; **p<0.01; ***p<0.001, ****p<0.0001).

FIGS. 4A, 4B, 4C, and 4D depict that CRISPR-EngCas12a editing enhanced anti-tumor activity of NK cells against SK-OV-3 ovarian tumor compared with unedited control NK cells in the in vitro spheroid assay at different effector cell to target cell (E:T) ratios. FIGS. 4A and 4B depict the tumor spheroid analysis at 10:1 E:T ratio in the presence of 10 ng/ml TGF-β, without and with the addition of 10 μg/mL trastuzumab, respectively, as analyzed across a minimum of 4 unique donors and 3 independent experiments. Red object intensity was measured every two hours for 5 days on an Incucyte imaging system. FIGS. 4C and 4D depict the tumor spheroid analysis at 1.25:1, 2.5:1, 5:1 and 10:1 E:T ratios in the presence of 10 ng/ml TGF-β, without and with the addition of 10 μg/mL trastuzumab, respectively, as analyzed across a minimum of 4 unique donors and 3 independent experiments. Red object intensity is shown at 100 hours following NK cell addition.

FIG. 5 depicts amplified tumor killing by NK cells through antibody-dependent cellular cytotoxicity in vitro. At low E:T ratio of 1.25:1, the addition of 10 μg/mL trastuzumab significantly increased killing of SK-OV-3 tumor spheroids by both unedited and CISH/TGFBR2 DKO NK cells, as analyzed across a minimum of 4 unique donors and 3 independent experiments.

FIGS. 6A, 6B, 6C, and 6D depict that CRISPR-EngCas12a-edited NK cells reduced SK-OV-3 ovarian tumor burden more effectively than unedited control NK cells, leading to an increased median survival time in an in vivo mouse model. NSG mice (n=8 per group in two independent experiments) were inoculated via intraperitoneal (i.p.) with 0.5 million (FIGS. 6A and 6C) or 1 million (FIGS. 6B and 6D) luciferase-expressing SK-OV-3 cells. Seven days later, the mice were administered 10 million unedited NK cells or 10 million DKO NK cells by i.p. infusion. Tumor burden measured by bioluminescence signal from SK-OV-3 cells are shown in FIGS. 6A and 6B, and overall survival of mice are shown in FIGS. 6C and 6D. Data are representative of two independent experiments. Statistical difference is the result of 2-way ANOVA (*p<0.05; **p<0.01; ***p<0.001) for bioluminescence and log rank test for overall survival.

FIGS. 7A, 7B, 7C and 7D depict that trastuzumab mediated antibody-dependent cellular toxicity in NK cell treatments of SK-OV-3 tumor bearing mice. NSG mice (n=8 per group) were inoculated via intraperitoneal (i.p.) with 0.5 million luciferase-expressing SK-OV-3 cells. On day 7, mice were treated with 2.5 mpk isotype, 2.5 mpk trastuzumab, 10 million unedited CD56+NK cells, 10 million DKO CD56+NK cells or the combination of DKO CD56+NK cells with trastuzumab. The average tumor volumes are shown as mean±SEM (****p<0.0001, **p<0.01, *p<0.05, 2-way analysis of variance) (FIGS. 7A and 7B). Kaplan-Meier survival curves shown for the treatment groups as indicated (*p<0.05; **p<0.01; Gehan-Wilcoxon test) (FIGS. 7C and 7D). FIGS. 7A and 7C show that the DKO NK cells are effective at controlling tumor growth and increased mouse lifespan. FIGS. 7B and 7D show that administration of trastuzumab further reduced SK-PV-3 ovarian tumor burden and extended lifespan of tumor-bearing mice in treatments with DKO NK cells.

FIGS. 8A and 8B depict that DKO NK cells demonstrate more robust serial killing of Raji tumor cells over a tested period of more than 7 days with multiple de novo additions of Raji tumor target cells relative to control NK cells, and that combination with rituximab improved killing by both control and DKO NK cells. FIG. 8A shows the experimental set up of the assay. 200 thousand NK cells were seeded in each well. 10 thousand Raji tumor cells were added to the NK cells at the beginning of the assay, and subsequently 5 thousand tumor cells and IL-15 were bolused into each well every 48 hours. Surviving tumor cells were quantified by normalized total red object area. FIG. 8B shows that DKO NK cells demonstrate increased killing of Raji tumor cells relative to control NK and that the addition of rituximab improved killing by both types of NK cells.

FIG. 9A depicts upregulation of granzyme transcripts, GZMB, GZLMA an GZMH in CISH^{-/-} NK cells as assessed by NanoString analysis.

FIG. 9B depicts that GZMB transcripts were upregulated 22-fold in CISH/TGFBR2 DKO NK cells as quantified by RT-qPCR. TBP (TATA box binding protein) was used as a reference transcript.

FIG. 9C depicts that CISH/TGFBR2 DKO NK cells demonstrated enhanced tumor cytotoxicity relative to unedited control NK cells. CISH/TGFBR2 DKO NK cells were co-cultured with SK-OV-3 tumor spheroids in the presence of 10 ng/mL TGF-β over a time period of 36 hours at a 5:1 effector tumor ratio. Error bars represent standard deviation.

FIG. 9D shows representative Incucyte images of SK-OV3::GzmB cells co-cultured with CISH/TGFBR2 DKO NK cells or unedited NK control cells for 4 hours. FIG. 9D depicts that CISH/TGFBR2 DKO NK cells released more GzmB than unedited control NK cells when co-cultured with SK-OV-3 tumor cells.

FIG. 9E depicts that CISH/TGFBR2 DKO NK cells demonstrated higher levels of GzmB granulation at earlier time points relative to unedited NK control cells.

FIG. 10A depicts that CISH/TGFBR2 DKO NK cells had enhanced cytotoxicity when compared to unedited control NK cells in unfavorable metabolic conditions in isolation. CISH/TGFBR2 DKO NK cells were co-cultured with SK-OV-3 tumor spheroids without TGF-β at a 10:1 effector tumor ratio.

FIG. 10B depicts that CISH/TGFBR2 DKO NK cells had enhanced cytotoxicity when compared to unedited control NK cells in multifactorially unfavorable metabolic conditions. The CISH/TGFBR2 DKO NK cells or the unedited control cells were co-cultured with SK-OV-3 tumor spheroids in the presence of 10 ng/mL TGF-β at a 5:1 effector tumor ratio.

FIG. 10C depicts that CISH/TGFBR2 DKO NK cells had enhanced cytotoxicity when compared to unedited control NK cells against tumor cells evolved to grow in unfavorable metabolic conditions. The CISH/TGFBR2 DKO NK cells or the unedited control cells were co-cultured with SK-OV-3 tumor spheroids that were selectively evolved to grow in unfavorable metabolic conditions in the presence of 10 ng/mL TGF-β at a 10:1 effector tumor ratio. EC50 was measured at 100 hours.

FIG. 10D depicts that CISH/TGFBR2 DKO NK cells had a greater cytotoxicity potential in unfavorable metabolic conditions than in control media compared to unedited control NK cells. The CISH/TGFBR2 DKO NK cells or the unedited control cells were co-cultured with SK-OV-3 tumor spheroids that were selectively evolved to grow in unfavorable metabolic conditions in the presence of 10 ng/mL TGF-β at 100 hours at various effector target ratios as indicated.

FIG. 10E depicts that CISH/TGFBR2 DKO NK cells exhibited significantly greater metabolic fitness (i.e., greater spare respiratory capacity (SRC)) than unedited control NK cells after overnight IL-15 starvation. *p<0.05.

FIGS. 11A and 11B depict that CISH/TGFBR2 DKO NK cells enhanced anti-tumor activity against Nalm6 cells in the presence of TGF-β, respectively, as analyzed across a minimum of 5 unique donors and 2 independent experiments. CISH/TGFBR2 DKO NK cells and unedited control NK cells were co-cultured with Nalm6 tumor cells at a 20:1 effector tumor ratio in the presence of 5 ng/mL IL-15, without and with the addition of 10 ng/mL TGF-β. Increased cytotoxicity was observed in all conditions while a greater increase was observed when TGF-β was added in the cell culture.

FIG. 12 depicts that CISH/TGFBR2 DKO NK cells demonstrate robus serial killing against Nalm6 cells over a tested period up to 20 days with multiple additions of Nalm6 cells relative to control NK cells.

FIG. 13 depicts that CISH/TGFBR2 DKO NK cells continually killed Nalm6 tumor cells for more than 8 days, whereas unedited NK cells had limited serial killing effect. Data are representative of NK cells from 6 unique donors in 2 independnent experiments.

FIG. 14 depicts that CISH/TGFBR2 DKO NK cells produced increased levels of inflammatory cytokines (IFN-γ and TFN-α) throughout the serial-killing assay in the presence of TGF-β relative to unedited control NK cells.

FIGS. 15A, 15B, and 15C depict that CISH/TGFBR2 DKO NK cells demonstrated sustained serial-killing activity against numerous other hematologic tumor cell lines, e.g., Raji (Burkitt's lymphoma) (FIG. 15A), RPM18226 (multiple myeloma) (FIG. 15B) and THP-1 cells (acute monocytic leukemia) (FIG. 15C), in the presence of TGF-β. Data are representative of NK cells from 5 unique donors in 5 independent experiments.

### DETAILED DESCRIPTION

The present disclosure provides modified NK cells (or other lymphocytes) that are useful in NK cell therapy, e.g., in the context of immunotherapeutic approaches, in combination with a therapeutic antibody, or antigen-binding portion thereof, to generate striking antibody-dependent cellular cytotoxicity (ADCC) effects, thereby surprisingly increasing the effectiveness of the modified NK cells in killing target cells, e.g. cancer cells. ADCC is a mechanism of cell-mediated immune defense, where an immune effector cell actively lyses a target cell after its membrane-surface antigens have been bound by specific antibodies. To participate in ADCC, the immune effector cells must express Fc-gamma receptors (FcγR) to be able to recognize the Fc region of the antibodies that bind to the target cells. Most immune effector cells have both activating and inhibitory FcγR. An advantage of using NK cells to target cancer cells via ADCC is that, unlike other effector cells, NK cells only have activating FcγRs (e.g., FcγR IIIa, also known as CD16a, and FcγR IIc, also known as CD32c) and are believed to be the most important effectors of ADCC in humans. Thus, the use of the modified NK cells disclosed herein and antibodies targeting cancer cell-specific antigens to elicit ADCC provides novel and surprisingly effective immunotherapies.

Some aspects of the present disclosure provide compositions, methods, and strategies for the generation of modified NK cells. In some embodiments, such modified NK cells are generated by editing the genome of NK cells, e.g., mature NK cells. In one embodiment, NK cells are obtained from a healthy donor, and then edited using the compositions and methods described herein to make modified NK cells. For example, NK cell expansion ex vivo is described at least in Myers and Miller, Exploring the NK cell platform for cancer immunotherapy, Nat Rev Clin Oncol (2020), https://doi.org/10.1038/s41571-020-0426-7, the entire contents of which are expressly incorporated herein by reference.

In other embodiments, modified NK cells are generated by editing the genome of a cell from which an NK cell is derived, either in vitro or in vivo. In some embodiments, the cell from which and NK cell is derived is a stem cell, for example, a hematopoietic stem cell (HSC), or a pluripotent stem cells, such as, e.g., an embryonic stem cell (ES cell) or an induced pluripotent stem cell (iPS cell). For example, in some embodiments, modified NK cells are generated by editing the genome of an ES cell, an iPS cell, or a hematopoietic stem cell, and subsequently differentiating the edited stem cell into an NK cell. In some embodiments, where the generation of modified NK cells involves differentiation of the modified NK cell from an iPS cell, the editing of the genome may take place at any suitable time during the generation, maintenance, or differentiation of the iPS cell. For example, where a donor cell is reprogrammed into an iPS cell, the donor cell, e.g., a somatic cell such as, for example, a fibroblast cell or a T lymphocyte, may be subjected to the gene editing approaches described herein before reprogramming to an iPS cell, during the reprogramming procedure, or after the donor cell has been reprogrammed to an iPS cell.

NK cells derived from iPS cells are also referred to herein as iNK cells. In some embodiments, the present disclosure provides compositions, methods, and strategies for generating iNK cells that have been derived from developmentally mature cells, also referred to as somatic cells, such as, for example, fibroblasts or peripheral blood cells.

In some embodiments, the present disclosure provides compositions, methods, and strategies for generating iNK cells that have been derived from developmentally mature T cells (T cells that have undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an induced pluripotent stem (iPS) cell, and throughout differentiation of the resulting iPS cell to a somatic cell.

One advantage of using T cells for the generation of iPS cells is that T cells can be edited with relative ease, e.g., by CRISPR-based methods or other gene-editing methods.

Another advantage of using T cells for the generation of iPS cells is that the rearranged TCR locus allows for genetic tracking of individual cells and their daughter cells. If the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population.

A third advantage of using T cells in generating iNK cells carrying multiple edits is that certain karyotypic aberrations associated with chromosomal translocations are selected against in T cell culture. Such aberrations pose a concern when editing cells by CRISPR technology, and in particular when generating cells carrying multiple edits.

A fourth advantage of using T cell derived iPS cells as a starting point for the derivation of therapeutic lymphocytes is that it allows for the expression of a pre-screened TCR in the lymphocytes, e.g., via selecting the T cells for binding activity against a specific antigen, e.g., a tumor antigen, reprogramming the selected T cells to iPS cells, and then deriving lymphocytes from these iPS cells that express the TCR (e.g., T cells). This strategy would also allow for activating the TCR in other cell types, e.g., by genetic or epigenetic strategies.

A fifth advantage of using T cell derived iPS cells as a starting point for iNK differentiation is that the T cells retain at least part of their “epigenetic memory” throughout the reprogramming process, and thus subsequent differentiation of the same or a closely related cell type, such as iNK cells will be more efficient and/or result in higher quality cell populations as compared to approaches using non-related cells, such as fibroblasts, as a starting point for iNK derivation.

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.”

The conjunctions “or” and “and/or” are used interchangeably as non-exclusive disjunctions.

“Subject” means a human or non-human animal. A human subject can be any age (e.g., an infant, child, young adult, or adult), and may suffer from a disease, or may be in need of alteration of a gene or a combination of specific genes. Alternatively, the subject may be an animal, which term includes, but is not limited to, a mammal, and, more particularly, a non-human primate, a rodent (e.g., a mouse, rat, hamster, etc.), a rabbit, a guinea pig, a dog, a cat, and so on. In certain embodiments of this disclosure, the subject is livestock, e.g., a cow, a horse, a sheep, or a goat. In certain embodiments, the subject is poultry.

The terms “treatment,” “treat,” and “treating,” refer to a clinical intervention aimed to reverse, alleviate, delay the onset of, or inhibit the progress, and/or prevent or delay the recurrence of a disease or disorder, or one or more symptoms thereof, as described herein. Treatment, e.g., in the form of a modified NK cell or a population of modified NK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, treatment may be administered to a susceptible individual prior to the onset of symptoms (e.g., in light of genetic or other susceptibility factors). Treatment may also be continued after symptoms have resolved, for example to prevent or delay their recurrence.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

The term “endogenous,” as used herein in the context of nucleic acids (e.g., genes, protein-encoding genomic regions, promoters), refers to a native nucleic acid or protein in its natural location, e.g., within the genome of a cell. In contrast, the term “exogenous,” as used herein in the context of nucleic acids, e.g., expression constructs, cDNAs, indels, and nucleic acid vectors, refers to nucleic acids that have artificially been introduced into the genome of a cell using, for example, gene-editing or genetic engineering techniques, e.g., CRISPR-based editing techniques.

The terms “RNA-guided nuclease” and “RNA-guided nuclease molecule” are used interexchangably herein. In some embodiments, the RNA-guided nuclease is a RNA-guided DNA endonuclease enzyme. In some embodiments, the RNA-guided nuclease is a CRISPR nuclease. Non-limiting examples of RNA-guided nucleases are listed in Table 2 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

TABLE2
RNA-Guided Nucleases

| | Length | | |
|---|---|---|---|
| Nuclease | (a.a.) | PAM | Reference |
| SpCas9 | 1368 | NGG | Cong et al., Science. 2013; 339(6121): 819-23 |
| SaCas9 | 1053 | NNGRRT | Ran et al., Nature. 2015; 520(7546): 186-91. |
| (KKH) | 1067 | NNNRRT | Kleinstiver et al., Nat Biotechnol. 2015; 33(12): |
| SaCas9 | | | 1293-1298 |
| AsCpf1 | 1353 | TTTV | Zetsche et al., Nat Biotechnol. 2017; 35(1): 31-34. |
| (AsCas12a) | | | |
| LbCpf1 | 1274 | TTTV | Zetsche et al., Cell. 2015; 163(3): 759-71. |
| (LbCas 12a) | | | |
| CasX | 980 | TTC | Burstein et al., Nature. 2017; 542(7640): 237-241. |
| CasY | 1200 | TA | Burstein et al., Nature. 2017; 542(7640): 237-241. |
| Cas12h1 | 870 | RTR | Yan et al., Science. 2019; 363(6422): 88-91. |
| Cas12i1 | 1093 | TTN | Yan et al., Science. 2019; 363(6422): 88-91. |
| Cas12c1 | unknown | TG | Yan et al., Science. 2019; 363(6422): 88-91. |
| Cas12c2 | unknown | TN | Yan et al., Science. 2019; 363(6422): 88-91. |
| eSpCas9 | 1423 | NGG | Chen et al., Nature. 2017; 550(7676): 407-410. |
| Cas9-HF1 | 1367 | NGG | Chen et al., Nature. 2017; 550(7676): 407-410. |
| HypaCas9 | 1404 | NGG | Chen et al., Nature. 2017; 550(7676): 407-410. |
| dCas9-Fok1 | 1623 | NGG | U.S. Pat. No. 9,322,037 |
| Sniper-Cas9 | 1389 | NGG | Lee et al., Nat Commun. 2018; 9(1): 3048. |
| xCas9 | 1786 | NGG, NG, | Wang et al., Plant Biotechnol J. 2018; pbi.13053. |
| | | GAA, | |
| | | GAT | |
| AaCas 12b | 1129 | TTN | Teng et al. Cell Discov. 2018;4:63. |
| evoCas9 | 1423 | NGG | Casini et al., Nat Biotechnol. 2018; 36(3): 265-271. |
| SpCas9-NG | 1423 | NG | Nishimasu et al., Science. 2018; 361(6408): 1259-1262. |
| VRQR | 1368 | NGA | Li et al., The CRISPR Journal, 2018; 01:01 |
| VRER | 1372 | NGCG | Kleinstiver et al., Nature. 2016; 529(7587): 490-5. |
| NmeCas9 | 1082 | NNNNGAT | Amrani et al., Genome Biol. 2018; 19(1): 214. |
| | | T | |
| CjCas9 | 984 | NNNNRYA | Kim et al., Nat Commun. 2017; 8: 14500. |
| | | C | |
| BhCas12b | 1108 | ATTN | Strecker et al., Nat Commun. 2019 Jan. 22; 10(1): 212. |
| BhCas12b | 1108 | ATTN | Strecker et al., Nat Commun. 2019 Jan. 22; 10(1): 212. |
| V4 | | | |
| CasΦ | | | Pausch et al., Science 2020; 369(6501): 333-337. |

Additional suitable RNA-guided nucleases, e.g., Cas9 and Cas12 nucleases, will be apparent to the skilled artisan in view of the present disclosure, and the disclosure is not limited by the exemplary suitable nucleases provided herein. In some embodiment, a suitable nuclease is a Cas9 or Cpf1 (Cas12a) nuclease. In some embodiments, the disclosure also embraces nuclease variants, e.g., Cas9 or Cpf1 nuclease variants. A nuclease variant refers to a nuclease comprising an amino acid sequence characterized by one or more amino acid substitutions, deletions, or additions as compared to the wild type amino acid sequence of the nuclease. Suitable nucleases and nuclease variants may also include purification tags (e.g., polyhistidine tags) and signaling peptides, e.g., comprising or consisting of a nuclear localization signal sequence. Some non-limiting examples of suitable nucleases and nuclease variants are described in more detail elsewhere herein, and also include those described in PCT application PCT/US2019/22374, filed Mar. 14, 2019, and entitled “Systems and Methods for the Treatment of Hemoglobinopathies,” the entire contents of which are incorporated herein by reference.

In some embodiments, the RNA-guided nuclease is an Acidaminococcus sp. Cpf1 variant (AsCpf1 variant). Suitable Cpf1 nuclease variants, including suitable AsCpf1 variants will be known or apparent to those of ordinary skill in the art based on the present disclosure, and include, but are not limited to, the Cpf1 variants disclosed herein or otherwise known in the art. For example, in some embodiments, the RNA-guided nuclease is a Acidaminococcus sp. Cpf1 RR variant (AsCpf1-RR). In another embodiment, the RNA-guided nuclease is a Cpf1 RVR variant. For example, suitable Cpf1 variants include those having an M537R substitution, an H800A substitution, and/or an F870L substitution, or any combination thereof (numbering scheme according to AsCpf1 wild-type sequence). In some embodiments, the RNA-guided nuclease is an Acidaminococcus sp. Cpf1 variant (AsCpf1 variant) having an M537R substitution, an H800A substitution, and an F870L substitution (numbering scheme according to AsCpf1 wild-type sequence).

The term “hematopoietic stem cell,” or “definitive hematopoietic stem cell” as used herein, refers to CD34+ stem cells capable of giving rise to both mature myeloid and lymphoid cell types including T cells, natural killer cells and B cells.

As used herein, the terms “reprogramming” or “dedifferentiation” or “increasing cell potency” or “increasing developmental potency” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state. In some embodiments, the term “reprogramming” refers to de-differentiating a somatic cell, or a multipotent stem cell, into a pluripotent stem cell, also referred to as an induced pluripotent stem cell, or iPS cell. Suitable methods for the generation of iPS cells from somatic or multipotent stem cells are well known to those of skill in the art.

As used herein, the term “differentiation” is the process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. For example, an iPS cell can be differentiated into various more differentiated cell types, for example, a neural or a hematopoietic stem cell, a lymphocyte, a cardiomyocyte, and other cell types, upon treatment with suitable differentiation factors in the cell culture medium. Suitable methods, differentiation factors, and cell culture media for the differentiation of pluri- and multipotent cell types into more differentiated cell types are well known to those of skill in the art. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

As used herein, the terms “differentiation marker,” “differentiation marker gene,” or “differentiation gene,” refers to genes or proteins whose expression are indicative of cell differentiation occurring within a cell, such as a pluripotent cell. Differentiation marker genes include, but are not limited to, the following genes: CD34, CD4, CD8, CD3, CD56 (NCAM), CD49, CD45; NK cell receptor (cluster of differentiation 16 (CD16)), natural killer group-2 member D (NKG2D), CD69, NKp30, NKp44, NKp46, CD158b, FOXA2, FGF5, SOX17, XIST, NODAL, COL3A1, OTX2, DUSP6, EOMES, NR2F2, NROB1, CXCR4, CYP2B6, GAT A3, GATA4, ERBB4, GATA6, HOXC6, INHA, SMAD6, RORA, NIPBL, TNFSF11, CDH11, ZIC4, GAL, SOX3, PITX2, APOA2, CXCL5, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CD1D FOXG1, LEFTY1, TUJ1, T gene (Brachyury), ZIC1, GATA1, GATA2, HDAC4, HDAC5, HDAC7, HDAC9, NOTCH1, NOTCH2, NOTCH4, PAX5, RBPJ, RUNX1, STAT1 and STAT3.

As used herein, the term “differentiation marker gene profile,” or “differentiation gene profile,” “differentiation gene expression profile,” “differentiation gene expression signature,” “differentiation gene expression panel,” “differentiation gene panel,” or “differentiation gene signature” refers to the expression or levels of expression of a plurality of differentiation marker genes.

As used herein in the context of cellular developmental potential, the term “potency” or “developmental potency” refers to the sum of all developmental options accessible to the cell (i.e., the developmental potency). The continuum of cell potency includes, but is not limited to, totipotent cells, pluripotent cells, multipotent cells, oligopotent cells, unipotent cells, and terminally differentiated cells.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

As used herein, the term “induced pluripotent stem cell” or, iPS cell refers to a stem cell obtained from a differentiated somatic, e.g., adult, neonatal, or fetal cell by a process referred to as reprogramming into cells capable of differentiating into tissues of all three germ or dermal layers: mesoderm, endoderm, and ectoderm. IPS cells are not found in nature.

As used herein, the term “embryonic stem cell” refers to pluripotent stem cells derived from the inner cell mass of the embryonic blastocyst. Embryonic stem cells are pluripotent and give rise during development to all derivatives of the three primary germ layers: ectoderm, endoderm and mesoderm. They do not contribute to the extra-embryonic membranes or the placenta, i.e., are not totipotent.

As used herein, the term “multipotent stem cell” refers to a cell that has the developmental potential to differentiate into cells of one or more germ layers (ectoderm, mesoderm and endoderm), but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, the term “multipotency” refers to a state of a cell with a degree of developmental potential that is less than totipotent and pluripotent.

Pluripotency can be determined, in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GC™-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “pluripotent stem cell morphology” refers to the classical morphological features of an embryonic stem cell. Normal embryonic stem cell morphology is characterized by being round and small in shape, with a high nucleus-to-cytoplasm ratio, the notable presence of nucleoli, and typical intercell spacing.

As used herein, the term “nutrient-depriving condition” refers to unfavorable growth or metabolic conditions where either a lower level of nutrients or a lack of nutrients is observed. Nutrient deprivation is one of the hallmark conditions of the tumor microenvironment. The rapid growth of the tumor leads to the development of a hypoxic and nutrient deprived microenvironement within the core of the tumor mass due to an insufficient blood supply. In some embodiments, the nutrient-depriving condition comprises a decreasing concentration of nutrients for cell metabolism, e.g., glucose or glutamine. In some embodiments, the nutrient-depriving condition comprises a decreasing concentration of glucose, e.g., a concentration of glucose from about 10 mM, about 9 mM, about 8 mM, about 7 mM, about 6 mM, about 5 mM, about 4 mM, about 3 mM, about 2 mM or about 1 mM to a concentration of glucose less than about 1 mM, e.g., about 0.9 mM, about 0.8 mM, about 0.7 mM, about 0.6 mM, about 0.5 mM, about 0.4 mM, about 0.3 mM, about 0.2 mM or about 0.1 mM. In some embodiments, the nutrient-depriving condition comprises a decreasing concentration of glutamine, e.g., a concentration of glutamine from about 10 mM, about 9 mM, about 8 mM, about 7 mM, about 6 mM, about 5 mM, about 4 mM, about 3 mM, about 2 mM or about 1 mM to a concentration of glutamine less than about 1 mM, e.g., about 0.9 mM, about 0.8 mM, about 0.7 mM, about 0.6 mM, about 0.5 mM, about 0.4 mM, about 0.3 mM, about 0.2 mM or about 0.1 mM. In some embodiments, the nutrient-depriving condition comprises an increasing concentration of inhibitory metabolic, e.g., lactate, e.g., a concentration of lactate from about 0 mM, about 0.1 mM, about 0.2 mM, about 0.3 mM, about 0.4 mM, about 0.5 mM, about 0.6 mM, about 0.7 mM, about 0.8 mM, about 0.9 mM or about 1 mM to a concentration of lactate about 10 mM, about 15 mM, about 20 mM, about 25 mM, about 30 mM, about 35 mM, about 40 mM, about 45 mM or about 50 mM. In another embodiment, the nutrient-depriving condition comprises a decreasing pH, e.g., from a pH about 7.5, about 7.4, about 7.3, about 7.2, about 7.1 or about 7 to a pH about 6.9, about 6.8, about 6.7, about 6.6 or about 6.5.

As used herein, the term “spare respiratory capacity” refers to a functional parameter for evaluation of mitochondrial reserve. Spare respiratory capacity is the difference between basal ATP production and its maximal activity. When cells are subjected to stress, energy demand increases, with more ATP required to maintain cellular functions. A cell with a larger spare respiratory capacity can produce more ATP and overcome more stress.

### Genome Editing Systems

The present disclosure relates to the generation of modified NK cells, e.g., NK cells the genome of which has been modified, or that are derived from a multipotent or pluripotent stem cell, e.g., an HSC, ES cell, or iPS cell, the genome of which has been modified. The NK cells and stem cells provided herein can be modified using any gene-editing technology known to those of ordinary skill in the art, including, for example, by using genome editing systems, e.g., CRISPR.

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”, incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g. fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g. GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, e.g., as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate that, although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

In some embodiments, the guide RNA used comprises a modification as compared to the standard gRNA scaffold. Such modifications may comprise, for example, chemical modifications of a part of the gRNA, e.g., of a nucleobase or backbone moiety. In some embodiments, such a modification may also include the presence of a DNA nucleotide within the gRNA, e.g., within or outside of the targeting domain. In some embodiments, the modification may include an extension of the gRNA scaffold, e.g., by addition of 1-100 nucleotides, including RNA and/or DNA nucleotides at the 3′ or the 5′ terminus of the guide RNA, e.g., at the terminus distal to the targeting domain.

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases. In certain embodiments, a DNA extension may comprise a sequence set forth in Table 3.

In certain embodiments, a gRNA used herein includes a DNA extension as well as one or more chemical modification, e.g., one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a DNA extension may comprise a sequence set forth in Table 3 that includes a DNA extension. Without wishing to be bound by theory, it is contemplated that any DNA extension may be used herein, so long as it does not hybridize to the target nucleic acid being targeted by the gRNA. In some embodiments, a gRNA with a DNA extension exhibits an increase in editing at the target nucleic acid site relative to a gRNA which does not include such a DNA extension. In some embodiments, a gRNA with a DNA extension exhibits more effective delivery into NK cells and/or stem cells relative to a gRNA which does not include such an extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, an RNA extension may comprise a sequence set forth in Table 3.

In certain embodiments, a gRNA used herein includes an RNA extension as well as one or more chemical modifications, e.g., one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth in Table 3 that includes an RNA extension. gRNAs including an RNA extension at the 5′ end of the gRNA may comprise a sequence disclosed herein. gRNAs including an RNA extension at the 3′ end of the gRNA may comprise a sequence disclosed herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end, and/or a chemical modification as disclosed herein, is complexed with a RNA-guided nuclease, e.g., an AsCpf1 nuclease, to form an RNP, which is then employed to edit a target cell, e.g., an NK cell.

Exemplary suitable 5′ extensions for Cpf1 guide RNAs are provided in the table below:

TABLE3
gRNA 5′ Extensions

| SEQ ID NO: | 5′ extension sequence | 5′ modification |
|---|---|---|
| | rCrUrUrUrU | +5 RNA |
| 1 | rArArGrArCrCrUrUrUrU | +10 RNA |
| 2 | rArUrGrUrGrUrUrUrUrUrGrUrCrArArArArGrArCrCrUrUrUrU | +25 RNA |
| 3 | rArGrGrCrCrArGrCrUrUrGrCrCrGrGrUrUrUrUrUrUrArGrUrCrGrUr | +60 RNA |
| | GrCrUrGrCrUrUrCrArUrGrUrGrUrUrUrUrUrGrUrCrArArArArGrAr | |
| | CrCrUrUrUrU | |
| | CTTTT | +5 DNA |
| 4 | AAGACCTTTT | +10 DNA |
| 5 | ATGTGTTTTTGTCAAAAGACCTTTT | +25 DNA |
| 6 | AGGCCAGCTTGCCGGTTTTTTAGTCGTGCTGCTTCATGTGTTT | +60 DNA |
| | TTGTCAAAAGACCTTTT | |
| 7 | TTTTTGTCAAAAGACCTTTT | +20 DNA |
| 8 | GCTTCATGTGTTTTTGTCAAAAGACCTTTT | +30 DNA |
| 9 | GCCGGTTTTTTAGTCGTGCTGCTTCATGTGTTTTTGTCAAAAG | +50 DNA |
| | ACCTTTT | |
| 10 | TAGTCGTGCTGCTTCATGTGTTTTTGTCAAAAGACCTTTT | +40 DNA |
| 11 | C*C*GAAGTTTTCTTCGGTTTT | +20 DNA + 2xPS |
| 12 | T*T*TTTCCGAAGTTTTCTTCGGTTTT | +25 DNA + 2xPS |
| 13 | A*A*CGCTTTTTCCGAAGTTTTCTTCGGTTTT | +30 DNA + 2xPS |
| 14 | G*C*GTTGTTTTCAACGCTTTTTCCGAAGTTTTCTTCGGTTTT | +41 DNA + 2xPS |
| 15 | G*G*CTTCTTTTGAAGCCTTTTTGCGTTGTTTTCAACGCTTTTT | +62 DNA + 2xPS |
| | CCGAAGTTTTCTTCGGTTTT | |
| 16 | A*T*GTGTTTTTGTCAAAAGACCTTTT | +25 DNA + 2xPS |
| 17 | AAAAAAAAAAAAAAAAAAAAAAAAA | +25 A |
| 18 | TTTTTTTTTTTTTTTTTTTTTTTTT | +25 T |
| 19 | mA*mU*rGrUrGrUrUrUrUrUrGrUrCrArArArArGrArCrCrUrUrUrU | +25 RNA + 2xPS |
| 20 | mA*mA*rArArArArArArArArArArArArArArArArArArArArArArA | PolyA RNA + 2×PS |
| 21 | mU*mU*rUrUrUrUrUrUrUrUrUrUrUrUrUrUrUrUrUrUrUrUrUrUrU | PolyU RNA + 2×PS |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

In certain embodiments, one or more or all of the nucleotides in a gRNA are modified. Strategies for modifying a gRNA are described in WO2019/152519, published Aug. 8, 2019, the entire contents of which are expressly incorporated herein by reference.

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf-1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and this contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO:22) would have a targeting domain of the corresponding RNA sequence UCUGCAGAAAUGUUCCCCGU (SEQ ID NO:23). As will be apparent to the skilled artisan, such a targeting sequence would be linked to a suitable guide RNA scaffold, e.g., a crRNA scaffold sequence or a chimeric crRNA/tracerRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO:24), added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO:25). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArUrUrCrUr GrCrArGrArArArUrGrUrUrCrCrCrCrGrU (SEQ ID NO:26), ATGTGTTTTTGTCAAAAGACCTTTTrUrArArUrUrUrCrUrArCrUrCrUrUrGrUrArGrArUrArCrUr GrArCrArGrCrGrUrGrArArCrArGrGrUrArG (SEQ ID NO: 1164), or ATGTGTTTTTGTCAAAAGACCTTTTrUrArArUrUrUrCrUrArCrUrCrUrUrGrUrArGrArUrUrGrAr UrGrUrGrArGrArUrUrUrUrCrCrArCrCrU (SEQ ID NO: 1166). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

In some embodiments the gRNA for use in the disclosure is a gRNA targeting TGFbetaR2 (TGFβR2 gRNA). In some embodiments, the gRNA targeting TGFbetaR2 is one or more of the gRNAs described in Table 4.

TABLE4
TGFbetaR2 gRNAs

| | gRNA Targeting Domain Sequence | | | |
|---|---|---|---|---|
| Name | (DNA) | Length | SEQ ID NO: | Enzyme |
| TGFBR24326 | CAGGACGATGTGCAGCGGCC | 20 | 540 | AsCpf1 RR |
| TGFBR24327 | ACCGCACGTTCAGAAGTCGG | 20 | 541 | AsCpf1 RR |
| TGFBR24328 | ACAACTGTGTAAATTTTGTG | 20 | 542 | AsCpf1 RR |
| TGFBR24329 | CAACTGTGTAAATTTTGTGA | 20 | 543 | AsCpf1 RR |
| TGFBR24330 | ACCTGTGACAACCAGAAATC | 20 | 544 | AsCpf1 RR |
| TGFBR24331 | CCTGTGACAACCAGAAATCC | 20 | 545 | AsCpf1 RR |
| TGFBR24332 | TGTGGCTTCTCACAGATGGA | 20 | 546 | AsCpf1 RR |
| TGFBR24333 | TCTGTGAGAAGCCACAGGAA | 20 | 547 | AsCpf1 RR |
| TGFBR24334 | AAGCTCCCCTACCATGACTT | 20 | 548 | AsCpf1 RR |
| TGFBR24335 | GAATAAAGTCATGGTAGGGG | 20 | 549 | AsCpf1 RR |
| TGFBR24336 | AGAATAAAGTCATGGTAGGG | 20 | 550 | AsCpf1 RR |
| TGFBR24337 | CTACCATGACTTTATTCTGG | 20 | 551 | AsCpf1 RR |
| TGFBR24338 | TACCATGACTTTATTCTGGA | 20 | 552 | AsCpf1 RR |
| TGFBR24339 | TAATGCACTTTGGAGAAGCA | 20 | 553 | AsCpf1 RR |
| TGFBR24340 | TTCATAATGCACTTTGGAGA | 20 | 554 | AsCpf1 RR |
| TGFBR24341 | AAGTGCATTATGAAGGAAAA | 20 | 555 | AsCpf1 RR |
| TGFBR24342 | TGTGTTCCTGTAGCTCTGAT | 20 | 556 | AsCpf1 RR |
| TGFBR24343 | TGTAGCTCTGATGAGTGCAA | 20 | 557 | AsCpf1 RR |
| TGFBR24344 | AGTGACAGGCATCAGCCTCC | 20 | 558 | AsCpf1 RR |
| TGFBR24345 | AGTGGTGGCAGGAGGCTGAT | 20 | 559 | AsCpf1 RR |
| TGFBR24346 | AGGTTGAACTCAGCTTCTGC | 20 | 560 | AsCpf1 RR |
| TGFBR24347 | CAGGTTGAACTCAGCTTCTG | 20 | 561 | AsCpf1 RR |
| TGFBR24348 | ACCTGGGAAACCGGCAAGAC | 20 | 562 | AsCpf1 RR |
| TGFBR24349 | CGTCTTGCCGGTTTCCCAGG | 20 | 563 | AsCpf1 RR |
| TGFBR24350 | GCGTCTTGCCGGTTTCCCAG | 20 | 564 | AsCpf1 RR |
| TGFBR24351 | TGAGCTTCCGCGTCTTGCCG | 20 | 565 | AsCpf1 RR |
| TGFBR24352 | GCGAGCACTGTGCCATCATC | 20 | 566 | AsCpf1 RR |
| TGFBR24353 | GGATGATGGCACAGTGCTCG | 20 | 567 | AsCpf1 RR |
| TGFBR24354 | AGGATGATGGCACAGTGCTC | 20 | 568 | AsCpf1 RR |
| TGFBR24355 | CGTGTGCCAACAACATCAAC | 20 | 569 | AsCpf1 RR |
| TGFBR24356 | GCTCAATGGGCAGCAGCTCT | 20 | 570 | AsCpf1 RR |
| TGFBR24357 | ACCAGGGTGTCCAGCTCAAT | 20 | 571 | AsCpf1 RR |
| TGFBR24358 | CACCAGGGTGTCCAGCTCAA | 20 | 572 | AsCpf1 RR |
| TGFBR24359 | CCACCAGGGTGTCCAGCTCA | 20 | 573 | AsCpf1 RR |
| TGFBR24360 | GCTTGGCCTTATAGACCTCA | 20 | 574 | AsCpf1 RR |
| TGFBR24361 | GAGCAGTTTGAGACAGTGGC | 20 | 575 | AsCpf1 RR |
| TGFBR24362 | AGAGGCATACTCCTCATAGG | 20 | 576 | AsCpf1 RR |
| TGFBR24363 | CTATGAGGAGTATGCCTCTT | 20 | 577 | AsCpf1 RR |
| TGFBR24364 | AAGAGGCATACTCCTCATAG | 20 | 578 | AsCpf1 RR |
| TGFBR24365 | TATGAGGAGTATGCCTCTTG | 20 | 579 | AsCpf1 RR |
| TGFBR24366 | GATTGATGTCTGAGAAGATG | 20 | 580 | AsCpf1 RR |
| TGFBR24367 | CTCCTCAGCCGTCAGGAACT | 20 | 581 | AsCpf1 RR |
| TGFBR24368 | GTTCCTGACGGCTGAGGAGC | 20 | 582 | AsCpf1 RR |
| TGFBR24369 | GCTCCTCAGCCGTCAGGAAC | 20 | 583 | AsCpf1 RR |
| TGFBR24370 | TGACGGCTGAGGAGCGGAAG | 20 | 584 | AsCpf1 RR |
| TGFBR24371 | TCTTCCGCTCCTCAGCCGTC | 20 | 585 | AsCpf1 RR |
| TGFBR24372 | AACTCCGTCTTCCGCTCCTC | 20 | 586 | AsCpf1 RR |
| TGFBR24373 | CAACTCCGTCTTCCGCTCCT | 20 | 587 | AsCpf1 RR |
| TGFBR24374 | CCAACTCCGTCTTCCGCTCC | 20 | 588 | AsCpf1 RR |
| TGFBR24375 | ACGCCAAGGGCAACCTACAG | 20 | 589 | AsCpf1 RR |
| TGFBR24376 | CGCCAAGGGCAACCTACAGG | 20 | 590 | AsCpf1 RR |
| TGFBR24377 | AGCTGATGACATGCCGCGTC | 20 | 591 | AsCpf1 RR |
| TGFBR24378 | GGGCGAGGGAGCTGCCCAGC | 20 | 592 | AsCpf1 RR |
| TGFBR24379 | CGGGCGAGGGAGCTGCCCAG | 20 | 593 | AsCpf1 RR |
| TGFBR24380 | CCGGGCGAGGGAGCTGCCCA | 20 | 594 | AsCpf1 RR |
| TGFBR24381 | TCGCCCGGGGGATTGCTCAC | 20 | 595 | AsCpf1 RR |
| TGFBR24382 | ACATGGAGTGTGATCACTGT | 20 | 596 | AsCpf1 RR |
| TGFBR24383 | CAGTGATCACACTCCATGTG | 20 | 597 | AsCpf1 RR |
| TGFBR24384 | TGTGGGAGGCCCAAGATGCC | 20 | 598 | AsCpf1 RR |
| TGFBR24385 | TGTGCACGATGGGCATCTTG | 20 | 599 | AsCpf1 RR |
| TGFBR24386 | CGAGGATATTGGAGCTCTTG | 20 | 600 | AsCpf1 RR |
| TGFBR24387 | ATATCCTCGTGAAGAACGAC | 20 | 601 | AsCpf1 RR |
| TGFBR24388 | GACGCAGGGAAAGCCCAAAG | 20 | 602 | AsCpf1 RR |
| TGFBR24389 | CTGCGTCTGGACCCTACTCT | 20 | 603 | AsCpf1 RR |
| TGFBR24390 | TGCGTCTGGACCCTACTCTG | 20 | 604 | AsCpf1 RR |
| TGFBR24391 | CAGACAGAGTAGGGTCCAGA | 20 | 605 | AsCpf1 RR |
| TGFBR24392 | GCCAGCACGATCCCACCGCA | 20 | 606 | AsCpf1 RVR |
| TGFBR24393 | AAGGAAAAAAAAAAGCCTGG | 20 | 607 | AsCpf1 RVR |
| TGFBR24394 | ACACCAGCAATCCTGACTTG | 20 | 608 | AsCpf1 RVR |
| TGFBR24395 | ACTAGCAACAAGTCAGGATT | 20 | 609 | AsCpf1 RVR |
| TGFBR24396 | GCAACTCCCAGTGGTGGCAG | 20 | 610 | AsCpf1 RVR |
| TGFBR24397 | TGTCATCATCATCTTCTACT | 20 | 611 | AsCpf1 RVR |
| TGFBR24398 | GACCTCAGCAAAGCGACCTT | 20 | 612 | AsCpf1 RVR |
| TGFBR24399 | AGGCCAAGCTGAAGCAGAAC | 20 | 613 | AsCpf1 RVR |
| TGFBR24400 | AGGAGTATGCCTCTTGGAAG | 20 | 614 | AsCpf1 RVR |
| TGFBR24401 | CCTCTTGGAAGACAGAGAAG | 20 | 615 | AsCpf1 RVR |
| TGFBR24402 | TTCTCATGCTTCAGATTGAT | 20 | 616 | AsCpf1 RVR |
| TGFBR24403 | CTCGTGAAGAACGACCTAAC | 20 | 617 | AsCpf1 RVR |
| TGFbR2036 | GGCCGCTGCACATCGTCCTG | 20 | 618 | SpyCas9 |
| TGFbR2037 | GCGGGGTCTGCCATGGGTCG | 20 | 619 | SpyCas9 |
| TGFbR2038 | AGTTGCTCATGCAGGATTTC | 20 | 620 | SpyCas9 |
| TGFbR2039 | CCAGAATAAAGTCATGGTAG | 20 | 621 | SpyCas9 |
| TGFbR2040 | CCCCTACCATGACTTTATTC | 20 | 622 | SpyCas9 |
| TGFbR2041 | AAGTCATGGTAGGGGAGCTT | 20 | 623 | SpyCas9 |
| TGFbR2042 | AGTCATGGTAGGGGAGCTTG | 20 | 624 | SpyCas9 |
| TGFbR2043 | ATTGCACTCATCAGAGCTAC | 20 | 625 | SpyCas9 |
| TGFbR2044 | CCTAGAGTGAAGAGATTCAT | 20 | 626 | SpyCas9 |
| TGFbR2045 | CCAATGAATCTCTTCACTCT | 20 | 627 | SpyCas9 |
| TGFbR2046 | AAAGTCATGGTAGGGGAGCT | 20 | 628 | SpyCas9 |
| TGFbR2047 | GTGAGCAATCCCCCGGGCGA | 20 | 629 | SpyCas9 |
| TGFbR2048 | GTCGTTCTTCACGAGGATAT | 20 | 630 | SpyCas9 |
| TGFbR2049 | GCCGCGTCAGGTACTCCTGT | 20 | 631 | SpyCas9 |
| TGFbR2050 | GACGCGGCATGTCATCAGCT | 20 | 632 | SpyCas9 |
| TGFbR2051 | GCTTCTGCTGCCGGTTAACG | 20 | 633 | SpyCas9 |
| TGFbR2052 | GTGGATGACCTGGCTAACAG | 20 | 634 | SpyCas9 |
| TGFbR2053 | GTGATCACACTCCATGTGGG | 20 | 635 | SpyCas9 |
| TGFbR2054 | GCCCATTGAGCTGGACACCC | 20 | 636 | SpyCas9 |
| TGFbR2055 | GCGGTCATCTTCCAGGATGA | 20 | 637 | SpyCas9 |
| TGFbR2056 | GGGAGCTGCCCAGCTTGCGC | 20 | 638 | SpyCas9 |
| TGFbR2057 | GTTGATGTTGTTGGCACACG | 20 | 639 | SpyCas9 |
| TGFbR2058 | GGCATCTTGGGCCTCCCACA | 20 | 640 | SpyCas9 |
| TGFbR2059 | GCGGCATGTCATCAGCTGGG | 20 | 641 | SpyCas9 |
| TGFbR2060 | GCTCCTCAGCCGTCAGGAAC | 20 | 642 | SpyCas9 |
| TGFbR2061 | GCTGGTGTTATATTCTGATG | 20 | 643 | SpyCas9 |
| TGFbR2062 | CCGACTTCTGAACGTGCGGT | 20 | 644 | SpyCas9 |
| TGFbR2063 | TGCTGGCGATACGCGTCCAC | 20 | 645 | SpyCas9 |
| TGFbR2064 | CCCGACTTCTGAACGTGCGG | 20 | 646 | SpyCas9 |
| TGFbR2065 | CCACCGCACGTTCAGAAGTC | 20 | 647 | SpyCas9 |
| TGFbR2066 | TCACCCGACTTCTGAACGTG | 20 | 648 | SpyCas9 |
| TGFbR2067 | CCCACCGCACGTTCAGAAGT | 20 | 649 | SpyCas9 |
| TGFbR2068 | CGAGCAGCGGGGTCTGCCAT | 20 | 650 | SpyCas9 |
| TGFbR2069 | ACGAGCAGCGGGGTCTGCCA | 20 | 651 | SpyCas9 |
| TGFbR2070 | AGCGGGGTCTGCCATGGGTC | 20 | 652 | SpyCas9 |
| TGFbR2071 | CCTGAGCAGCCCCCGACCCA | 20 | 653 | SpyCas9 |
| TGFbR2072 | CCATGGGTCGGGGGCTGCTC | 20 | 654 | SpyCas9 |
| TGFbR2073 | AACGTGCGGTGGGATCGTGC | 20 | 655 | SpyCas9 |
| TGFbR2074 | GGACGATGTGCAGCGGCCAC | 20 | 656 | SpyCas9 |
| TGFbR2075 | GTCCACAGGACGATGTGCAG | 20 | 657 | SpyCas9 |
| TGFbR2076 | CATGGGTCGGGGGCTGCTCA | 20 | 658 | SpyCas9 |
| TGFbR2077 | CAGCGGGGTCTGCCATGGGT | 20 | 659 | SpyCas9 |
| TGFbR2078 | ATGGGTCGGGGGCTGCTCAG | 20 | 660 | SpyCas9 |
| TGFbR2079 | CGGGGTCTGCCATGGGTCGG | 20 | 661 | SpyCas9 |
| TGFbR2080 | AGGAAGTCTGTGTGGCTGTA | 20 | 662 | SpyCas9 |
| TGFbR2081 | CTCCATCTGTGAGAAGCCAC | 20 | 663 | SpyCas9 |
| TGFbR2082 | ATGATAGTCACTGACAACAA | 20 | 664 | SpyCas9 |
| TGFbR2083 | GATGCTGCAGTTGCTCATGC | 20 | 665 | SpyCas9 |
| TGFbR2084 | ACAGCCACACAGACTTCCTG | 20 | 666 | SpyCas9 |
| TGFbR2085 | GAAGCCACAGGAAGTCTGTG | 20 | 667 | SpyCas9 |
| TGFbR2086 | TTCCTGTGGCTTCTCACAGA | 20 | 668 | SpyCas9 |
| TGFbR2087 | CTGTGGCTTCTCACAGATGG | 20 | 669 | SpyCas9 |
| TGFbR2088 | TCACAAAATTTACACAGTTG | 20 | 670 | SpyCas9 |
| TGFbR2089 | GACAACATCATCTTCTCAGA | 20 | 671 | SpyCas9 |
| TGFbR2090 | TCCAGAATAAAGTCATGGTA | 20 | 672 | SpyCas9 |
| TGFbR2091 | GGTAGGGGAGCTTGGGGTCA | 20 | 673 | SpyCas9 |
| TGFbR2092 | TTCTCCAAAGTGCATTATGA | 20 | 674 | SpyCas9 |
| TGFbR2093 | CATCTTCCAGAATAAAGTCA | 20 | 675 | SpyCas9 |
| TGFbR2094 | CACATGAAGAAAGTCTCACC | 20 | 676 | SpyCas9 |
| TGFbR2095 | TTCCAGAATAAAGTCATGGT | 20 | 677 | SpyCas9 |
| TGFbR2096 | TTTTCCTTCATAATGCACTT | 20 | 678 | SpyCas9 |
| TGFBR24024 | CACAGTTGTGGAAACTTGAC | 20 | 679 | AsCpf1 |
| TGFBR24039 | CCCAACTCCGTCTTCCGCTC | 20 | 680 | AsCpf1 |
| TGFBR24040 | GGCTTTCCCTGCGTCTGGAC | 20 | 681 | AsCpf1 |
| TGFBR24036 | CTGAGGTCTATAAGGCCAAG | 20 | 682 | AsCpf1 |
| TGFBR24026 | TGATGTGAGATTTTCCACCT | 20 | 683 | AsCpf1 |
| TGFBR38402 | TGATGTGAGATTTTCCACCTG | 21 | 1173 | AsCpf1 |
| TGFBR24038 | CCTATGAGGAGTATGCCTCT | 20 | 684 | AsCpf1 |
| TGFBR24033 | AAGTGACAGGCATCAGCCTC | 20 | 685 | AsCpf1 |
| TGFBR24028 | CCATGACCCCAAGCTCCCCT | 20 | 686 | AsCpf1 |
| TGFBR24031 | CTTCATAATGCACTTTGGAG | 20 | 687 | AsCpf1 |
| TGFBR24032 | TTCATGTGTTCCTGTAGCTC | 20 | 688 | AsCpf1 |
| TGFBR24029 | TTCTGGAAGATGCTGCTTCT | 20 | 689 | AsCpf1 |
| TGFBR24035 | CCCACCAGGGTGTCCAGCTC | 20 | 690 | AsCpf1 |
| TGFBR24037 | AGACAGTGGCAGTCAAGATC | 20 | 691 | AsCpf1 |
| TGFBR24041 | CCTGCGTCTGGACCCTACTC | 20 | 692 | AsCpf1 |
| TGFBR24025 | CACAACTGTGTAAATTTTGT | 20 | 693 | AsCpf1 |
| TGFBR24030 | GAGAAGCAGCATCTTCCAGA | 20 | 694 | AsCpf1 |
| TGFBR24027 | TGGTTGTCACAGGTGGAAAA | 20 | 695 | AsCpf1 |
| TGFBR24034 | CCAGGTTGAACTCAGCTTCT | 20 | 696 | AsCpf1 |
| TGFBR24043 | ATCACAAAATTTACACAGTTG | 21 | 697 | SauCas9 |
| TGFBR24065 | GGCATCAGCCTCCTGCCACCA | 21 | 698 | SauCas9 |
| TGFBR24110 | GTTAGCCAGGTCATCCACAGA | 21 | 699 | SauCas9 |
| TGFBR24099 | GCTGGGCAGCTCCCTCGCCCG | 21 | 700 | SauCas9 |
| TGFBR24064 | CAGGAGGCTGATGCCTGTCAC | 21 | 701 | SauCas9 |
| TGFBR24094 | GAGGAGCGGAAGACGGAGTTG | 21 | 702 | SauCas9 |
| TGFBR24108 | CGTCTGGACCCTACTCTGTCT | 21 | 703 | SauCas9 |
| TGFBR24058 | TTTTTCCTTCATAATGCACTT | 21 | 704 | SauCas9 |
| TGFBR24075 | CCATTGAGCTGGACACCCTGG | 21 | 705 | SauCas9 |
| TGFBR24057 | CTTCTCCAAAGTGCATTATGA | 21 | 706 | SauCas9 |
| TGFBR24103 | GCCCAAGATGCCCATCGTGCA | 21 | 707 | SauCas9 |
| TGFBR24060 | TCATGTGTTCCTGTAGCTCTG | 21 | 708 | SauCas9 |
| TGFBR24048 | GTGATGCTGCAGTTGCTCATG | 21 | 709 | SauCas9 |
| TGFBR24087 | TCTCATGCTTCAGATTGATGT | 21 | 710 | SauCas9 |
| TGFBR24081 | TCCCTATGAGGAGTATGCCTC | 21 | 711 | SauCas9 |
| TGFBR24044 | CATCACAAAATTTACACAGTT | 21 | 712 | SauCas9 |
| TGFBR24077 | ATTGAGCTGGACACCCTGGTG | 21 | 713 | SauCas9 |
| TGFBR24080 | CAGTCAAGATCTTTCCCTATG | 21 | 714 | SauCas9 |
| TGFBR24046 | AGGATTTCTGGTTGTCACAGG | 21 | 715 | SauCas9 |
| TGFBR24101 | TCCACAGTGATCACACTCCAT | 21 | 716 | SauCas9 |
| TGFBR24079 | AGCAGAACACTTCAGAGCAGT | 21 | 717 | SauCas9 |
| TGFBR24072 | CCGGCAAGACGCGGAAGCTCA | 21 | 718 | SauCas9 |
| TGFBR24074 | GATGTCAGAGCGGTCATCTTC | 21 | 719 | SauCas9 |
| TGFBR24062 | TCATTGCACTCATCAGAGCTA | 21 | 720 | SauCas9 |
| TGFBR24054 | CTTCCAGAATAAAGTCATGGT | 21 | 721 | SauCas9 |
| TGFBR24045 | AGATTTTCCACCTGTGACAAC | 21 | 722 | SauCas9 |
| TGFBR24049 | ACTGCAGCATCACCTCCATCT | 21 | 723 | SauCas9 |
| TGFBR24098 | AGCTGGGCAGCTCCCTCGCCC | 21 | 724 | SauCas9 |
| TGFBR24090 | TGACGGCTGAGGAGCGGAAGA | 21 | 725 | SauCas9 |
| TGFBR24076 | CATTGAGCTGGACACCCTGGT | 21 | 726 | SauCas9 |
| TGFBR24078 | AGCAAAGCGACCTTTCCCCAC | 21 | 727 | SauCas9 |
| TGFBR24067 | CGCGTTAACCGGCAGCAGAAG | 21 | 728 | SauCas9 |
| TGFBR24063 | GAAATATGACTAGCAACAAGT | 21 | 729 | SauCas9 |
| TGFBR24107 | AGACAGAGTAGGGTCCAGACG | 21 | 730 | SauCas9 |
| TGFBR24047 | CAGGATTTCTGGTTGTCACAG | 21 | 731 | SauCas9 |
| TGFBR24096 | CTCCTGTAGGTTGCCCTTGGC | 21 | 732 | SauCas9 |
| TGFBR24105 | ACAGAGTAGGGTCCAGACGCA | 21 | 733 | SauCas9 |
| TGFBR24056 | GCTTCTCCAAAGTGCATTATG | 21 | 734 | SauCas9 |
| TGFBR24068 | GCAGCAGAAGCTGAGTTCAAC | 21 | 735 | SauCas9 |
| TGFBR24093 | TGAGGAGCGGAAGACGGAGTT | 21 | 736 | SauCas9 |
| TGFBR24055 | CTTTGGAGAAGCAGCATCTTC | 21 | 737 | SauCas9 |
| TGFBR24053 | CTCCCCTACCATGACTTTATT | 21 | 738 | SauCas9 |
| TGFBR24106 | GACAGAGTAGGGTCCAGACGC | 21 | 739 | SauCas9 |
| TGFBR24092 | CTGAGGAGCGGAAGACGGAGT | 21 | 740 | SauCas9 |
| TGFBR24102 | GGGCATCTTGGGCCTCCCACA | 21 | 741 | SauCas9 |
| TGFBR24082 | CCAAGAGGCATACTCCTCATA | 21 | 742 | SauCas9 |
| TGFBR24051 | AGAATGACGAGAACATAACAC | 21 | 743 | SauCas9 |
| TGFBR24097 | CCTGACGCGGCATGTCATCAG | 21 | 744 | SauCas9 |
| TGFBR24073 | AGCGAGCACTGTGCCATCATC | 21 | 745 | SauCas9 |
| TGFBR24104 | GCAGGTTAGGTCGTTCTTCAC | 21 | 746 | SauCas9 |
| TGFBR24050 | ACCTCCATCTGTGAGAAGCCA | 21 | 747 | SauCas9 |
| TGFBR24052 | TAAAGTCATGGTAGGGGAGCT | 21 | 748 | SauCas9 |
| TGFBR24061 | TCAGAGCTACAGGAACACATG | 21 | 749 | SauCas9 |
| TGFBR24086 | TCTCAGACATCAATCTGAAGC | 21 | 750 | SauCas9 |
| TGFBR24066 | CATCAGCCTCCTGCCACCACT | 21 | 751 | SauCas9 |
| TGFBR24089 | CGCTCCTCAGCCGTCAGGAAC | 21 | 752 | SauCas9 |
| TGFBR24071 | AACCTGGGAAACCGGCAAGAC | 21 | 753 | SauCas9 |
| TGFBR24095 | TCCACGCCAAGGGCAACCTAC | 21 | 754 | SauCas9 |
| TGFBR24100 | GAGGTGAGCAATCCCCCGGGC | 21 | 755 | SauCas9 |
| TGFBR24069 | CAGCAGAAGCTGAGTTCAACC | 21 | 756 | SauCas9 |
| TGFBR24083 | TCCAAGAGGCATACTCCTCAT | 21 | 757 | SauCas9 |
| TGFBR24070 | AGCAGAAGCTGAGTTCAACCT | 21 | 758 | SauCas9 |
| TGFBR24088 | CCAGTTCCTGACGGCTGAGGA | 21 | 759 | SauCas9 |
| TGFBR24085 | AGGAGTATGCCTCTTGGAAGA | 21 | 760 | SauCas9 |
| TGFBR24084 | TTCCAAGAGGCATACTCCTCA | 21 | 761 | SauCas9 |
| TGFBR24042 | CAACTGTGTAAATTTTGTGAT | 21 | 762 | SauCas9 |
| TGFBR24059 | TGAAGGAAAAAAAAAAGCCTG | 21 | 763 | SauCas9 |
| TGFBR24091 | CGTCTTCCGCTCCTCAGCCGT | 21 | 764 | SauCas9 |
| TGFBR24109 | CCAGGTCATCCACAGACAGAG | 21 | 765 | SauCas9 |
| TGFBR2736 | GCCTAGAGTGAAGAGATTCAT | 21 | 766 | SpyCas9 |
| TGFBR2737 | GTTCTCCAAAGTGCATTATGA | 21 | 767 | SpyCas9 |
| TGFBR2738 | GCATCTTCCAGAATAAAGTCA | 21 | 768 | SpyCas9 |

In some embodiments the gRNA for use in the disclosure is a gRNA targeting CISH (CISH gRNA). In some embodiments, the gRNA targeting CISH is one or more of the gRNAs described in Table 5.

TABLE5
CISH gRNAs

| | gRNA Targeting Domain Sequence | | | |
|---|---|---|---|---|
| Name | (DNA) | Length | SEQ ID NO: | Enzyme |
| CISH0873 | CAACCGTCTGGTGGCCGACG | 20 | 769 | SpyCas9 |
| CISH0874 | CAGGATCGGGGCTGTCGCTT | 20 | 770 | SpyCas9 |
| CISH0875 | TCGGGCCTCGCTGGCCGTAA | 20 | 771 | SpyCas9 |
| CISH0876 | GAGGTAGTCGGCCATGCGCC | 20 | 772 | SpyCas9 |
| CISH0877 | CAGGTGTTGTCGGGCCTCGC | 20 | 773 | SpyCas9 |
| CISH0878 | GGAGGTAGTCGGCCATGCGC | 20 | 774 | SpyCas9 |
| CISH0879 | GGCATACTCAATGCGTACAT | 20 | 775 | SpyCas9 |
| CISH0880 | CCGCCTTGTCATCAACCGTC | 20 | 776 | SpyCas9 |
| CISH0881 | AGGATCGGGGCTGTCGCTTC | 20 | 777 | SpyCas9 |
| CISH0882 | CCTTGTCATCAACCGTCTGG | 20 | 778 | SpyCas9 |
| CISH0883 | TACTCAATGCGTACATTGGT | 20 | 779 | SpyCas9 |
| CISH0884 | GGGTTCCATTACGGCCAGCG | 20 | 780 | SpyCas9 |
| CISH0885 | GGCACTGCTTCTGCGTACAA | 20 | 781 | SpyCas9 |
| CISH0886 | GGTTGATGACAAGGCGGCAC | 20 | 782 | SpyCas9 |
| CISH0887 | TGCTGGGGCCTTCCTCGAGG | 20 | 783 | SpyCas9 |
| CISH0888 | TTGCTGGCTGTGGAGCGGAC | 20 | 784 | SpyCas9 |
| CISH0889 | TTCTCCTACCTTCGGGAATC | 20 | 785 | SpyCas9 |
| CISH0890 | GACTGGCTTGGGCAGTTCCA | 20 | 786 | SpyCas9 |
| CISH0891 | CATGCAGCCCTTGCCTGCTG | 20 | 787 | SpyCas9 |
| CISH0892 | AGCAAAGGACGAGGTCTAGA | 20 | 788 | SpyCas9 |
| CISH0893 | GCCTGCTGGGGCCTTCCTCG | 20 | 789 | SpyCas9 |
| CISH0894 | CAGACTCACCAGATTCCCGA | 20 | 790 | SpyCas9 |
| CISH0895 | ACCTCGTCCTTTGCTGGCTG | 20 | 791 | SpyCas9 |
| CISH0896 | CTCACCAGATTCCCGAAGGT | 20 | 792 | SpyCas9 |
| CISH7048 | TACGCAGAAGCAGTGCCCGC | 20 | 793 | AsCpf1 |
| CISH7049 | AGGTGTACAGCAGTGGCTGG | 20 | 794 | AsCpf1 |
| CISH7050 | GGTGTACAGCAGTGGCTGGT | 20 | 795 | AsCpf1 |
| CISH7051 | CGGATGTGGTCAGCCTTGTG | 20 | 796 | AsCpf1 |
| CISH7052 | CACTGACAGCGTGAACAGGT | 20 | 797 | AsCpf1 |
| CISH7053 | ACTGACAGCGTGAACAGGTA | 20 | 798 | AsCpf1 |
| CISH7054 | GCTCACTCTCTGTCTGGGCT | 20 | 799 | AsCpf1 |
| CISH7055 | CTGGCTGTGGAGCGGACTGG | 20 | 800 | AsCpf1 |
| CISH7056 | GCTCTGACTGTACGGGGCAA | 20 | 801 | AsCpf1 RR |
| CISH7057 | AGCTCTGACTGTACGGGGCA | 20 | 802 | AsCpf1 RR |
| CISH7058 | ACAGTACCCCTTCCAGCTCT | 20 | 803 | AsCpf1 RR |
| CISH7059 | CGTCGGCCACCAGACGGTTG | 20 | 804 | AsCpf1 RR |
| CISH7060 | CCAGCCACTGCTGTACACCT | 20 | 805 | AsCpf1 RR |
| CISH7061 | ACCCCGGCCCTGCCTATGCC | 20 | 806 | AsCpf1 RR |
| CISH7062 | GGTATCAGCAGTGCAGGAGG | 20 | 807 | AsCpf1 RR |
| CISH7063 | GATGTGGTCAGCCTTGTGCA | 20 | 808 | AsCpf1 RR |
| CISH7064 | GGATGTGGTCAGCCTTGTGC | 20 | 809 | AsCpf1 RR |
| CISH7065 | GGCCACGCATCCTGGCCTTT | 20 | 810 | AsCpf1 RR |
| CISH7066 | GAAAGGCCAGGATGCGTGGC | 20 | 811 | AsCpf1 RR |
| CISH7067 | ACTGCTTGTCCAGGCCACGC | 20 | 812 | AsCpf1 RR |
| CISH7068 | TCTGGACTCCAACTGCTTGT | 20 | 813 | AsCpf1 RR |
| CISH7069 | GTCTGGACTCCAACTGCTTG | 20 | 814 | AsCpf1 RR |
| CISH7070 | GCTTCCGTCTGGACTCCAAC | 20 | 815 | AsCpf1 RR |
| CISH7071 | GACGGAAGCTGGAGTCGGCA | 20 | 816 | AsCpf1 RR |
| CISH7072 | CGCTGTCAGTGAAAACCACT | 20 | 817 | AsCpf1 RR |
| CISH7073 | CTGACAGCGTGAACAGGTAG | 20 | 818 | AsCpf1 RR |
| CISH38401 | ACTGACAGCGTGAACAGGTAG | 21 | 1174 | AsCpf1 RR |
| CISH7074 | TTACGGCCAGCGAGGCCCGA | 20 | 819 | AsCpf1 RR |
| CISH7075 | ATTACGGCCAGCGAGGCCCG | 20 | 820 | AsCpf1 RR |
| CISH7076 | GGAATCTGGTGAGTCTGAGG | 20 | 821 | AsCpf1 RR |
| CISH7077 | CCCTCAGACTCACCAGATTC | 20 | 822 | AsCpf1 RR |
| CISH7078 | CGAAGGTAGGAGAAGGTCTT | 20 | 823 | AsCpf1 RR |
| CISH7079 | GAAGGTAGGAGAAGGTCTTG | 20 | 824 | AsCpf1 RR |
| CISH7080 | GCACCTTTGGCTCACTCTCT | 20 | 825 | AsCpf1 RR |
| CISH7081 | TCGAGGAGGTGGCAGAGGGT | 20 | 826 | AsCpf1 RR |
| CISH7082 | TGGAACTGCCCAAGCCAGTC | 20 | 827 | AsCpf1 RR |
| CISH7083 | AGGGACGGGGCCCACAGGGG | 20 | 828 | AsCpf1 RR |
| CISH7084 | GGGACGGGGCCCACAGGGGC | 20 | 829 | AsCpf1 RR |
| CISH7085 | CTCCACAGCCAGCAAAGGAC | 20 | 830 | AsCpf1 RR |
| CISH7086 | CAGCCAGCAAAGGACGAGGT | 20 | 831 | AsCpf1 RR |
| CISH7087 | CTGCCTTCTAGACCTCGTCC | 20 | 832 | AsCpf1 RR |
| CISH7088 | CCTAAGGAGGATGCGCCTAG | 20 | 833 | AsCpf1 RVR |
| CISH7089 | TGGCCTCCTGCACTGCTGAT | 20 | 834 | AsCpf1 RVR |
| CISH7090 | AGCAGTGCAGGAGGCCACAT | 20 | 835 | AsCpf1 RVR |
| CISH7091 | CCGACTCCAGCTTCCGTCTG | 20 | 836 | AsCpf1 RVR |
| CISH7092 | GGGGTTCCATTACGGCCAGC | 20 | 837 | AsCpf1 RVR |
| CISH7093 | CACAGCAGATCCTCCTCTGG | 20 | 838 | AsCpf1 RVR |
| CISH7094 | ATTGCCCCGTACAGTCAGAG | 21 | 839 | SauCas9 |
| CISH7095 | CCCGTACAGTCAGAGCTGGA | 21 | 840 | SauCas9 |
| CISH7096 | TGGTGGAGGAGCAGGCAGTG | 21 | 841 | SauCas9 |
| CISH7097 | TCCTTAGGCATAGGCAGGGC | 21 | 842 | SauCas9 |
| CISH7098 | CGGCCCTGCCTATGCCTAAG | 21 | 843 | SauCas9 |
| CISH7099 | TAGGCATAGGCAGGGCCGGG | 21 | 844 | SauCas9 |
| CISH7100 | AGGCAGGGCCGGGGTGGGAG | 21 | 845 | SauCas9 |
| CISH7101 | GCAGGATCGGGGCTGTCGCT | 21 | 846 | SauCas9 |
| CISH7102 | CTGCACAAGGCTGACCACAT | 21 | 847 | SauCas9 |
| CISH7103 | TGCACAAGGCTGACCACATC | 21 | 848 | SauCas9 |
| CISH7104 | CTGACCACATCCGGAAAGGC | 21 | 849 | SauCas9 |
| CISH7105 | GGCCACGCATCCTGGCCTTT | 21 | 850 | SauCas9 |
| CISH7106 | GCGTGGCCTGGACAAGCAGT | 21 | 851 | SauCas9 |
| CISH7107 | GACAAGCAGTTGGAGTCCAG | 21 | 852 | SauCas9 |
| CISH7108 | GTTGGAGTCCAGACGGAAGC | 21 | 853 | SauCas9 |
| CISH7109 | ATGCGTACATTGGTGGGGCC | 21 | 854 | SauCas9 |
| CISH7110 | TGGCCCCACCAATGTACGCA | 21 | 855 | SauCas9 |
| CISH7111 | GCTACCTGTTCACGCTGTCA | 21 | 856 | SauCas9 |
| CISH7112 | TGACAGCGTGAACAGGTAGC | 21 | 857 | SauCas9 |
| CISH7113 | GTCGGGCCTCGCTGGCCGTA | 21 | 858 | SauCas9 |
| CISH7114 | GCACTTGCCTAGGCTGGTAT | 21 | 859 | SauCas9 |
| CISH7115 | GGGAATCTGGTGAGTCTGAG | 21 | 860 | SauCas9 |
| CISH7116 | CTCACCAGATTCCCGAAGGT | 21 | 861 | SauCas9 |
| CISH7117 | CTCCTACCTTCGGGAATCTG | 21 | 862 | SauCas9 |
| CISH7118 | CAAGACCTTCTCCTACCTTC | 21 | 863 | SauCas9 |
| CISH7119 | CCAAGACCTTCTCCTACCTT | 21 | 864 | SauCas9 |
| CISH7120 | GCCAAGACCTTCTCCTACCT | 21 | 865 | SauCas9 |
| CISH7121 | TATGCACAGCAGATCCTCCT | 21 | 866 | SauCas9 |
| CISH7122 | CAAAGGTGCTGGACCCAGAG | 21 | 867 | SauCas9 |
| CISH7123 | GGCTCACTCTCTGTCTGGGC | 21 | 868 | SauCas9 |
| CISH7124 | AGGGTACCCCAGCCCAGACA | 21 | 869 | SauCas9 |
| CISH7125 | AGAGGGTACCCCAGCCCAGA | 21 | 870 | SauCas9 |
| CISH7126 | GTACCCTCTGCCACCTCCTC | 21 | 871 | SauCas9 |
| CISH7127 | CCTTCCTCGAGGAGGTGGCA | 21 | 872 | SauCas9 |
| CISH7128 | ATGACTGGCTTGGGCAGTTC | 21 | 873 | SauCas9 |
| CISH7129 | GGCCCCTGTGGGCCCCGTCC | 21 | 874 | SauCas9 |
| CISH7130 | AGGACGAGGTCTAGAAGGCA | 21 | 875 | SauCas9 |

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (11) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. For example, Cas9 nucleases recognize PAM sequences that are 3′ of the protospacer, while

Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

### Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, —II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain or of a Cas9 HNH domain results in a nickase.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

Exemplary suitable nuclease variants include, but are not limited to, AsCpf1 variants comprising an M537R substitution, an H800A substitution, and/or an F870L substitution, or any combination thereof (numbering scheme according to AsCpf1 wild-type sequence). Other suitable modifications of the AsCpf1 amino acid sequence are known to those of ordinary skill in the art. Some exemplary sequences of wild-type AsCpf1 and AsCpf1 variants are provided below.

His-AsCpf1-sNLS-sNLS H800A amino acid sequence (SEQ ID NO: 1142)
MGHHHHHHGSTQFEGFTNLYQVSKTLRFELIPQGKTLKHIQEQGFIEEDKARNDHYKELKPIIDRIYK
TYADQCLQLVqLDWENLSAAIDSYRKEKTEETRNALIEEQATYRNAIHDYFIGRTDNLTDAINKRHAE
IYKGLFKAELFNGKVLKQLGTVTTTEHENALLRSFDKFTTYFSGFYENRKNVFSAEDISTAIPHRIVQ
DNFPKFKENCHIFTRLITAVPSLREHFENVKKAIGIFVSTSIEEVFSFPFYNQLLTQTQIDLYNQLLG
GISREAGTEKIKGLNEVLNLAIQKNDETAHIIASLPHRFIPLFKQILSDRNTLSFILEEFKSDEEVIQ
SFCKYKTLLRNENVLETAEALFNELNSIDLTHIFISHKKLETISSALCDHWDTLRNALYERRISELTG
KITKSAKEKVQRSLKHEDINLQEIISAAGKELSEAFKQKTSEILSHAHAALDQPLPTTLKKQEEKEIL
KSQLDSLLGLYHLLDWFAVDESNEVDPEFSARLTGIKLEMEPSLSFYNKARNYATKKPYSVEKFKLNF
QMPTLASGWDVNKEKNNGAILFVKNGLYYLGIMPKQKGRYKALSFEPTEKTSEGFDKMYYDYFPDAAK
MIPKCSTQLKAVTAHFQTHTTPILLSNNFIEPLEITKEIYDLNNPEKEPKKFQTAYAKKTGDQKGYRE
ALCKWIDFTRDFLSKYTKTTSIDLSSLRPSSQYKDLGEYYAELNPLLYHISFQRIAEKEIMDAVETGK
LYLFQIYNKDFAKGHHGKPNLHTLYWTGLFSPENLAKTSIKLNGQAELFYRPKSRMKRMAARLGEKML
NKKLKDQKTPIPDTLYQELYDYVNHRLSHDLSDEARALLPNVITKEVSHEIIKDRRFTSDKFFFHVPI
TLNYQAANSPSKFNQRVNAYLKEHPETPIIGIDRGERNLIYITVIDSTGKILEQRSLNTIQQFDYQKK
LDNREKERVAARQAWSVVGTIKDLKQGYLSQVIHEIVDLMIHYQAVVVLENLNFGFKSKRTGIAEKAV
YQQFEKMLIDKLNCLVLKDYPAEKVGGVLNPYQLTDQFTSFAKMGTQSGFLFYVPAPYTSKIDPLTGF
VDPFVWKTIKNHESRKHFLEGFDFLHYDVKTGDFILHFKMNRNLSFQRGLPGFMPAWDIVFEKNETQF
DAKGTPFIAGKRIVPVIENHRFTGRYRDLYPANELIALLEEKGIVFRDGSNILPKLLENDDSHAIDTM
VALIRSVLQMRNSNAATGEDYINSPVRDLNGVCFDSRFQNPEWPMDADANGAYHIALKGQLLLNHLKE
SKDLKLONGISNQDWLAYIQELRNGSPKKKRKVGSPKKKRKV
Cpf1 variant 1 amino acid sequence (SEQ ID NO: 1143)
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQRPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAHRLGEKMLNKKLKDQKT
PIPDTLYQELYDYVNHRLSHDLSDEARALLPNVITKEVSHEIIKDRRFTSDKFLFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGGSLEHHH
HHH
Cpf1 variant 2 amino acid sequence (SEQ ID NO: 1144)
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQMPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAHRLGEKMLNKKLKDQKT
PIPDTLYQELYDYVNHRLSHDLSDEARALLPNVITKEVSHEIIKDRRFTSDKFFFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGGSLEHHH
HHH
Cpf1 variant 3 amino acid sequence (SEQ ID NO: 1145)
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQRPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAARLGEKMLNKKLKDQKT
PIPDTLYQELYDYVNHRLSHDLSDEARALLPNVITKEVSHEIIKDRRFTSDKFLFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVERDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGGSLEHHH
HHH
Cpf1 variant 4 amino acid sequence (SEQ ID NO: 1146)
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQRPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAARLGEKMLNKKLKDQKT
PIPDTLYQELYDYVNHRLSHDLSDEARALLPNVITKEVSHEIIKDRRFTSDKFLFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKV
Cpf1 variant 5 amino acid sequence (SEQ ID NO: 1147)
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQRPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAHRLGEKMLNKKLKDQKT
PIPDTLYQELYDYVNHRLSHDLSDEARALLPNVITKEVSHEIIKDRRFTSDKFLFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKV
Cpf1 variant 6 amino acid sequence (SEQ ID NO: 1148)
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQRPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAHRLGEKMLNKKLKDQKT
PIPDTLYQELYDYVNHRLSHDLSDEARALLPNVITKEVSHEIIKDRRFTSDKFLFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRNGRSSDDEATADSQHAAPPKKKRKVGGSGGSGGSGGSGGSGGSGGSGGSLEHHH
HHH
Cpf1 variant 7 amino acid sequence (SEQ ID NO: 1149)
MGRDPGKPIPNPLLGLDSTAPKKKRKVGIHGVPAATQFEGFTNLYQVSKTLRFELIPQGKTLKHIQEQ
GFIEEDKARNDHYKELKPIIDRIYKTYADQCLQLVQLDWENLSAAIDSYRKEKTEETRNALIEEQATY
RNAIHDYFIGRTDNLTDAINKRHAEIYKGLFKAELFNGKVLKQLGTVTTTEHENALLRSFDKFTTYFS
GFYENRKNVFSAEDISTAIPHRIVQDNFPKFKENCHIFTRLITAVPSLREHFENVKKAIGIFVSTSIE
EVFSFPFYNQLLTQTQIDLYNQLLGGISREAGTEKIKGLNEVLNLAIQKNDETAHIIASLPHRFIPLF
KQILSDRNTLSFILEEFKSDEEVIQSFCKYKTLLRNENVLETAEALFNELNSIDLTHIFISHKKLETI
SSALCDHWDTLRNALYERRISELTGKITKSAKEKVQRSLKHEDINLQEIISAAGKELSEAFKQKTSEI
LSHAHAALDQPLPTTLKKQEEKEILKSQLDSLLGLYHLLDWFAVDESNEVDPEFSARLTGIKLEMEPS
LSFYNKARNYATKKPYSVEKFKLNFQMPTLASGWDVNKEKNNGAILFVKNGLYYLGIMPKQKGRYKAL
SFEPTEKTSEGFDKMYYDYFPDAAKMIPKCSTQLKAVTAHFQTHTTPILLSNNFIEPLEITKEIYDLN
NPEKEPKKFQTAYAKKTGDQKGYREALCKWIDFTRDFLSKYTKTTSIDLSSLRPSSQYKDLGEYYAEL
NPLLYHISFQRIAEKEIMDAVETGKLYLFQIYNKDFAKGHHGKPNLHTLYWTGLFSPENLAKTSIKLN
GQAELFYRPKSRMKRMAHRLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHPETPIIGIDRGERNLIYIT
VIDSTGKILEQRSLNTIQQFDYQKKLDNREKERVAARQAWSVVGTIKDLKQGYLSQVIHEIVDLMIHY
QAVVVLENLNFGFKSKRTGIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFLHYDVKTGDFILHFKMNRN
LSFQRGLPGFMPAWDIVFEKNETQFDAKGTPFIAGKRIVPVIENHRFTGRYRDLYPANELIALLEEKG
IVFRDGSNILPKLLENDDSHAIDTMVALIRSVLQMRNSNAATGEDYINSPVRDLNGVCFDSRFQNPEW
PMDADANGAYHIALKGQLLLNHLKESKDLKLQNGISNQDWLAYIQELRNPKKKRKVKLAAALEHHHHH
H
Exemplary AsCpf1 wild-type amino acid sequence (SEQ ID NO: 1150):
MTQFEGFTNLYQVSKTLRFELIPQGKTLKHIQEQGFIEEDKARNDHYKELKPIIDRIYKTYADQCLQL
VQLDWENLSAAIDSYRKEKTEETRNALIEEQATYRNAIHDYFIGRTDNLTDAINKRHAEIYKGLFKAE
LFNGKVLKQLGTVTTTEHENALLRSFDKFTTYFSGFYENRKNVFSAEDISTAIPHRIVQDNFPKFKEN
CHIFTRLITAVPSLREHFENVKKAIGIFVSTSIEEVFSFPFYNQLLTQTQIDLYNQLLGGISREAGTE
KIKGLNEVLNLAIQKNDETAHIIASLPHRFIPLFKQILSDRNTLSFILEEFKSDEEVIQSFCKYKTLL
RNENVLETAEALFNELNSIDLTHIFISHKKLETISSALCDHWDTLRNALYERRISELTGKITKSAKEK
VQRSLKHEDINLQEIISAAGKELSEAFKQKTSEILSHAHAALDQPLPTTLKKQEEKEILKSQLDSLLG
LYHLLDWFAVDESNEVDPEFSARLTGIKLEMEPSLSFYNKARNYATKKPYSVEKFKLNFQMPTLASGW
DVNKEKNNGAILFVKNGLYYLGIMPKQKGRYKALSFEPTEKTSEGFDKMYYDYFPDAAKMIPKCSTQL
KAVTAHFQTHTTPILLSNNFIEPLEITKEIYDLNNPEKEPKKFQTAYAKKTGDQKGYREALCKWIDFT
RDFLSKYTKTTSIDLSSLRPSSQYKDLGEYYAELNPLLYHISFQRIAEKEIMDAVETGKLYLFQIYNK
DFAKGHHGKPNLHTLYWTGLFSPENLAKTSIKLNGQAELFYRPKSRMKRMAHRLGEKMLNKKLKDQKT
PIPDTLYQELYDYVNHRLSHDLSDEARALLPNVITKEVSHEIIKDRRFTSDKFFFHVPITLNYQAANS
PSKFNQRVNAYLKEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAEKAVYQQFEKMLI
DKLNCLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLENDDSHAIDTMVALIRSVLQ
MRNSNAATGEDYINSPVRDLNGVCFDSRFQNPEWPMDADANGAYHIALKGQLLLNHLKESKDLKLQNG
ISNQDWLAYIQELRN

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

As an example, the nucleic acid sequence for Cpf1 variant 4 is set forth below as SEQ ID NO:1175:

ATGACCCAGTTTGAAGGTTTCACCAATCTGTATCAGGTTAGCAAAACCCT
GCGTTTTGAACTGATTCCGCAGGGTAAAACCCTGAAACATATTCAAGAAC
AGGGCTTCATCGAAGAGGATAAAGCACGTAACGATCACTACAAAGAACTG
AAACCGATTATCGACCGCATCTATAAAACCTATGCAGATCAGTGTCTGCA
GCTGGTTCAGCTGGATTGGGAAAATCTGAGCGCAGCAATTGATAGTTATC
GCAAAGAAAAAACCGAAGAAACCCGTAATGCACTGATTGAAGAACAGGCA
ACCTATCGTAATGCCATCCATGATTATTTCATTGGTCGTACCGATAATCT
GACCGATGCAATTAACAAACGTCACGCCGAAATCTATAAAGGCCTGTTTA
AAGCCGAACTGTTTAATGGCAAAGTTCTGAAACAGCTGGGCACCGTTACC
ACCACCGAACATGAAAATGCACTGCTGCGTAGCTTTGATAAATTCACCAC
CTATTTCAGCGGCTTTTATGAGAATCGCAAAAACGTGTTTAGCGCAGAAG
ATATTAGCACCGCAATTCCGCATCGTATTGTGCAGGATAATTTCCCGAAA
TTCAAAGAGAACTGCCACATTTTTACCCGTCTGATTACCGCAGTTCCGAG
CCTGCGTGAACATTTTGAAAACGTTAAAAAAGCCATCGGCATCTTTGTTA
GCACCAGCATTGAAGAAGTTTTTAGCTTCCCGTTTTACAATCAGCTGCTG
ACCCAGACCCAGATTGATCTGTATAACCAACTGCTGGGTGGTATTAGCCG
TGAAGCAGGCACCGAAAAAATCAAAGGTCTGAATGAAGTGCTGAATCTGG
CCATTCAGAAAAATGATGAAACCGCACATATTATTGCAAGCCTGCCGCAT
CGTTTTATTCCGCTGTTCAAACAAATTCTGAGCGATCGTAATACCCTGAG
CTTTATTCTGGAAGAATTCAAATCCGATGAAGAGGTGATTCAGAGCTTTT
GCAAATACAAAACGCTGCTGCGCAATGAAAATGTTCTGGAAACTGCCGAA
GCACTGTTTAACGAACTGAATAGCATTGATCTGACCCACATCTTTATCAG
CCACAAAAAACTGGAAACCATTTCAAGCGCACTGTGTGATCATTGGGATA
CCCTGCGTAATGCCCTGTATGAACGTCGTATTAGCGAACTGACCGGTAAA
ATTACCAAAAGCGCGAAAGAAAAAGTTCAGCGCAGTCTGAAACATGAGGA
TATTAATCTGCAAGAGATTATTAGCGCAGCCGGTAAAGAACTGTCAGAAG
CATTTAAACAGAAAACCAGCGAAATTCTGTCACATGCACATGCAGCACTG
GATCAGCCGCTGCCGACCACCCTGAAAAAACAAGAAGAAAAAGAAATCCT
GAAAAGCCAGCTGGATAGCCTGCTGGGTCTGTATCATCTGCTGGACTGGT
TTGCAGTTGATGAAAGCAATGAAGTTGATCCGGAATTTAGCGCACGTCTG
ACCGGCATTAAACTGGAAATGGAACCGAGCCTGAGCTTTTATAACAAAGC
CCGTAATTATGCCACCAAAAAACCGTATAGCGTCGAAAAATTCAAACTGA
ACTTTCAGCGTCCGACCCTGGCAAGCGGTTGGGATGTTAATAAAGAAAAA
AACAACGGTGCCATCCTGTTCGTGAAAAATGGCCTGTATTATCTGGGTAT
TATGCCGAAACAGAAAGGTCGTTATAAAGCGCTGAGCTTTGAACCGACGG
AAAAAACCAGTGAAGGTTTTGATAAAATGTACTACGACTATTTTCCGGAT
GCAGCCAAAATGATTCCGAAATGTAGCACCCAGCTGAAAGCAGTTACCGC
ACATTTTCAGACCCATACCACCCCGATTCTGCTGAGCAATAACTTTATTG
AACCGCTGGAAATCACCAAAGAGATCTACGATCTGAATAACCCGGAAAAA
GAGCCGAAAAAATTCCAGACCGCATATGCAAAAAAAACCGGTGATCAGAA
AGGTTATCGTGAAGCGCTGTGTAAATGGATTGATTTCACCCGTGATTTTC
TGAGCAAATACACCAAAACCACCAGTATCGATCTGAGCAGCCTGCGTCCG
AGCAGCCAGTATAAAGATCTGGGCGAATATTATGCAGAACTGAATCCGCT
GCTGTATCATATTAGCTTTCAGCGTATTGCCGAGAAAGAAATCATGGACG
CAGTTGAAACCGGTAAACTGTACCTGTTCCAGATCTACAATAAAGATTTT
GCCAAAGGCCATCATGGCAAACCGAATCTGCATACCCTGTATTGGACCGG
TCTGTTTAGCCCTGAAAATCTGGCAAAAACCTCGATTAAACTGAATGGTC
AGGCGGAACTGTTTTATCGTCCGAAAAGCCGTATGAAACGTATGGCAGCT
CGTCTGGGTGAAAAAATGCTGAACAAAAAACTGAAAGACCAGAAAACCCC
GATCCCGGATACACTGTATCAAGAACTGTATGATTATGTGAACCATCGTC
TGAGCCATGATCTGAGTGATGAAGCACGTGCCCTGCTGCCGAATGTTATT
ACCAAAGAAGTTAGCCACGAGATCATTAAAGATCGTCGTTTTACCAGCGA
CAAATTCCTGTTTCATGTGCCGATTACCCTGAATTATCAGGCAGCAAATA
GCCCGAGCAAATTTAACCAGCGTGTTAATGCATATCTGAAAGAACATCCA
GAAACGCCGATTATTGGTATTGATCGTGGTGAACGTAACCTGATTTATAT
CACCGTTATTGATAGCACCGGCAAAATCCTGGAACAGCGTAGCCTGAATA
CCATTCAGCAGTTTGATTACCAGAAAAAACTGGATAATCGCGAGAAAGAA
CGTGTTGCAGCACGTCAGGCATGGTCAGTTGTTGGTACAATTAAAGACCT
GAAACAGGGTTATCTGAGCCAGGTTATTCATGAAATTGTGGATCTGATGA
TTCACTATCAGGCCGTTGTTGTGCTGGAAAACCTGAATTTTGGCTTTAAA
AGCAAACGTACCGGCATTGCAGAAAAAGCAGTTTATCAGCAGTTCGAGAA
AATGCTGATTGACAAACTGAATTGCCTGGTGCTGAAAGATTATCCGGCTG
AAAAAGTTGGTGGTGTTCTGAATCCGTATCAGCTGACCGATCAGTTTACC
AGCTTTGCAAAAATGGGCACCCAGAGCGGATTTCTGTTTTATGTTCCGGC
ACCGTATACGAGCAAAATTGATCCGCTGACCGGTTTTGTTGATCCGTTTG
TTTGGAAAACCATCAAAAACCATGAAAGCCGCAAACATTTTCTGGAAGGT
TTCGATTTTCTGCATTACGACGTTAAAACGGGTGATTTCATCCTGCACTT
TAAAATGAATCGCAATCTGAGTTTTCAGCGTGGCCTGCCTGGTTTTATGC
CTGCATGGGATATTGTGTTTGAGAAAAACGAAACACAGTTCGATGCAAAA
GGCACCCCGTTTATTGCAGGTAAACGTATTGTTCCGGTGATTGAAAATCA
TCGTTTCACCGGTCGTTATCGCGATCTGTATCCGGCAAATGAACTGATCG
CACTGCTGGAAGAGAAAGGTATTGTTTTTCGTGATGGCTCAAACATTCTG
CCGAAACTGCTGGAAAATGATGATAGCCATGCAATTGATACCATGGTTGC
ACTGATTCGTAGCGTTCTGCAGATGCGTAATAGCAATGCAGCAACCGGTG
AAGATTACATTAATAGTCCGGTTCGTGATCTGAATGGTGTTTGTTTTGAT
AGCCGTTTTCAGAATCCGGAATGGCCGATGGATGCAGATGCAAATGGTGC
ATATCATATTGCACTGAAAGGACAGCTGCTGCTGAACCACCTGAAAGAAA
GCAAAGATCTGAAACTGCAAAACGGCATTAGCAATCAGGATTGGCTGGCA
TATATCCAAGAACTGCGTAACGGTCGTAGCAGTGATGATGAAGCAACCGC
AGATAGCCAGCATGCAGCACCGCCTAAAAAGAAACGTAAAGTT

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

### Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7° 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

### Gene Constructs

In some aspects, the present disclosure provides complex editing strategies, and resulting modified cells having complex genomic alterations, that allow for the generation of advanced NK cell products for clinical applications, e.g., for immunooncology therapeutic approaches.

In some embodiments, the genomic alterations are introduced by use of one or more HDR expression constructs. In some embodiments, the genomic alterations are introduced by use of one or more HDR expression constructs. In some embodiments, the one or more HDR expression constructs comprise one or more donor HDR templates. In some embodiments, the one or more donor HDR templates comprise one or more expression cassettes encoding one or more cDNAs. In some embodiments, the donor HDR template comprises one expression cassette. In some embodiments, the donor HDR template comprises two expression cassettes. In some embodiments, the donor HDR template comprises three expression cassettes. In some embodiments, the donor HDR template comprises four expression cassettes. In some embodiments, the donor HDR template comprises five expression cassettes. In some embodiments, the donor HDR template comprises six expression cassettes. In some embodiments, the donor HDR template comprises seven expression cassettes. In some embodiments, the donor HDR template comprises eight expression cassettes. In some embodiments, the donor HDR template comprises nine expression cassettes. In some embodiments, the donor HDR template comprises ten expression cassettes. In some embodiments, the one or more expression cassette is monocistronic. In some embodiments, the one or more expression cassette is bicistronic.

In some embodiments, the one or more expression cassettes comprise one cDNA. In some embodiments, the one or more expression cassettes comprise two cDNAs. In some embodiments, the one or more expression cassettes comprise three cDNAs. In some embodiments, the one or more expression cassettes comprise four cDNAs. In some embodiments, the one or more expression cassettes comprise five cDNAs. In some embodiments, the one or more expression cassettes comprise six cDNAs. In some embodiments, the one or more expression cassettes comprise seven cDNAs. In some embodiments, the one or more expression cassettes comprise eight cDNAs. In some embodiments, the one or more expression cassettes comprise nine cDNAs. In some embodiments, the one or more expression cassettes comprise ten cDNAs. In some embodiments, the one or more expression cassettes comprise one or more cDNAs separated by a 2A sequence. In some embodiments, the one or more expression cassettes comprise two cDNAs separated by a 2A sequence. In some embodiments, the one or more expression cassettes comprise three cDNAs separated by a 2A sequence.

In some embodiments, the HDR expression construct comprises one or more cDNAs driven by a heterologous promoter.

In some embodiments, the HDR expression construct comprises one or more donor templates for inserting an inactivating mutation in a target gene, wherein the gene product has less, or no, function (being partially or wholly inactivated). In some embodiments, the HDR expression construct comprises one or more donor templates for inserting an inactivating mutation in a target gene, wherein the gene product has no function (wholly inactivated).

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. +1, +2, +3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino. In some embodiments, where multiple edits are made in the genome of an NK cell, or a cell that an NK cell is derived from, the edits are made at the same time or in close temporal proximity. In some such embodiments, two or more genomic edits are effected by two or more different RNA-guided nucleases. For example, one of the genomic edits may be effected by saCas9 (in connection with the respective saCas9 guide RNA), and a different genomic edit may be effected by Cpf1 (in connection with the respective Cpf1 guide RNA). In some embodiments, using different RNA-guided nucleases in the context of multiplex genomic editing approaches is advantageous as compared to using the same RNA-guided nuclease for two or more edits, e.g., in that it allows to decrease the likelihood or frequency of undesirable effects, such as, for example, off-target cutting, and the occurrence of genomic translocations.

### Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:

[5′ homology arm]-[replacement sequence]--[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### Quantitative Measurement of On-Target Gene Editing

It should be noted that the genome editing systems of the present disclosure allow for the detection and quantitative measurement of on-target gene editing outcomes, including targeted integration. The compositions and methods described herein can rely on the use of donor templates comprising a 5′ homology arm, a cargo, a one or more priming sites, a 3′ homology arm, and optionally stuffer sequence. For example, International Patent Publication No. WO2019/014564 by Ramusino et al. (Ramusino), which is incorporated by reference herein in its entirety, describes compositions and methods which allow for the quantitative analysis of on-target gene editing outcomes, including targeted integration events, by embedding one or more primer binding sites (i.e., priming sites) into a donor template that are substantially identical to a priming site present at the targeted genomic DNA locus (i.e., the target nucleic acid). The priming sites are embedded into the donor template such that, when homologous recombination of the donor template with a target nucleic acid occurs, successful targeted integration of the donor template integrates the priming sites from the donor template into the target nucleic acid such that at least one amplicon can be generated in order to quantitatively determine the on-target editing outcomes.

In some embodiments, the target nucleic acid comprises a first priming site (P1) and a second priming site (P2), and the donor template comprises a cargo sequence, a first priming site (P1′), and a second priming site (P2′), wherein P2′ is located 5′ from the cargo sequence, wherein P1′ is located 3′ from the cargo sequence (i.e., A1--P2′--N--P1′--A2), wherein P1′ is substantially identical to P1, and wherein P2′ is substantially identical to P2. After accurate homology-driven targeted integration, three amplicons are produced using a single PCR reaction with two oligonucleotide primers. The first amplicon, Amplicon X, is generated from the primer binding sites originally present in the genomic DNA (P1 and P2), and may be sequenced to analyze on-target editing events that do not result in targeted integration (e.g., insertions, deletions, gene conversion). The remaining two amplicons are mapped to the 5′ and 3′ junctions after homology-driven targeted integration. The second amplicon, Amplicon Y, results from the amplification of the nucleic acid sequence between P1 and P2′ following a targeted integration event at the target nucleic acid, thereby amplifying the 5′ junction. The third amplicon, Amplicon Z, results from the amplification of the nucleic acid sequence between P1′ and P2 following a targeted integration event at the target nucleic acid, thereby amplifying the 3′ junction. Sequencing of these amplicons provides a quantitative assessment of targeted integration at the target nucleic acid, in addition to information about the fidelity of the targeted integration. To avoid any biases inherent to amplicon size, stuffer sequence may optionally be included in the donor template to keep all three expected amplicons the same length.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. The genome editing systems according to this disclosure can incorporate multiple gRNAs, multiple RNA-guided nucleases, and other components such as proteins, and a variety of implementations will be evident to the skilled artisan based on the principles illustrated in systems of the disclosure. In some embodiments the genome editing system of the disclosure are delivered into cells as an ribonucleoprotein (RNP) complex. In some embodiments, one or more RNP complexes are delivered to the cell sequentially in any order, or simultaneously.

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof. In some embodiments the genome editing system of the disclosure are delivered by AAV.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). In some embodiments the genome editing system of the disclosure are delivered into cells by electroporation.

One promising solution to improve cell therapy processes consists on the direct delivery of active proteins into human cells. A protein delivery agent, the Feldan Shuttle, is a protein-based delivery agent, which is designed for cell therapy (Del'Guidice et al., PLoS One. 2018 Apr. 4; 13(4):e0195558; incorporated in its entirety herein by reference). In some embodiments the genome editing system of the disclosure are delivered into cells by the Feldan Shuttle.

The modified cells of the disclosure can be administered by any known routes of administration known to a person of skill in the art, at the time of filing this application. In some embodiments the modified cells of the disclosure are administered intravenously (IV). In some embodiments the modified NK cells of the disclosure are administered intravenously (IV).

As used herein, “dose” refers to a specific quantity of a pharmacologically active material for administration to a subject for a given time. Unless otherwise specified, the doses recited refer to NK cells having complex genomic alterations, that allow for the generation of advanced NK cell products for clinical applications. In some embodiments, a dose of modified NK cells refers to an effective amount of modified NK cells. For example, in some embodiments a dose or effective amount of modified NK cells refers to about 1×10⁹-5×10⁹ modified NK cells, or about 2×10⁹-5×10⁹ modified NK cells per dose. In some embodiments a dose or effective amount of modified NK cells refers to about 3×10⁹-5×10⁹ modified NK cells, or about 4×10⁹-5×10⁹ modified NK cells per dose.

### Generation of Modified iNK Cells

Some aspects of this disclosure relate to the generation of genetically modified NK cells that are derived from stem cells, e.g., from multipotent cells, such as, e.g., HSCs, or from pluripotent stem cells, such as, e.g., ES cells or iPS cells. In some embodiments, where genetically modified iNK cells are derived from iPS cells, the iPS cells are derived from a somatic donor cell. In some embodiments, where genetically modified iNK cells are derived from iPS cells, the iPS cells are derived from a multipotent donor cell, e.g., from an HSC.

The genomic edits present in the final iNK cell can be made at any stage of the process of reprogramming the donor cell to the iPS cell state, during the iPS cell state, and/or at any stage of the process of differentiating the iPS cell to an iNK state, e.g., at an intermediary state, such as, for example, an iPS cell-derived HSC state, or even up to or at the final iNK cell state. In some embodiments, one or more genomic edits present in a modified iNK cell provided herein is made before reprogramming the donor cell to the iPS cell state. In some embodiments, all edits present in a modified iNK cell provided herein are made at the same time, in close temporal proximity, and/or at the same cell stage of the reprogramming/differentiation process, e.g., at the donor cell stage, during the reprogramming process, at the iPS cell stage, or during the differentiation process. In some embodiments, two or more edits present in a modified iNK cell provided herein are made at different times and/or at different cell stages of the reprogramming/differentiation process. For example, in some embodiments, an edit is made at the donor cell stage and an different edit is made at the iPS cell stage; in some embodiments, an edit is made at the reprogramming stage and a different edit is made at the iPS cell stage. These examples are provided to illustrate some of the strategies provided herein, and are not meant to be limiting.

A variety of cell types can be used as a donor cell that can be subjected to the reprogramming, differentiation, and genomic editing strategies provided herein for the derivation of modified iNK cells. The donor cell to be subjected to the reprogramming, differentiation, and genomic editing strategies provided herein can be any suitable cell type. For example, the donor cell can be a pluripotent stem cell or a differentiated cell, e.g., a somatic cell, such as, for example, a fibroblast or a T lymphocyte.

In some embodiments, the donor cell is a human cell. In some embodiments, the donor cell is a non-human primate cell. In some embodiments, the donor cell is a mammalian cell. In some embodiments, the donor cell is a somatic cell. In some embodiments, the donor cell is a stem or progenitor cell. In certain embodiments, the donor cell is not part of a human embryo and its derivation does not involve the destruction of a human embryo.

In some embodiments, iNK cells, and methods of deriving such iNK cells, having one or more genomic alterations (e.g., a knock-out of a gene undesirable for immunooncology therapeutic approaches, and/or a knock-in of an exogenous nucleic acid, e.g. an expression construct encoding a gene product desirable for immunooncology therapeutic approaches) are provided herein. In some embodiments, the iNK cells are derived from an iPS cell, which in turn is derived from a somatic donor cell. Any suitable somatic cell can be used in the generation of iPS cells, and in turn, the generation of iNK cells. Suitable strategies for deriving iPS cells from various somatic donor cell types have been described and are known in the art. In some embodiments, the somatic donor cell is a fibroblast cell. In some embodiments, the somatic donor cell is a mature T cell.

For example, in some embodiments, the somatic donor cell, from which an iPS cell, and subsequently an iNK cell is derived, is a developmentally mature T cell (a T cell that has undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an induced pluripotent stem (iPS) cell, and throughout differentiation of the resulting iPS cell to a somatic cell.

In certain embodiments, the somatic donor cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4⁺ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, or a CD4⁺ CD25⁺ CD127⁻ Foxp3⁺ T cell.

One advantage of using T cells for the generation of iPS cells is that T cells can be edited with relative ease, e.g., by CRISPR-based methods or other gene-editing methods. Another advantage of using T cells for the generation of iPS cells is that the rearranged TCR locus allows for genetic tracking of individual cells and their daughter cells. If the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population.

A third advantage of using T cells in generating iNK cells carrying multiple edits is that certain karyotypic aberrations associated with chromosomal translocations are selected against in T cell culture. Such aberrations pose a concern when editing cells by CRISPR technology, and in particular when generating cells carrying multiple edits.

A fourth advantage of using T cell derived iPS cells as a starting point for the derivation of therapeutic lymphocytes is that it allows for the expression of a pre-screened TCR in the lymphocytes, e.g., via selecting the T cells for binding activity against a specific antigen, e.g., a tumor antigen, reprogramming the selected T cells to iPS cells, and then deriving lymphocytes from these iPS cells that express the TCR (e.g., T cells). This strategy would also allow for activating the TCR in other cell types, e.g., by genetic or epigenetic strategies.

A fifth advantage of using T cell derived iPS cells as a starting point for iNK differentiation is that the T cells retain at least part of their “epigenetic memory” throughout the reprogramming process, and thus subsequent differentiation of the same or a closely related cell type, such as iNK cells will be more efficient and/or result in higher quality cell populations as compared to approaches using non-related cells, such as fibroblasts, as a starting point for iNK derivation.

In certain embodiments, the donor cell being manipulated, e.g., the cell being reprogrammed and/or the cell, the genome of which is being edited, is a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In certain embodiments, the donor cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In certain embodiments, the donor cell is a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In certain embodiments, the donor cell is a myeloid progenitor cell (e.g., a common myeloid progenitor (CMP) cell or granulocyte macrophage progenitor (GMP) cell). In certain embodiments, the donor cell is a lymphoid progenitor cell, e.g., a common lymphoid progenitor (CLP) cell. In certain embodiments, the donor cell is an erythroid progenitor cell (e.g., an MEP cell). In certain embodiments, the donor cell is a hematopoietic stem/progenitor cell (e.g., a long term HSC (LT-HSC), short term HSC (ST-HSC), MPP cell, or lineage restricted progenitor (LRP) cell). In certain embodiments, the donor cell is a CD34⁺ cell, CD34⁺ CD90 cell, CD34⁺ CD38⁻ cell, CD34⁺ CD90⁺ CD49f⁻CD38⁻CD45RA⁻ cell, CD105⁺ cell, CD31⁺, or CD133⁺ cell, or a CD34⁺ CD90⁺ CD133⁺ cell. In certain embodiments, the donor cell is an umbilical cord blood CD34+ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In certain embodiments, the donor cell is a mobilized peripheral blood hematopoietic CD34⁺ cell (after the patient is treated with a mobilization agent, e.g., G-CSF or Plerixafor). In certain embodiments, the donor cell is a peripheral blood endothelial cell.

In some embodiments, the donor cell is a dividing cell. In other embodiments, the donor cell is a non-dividing cell.

In some embodiments, the modified iNK cells resulting from the methods and strategies of reprogramming, differentiating, and editing provided herein, are administered to a subject in need thereof, e.g., in the context of an immunooncology therapeutic approach. In some embodiments, donor cells, or any cells of any stage of the reprogramming, differentiating, and editing strategies provided herein can be maintained in culture or stored (e.g., frozen in liquid nitrogen) using any suitable method known in the art, e.g., for subsequent characterization or administration to a subject in need thereof.

### Cell Reprogramming

A cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The reprogramming of the cells of the disclosure can be performed by utilizing several methods. Examples of some methods for reprogramming somatic cells of the disclosure are described in, but are not limited to, Valamehr et al. WO2017/078807 (“Valamehr”) and Mendlein et al. WO2010/108126 (“Mendlein”), which are hereby incorporated by reference in their entireties.

Briefly, a method for directing differentiation of pluripotent stem cells into cells of a definitive hematopoietic lineage, may comprise: (i) contacting pluripotent stem cells with a composition comprising a BMP activator, and optionally bFGF, to initiate differentiation and expansion of mesodermal cells from the pluripotent stem cells; (ii) contacting the mesodermal cells with a composition comprising a BMP activator, bFGF, and a GSK3 inhibitor, wherein the composition is optionally free of TGFβ receptor/ALK inhibitor, to initiate differentiation and expansion of mesodermal cells having definitive HE potential from the mesodermal cells; (iii) contacting the mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is optionally free of TGFβ receptor/ALK inhibitor, to initiate differentiation and expansion of definitive hemogenic endothelium from pluripotent stem cell-derived mesodermal cells having definitive hemogenic endothelium potential; and optionally, subjecting pluripotent stem cells, pluripotent stem cell-derived mesodermal cells, mesodermal cells having hemogenic endothelium, and/or definitive hemogenic endothelium under low oxygen tension between about 2% to about 10%.

In some embodiments of the method for directing differentiation of pluripotent stem cells into cells of a hematopoietic lineage, the method further comprises contacting pluripotent stem cells with a composition comprising a MEK inhibitor, a GSK3 inhibitor, and a ROCK inhibitor, wherein the composition is free of TGFβ receptor/ALK inhibitors, to seed and expand the pluripotent stem cells. In some embodiments, the pluripotent stem cells are iPSCs. In some embodiments, the iPSCs are naïve iPSCs. In some embodiments, the iPSC comprises one or more genetic imprints, and wherein the one or more genetic imprints comprised in the iPSC are retained in the pluripotent stem cell derived hematopoietic cells differentiated therefrom.

In some embodiments of the method for directing differentiation of pluripotent stem cells into cells of a hematopoietic lineage, the differentiation of the pluripotent stem cells into cells of hematopoietic lineage is void of generation of embryoid bodies, and is in a monolayer culturing form.

In some embodiments of the above method, the obtained pluripotent stem cell-derived definitive hemogenic endothelium cells are CD34+. In some embodiments, the obtained definitive hemogenic endothelium cells are CD34+CD43−. In some embodiments, the definitive hemogenic endothelium cells are CD34+CD43−CXCR4−CD73−. In some embodiments, the definitive hemogenic endothelium cells are CD34+CXCR4−CD73−. In some embodiments, the definitive hemogenic endothelium cells are CD34+CD43−CD93−. In some embodiments, the definitive hemogenic endothelium cells are CD34+CD93−.

In some embodiments of the above method, the method further comprises (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate the differentiation of the definitive hemogenic endothelium to pre-T cell progenitors; and optionally, (ii) contacting the pre-T cell progenitors with a composition comprising one or more growth factors and cytokines selected from the group consisting of SCF, Flt3L, and IL7, but free of one or more of VEGF, bFGF, TPO, BMP activators and ROCK inhibitors, to initiate the differentiation of the pre-T cell progenitors to T cell progenitors or T cells. In some embodiments of the method, the pluripotent stem cell-derived T cell progenitors are CD34+CD45+CD7+. In some embodiments of the method, the pluripotent stem cell-derived T cell progenitors are CD45+CD7+.

In yet some embodiments of the above method for directing differentiation of pluripotent stem cells into cells of a hematopoietic lineage, the method further comprises: (i) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15; and optionally, a BMP activator, to initiate differentiation of the definitive hemogenic endothelium to pre-NK cell progenitor; and optionally, (ii) contacting pluripotent stem cells-derived pre-NK cell progenitors with a composition comprising one or more growth factors and cytokines selected from the group consisting of SCF, Flt3L, IL3, IL7, and IL15, wherein the medium is free of one or more of VEGF, bFGF, TPO, BMP activators and ROCK inhibitors, to initiate differentiation of the pre-NK cell progenitors to NK cell progenitors or NK cells. In some embodiments, the pluripotent stem cell-derived NK progenitors are CD3−CD45+CD56+CD7+. In some embodiments, the pluripotent stem cell-derived NK cells are CD3−CD45+CD56+, and optionally further defined by NKp46+, CD57+ and CD16+.

In yet some embodiments of the above method for directing differentiation of pluripotent stem cells into NK cells, the method further comprises knocking out the gene Nrg1 in the pluripotent stem cells.

In some embodiments, the disclosure provides a method for generating pluripotent stem cell-derived T lineage cells, which comprises: (i) contacting pluripotent stem cells with a composition comprising a BMP activator, and optionally bFGF, to initiate differentiation and expansion of mesodermal cells from pluripotent stem cells; (ii) contacting the mesodermal cells with a composition comprising a BMP activator, bFGF, and a GSK3 inhibitor, but free of TGFβ receptor/ALK inhibitor, to initiate differentiation and expansion of the mesodermal cells having definitive HE potential from the mesodermal cells; (iii) contacting mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator; wherein the composition is free of TGFβ receptor/ALK inhibitor, to initiate differentiation and expansion of definitive hemogenic endothelium from mesodermal cells having definitive HE potential; (iv) contacting definitive hemogenic endothelium with a composition comprising a ROCK inhibitor; one or more growth factors and cytokines selected from the group consisting of VEGF, bFGF, SCF, Flt3L, TPO, and IL7; and optionally a BMP activator; to initiate differentiation of the definitive hemogenic endothelium to pre-T cell progenitors; and (v) contacting the pre-T cell progenitors with a composition comprising one or more growth factors and cytokines selected from the group consisting of SCF, Flt3L, and IL7, wherein the composition is free of one or more of VEGF, bFGF, TPO, BMP activators and ROCK inhibitors; to initiate differentiation of the pre-T cell progenitors to T cell progenitors or T cells; and optionally, the seeded pluripotent stem cells, mesodermal cells, mesodermal cells having definitive HE potential, and/or definitive hemogenic endothelium may be subject to low oxygen tension between about 2% to about 10%. In some embodiments, group II of the above method further comprises: contacting iPSCs with a composition comprising a MEK inhibitor, a GSK3 inhibitor, and a ROCK inhibitor, but free of TGFβ receptor/ALK inhibitors, to seed and expand pluripotent stem cells; and/or wherein the pluripotent stem cells. In some embodiments, the pluripotent stem cells are iPSCs. In some embodiments, the iPSCs are naïve iPSC. In some embodiments of the method, the differentiation of the pluripotent stem cells into T cell lineages is void of generation of embryoid bodies, and is in a monolayer culturing format.

In some embodiments, the disclosure provides a method for generating pluripotent stem cell-derived NK lineage cells, which comprises: (i) contacting pluripotent stem cells with a composition comprising a BMP activator, and optionally bFGF, to initiate differentiation and expansion of mesodermal cells from the pluripotent stem cells; (ii) contacting mesodermal cells with a composition comprising a BMP activator, bFGF, and a GSK3 inhibitor, and optionally free of TGFβ receptor/ALK inhibitor, to initiate differentiation and expansion of mesodermal cells having definitive HE potential from mesodermal cells; (iii) contacting mesodermal cells having definitive HE potential with a composition comprising one or more growth factors and cytokines selected from the group consisting of bFGF, VEGF, SCF, IGF, EPO, IL6, and IL11; a ROCK inhibitor; optionally a Wnt pathway activator; and optionally free of TGFβ receptor/ALK inhibitor, to initiate differentiation and expansion of pluripotent stem cell-derived definitive hemogenic endothelium from the pluripotent stem cell-derived mesodermal cells having definitive HE potential; (iv) contacting pluripotent stem cell-derived definitive hemogenic endothelium with a composition comprising a ROCK inhibitor; one or more growth factors and cytokines selected from the group consisting of VEGF, bFGF, SCF, Flt3L, TPO, IL3, IL7, and IL15, and optionally, a BMP activator, to initiate differentiation of the pluripotent stem cell-derived definitive hemogenic endothelium to pre-NK cell progenitors; and (v) contacting pluripotent stem cell-derived pre-NK cell progenitors with a composition comprising one or more growth factors and cytokines selected from the group consisting of SCF, Flt3L, IL3, IL7, and IL15, but free of one or more of VEGF, bFGF, TPO, BMP activators and ROCK inhibitors, to initiate differentiation of the pluripotent stem cell-derived pre-NK cell progenitors to pluripotent stem cell-derived NK cell progenitors or NK cells; and optionally, subjecting seeded pluripotent stem cells, pluripotent stem cell-derived-mesodermal cells, and/or definitive hemogenic endothelium under low oxygen tension between about 2% to about 10%. In some embodiments, the method for generating pluripotent stem cell-derived NK lineage cells of group II further comprises contacting iPSCs with a composition comprising a MEK inhibitor, a GSK3 inhibitor, and a ROCK inhibitor, but free of TGFβ receptor/ALK inhibitors, to seed and expand the iPSCs. In some embodiments, the iPSCs are naïve iPSCs. In some embodiments, the method for generating pluripotent stem cell-derived NK lineage cells is void of generation of embryoid bodies, and is in a monolayer culturing format.

In some embodiments, the disclosure provides a method for generating pluripotent stem cell-derived definitive hemogenic endothelium, the method comprises: (i) contacting iPSCs with a composition comprising a BMP activator, and optionally bFGF, to initiate differentiation and expansion of pluripotent stem cell-derived mesodermal cells from pluripotent stem cells; (ii) contacting pluripotent stem cell-derived mesodermal cells with a composition comprising a BMP activator, bFGF, and a GSK3 inhibitor, and optionally free of TGFβ receptor/ALK inhibitor, to initiate differentiation and expansion of pluripotent stem cell-derived mesodermal cells having definitive HE potential from pluripotent stem cell-derived mesodermal cells; (iii) contacting pluripotent stem cell-derived mesodermal cells having definitive HE potential with a composition comprising one or more growth factors and cytokines selected from the group consisting of bFGF, VEGF, SCF, IGF, EPO, IL6, and IL11; a ROCK inhibitor; and optionally a Wnt pathway activator, and optionally free of TGFβ receptor/ALK inhibitor, to initiate differentiation and expansion of pluripotent stem cell-derived definitive hemogenic endothelium from the pluripotent stem cell-derived mesodermal cells having definitive HE potential; and optionally, subjecting seeded pluripotent stem cells, pluripotent stem cell-derived mesodermal cells, and/or definitive hemogenic endothelium under low oxygen tension between about 2% to about 10%. In some embodiments, the above method for generating pluripotent stem cell-derived definitive hemogenic endothelium, further comprises: contacting iPSCs with a composition comprising a MEK inhibitor, a GSK3 inhibitor, and a ROCK inhibitor, but free of TGFβ receptor/ALK inhibitors, to seed and expand the iPSCs; and/or wherein the iPSCs are naïve iPSCs. In some embodiments, the iPSC comprises one or more genetic imprints, and wherein the one or more genetic imprints comprised in the iPSC are retained in the pluripotent stem cell derived definitive hemogenic endothelium cells differentiated therefrom. In some embodiments, the above method of differentiating iPSCs into cells of a definitive hemogenic endothelium is void of generation of embryoid bodies, and is in monolayer culturing format.

In some embodiments, the disclosure provides a method for generating pluripotent stem cell-derived multipotent progenitors of hematopoietic lineage, comprising: (i) contacting iPSCs with a composition comprising a BMP activator, and optionally bFGF, to initiate differentiation and expansion of pluripotent stem cell-derived mesodermal cells from iPSCs; (ii) contacting pluripotent stem cell-derived mesodermal cells with a composition comprising a BMP activator, bFGF, and a GSK3 inhibitor, but free of TGFβ receptor/ALK inhibitor, to initiate differentiation and expansion of the mesodermal cells having definitive HE potential from the mesodermal cells; (iii) contacting mesodermal cells having definitive HE potential with a composition comprising a ROCK inhibitor; one or more growth factors and cytokines selected from the group consisting of bFGF, VEGF, SCF, IGF, EPO, IL6, and IL11; and optionally, a Wnt pathway activator, wherein the composition is free of TGFβ receptor/ALK inhibitor, to initiate differentiation and expansion of definitive hemogenic endothelium from mesodermal cells having definitive HE potential; (iv) contacting definitive hemogenic endothelium with a composition comprising a BMP activator, a ROCK inhibitor, one or more growth factors and cytokines selected from the group consisting of TPO, IL3, GMCSF, EPO, bFGF, VEGF, SCF, IL6, Flt3L and IL11, to initiate differentiation of definitive hemogenic endothelium to pre-HSC; and (v) contacting pre-HSC with a composition comprising a BMP activator, one or more growth factors and cytokines selected from the group consisting of TPO, IL3, GMCSF, EPO, bFGF, VEGF, SCF, IL6, and IL11, but free of ROCK inhibitor, to initiate differentiation of the pre-HSC to hematopoietic multipotent progenitors; and optionally, subjecting seeded pluripotent stem cells, mesodermal cells, and/or definitive hemogenic endothelium under low oxygen tension between about 2% to about 10%. In some embodiments, the above method for generating pluripotent stem cell-derived hematopoiesis multipotent progenitors further comprises contacting pluripotent stem cells with a composition comprising a MEK inhibitor, a GSK3 inhibitor, and a ROCK inhibitor, but free of TGFβ receptor/ALK inhibitors, to seed and expand the pluripotent stem cells. In some embodiments, the pluripotent stem cells are iPSCs. In some embodiments, the iPSCs are naïve iPSCs. In some embodiments, the iPSC comprises one or more genetic imprints, and wherein the one or more genetic imprints comprised in the iPSC are retained in the pluripotent stem cell derived hematopoietic multipotent progenitor cells differentiated therefrom. In some embodiments, the differentiation of the pluripotent stem cells into hematopoiesis multipotent progenitors using the above method is void of generation of embryoid bodies, and is in monolayer culturing format.

In some embodiments, the disclosure provides a composition comprising: one or more cell populations generated from the culture platform disclosed herein: pluripotent stem cells-derived (i) CD34+ definitive hemogenic endothelium (iCD34), wherein the iCD34 cells have capacity to differentiate into multipotent progenitor cells, T cell progenitors, NK cell progenitors, T cells, NK cells, NKT cells and B cells, and wherein the iCD34 cells are CD34+CD43−; (ii) definitive hemogenic endothelium (iHE), wherein the iHE cells are CD34+, and at least one of CD43−, CD93−, CXCR4−, CD73−, and CXCR4−CD73−; (iii) pluripotent stem cell-derived definitive HSCs, wherein the iHSC is CD34+CD45+; (iv) hematopoietic multipotent progenitor cells, wherein the iMPP cells are CD34+CD45+; (v) T cell progenitors, wherein the T cell progenitors are CD34+CD45+CD7+ or CD34−CD45+CD7+; (vi) T cells, wherein the T cells are CD45+CD3+CD4+ or CD45+CD3+CD8+; (vii) NK cell progenitors, wherein the NK cell progenitors are CD45+CD56+CD7+; (viii) NK cells, wherein the NK cells are CD3-CD45+CD56+, and optionally further defined by NKp46+, CD57+, and CD16+; (ix) NKT cells, wherein the NKT cells are CD45+Vα24Jα18+CD3+; and (x) B cells, wherein the B cells are CD45+CD19+.

In some embodiments, the disclosure provides one or more cell lines, or clonal cells generated using the methods disclosed herein: pluripotent stem cell-derived (i) CD34+ definitive hemogenic endothelium (iCD34), wherein the iCD34 cells have capacity to differentiate into multipotent progenitor cells, T cell progenitors, NK cell progenitors, T cells, NK cells, and NKT cells, and wherein the iCD34 cells are CD34+CD43−; (ii) definitive hemogenic endothelium (iHE), wherein the iHE cell line or clonal cells are CD34+, and at least one of CD43−, CD93−, CXCR4−, CD73−, and CXCR4−CD73−; (iii) definitive HSCs, wherein the iHSCs is CD34+CD45+; (iv) hematopoietic multipotent progenitor cells (iMPP), wherein the iMPP cells are CD34+CD45+; (v) T cell progenitors, wherein the T cell progenitors are CD34+CD45+CD7+ or CD34−CD45+CD7+; (vi) T cells, wherein the T cells are CD45+CD3+CD4+ or CD45+CD3+CD8+; (vii) NK cell progenitors, wherein the NK cell progenitors are CD45+CD56+CD7+; (viii) NK cells, wherein the NK cells are CD3-CD45+CD56+, and optionally further defined by NKp46+, CD57+, and CD16+; (ix) NKT cells, wherein the NKT cells are CD45+Vα24Jα18+CD3+; and (x) B cells, wherein the B cells are CD45+CD19+.

In some embodiments, the present disclosure provides a method of promoting hematopoietic self-renewal, reconstitution or engraftment using one or more of cell populations, cell lines or clonal cells generated using methods as disclosed: pluripotent stem cell-derived (i) CD34+ definitive hemogenic endothelium (iCD34), wherein the iCD34 cells have capacity to differentiate into multipotent progenitor cells, T cell progenitors, NK cell progenitors, T cells NK cells and NKT cells, and wherein the iCD34 cells are CD34+CD43−; (ii) definitive hemogenic endothelium (iHE), wherein the iHE cell line or clonal cells are CD34+, and at least one of CD43−, CD93−, CXCR4−, CD73−, and CXCR4−CD73−; (iii) definitive HSCs, wherein the iHSCs are CD34+CD45+; (iv) hematopoietic multipotent progenitor cells, wherein the iMPP cells are CD34+CD45+; (v) T cell progenitors, wherein the T cell progenitors are CD34+CD45+CD7+ or CD34-CD45+CD7+; (vi) T cells, wherein the T cells are CD45+CD3+CD4+ or CD45+CD3+CD8+; (vii) NK cell progenitors, wherein the NK cell progenitors are CD45+CD56+CD7+; (viii) NK cells, wherein the NK cells are CD3-CD45+CD56+, and optionally further defined by NKp46+, CD57+, and CD16+; (ix) NKT cells, wherein the NKT cells are CD45+Vα24Jα18+CD3+; and (x) B cells, wherein the B cells are CD45+CD19+.

In some embodiments, the present disclosure provides a method of generating hematopoietic lineage cells with enhanced therapeutic properties, and the method comprises: obtaining iPSCs comprising one or more genetic imprints; and directing differentiation of iPSCs to hematopoietic lineage cells. The step of directed differentiation further comprises: (i) contacting the pluripotent stem cells with a composition comprising a BMP pathway activator, and optionally bFGF, to obtain mesodermal cells; and (ii) contacting the mesodermal cells with a composition comprising a BMP pathway activator, bFGF, and a WNT pathway activator, to obtain mesodermal cells having definitive hemogenic endothelium (HE) potential, wherein the mesodermal cells having definitive hemogenic endothelium (HE) potential are capable of providing hematopoietic lineage cells. Preferably, the mesodermal cells and mesodermal cells having definitive HE potential are obtained in steps (i) and (ii) without the step of forming embryoid bodies, and the obtained hematopoietic lineage cells comprise definitive hemogenic endothelium cells, hematopoietic stem and progenitor cells (HSC), hematopoietic multipotent progenitor cell (MPP), pre-T cell progenitor cells, pre-NK cell progenitor cells, T cell progenitor cells, NK cell progenitor cells, T cells, NK cells, NKT cells, or B cells. Moreover, the hematopoietic lineage cells retain the genetic imprints comprised in the iPSCs for directed differentiation.

In some embodiments, the step of directed differentiation of the above method further comprises: (i) contacting the mesodermal cells having definitive HE potential with a composition comprising bFGF and a ROCK inhibitor to obtain definitive HE cells; (ii) contacting the definitive HE cells with a composition comprising a BMP activator, and optionally a ROCK inhibitor, and one or more growth factors and cytokines selected from the group consisting of TPO, IL3, GMCSF, EPO, bFGF, VEGF, SCF, IL6, Flt3L and IL11 to obtain hematopoietic multipotent progenitor cells (MPP); (iii) contacting the definitive HE cells with a composition comprising one or more growth factors and cytokines selected from the group consisting of SCF, Flt3L, and IL7; and optionally one or more of a BMP activator, a ROCK inhibitor, TPO, VEGF and bFGF to obtain pre-T cell progenitors, T cell progenitors, and/or T cells; or (iv) contacting the definitive HE cells with a composition comprising one or more growth factors and cytokines selected from the group consisting of SCF, Flt3L, TPO, IL7 and IL15, and optionally one or more of a BMP activator, a ROCK inhibitor, VEGF and bFGF to obtain pre-NK cell progenitors, NK cell progenitors, and/or NK cells.

Briefly, the method may comprise reprogramming a mature source T or B cell to obtain induced pluripotent stem cells (iPSCs); and detecting the presence, in the iPSCs or the hematopoietic lineage cells derived therefrom, of a specific V(D)J recombination that is same as the one comprised in the mature T or B cell for generating the iPSC. In some embodiments, the above method further comprises isolating iPSCs or hematopoietic lineage cells comprising the same V(D)J recombination as that of the mature source T or B cell. In some embodiments, the above method comprises, prior to reprogramming the source cells, obtaining a mature source T or B cell for reprogramming; and determining V(D)J recombination comprised in immunoglobulins (Ig) or T cell receptors (TCR) that is specific to the mature source T or B cell.

A “pluripotency factor,” or “reprogramming factor,” refers to an agent capable of increasing the developmental potency of a cell, either alone or in combination with other agents. Pluripotency factors include, without limitation, polynucleotides, polypeptides, and small molecules capable of increasing the developmental potency of a cell. Exemplary pluripotency factors include, for example, transcription factors and small molecule reprogramming agents.

A number of various cell types from all three germ layers have been shown to be suitable for somatic cell reprogramming, including, but not limited to liver and stomach (Aoi et al., 2008); pancreatic f cells (Stadtfeld et al., 2008); mature B lymphocytes (Hanna et al., 2008); human dermal fibroblasts (Takahashi et al., 2007; Yu et al., 2007; Lowry et al., 2008; Aasen et al., 2008); meningiocytes (Qin et al., 2008); neural stem cells (DiSteffano et al., 2008); and neural progenitor cells (Eminli et al., 2008). Thus, the present disclosure contemplates, in part, methods to reprogram and/or program cells from any cell lineage.

The present disclosure contemplates, in part, to alter the potency of a cell by contacting the cell with one or more repressors and/or activators to modulate the epigenetic state, chromatin structure, transcription, mRNA splicing, post-transcriptional modification, mRNA stability and/or half-life, translation, post-translational modification, protein stability and/or half-life and/or protein activity of a component of a cellular pathway associated with determining or influencing cell potency.

Thus, in various embodiments, the present disclosure uses predictable and highly controlled methods for gene expression, as discussed elsewhere herein, that enable the reprogramming or de-differentiation and programming or differentiation of somatic cells ex vivo or in vivo. As, noted above, the intentional genetic engineering of cells, however, is not preferred, since it alters the cellular genome and would likely result in genetic or epigenetic abnormalities. In contrast, the compositions and methods of the present disclosure provide repressors and/or activators that non-genetically alter the potency of a cell by mimicking the cell's endogenous developmental potency pathways to achieve reprogramming and/or programming of the cell.

### Small Molecules in Reprogramming

Reprogramming of somatic cells into induced pluripotent stem cells has also been achieved by retroviral infection of defined genes (e.g., Oct-3/4, Sox-2, Klf-4, c-Myc, and Lin28, and the like) in combination with small molecules.

In some embodiments, the present disclosure provides a method of altering the potency of a cell that comprises contacting the cell with one or more repressors and/or activators or a composition comprising the same, wherein said one or more repressors and/or activators modulates at least one component of a cellular pathway associated with the potency of the cell, thereby altering the potency of the cell. In particular embodiments, the one or more repressors and/or activators modulate one or more components of a cellular pathway associated with the potency of the cell and thereby alter the potency of the cell. In certain embodiments, the one or more repressors and/or activators modulate one or more components of one or more cellular pathways associated with the potency of the cell and thereby alter the potency of the cell. In certain related embodiments, the modulation of the component(s) is synergistic and increases the overall efficacy of altering the potency of a cell. The potency of the cell can be altered, compared to the ground potency state, to a more potent state (e.g., from a differentiated cell to a multipotent, pluripotent, or totipotent cell) or a less potent state (e.g., from a totipotent, pluripotent, or multipotent cell to a differentiated somatic cell). In still yet other embodiments, the potency of a cell may be altered more than once. For example, a cell may first be reprogrammed to a more potent state, then programmed to a particular somatic cell.

In another embodiment, the methods of the present disclosure provide for increasing the potency a cell, wherein the cell is reprogrammed or dedifferentiated to a totipotent state, comprising contacting the cell with a composition comprising one or more repressors and/or activators, wherein the one or more repressors and/or activators modulates at least one component of a cellular pathway associated with the totipotency of the cell, thereby increasing the potency of the cell to a totipotent state.

In a particular embodiment, a method of increasing the potency a cell to a pluripotent state comprises contacting the cell with one or more repressors and/or activators, wherein the one or more repressors and/or activators modulates at least one component of a cellular pathway associated with the potency of the cell, thereby increasing the potency of the cell to a pluripotent state.

In another particular embodiment, a method of increasing the potency a cell to a multipotent state comprises contacting the cell with one or more repressors and/or activators, wherein the one or more repressors and/or activators modulates at least one component of a cellular pathway associated with the potency of the cell, thereby increasing the potency of the cell to a multipotent state.

In certain embodiments, a method of increasing the potency of a cell further comprises a step of contacting the totipotent cell, the pluripotent cell or the multipotent cell with a second composition, wherein the second composition modulates the at least one component of a cellular potency pathway to decrease the totipotency, pluripotency or multipotency of the cell and differentiate the cell to a mature somatic cell.

In another related embodiment, the present disclosure provides a method of reprogramming a cell that comprises contacting the cell with a composition comprising one or more repressors and/or activators, wherein the one or more repressors and/or activators modulates at least one component of a cellular pathway or pathways associated with the reprogramming of a cell, thereby reprogramming the cell.

In other embodiments, the present disclosure provides a method of dedifferentiating a cell to a more potent state, comprising contacting the cell with the composition comprising one/or more activators, wherein the one or more repressors and/or activators modulates at least one component of a cellular pathway or pathways associated with the dedifferentiation of the cell to the more potent state, thereby dedifferentiating the cell to an impotent state.

According to various embodiments of the present disclosure a repressor can be an antibody or an antibody fragment, an intrabody, a transbody, a DNAzyme, an ssRNA, a dsRNA, an mRNA, an antisense RNA, a ribozyme, an antisense oligonucleotide, a pri-miRNA, an shRNA, an antagomir, an aptamer, an siRNA, a dsDNA, a ssDNA; a polypeptide or an active fragment thereof, a peptidomimetic, a peptoid, or a small organic molecule. Polypeptide-based repressors include, but are not limited to fusion polypeptides. Polypeptide-based repressors also include transcriptional repressors, which can further be fusion polypeptides and/or artificially designed transcriptional repressors as described elsewhere herein.

According to other various embodiments, an activator can be an antibody or an antibody fragment, an mRNA, a bifunctional antisense oligonucleotide, a dsDNA, a polypeptide or an active fragment thereof, a peptidomimetic, a peptoid, or a small organic molecule.

In some embodiments, repressors modulate at least one component of a cellular potency pathway by a) repressing the at least one component; b) de-repressing a repressor of the at least one component; or c) repressing an activator of the at least one component. In related embodiments, one or more repressors can modulate at least one component of a pathway associated with the potency of a cell by a) de-repressing the at least one component; b) repressing a repressor of the at least one component; or c) de-repressing an activator of the at least one component.

In certain embodiments, one or more repressors modulates at least one component of a cellular pathway associated with the potency of a cell by a) repressing a histone methyltransferase or repressing the at least one component's epigenetic state, chromatin structure, transcription, mRNA splicing, post-transcriptional modification, mRNA stability and/or half-life, translation, post-translational modification, protein stability and/or half-life and/or protein activity; or b) de-repressing a demethylase or activating the at least one component's epigenetic state, chromatin structure, transcription, mRNA splicing, post-transcriptional modification, mRNA stability and/or half-life, translation, post-translational modification, protein stability and/or half-life and/or protein activity.

In related embodiments, activators modulate at least one component of a cellular pathway associated with the potency of a cell by a) activating the at least one component; b) activating a repressor of a repressor of the at least one component; or c) activating an activator of the at least one component.

In certain embodiments, one or more activators modulates at least one component by a) activating a histone demethylase or activating the at least one component's epigenetic state, chromatin structure, transcription, mRNA splicing, post-transcriptional modification, mRNA stability and/or half-life, translation, post-translational modification, protein stability and/or half-life and/or protein activity; or b) activating a repressor of a histone methyltransferase or activating a repressor of the at least one component's epigenetic state, chromatin structure, transcription, mRNA splicing, post-transcriptional modification, mRNA stability and/or half-life, translation, post-translational modification, protein stability and/or half-life and/or protein activity.

In various other embodiments, the present disclosure contemplates, in part, a method of reprogramming a cell, comprising contacting the cell with one or more repressors, wherein the one or more repressors modulates at least one component of a cellular pathway associated with the reprogramming of a cell, thereby reprogramming the cell.

In various other embodiments, the present disclosure contemplates, in part, a method of reprogramming a cell, comprising contacting the cell with a composition comprising one or more activators, wherein the one or more activators modulates at least one component of a cellular pathway associated with the reprogramming of a cell, thereby re-programming the cell.

While some exemplary methods for reprogramming/NK cell differentiation are provided herein, these are exemplary and not meant to limit the scope of the present disclosure. Additional suitable methods for reprogramming/NK cell differentiation will be apparent to those of skill in the art based on the present disclosure in view of the knowledge in the art.

Methods for culturing NK cells on feeder layers or with feeder cells are described in detail in, for e.g., EP3184109 by Valamehr et al. (“Valamehr”) incorporated in its entirety herein by reference.

In general, any type of NK cell population can be cultured using a variety of methods and devices. Selection of culture apparatus is usually based on the scale and purpose of the culture. Scaling up of cell culture preferably involves the use of dedicated devices. Apparatus for large scale, clinical grade NK cell production is detailed, for example, in Spanholtz et al. (PLoS ONE 2010; 5:e9221) and Sutlu et al. (Cytotherapy 2010, Early Online 1-12).

The methods described hereinabove for ex vivo culturing NK cells populations can result, inter alia, in a cultured population of NK cells.

### Types of Edits

Some aspects of the present disclosure provide complex editing strategies, and resulting NK cells having complex genomic alterations, that allow for the generation of advanced NK cell products for clinical applications, e.g., for immunooncology therapeutic approaches. In some embodiments, the modified NK cells provided herein can serve as an off-the-shelf clinical solution for patients having, or having been diagnosed with, a hyperproliferative disease, such as, for example, a cancer. In some embodiments, the modified NK cells exhibit an enhanced survival, proliferation, NK cell response level, NK cell response duration, resistance against NK cell exhaustion, and/or target recognition as compared to non-modified NK cells. For example, the modified NK cells provided herein may comprise genomic edits that result in: a loss-of-function in TGF beta receptor 2 (TGFbetaR2) and/or a loss-of-function of CISH in the modified NK cell.

The modified NK cells may exhibit one or more edits in their genome that results in a loss-of-function in a target gene, and/or one or more modifications that results in a gain-of-function, or an overexpression, of a gene product, e.g., of a protein, from an exogenous nucleic acid construct, e.g., from an expression construct comprising a cDNA encoding for the gene product that is integrated into the genome of the modified NK cell or provided in an extrachromosomal manner, e.g., in the form of an episomal expression construct.

A loss-of-function of a target gene is characterized by a decrease in the expression of a target gene based on a genomic modification, e.g., an RNA-guided nuclease-mediated cut in the target gene that results in an inactivation, or in diminished expression or function, of the encoded gene product.

A gain-of-function of a gene product is characterized by an increased expression (also referred to herein as overexpression) of a gene product, e.g., of a protein, in a cell, which can include, for example, an increased expression level of the gene product, or expression of the gene product in a cell that does not express the gene product endogenously, e.g., from an endogenous gene.

In some embodiments, increased expression of a gene product is effected by introducing an exogenous nucleic acid construct that encodes the gene product into a cell, e.g., an exogenous nucleic acid construct that comprises a cDNA encoding the gene product under the control of a heterologous promoter. In some embodiments, the exogenous nucleic acid construct is integrated into a specific locus, e.g., via HDR-mediated gene editing, as described in more detail elsewhere herein. Methods for effecting loss-of-function edits as well as methods for effecting increased expression of gene products, e.g., via RNA-guided nuclease technology are well known to one of ordinary skill in the art.

The present disclosure embraces modified NK cells exhibiting any of the edits and/or increased expression of gene products listed in TABLE 4 and TABLE 5 combined, as well as any combination of such edits and/or increased expression of gene products listed in these tables.

It is to be understood that the exemplary embodiments provided herein are meant to illustrate some examples of NK cells embraced by the present disclosure. Additional configurations are embraced that are not described here in detail for the sake of brevity, but such embodiments will be immediately apparent to those of skill in the art based on the present disclosure.

### Knock-Ins and Knock-Outs

In some embodiments, a modified cell may express one or more of a loss of function in TGFbetaR2 and/or a loss of function in CISH.

As used herein, the term “express” or “expression” refers to the process to produce a polypeptide, including transcription and translation. Expression may be, e.g., increased by a number of approaches, including: increasing the number of genes encoding the polypeptide, increasing the transcription of the gene (such as by placing the gene under the control of a constitutive promoter), increasing the translation of the gene, knocking out of a competitive gene, or a combination of these and/or other approaches.

As used herein, “knock-in” refers to the addition of a target gene into a genetic locus of a cell.

As used herein, the term “knock-out” refers to an inactivating mutation in a target gene, wherein the product of the target gene comprises a loss of function.

As used herein, the term “loss of function” refers to an inactivating mutation in a target gene, wherein the gene product has less, or no, function (being partially or wholly inactivated). As used herein the term “complete loss of function” refers to an inactivating mutation in a target gene, wherein the gene product has no function (wholly inactivated).

As used herein, the term “TGFβRII” or “TGFbetaR2” refers to a transmembrane protein that has a protein kinase domain, forms a heterodimeric complex with TGF-beta receptor type-1, and binds TGF-beta. This receptor/ligand complex phosphorylates proteins, which then enter the nucleus and regulate the transcription of genes related to cell proliferation, cell cycle arrest, wound healing, immunosuppression, and tumorigenesis. Exemplary sequences of TGFβRII are set forth in KR710923.1, NM_001024847.2, and NM_003242.5.

As used herein, the term “CISH” refers to the Cytokine Inducible SH2 Containing Protein, for e.g., see Delconte et al., Nat Immunol. 2016 July; 17(7):816-24; incorporated in its entirety herein by reference. Exemplary sequences for CISH are set forth as NG_023194.1.

As used herein, the term “IL-15/IL15RA” or “Interleukin-15” (IL-15) refers to a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells. IL-15 Receptor alpha (IL15RA) specifically binds IL15 with very high affinity, and is capable of binding IL-15 independently of other subunits. It is suggested that this property allows IL-15 to be produced by one cell, endocytosed by another cell, and then presented to a third party cell. IL15RA is reported to enhance cell proliferation and expression of apoptosis inhibitor BCL2L1/BCL2-XL and BCL2. Exemplary sequences of IL-15 are provided in NG_029605.2, and exemplary sequences of IL-15RA are provided in NM_002189.4.

IL-15 is a key cytokine in promoting NK cell growth and homeostatic maintenance of memory T cells. IL-15 and its receptor chain, IL-15Ra, are essential for NK survival and do not stimulate regulatory T cells. IL-15/IL-15Ra binds to the beta and gamma subunits of IL-2 receptor and thereby activates JAK1/3 and STAT5. In some embodiments, the modified cell of the disclosure (for e.g., an NK cell) expresses an exogenous IL-15/IL-15Ra. In some embodiments, the exogenous IL-15/IL-15Ra is expressed as a membrane-bound IL15.IL15Ra complex, as described in Imamura et al., Blood. 2014 Aug. 14; 124(7):1081-8 and Hurton L V et al., PNAS, 2016; incorporated in their entirety herein by reference. In some embodiments, the exogenous IL-15/IL-15Ra is expressed as a soluble IL15Ra.IL15 complex, as described in Mortier E et al, JBC 2006; Bessard A, Mol Cancer Ther 2009; and Desbois M, JI 2016; incorporated in their entirety herein by reference. In some embodiments, the modified cell of the disclosure (for e.g., an NK cell) expresses a membrane-bound IL15.IL15Ra complex and a soluble IL15Ra.IL15 complex. In some embodiments, the modified cell of the disclosure (for e.g., an NK cell) express a membrane-bound form of IL15.IL15Ra complex with a cleavable linker. A knockout of CISH is associated with further promoting the IL-15 signaling, as described in Delconte P, Nat Immunol 2016; incorporated in its entirety herein by reference. In some embodiments, the modified cell of the disclosure (for e.g., an NK cell) expresses a loss of function in CISH. In some embodiments, the modified cell of the disclosure (for e.g., an NK cell) express exogenous IL-15/IL-15Ra and a loss of function in CISH.

The disclosure specifically encompasses variants of the above genes, including variants having at least 75%, 80%, 85%, 90%, 91%, 92%, 93%, 94%, 95%, 96%, 97%, 98%, or 99% percent identity to the above-identified gene sequences. As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Knock-ins and knock-outs can be effected by genome editing technologies known to those of skill in the art and include CRISPR/Cas technologies. Single-cut as well as multiplex editing strategies are suitable to achieve the desired product configurations provided herein, and such strategies are described herein or otherwise known to those of ordinary skill in the art.

In some embodiments, exemplary modified cells, e.g., modified pluripotent cells or differentiated progeny thereof, e.g., iNK cells or other modified lymphocyte types, are evaluated for their ability to escape the immune system of a non-autologous host, e.g., a patient in need of immunotherapy. In some embodiments, such an evaluation includes an in vitro assay. Suitable in vitro assays for such evaluations are known to those of ordinary skill in the relevant art, and include, without limitation, mixed lymphocyte reactivity (MLR) assays. This assay and other suitable assays are described, e.g., in Abbas et al., Cellular and Molecular Immunology, 7^{th} edition, ISBN 9781437735734, the entire contents of which are incorporated herein by reference. Other suitable assays will be apparent to the skilled artisan in view of the present disclosure.

### Methods of Use

A variety of diseases may be ameliorated by introducing the modified cells of the invention to a subject. Examples of diseases are, including but not limited to, cancer, including but not limited to solid tumors, including but not limited to, tumor of the brain, prostate, breast, lung, colon, uterus, skin, liver, bone, pancreas, ovary, testes, bladder, kidney, head, neck, stomach, cervix, rectum, larynx, or esophagus; and hematological malignancies, including but not limited to, acute and chronic leukemias, lymphomas, multiple myeloma and myelodysplastic syndromes.

Particular embodiments of the present invention are directed to methods of treating a subject in need thereof by administering to the subject a composition comprising any of the cells described herein. In particular embodiments, the terms “treating,” “treatment,” and the like are used herein to generally mean obtaining a desired pharmacologic and/or physiologic effect. The effect may be prophylactic in terms of completely or partially preventing a disease and/or may be therapeutic in terms of a partial or complete cure for a disease and/or adverse effect attributable to the disease. “Treatment” as used herein covers any treatment of a disease in a mammal, and includes: preventing the disease from occurring in a subject which may be predisposed to the disease but has not yet been diagnosed as having it; inhibiting the disease, i.e., arresting its development; or relieving the disease, i.e., causing regression of the disease. The therapeutic agent or composition may be administered before, during or after the onset of disease or injury. The treatment of ongoing disease, where the treatment stabilizes or reduces the undesirable clinical symptoms of the patient, is of particular interest.

In particular embodiments, the subject has a disease, condition, and/or an injury that can be treated, ameliorated, and/or improved by a cell therapy. Some embodiments contemplate that a subject in need of cell therapy is a subject with an injury, disease, or condition, whereby a cell therapy, e.g., a therapy in which a cellular material is administered to the subject, can treat, ameliorate, improve, and/or reduce the severity of at least one symptom associated with the injury, disease, or condition. Certain embodiments contemplate that a subject in need of cell therapy, includes, but is not limited to, a candidate for bone marrow or stem cell transplantation, a subject who has received chemotherapy or irradiation therapy, a subject who has or is at risk of having a hyperproliferative disorder or a cancer, e.g. a hyperproliferative disorder or a cancer of hematopoietic system, a subject having or at risk of developing a tumor, e.g., a solid tumor, a subject who has or is at risk of having a viral infection or a disease associated with a viral infection.

According, the embodiments described herein further provide pharmaceutical compositions comprising the cells made by the methods and composition disclosed herein, wherein the pharmaceutical compositions further comprise a pharmaceutically acceptable medium. In some embodiments, the pharmaceutical composition comprises the NK cells made by the methods and composition disclosed herein.

Additionally, the embodiments described herein provide therapeutic use of the above pharmaceutical compositions by introducing the composition to a subject suitable for adoptive cell therapy, wherein the subject has a solid tumor; a hematological malignancy; an autoimmune disorder; or an infection associated with viral, bacterial, fungal and/or helminth infections, including but not limited to, HIV, RSV, EBV, CMV, adenovirus, or BK polyomavirus infections.

Particular embodiments described herein are also directed to methods of treating a subject in need thereof by administering to the subject a composition comprising any of the cells described herein with one or more antibodies, or fragments thereof, to induce and/or increase an antibody-dependent cellular cytotoxicity (ADCC) effect in the subject. In some embodiments, the modified NK cells described herein exhibit greater ADCC activity when administered with one or more antibodies, or fragments thereof, to a subject in need thereof, e.g., a subject with a cancer, relative to unmodified NK cells that are administered with the same one or more antibodies, or fragments thereof, to a subject in need thereof. In some embodiments, the modified NK cells described herein kill a greater number of cancer cells when administered with one or more antibodies, or fragments thereof, to a subject in need thereof, e.g., a subject with cancer, relative to unmodified NK cells that are administered with the same one or more antibodies, or fragments thereof, to a subject.

### Cancers

Cancers that are suitable therapeutic targets of the present disclosure include cancer cells from the bladder, blood, bone, bone marrow, brain, breast, colon, esophagus, eye, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; the coma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is head and neck cancer.

In some embodiments, the cancer is a breast cancer. In another embodiment, the cancer is colon cancer. In another embodiment, the cancer is gastric cancer. In another embodiment, the cancer is RCC. In another embodiment, the cancer is non-small cell lung cancer (NSCLC).

In some embodiments, solid cancer indications that can be treated with the modified NK cells provided herein, either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas.

In some embodiments, hematological cancer indications that can be treated with the modified NK cells provided herein, either alone or in combination with one or more additional cancer treatment modality, include: ALL, CLL, NHL, DLBCL, AML, CML, multiple myeloma (MM).

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Examples of cellular proliferative and/or differentiative disorders of the lung include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

Examples of cellular proliferative and/or differentiative disorders involving the colon include, but are not limited to, tumors of the colon, such as non-neoplastic polyps, adenomas, familial syndromes, colorectal carcinogenesis, colorectal carcinoma, and carcinoid tumors.

Examples of cancers or neoplastic conditions, in addition to the ones described above, include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

Contemplated useful secondary or adjunctive therapeutic agents in this context include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™) and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

Other compounds that are effective in treating cancer are known in the art and described herein that are suitable for use with the compositions and methods of the present disclosure are described, for example, in the “Physicians Desk Reference, 62nd edition. Oradell, N.J.: Medical Economics Co., 2008”, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000.”, and “The Merck Index, Fourteenth Edition. Whitehouse Station, N.J.: Merck Research Laboratories, 2006”, incorporated herein by reference in relevant parts.

### Antibody-Dependent Cellular Cytotoxicity (ADCC)

The present disclosure provides modified NK cells (or other lymphocytes) that are useful in NK cell therapy, e.g., in the context of immunotherapeutic approaches, particularly in combination with an antibody, or antigen-binding portion thereof, to generate striking antibody-dependent cellular cytotoxicity (ADCC) effects, thereby surprisingly increasing the effectiveness of the modified NK cells in killing target cells, e.g. cancer cells. ADCC is a mechanism of cell-mediated immune defense, where an immune effector cell actively lyses a target cell after its membrane-surface antigens have been bound by specific antibodies. To participate in ADCC, the immune effector cells must express Fc-gamma receptors (FcγR) to be able to recognize the Fc region of the antibodies that bind to the target cells. Most immune effector cells have both activating and inhibitory FcγR. An advantage of using NK cells to target cancer cells via ADCC is that, unlike other effector cells, NK cells only have activating FcγRs (e.g., FcγR IIIa, also known as CD16a, and FcγR IIc, also known as CD32c) and are believed to be the most important effectors of ADCC in humans. Thus, the use of the modified NK cells disclosed herein and antibodies targeting cancer cell-specific antigens to elicit ADCC provides novel and surprisingly effective immunotherapies.

In one embodiment, the molecule comprising an Fc domain that binds cancer cells, e.g., antibody, or antigen-binding portion thereof, binds an antigen on a cancer cell, or a “cancer antigen.” In one embodiment, the antigen on the cancer cell is epidermal growth factor receptor (EGFR), HER2, CD20, PD-L1, PD-1 (PEMBRO and NIVO), CTLA-4 (IPI), CD73, TIGIT, GD2, VEGF-A, VEGFR-2, PDGFR-2, PDGFRa, RANKL, CD19, CD3. In one embodiment, the antibody is cetuximab, trastuzumab, rituximab, pertuzumab, panitumumab, necitumumab, dinutuximab, bevacizumab, ramucirumab, olaratumab, ipilimumab, nivolumab, blinatumomab, alemtuzumab, bevacizumab, brentuximab, cetuximab, gemtuzumab, ipilimumab, ofatumumab, panitumumab, rituximab, tositumomab, inotuzumab, glembatumumab, lovortuzumab or trastuzumab, or an antigen-binding portion thereof. Additional antibodies include adecatumumab, afutuzumab, bavituximab, belimumab, bivatuzumab, cantuzumab, citatuzumab, cixutumumab, conatumumab, dacetuzumab, elotuzumab, etaracizumab, farletuzumab, figitumumab, iratumumab, labetuzumab, lexatumumab, lintuzumab, lucatumumab, mapatumumab, matuzumab, milatuzumab, necitumumab, nimotuzumab, olaratumab, oportuzumab, pertuzumab, pritumumab, ranibizumab, robatumumab, sibrotuzumab, siltuximab, tacatuzumab, tigatuzumab, tucotuzumab, veltuzumab votumumab, and zalutumumab, or an antigen-binding portion thereof.

In one embodiment, the antibody is cetuximab, or an antigen-binding portion thereof. In one embodiment, the antibody is trastuzumab, or an antigen-binding portion thereof. In one embodiment, the antibody is rituximab, or an antigen-binding portion thereof. In one embodiment, the antibody is pertuzumab, or an antigen-binding portion thereof. In one embodiment, the antibody is panitumumab, or an antigen-binding portion thereof. In one embodiment, the antibody is necitumumab, or an antigen-binding portion thereof. In one embodiment, the antibody is dinutuximab, or an antigen-binding portion thereof. In one embodiment, the antibody is bevacizumab, or an antigen-binding portion thereof. In one embodiment, the antibody is ramucirumab, or an antigen-binding portion thereof. In one embodiment, the antibody is olaratumab, or an antigen-binding portion thereof. In one embodiment, the antibody is ipilimumab, or an antigen-binding portion thereof. In one embodiment, the antibody is nivolumab, or an antigen-binding portion thereof. In one embodiment, the antibody is blinatumomab, or an antigen-binding portion thereof. In one embodiment, the antibody is alemtuzumab, or an antigen-binding portion thereof. In one embodiment, the antibody is bevacizumab, or an antigen-binding portion thereof. In one embodiment, the antibody is brentuximab, or an antigen-binding portion thereof. In one embodiment, the antibody is gemtuzumab, or an antigen-binding portion thereof. In one embodiment, the antibody is ipilimumab, or an antigen-binding portion thereof. In one embodiment, the antibody is ofatumumab, or an antigen-binding portion thereof. In one embodiment, the antibody is panitumumab, or an antigen-binding portion thereof. In one embodiment, the antibody is tositumomab, or an antigen-binding portion thereof. In one embodiment, the antibody is inotuzumab, or an antigen-binding portion thereof. In one embodiment, the antibody is glembatumumab, or an antigen-binding portion thereof. In one embodiment, the antibody is lovortuzumab, or an antigen-binding portion thereof. In one embodiment, the antibody is adecatumumab, or an antigen-binding portion thereof. In one embodiment, the antibody is afutuzumab, or an antigen-binding portion thereof. In one embodiment, the antibody is bavituximab, or an antigen-binding portion thereof. In one embodiment, the antibody is belimumab, or an antigen-binding portion thereof. In one embodiment, the antibody is bivatuzumab, or an antigen-binding portion thereof. In one embodiment, the antibody is cantuzumab, or an antigen-binding portion thereof. In one embodiment, the antibody is citatuzumab, or an antigen-binding portion thereof. In one embodiment, the antibody is cixutumumab, or an antigen-binding portion thereof. In one embodiment, the antibody is conatumumab, or an antigen-binding portion thereof. In one embodiment, the antibody is dacetuzumab, or an antigen-binding portion thereof. In one embodiment, the antibody is elotuzumab, or an antigen-binding portion thereof. In one embodiment, the antibody is etaracizumab, or an antigen-binding portion thereof. In one embodiment, the antibody is farletuzumab, or an antigen-binding portion thereof. In one embodiment, the antibody is figitumumab, or an antigen-binding portion thereof. In one embodiment, the antibody is iratumumab, or an antigen-binding portion thereof. In one embodiment, the antibody is labetuzumab, or an antigen-binding portion thereof. In one embodiment, the antibody is lexatumumab, or an antigen-binding portion thereof. In one embodiment, the antibody is lintuzumab, or an antigen-binding portion thereof. In one embodiment, the antibody is lucatumumab, or an antigen-binding portion thereof. In one embodiment, the antibody is mapatumumab, or an antigen-binding portion thereof. In one embodiment, the antibody is matuzumab, or an antigen-binding portion thereof. In one embodiment, the antibody is milatuzumab, or an antigen-binding portion thereof. In one embodiment, the antibody is necitumumab, or an antigen-binding portion thereof. In one embodiment, the antibody is nimotuzumab, or an antigen-binding portion thereof. In one embodiment, the antibody is olaratumab, or an antigen-binding portion thereof. In one embodiment, the antibody is oportuzumab, or an antigen-binding portion thereof. In one embodiment, the antibody is pertuzumab, or an antigen-binding portion thereof. In one embodiment, the antibody is pritumumab, or an antigen-binding portion thereof. In one embodiment, the antibody is ranibizumab, or an antigen-binding portion thereof. In one embodiment, the antibody is robatumumab, or an antigen-binding portion thereof. In one embodiment, the antibody is sibrotuzumab, or an antigen-binding portion thereof. In one embodiment, the antibody is siltuximab, or an antigen-binding portion thereof. In one embodiment, the antibody is tacatuzumab, or an antigen-binding portion thereof. In one embodiment, the antibody is tigatuzumab, or an antigen-binding portion thereof. In one embodiment, the antibody is tucotuzumab, or an antigen-binding portion thereof. In one embodiment, the antibody is veltuzumab, or an antigen-binding portion thereof. In one embodiment, the antibody is votumumab, or an antigen-binding portion thereof. In one embodiment, the antibody is zalutumumab, or an antigen-binding portion thereof.

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of indicates that the listed elements are required or mandatory, and that no other elements may be present. By “consisting essentially of is meant including any elements listed after the phrase, and limited to other elements that do not interfere with or contribute to the activity or action specified in the disclosure for the listed elements. Thus, the phrase “consisting essentially of indicates that the listed elements are required or mandatory, but that no other elements are optional and may or may not be present depending upon whether or not they affect the activity or action of the listed elements.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### EXAMPLES

The following Examples are merely illustrative and are not intended to limit the scope or content of the disclosure in any way.

### Example 1: CRISPR-EngCas12a Demonstrated Efficient Editing of CISH and TGFBR2 in NK Cells, and Edited NK Cells Exhibited Improved Effector Functions

Natural killer (NK) cells distinguish tumor from healthy tissue via multiple mechanisms, including recognition of stress ligands and loss of MHC class I expression. However, effector function of allogeneic NK cells can be diminished by the lack of functional persistence, as well as tumor-intrinsic immunosuppressive mechanisms, such as production of TGF-β. Described herein is a next-generation allogeneic NK cell therapy using CRISPR-Cas12a gene editing to enhance NK cell function through knockout of the CISH and TGFBR2 genes. Knockout of CISH, a negative regulator of IL-2/IL-15 signaling, improves NK cell effector function, while knockout of the TGF-β receptor gene, TGFBR2, renders NK cells resistant to TGF-β mediated suppression.

Specifically, NK cells derived from healthy human donor NK cells were edited using engineered Cas12a (“EngCas12a”; Cpf1 variant 4 amino acid sequence (SEQ ID NO: 1146)). CD3-depleted peripheral blood mononuclear cells were thawed into IL-15-containing NK MACS media and cultured for 14 days in GREX plates. CRISPR-EngCas12a gene editing was performed by ribonucleoprotein electroporation and cells were cultured for an additional 72 hours prior to analysis or functional assays.

The following guide RNA sequences were used for editing of CISH and TGFBR2.

TABLE6
gRNA sequences

| gRNA | 5′ DNA Extension | Cas12A-binding Sequence | Targeting Domain |
|---|---|---|---|
| (Target) | Sequence | (RNA) | Sequence (RNA) |
| CISH8401 | ATGTGTTTTTGTCAAAA | UAAUUUCUACUCUUGUAGAU | ACUGACAGCGUGAACAGGUAG |
| | GACCTTTT | (SEQ ID NO: 24) | (SEQ ID NO: 1169) |
| | (SEQ ID NO: 5) | | |
| | Full Length gRNA Sequence | | |
| | 5′ | | |
| | ATGTGTTTTTGTCAAAAGACCTTTTUAAUUUCUACUCUUGUAGAUACUGACAGCGUGAACAGGUAG 3′ | | |
| | (SEQ ID NO: 1170) | | |
| TGFBR238 | ATGTGTTTTTGTCAAAA | UAAUUUCUACUCUUGUAGAU | UGAUGUGAGAUUUUCCACCUG |
| 402 | GACCTTTT | (SEQ ID NO: 24) | (SEQ ID NO: 1171) |
| | (SEQ ID NO: 5) | | |

Full Length gRNA Sequence
5′
ATGTGTTTTTGTCAAAAGACCTTTTUAAUUUCUACUCUUGUAGAUUGAUGUGAGAUUUUCCACCUG-3′
(SEQ ID NO: 1172)

Indel analysis was performed by polymerase chain reaction amplification of the genomic region surrounding the CRISPR-EngCas12a cut site for each target followed by next-generation sequencing (NGS) and comparison to a reference genome to obtain percentage editing (indels).

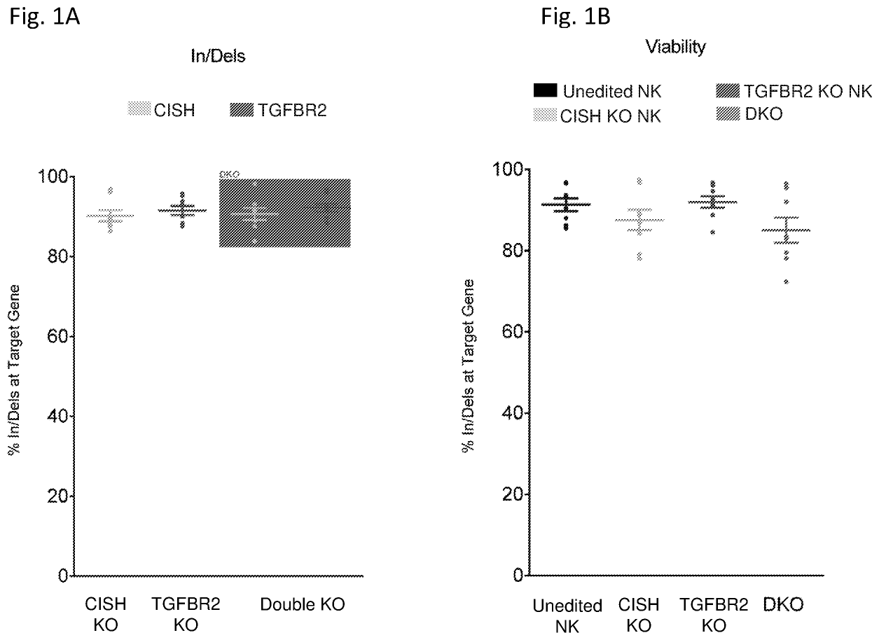

As demonstrated in FIGS. 1A and 1B, robust single and double-gene editing of TGFBR2 and CISH was achieved in NK cells. Greater than 80% indels at both targets in NK cells in both single and double gene knockout (KO, DKO) contexts were achieved.

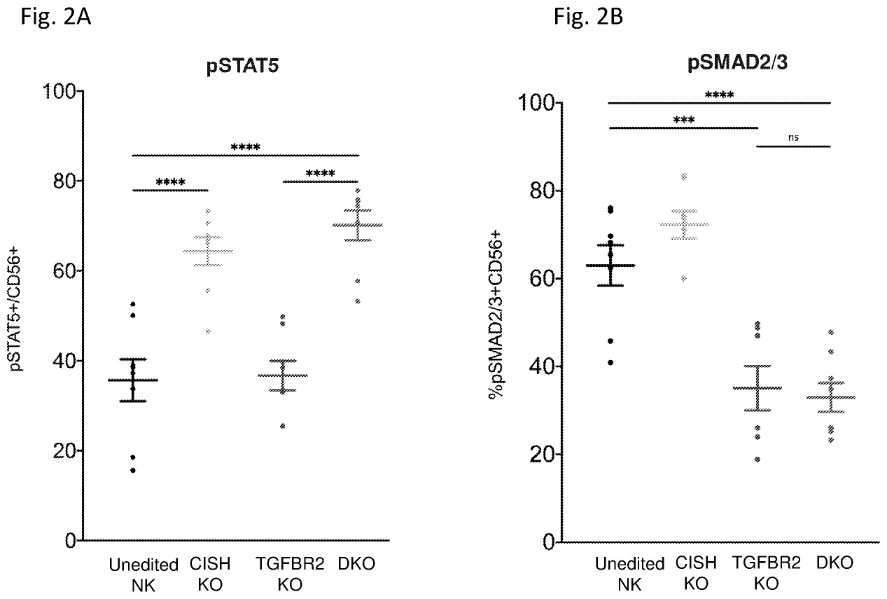

Phosphoflow cytometry assay was performed to determine the phosphorylated state of STAT5 (pSTAT5) and SMAD2/3 (pSMAD2/3) in NK cells. Knockout (KO) of CISH increased pSTAT5 (FIG. 2A) and pSTAT3 levels (data not shown) upon IL-15 stimulation, and KO of TGFBR2 decreased pSMAD2/3 levels upon TGF-β stimulation (FIG. 2B) in both single and double KO NK cells, as compared to unedited NK cells. These data suggest that double KO of CISH and TGFBR2 by CRISPR-EngCas12a increased NK cells' sensitivity to IL-15 and resistance to TGF-β mediated immunosuppression.

Spheroids were formed by seeding 5,000 SK-OV-3 or PC-3 cells in 96 well ultra low attachment plates. Spheroids were incubated at 37° C. before addition of effector cells and 10 ng/mL TGF-β. AlphaLISA was performed to analyze for TNF-α and IFN-γ secretion after co-culturing of effector cells with tumor spheroids and TGF-β for 120 hrs.

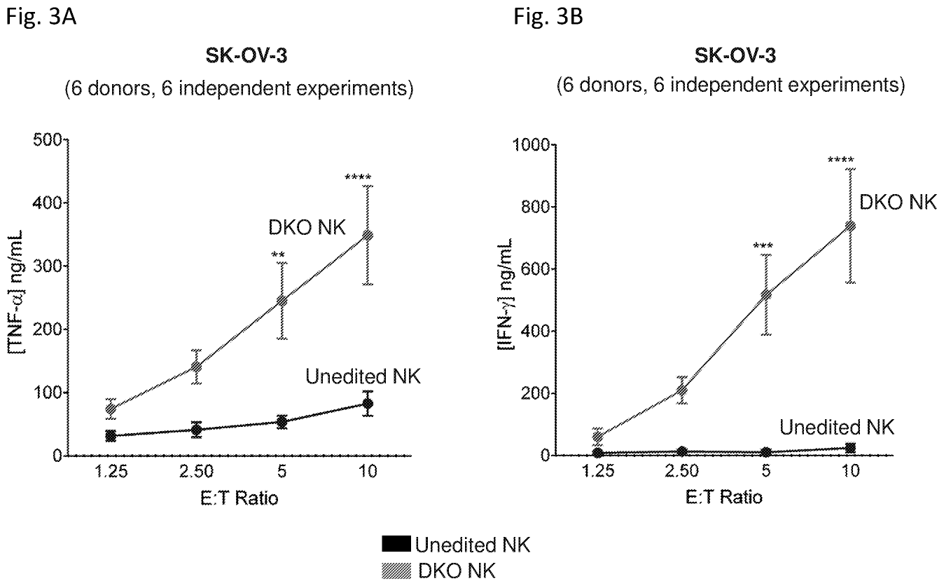

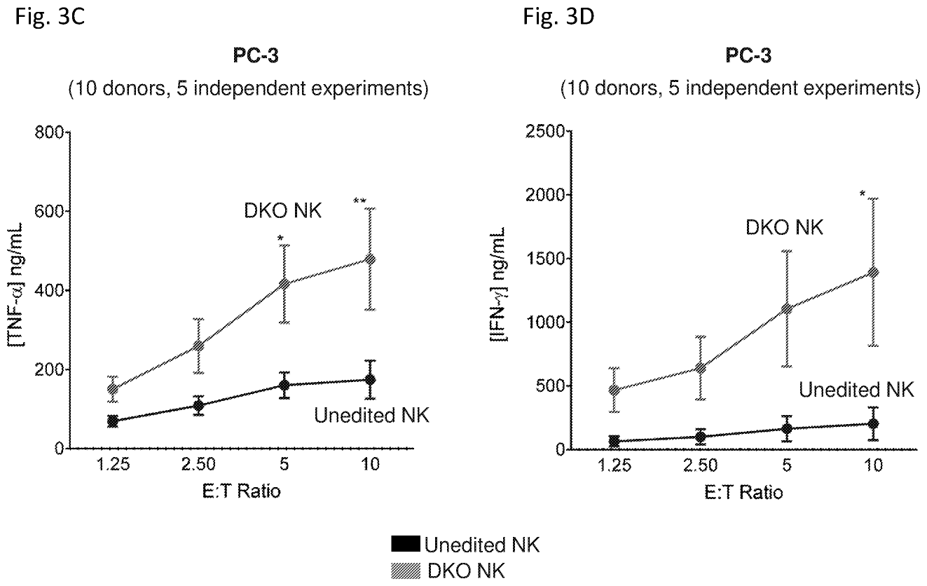

As shown in FIGS. 3A-3D, double KO (DKO) of CISH and TGFBR2 by CRISPR-EngCas12a increased the secretion of inflammatory cytokines TNF-α and IFN-γ at each of the E:T ratios tested in both SK-OV-3 and PC-3 cells as compared to unedited NK cells.

These results demonstrate efficient editing of healthy NK cells by CRISPR-EngCas12a, and editing at CISH and TGFBR2 enhanced effector functions of NK cells.

### Example 2: CISH/TGFBR2 DKO NK Cells Exhibit Enhanced Anti-Tumor Activity and Antibody-Dependent Cellular Cytotoxicity (ADCC) In Vitro

Spheroids were formed by seeding 5,000 NucLight Red labeled SK-OV-3 cells in 96 well ultra low attachment plates. Spheroids were incubated at 37° C. before addition of effector cells and 10 ng/mL TGF-β, followed by imaging of every 2 hours on the Incucyte S3 system for up to 120 hours. Data shown are normalized to the red object intensity at time of effector addition. Normalization of spheroid curves maintains the same efficacy patterns observed in non-normalized data.

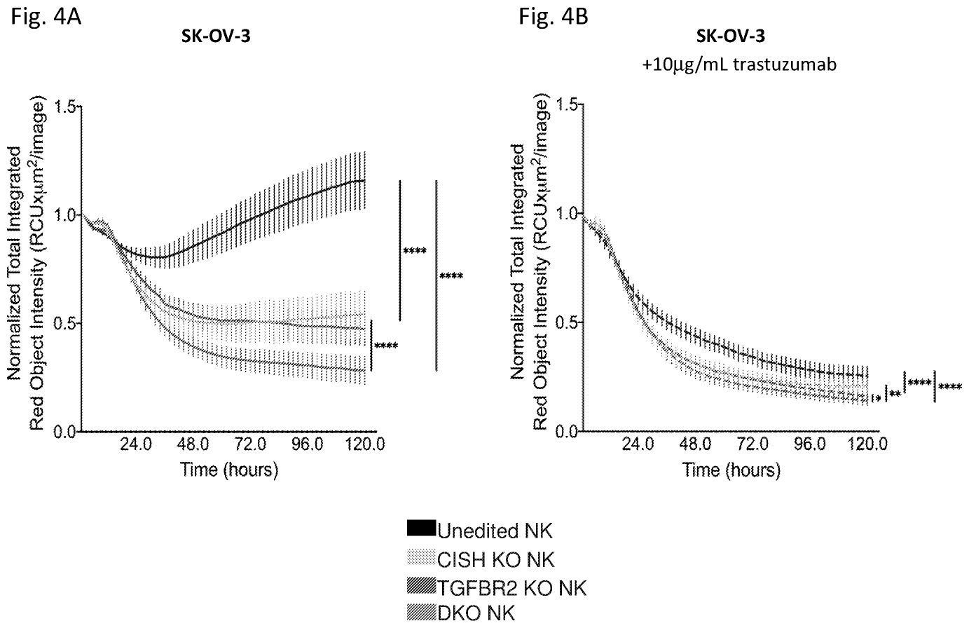

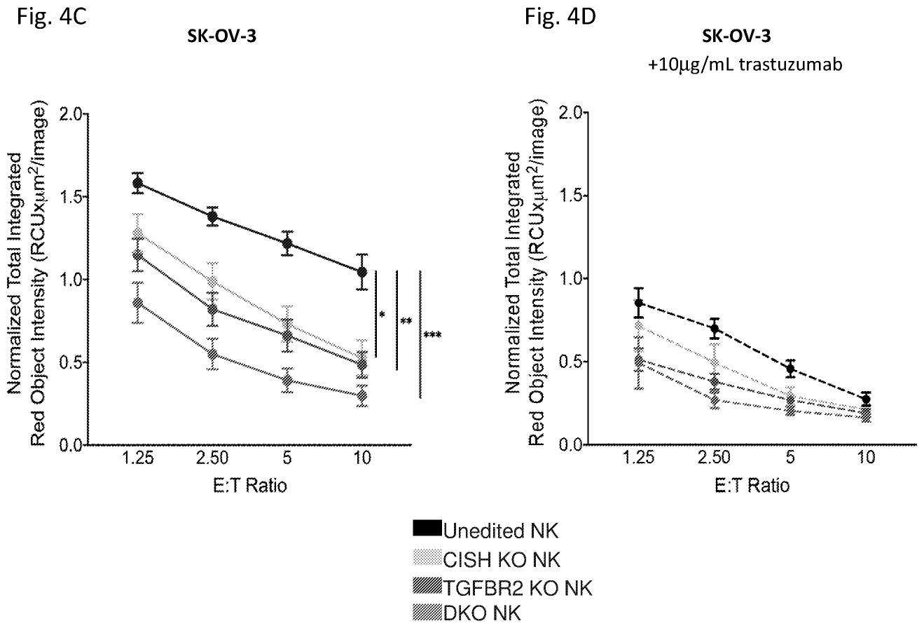

As depicted in FIGS. 4A-4D, both single knockouts (TGFBR2 KO and CISH NK) demonstrated improved cytotoxicity against tumor targets in the presence of exogenous TGF-β relative to unedited control NK cells (p<0.0001 for both single KOs). Furthermore, CISH KO NK cells unexpectedly perform killing at similar level to TGFBR2 KO NK cells, suggesting that knocking out CISH also helped NK cells overcome TGF-β immunosuppression. CISH/TGFBR2 DKO NK cells demonstrated superior rapid and sustained killing of ovarian tumor spheroids SK-OV-3 compared to either single knockouts or unedited control NK cells at the range of tested E:T ratios (n=7 independent experiments, 4 unique NK cell donors, p<0.0001), demonstrating additive effects of simultaneously targeting both pathways. The unedited, single KO and double KO NK cells also killed PC-3 prostate tumor spheroids in a similar trend (data not shown).

These data suggest that CISH/TGFBR2 DKO NK cells are very effective at targeting multiple types of tumors.

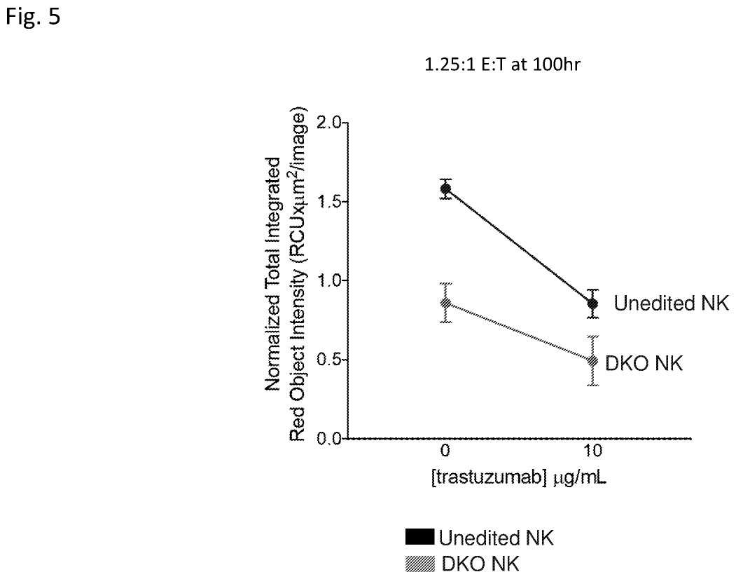

Additionally, killing of SK-OV-3 tumor spheroids by the NK cells were examined in the presence of traszutumab, a monoclonal antibody targeting HER2. The addition of trastuzumab (10 μg/ml) surprisingly increased killing by the unedited NK cells at a low E:T ratio of 1.25:1 to a great extent, and trastuzumab also significantly enhanced killing by the already effective DKO NK cells (see FIG. 5), which resulted in the greatest amount of tumor spheroid killing. This data shows that trastuzumab and NK cells have a strong antibody-dependent cellular toxicity (ADCC), and the combination of trastuzumab and NK cells, particularly the CISH/TGFBR2 DKO NK cells, has the potential to be an effective oncotherapy. The CISH/TGFBR2 DKO cells also killed the greatest amount of PC-3 prostate tumor spheroids in the presence of certuximab in a similar trend (i.e., more than unedited NK cells or single CISH KO or TGFBR2 KO cells in the presence of certuximab; data not shown).

### Example 3: CISH/TGFBR2 DKO NK Cells Exhibit Enhanced Anti-Tumor Activity In Vivo

In an in vivo NSG mouse xenograft model, 0.5 or 1 million fLuc-SK-OV-3 cells (expressing luciferase) were injected intraperitoneally (i.p.). At 7 days post tumor cell injection, 10 million cells of either unedited control NK cells or DKO NK cells were injected via i.p. Bioluminescence imaging using the IVIS system was performed weekly to monitor tumor burden.

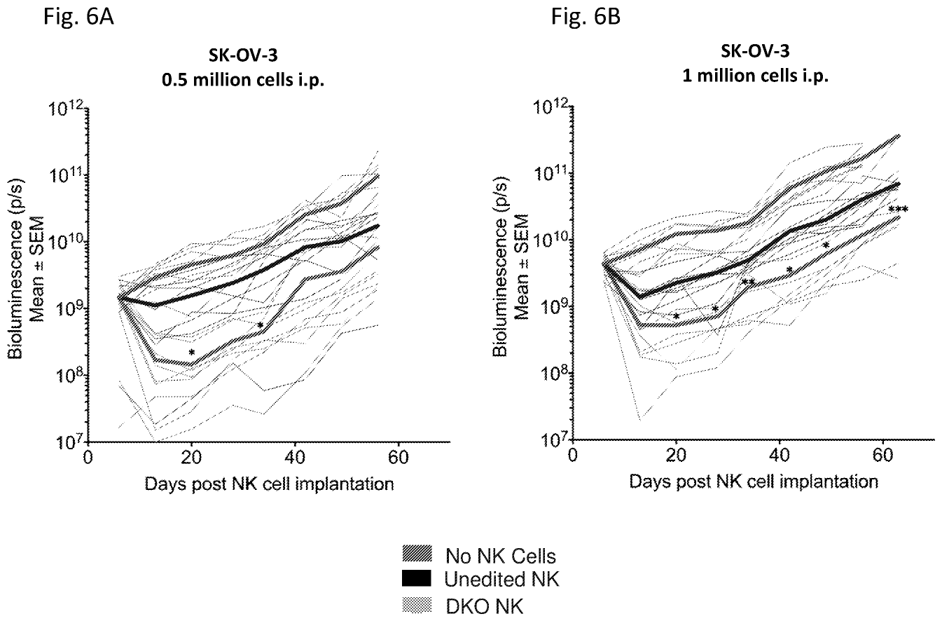

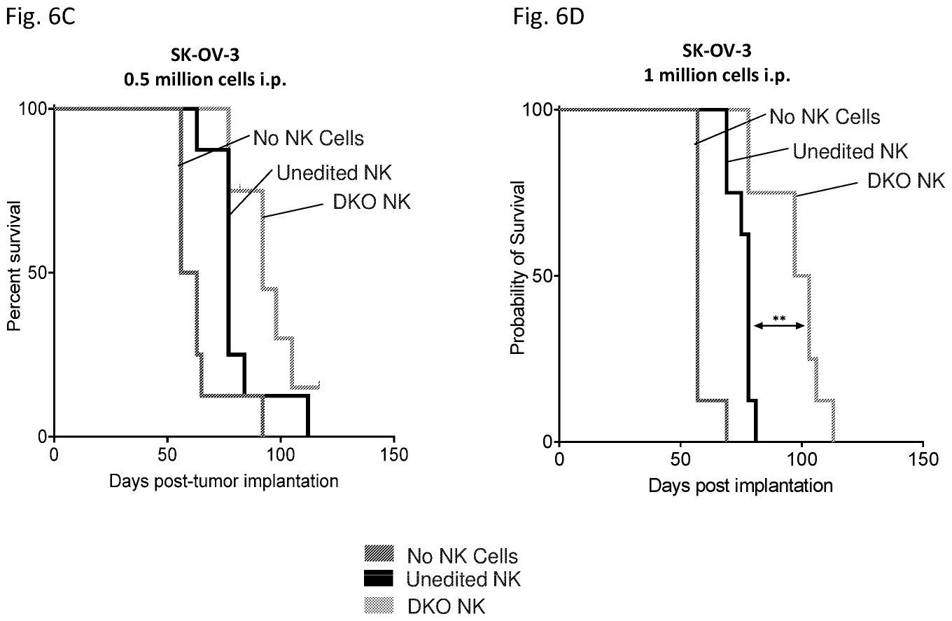

A single dose of DKO NK cells reduced tumor burden more effectively than unedited control NK cells (FIGS. 6A and 6B), leading to a statistically significant increase in median survival time and lower tumor burden (FIGS. 6C-6D)

This result suggests that the CISH/TGFBR2 DKO NK cells are promising as cell-based medicine for cancer.

### Example 4: Antibody-Dependent-Cellular Cytotoxicity (ADDC) Further Enhanced Anti-Tumor Activity by CISH/TGFBR2 DKO NK In Vivo

NSG mice (n=8 per group) were inoculated via i.p. with 0.5 million luciferase-expressing SK-OV-3 cells. On day 6 post-tumor inoculation, tumor bearing mice were randomized into groups with comparable tumor burden. A day later, mice were injected via i.p with 2.5 mpk isotype, 2.5 mpk trastuzumab, 10 million unedited CD56+NK cells, 10 million DKO CD56+NK cells or the combination of DKO CD56+NK cells with trastuzumab.

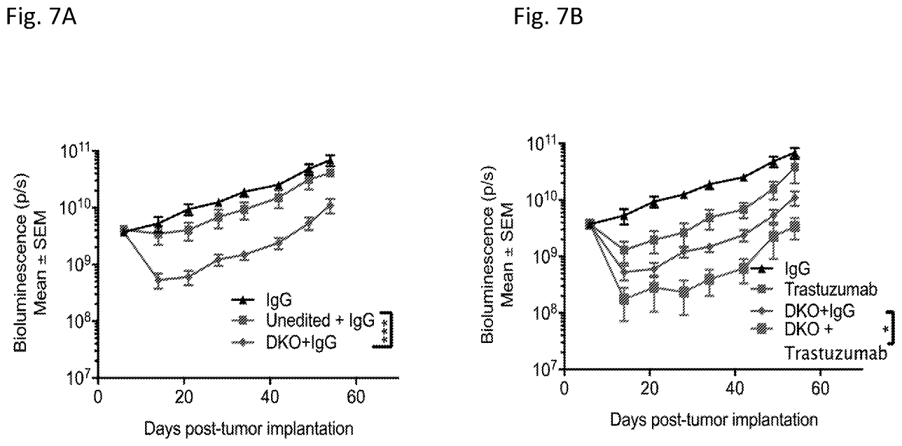

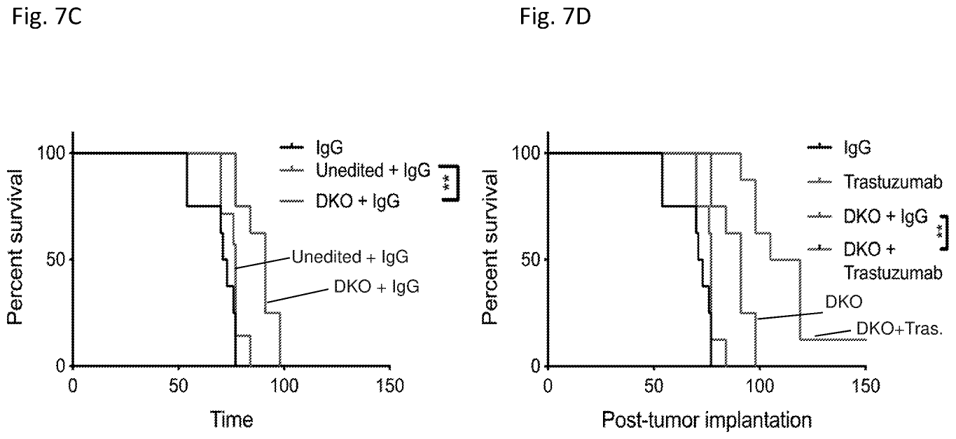

FIGS. 7A and 7C again show that DKO NK cells were significantly more effective at controlling tumor growth and increased lifespan of mice. Trastuzumab significantly increased these effects of DKO NK treatments, as shown in FIGS. 7B and 7D.

This data show that traszutumab can mediate ADCC and promote tumor killing by the DKO NK cells in vivo, and strongly suggest that combination therapy of traszutumab and the DKO NK cells can be very effective treatment for cancers, such as ovarian cancer.

### Example 5: ADCC Effect was Also Observed in Combination Treatment of Rituximab and NK Cells in a Serial Killing Assay

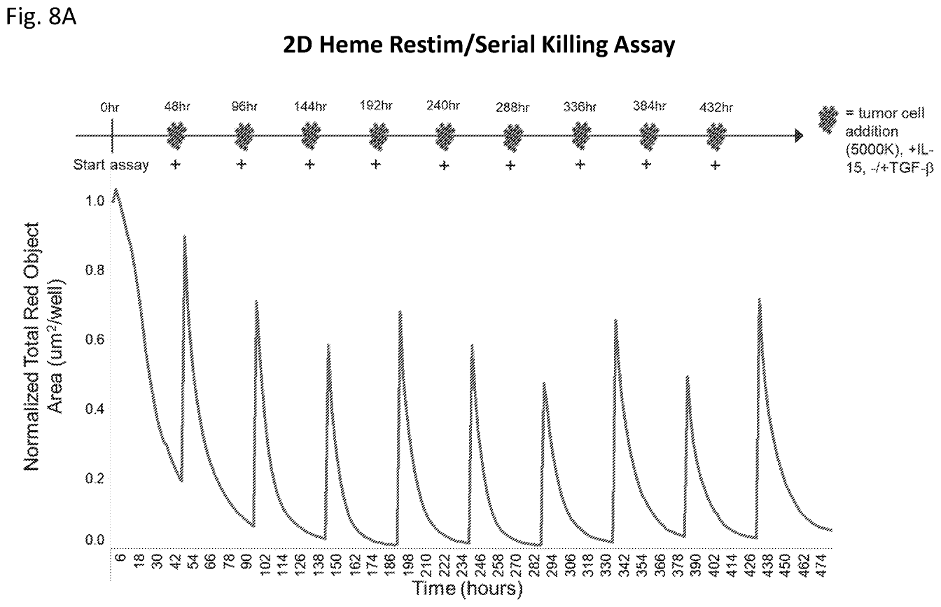

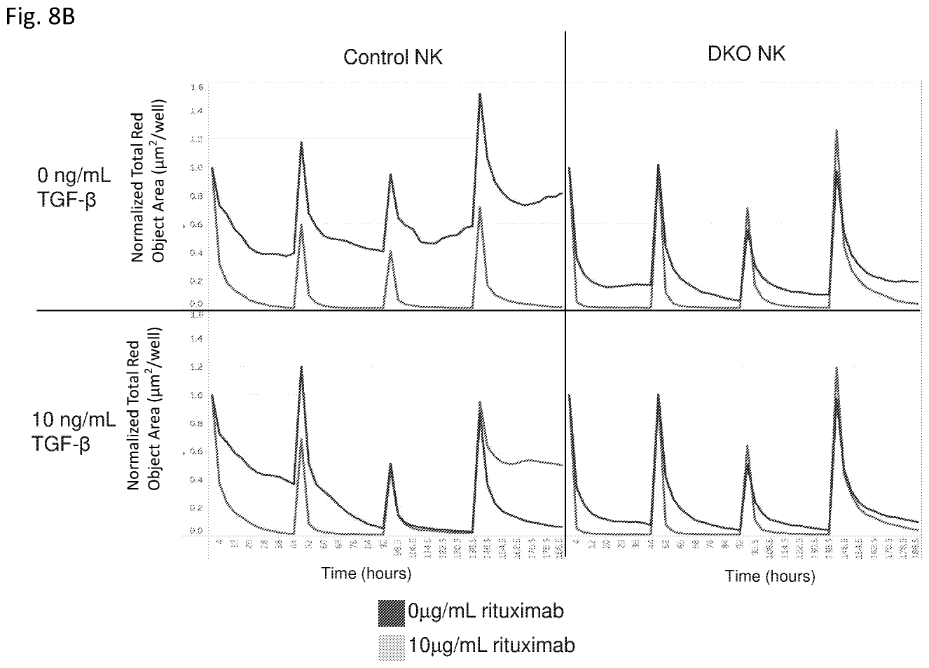

A 2D Heme Restimulation/Serial Killing Assay was used to determine the endurance of NK cells in serial tumor killing. Specifically, 200 thousand unedited control NK cells or CISH/TGFBR2 DKO NK cells were seeded in each well. 10 thousand Raji tumor cells (a hematological malignant cell line) were added to the NK cells at the beginning of the assay, and subsequently 5 thousand tumor cells and IL-15 were spiked into each well every 48 hours. Surviving tumor cells were quantified by normalized total red object area (see FIG. 8A).

Rituximab alone did not kill tumor cells without the presence of NK cells (data not shown). For unedited NK cells, the addition of rituximab improved tumor cell killing in both the absence and presence of TGF-β (FIG. 8B, left 2 panels). DKO NK cells were already much more effective than unedited NK cells in killing tumor cells (FIG. 8B, comparing top 2 panels), and the addition of rituximab further enhanced tumor cell killing by DKO NK cells (FIG. 8B, right 2 panels). NK cells were still effective at killing the tumor cells after 7 days in this serial killing assay.

This experiment shows that rituximab mediates ADCC in the Raji cell killing by NK cells. The combination of rituximab and CISH/TGFBR2 DKO NK cells were most effective at serially killing tumor cells in the presence or absence of TGF-β for at least 7 days in this assay, suggesting that this is an effective combination therapy for cancers, such as hematologic cancer.

Overall, the experimental results showing that CISH/TGFBR2 DKO NK cells exhibit improved ADCC and effector function in the presence of different therapeutic antibodies for cancer, including trastuzumab and certuximab (Examples 2 and 4) and rituximab (Example 5), show that the CISH/TGFBR2 DKO cells could be combined with a variety of cancer treating antibodies to improve treatment outcomes for a variety of cancers.

### Example 6: Functional Characterization of CISH/TGFBR2 DKO NK Cells Reveals Increased Granzyme B and Degranulation Supporting Improved Serial Killing Capacity

As described above, CISH/TGFBR2 DKO NK cells have increased effector function and are resistant to TGF-β inhibition. These combined activities enable this healthy donor derived NK cell therapy to kill tumor cells more efficiently and for a longer duration than control NK cells in the presence of TGF-β.

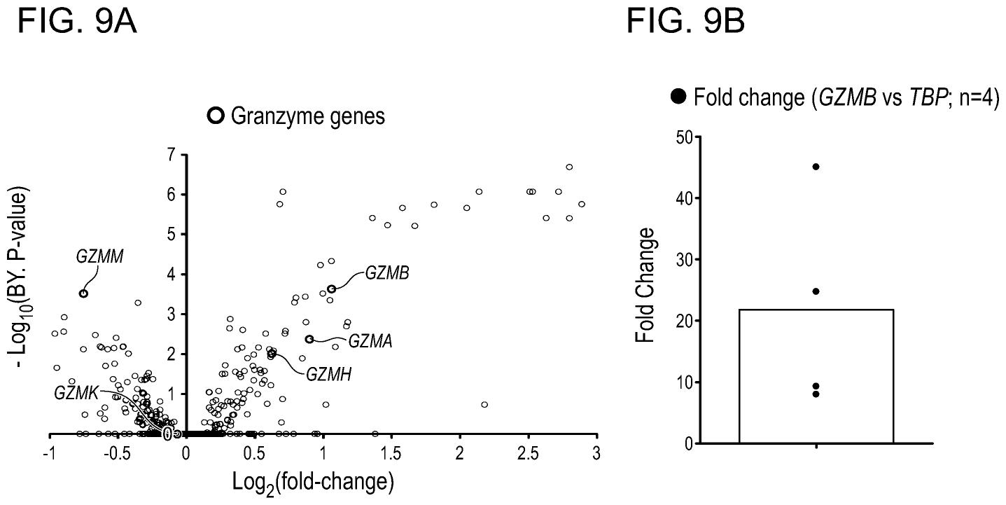

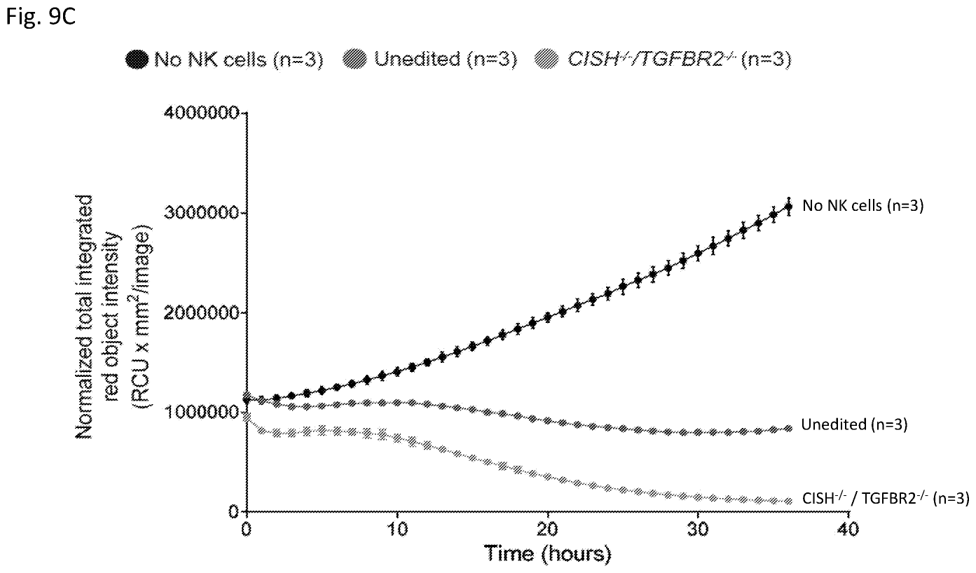

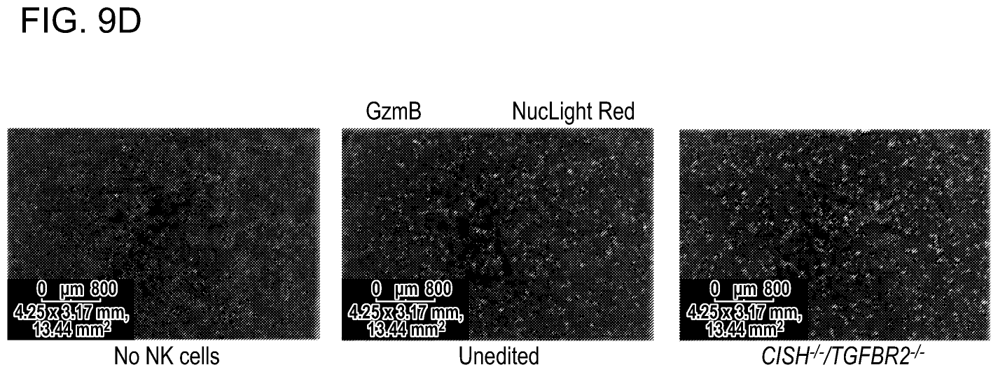

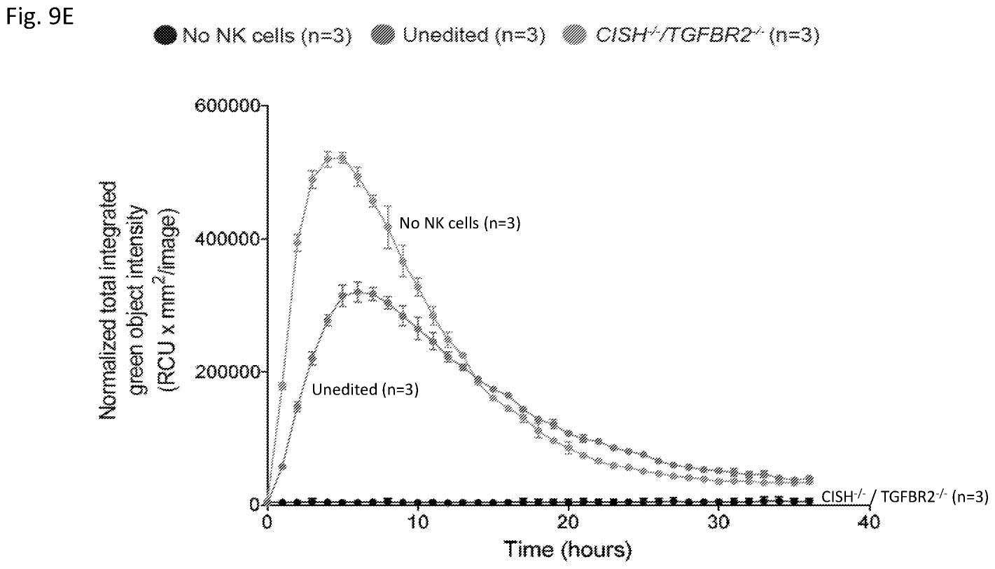

To further investigate the mechanism by which CISH/TGFBR2 double knockout (DKO) NK cells (produced as described in Example 1) have increased serial killing capacity, the transcriptional changes contributed by each gene edit was first explored with a focus on transcripts critical for NK cell effector function and metabolism using Nanostring analysis. Unedited, mock electroporated, and control edited (targeting a biologically irrelevant site) NK cells were included as controls in addition to CISH and TGFBR2 single and double gene knockout (KO) NK cells to interrogate the potential impact of electroporation and double-stranded DNA breaks on NK cell function. All samples included in the analysis were cultured for 3 days in IL-15 (10 ng/mL) post-electroporation. Interestingly, no significant transcriptional changes were detected in all control conditions, while samples that contained CISH editing clearly upregulated transcripts relevant for NK cell effector function, including contents of cytolytic granules (GZMB, GZMA, and GZMH) (FIG. 9A). Furthermore, an average of 22 fold more GZMB transcript was expressed in CISH/TGFBR2 DKO NK cells than control NK cells as measured by RT-qPCR in four unique NK cell donors (FIG. 9B).

Next, whether the increase in cytolytic signature could be one potential mechanism whereby CISH/TGFBR2 DKO NK cells were functionally superior relative to control NK cells was tested. Consistent with this hypothesis, CISH/TGFBR2 DKO NK cells showed significantly higher levels of CD107a, a marker of degranulation, after 14 hrs of co-culture with SKOV-3 tumor cells, suggesting that CISH/TGFBR2 DKO NK cells had an increased capacity to degranulate relative to control NK cells. To determine the presence of granzyme proteins within tumor cells post engagement with NK cells, a novel GzmB reporter gene was developed and lentiviral vectors were used to introduce this reporter into tumor cell lines (SK-OV-3::GzmB). SK-OV-3 tumor cells were transduced with the reporter, and then co-cultured with CISH/TGFBR2 DKO NK cells or control NK cells. 10⁶NK cells were co-cultured with 5000 SK-OV-3::GzmB cells labelled with NucLight Red; and imaged every 2 hours on the Incucyte S3 system for up to 36 hours (FIG. 9D). GzmB activity was identified 4 hours sooner in the SK-OV-3 tumor cells transduced with the GzmB reporter that were co-cultured with the CISH/TGFBR2 DKO NK cells relative to transduced tumor cells co-cultured with control NK cells. In addition, CISH/TGFBR2 DKO NK cells affected 80% more SK-OV-3 tumor cells with granzyme B compared to control NK cells over a 36-hour period (FIGS. 9C and 9E). Significantly, these data demonstrated that CISH/TGFBR2 DKO NK cells not only released GzmB more rapidly than control NK cells, but also the amount of GzmB degranulated was greater as well (relative to control NK cells), confirming that enhanced degranulation is a key mechanism by which CISH/TGFBR2 DKO NK cells have superior functional capacity relative to control NK cells.

Together, these data demonstrate that CISH/TGFBR2 DKO NK cells expressed high levels of GzmB and had more rapid and enhanced degranulation activity than unedited NK cells, suggesting this as a potential mechanism by which CISH/TGFBR2 DKO NK cells demonstrate superior cytotoxicity during in vitro killing of SK-OV-3 tumor targets.

### Example 7: CISH/TGFBR2 DKO NK Cells Demonstrate Superior Function During Tumor Target Killing in Nutrient Deprived Conditions Through Increased Spare Respiratory Capacity

Natural killer (NK) cells distinguish tumor from healthy tissue via multiple mechanisms, including recognition of stress ligands and loss of MHC class I expression. As described above, CISH/TGFBR2 DKO NK cells were produced via CRISPR-Cas12a mediated CISH and TGFBR2 double gene knockout in NK cells derived from healthy donors (see Example 1). These cells demonstrated resistance to TGF-β inhibition and increased tumor control both in vitro and in vivo.

Anti-tumor activity by effector cells requires significant energy expenditure and is constrained by nutrients available in the tumor microenvironment (TME). The TME is known to be nutrient-deprived due to active tumor cell metabolism leading to competition for essential nutrients with infiltrating effector cells, while at the same time being enriched in immunosuppressive metabolites such as lactic acid due to Warburg Metabolism. To explore whether CISH/TGFBR2 DKO NK cells are functional in such hostile metabolic conditions, the metabolic microenvironment was modelled in the established SK-OV-3 ovarian tumor spheroid model.

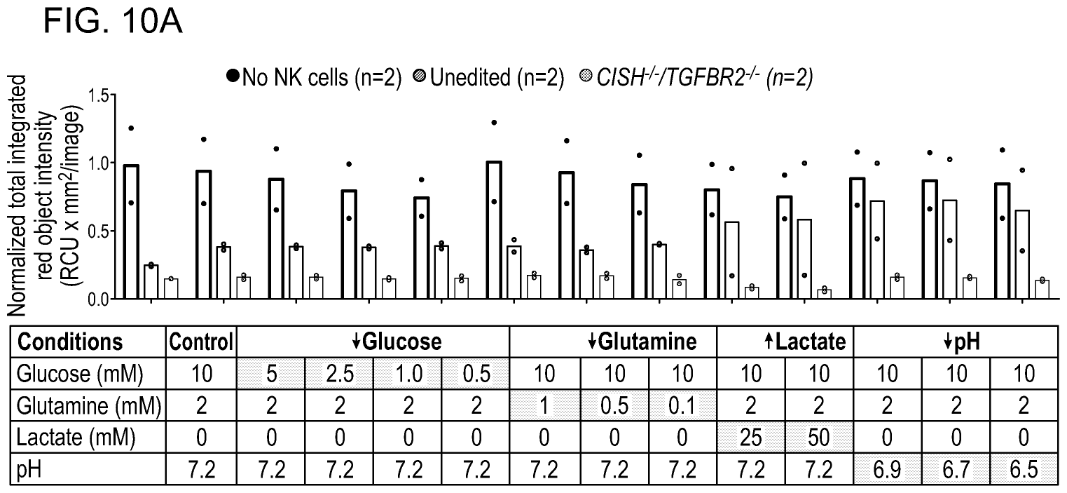

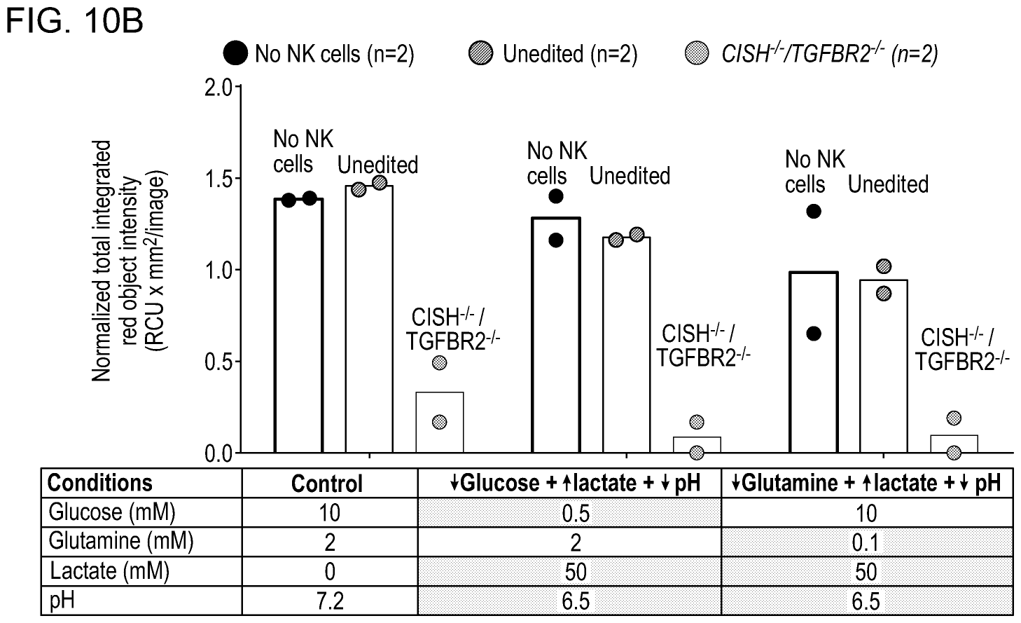

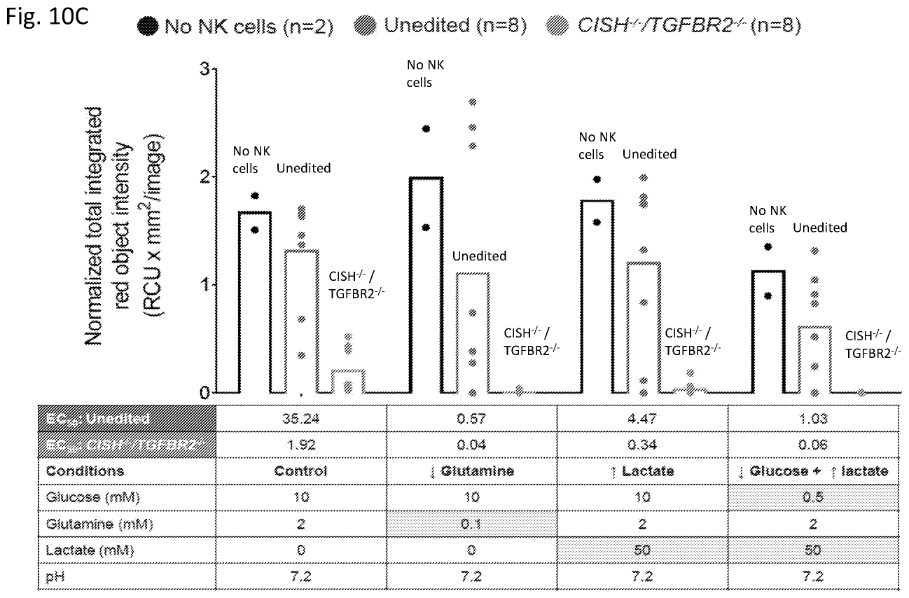

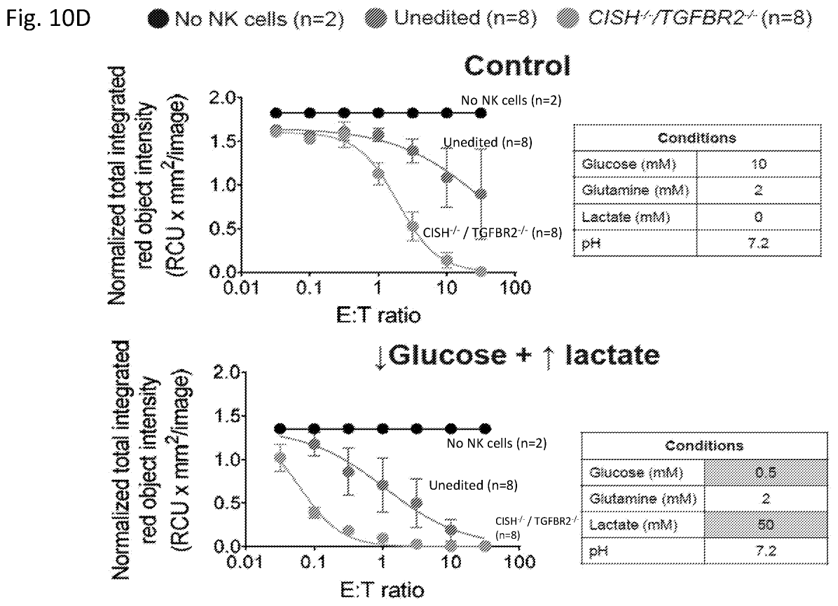

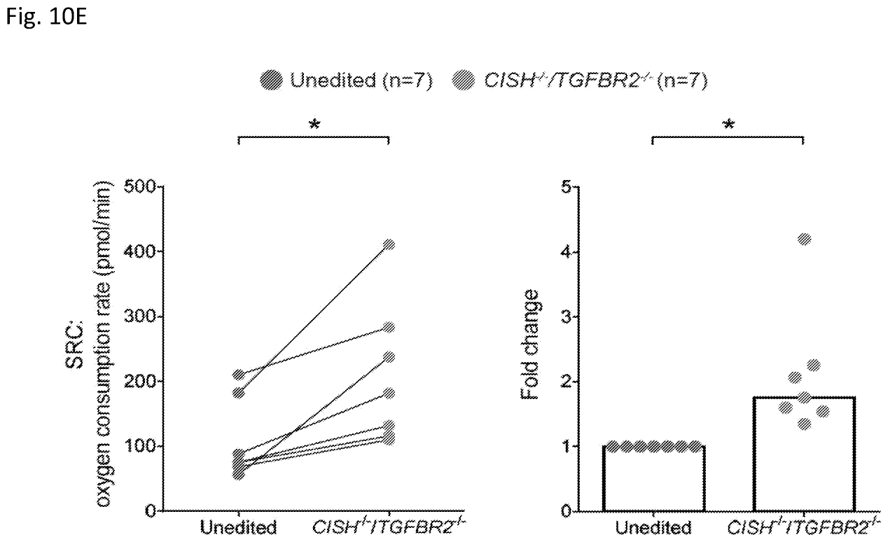

To model this hostile microenvironment in vitro, SK-OV-3 ovarian tumor spheroids were generated in decreasing concentrations of glucose (10-0.5 mM, e.g., 10 mM (control), 5 mM, 2.5 mM, 1.0 mM or 0.5 mM) or glutamine (2-0.1 mM, e.g., 2 mM (control), 1 mM, 0.5 mM or 0.1 mM), two important fuels for NK cell metabolism, as well as increasing concentrations of inhibitory metabolite lactate (0-50 mM, e.g., 0.0 mM (control), 25 mM or 50 mM), or decreasing pH (7.2-6.5, e.g., 7.2 (control), 6.9, 6.7, or 6.5). Each of these metabolic conditions are known to suppress effector cell function, and the system was further stressed by performing spheroid cells co-cultures in the absence of TGF-β at a 10:1 effector:target ratio (FIG. 10A). In all of the above conditions, SK-OV-3 tumor spheroids formed at similar rates relative to spheroids formed in standard culture media. Significantly, it was found that in each of these conditions, CISH/TGFBR2 DKO NK cells demonstrated rapid and sustained tumor killing in the absence of critical nutrients or in unfavorable growth conditions relative to control unedited NK cells.

To further model the complexity of the metabolic conditions in the TME, a multifactorial matrix of metabolic conditions was created where deprivation of multiple nutrients was combined in the presence of lactate and/or acidic cell culture media. Specifically, the cytotoxicity of NK cells was assayed with SK-OV3-tumor spheroid in the presence of 10 ng/mL TGF-β at a 5:1 effector:target ratio (FIG. 10B). The cytotoxicity of NK cells was also assayed with SK-OV3-tumor spheroid that were selectively evolved to grow in nutrient-deprived and/or high lactate media in the presence of 10 ng/mL TGF-β at a 10:1 effector:target ratio at 100 hours (FIG. 10C) or at varying effector:target ratios (FIG. 10D) at 100 hours. Remarkably, in all the matrixed conditions tested, it was surprisingly found that CISH/TGFBR2 DKO NK cells demonstrated increased cytotoxicity against SK-OV-3 spheroids relative to control NK cells, suggesting a clear and robust metabolic advantage of CISH/TGFBR2 DKO NK cells over control NK cells. A corresponding increase in the concentrations of IFN-γ and TNF-α was further observed by CISH/TGFBR2 DKO NK cells in all of these conditions relative to control NK cells.

Given that mitochondrial respiration is key to NK cell persistence and function, the mitochondrial function of CISH/TGFBR2 DKO NK cells was next interrogated. CISH/TGFBR2 DKO NK cells consistently demonstrated greater spare respiratory capacity (SRC) relative to control NK cells after overnight IL-15 starvation, suggesting enhanced mitochondrial reserve as a result of CISH and TGFBR2 knockout (FIG. 10E). SRC is a function of mitochondrial mass and fitness. A cell with a larger SRC can produce more ATP and overcome more stress, including oxidative stress. Similar results were observed in NK cells cultured with IL-15 overnight. The increase in SRC likely enables CISH/TGFBR2 DKO NK cells to meet enhanced energy demands necessary to mediate effector function in metabolically challenging conditions, thus sustaining superior cytotoxic capacity and cytokine production.

In summary, a complex multifactorial in vitro tumor spheroid model was developed to more realistically probe the TME likely to be encountered in vivo. These data demonstrate that enhanced metabolic function of CRISPR-Cas12a CISH and TGFBR2 gene edited NK cells results in superior cytotoxicity during in vitro killing of SK-OV-3 spheroids in metabolically unfavorable conditions that are similar to those experienced by effector cells in tumors. These data further demonstrate the potential of CISH/TGFBR2 DKO NK cells as a novel cell therapy for cancer.

### Example 8. CISH/TGFBR2 DKO NK Cells Demonstrated Enhanced Anti-Tumor Activity and Sustained Serial Killing Against Other Tumor Cell Lines

The anti-tumor activity of CISH/TGFBR2 double knockout NK cells (produced as described in Example 1) was further tested against numerous other tumor cells lines, such as Nalm6 tumor cells and other hematologic tumor cell lines.

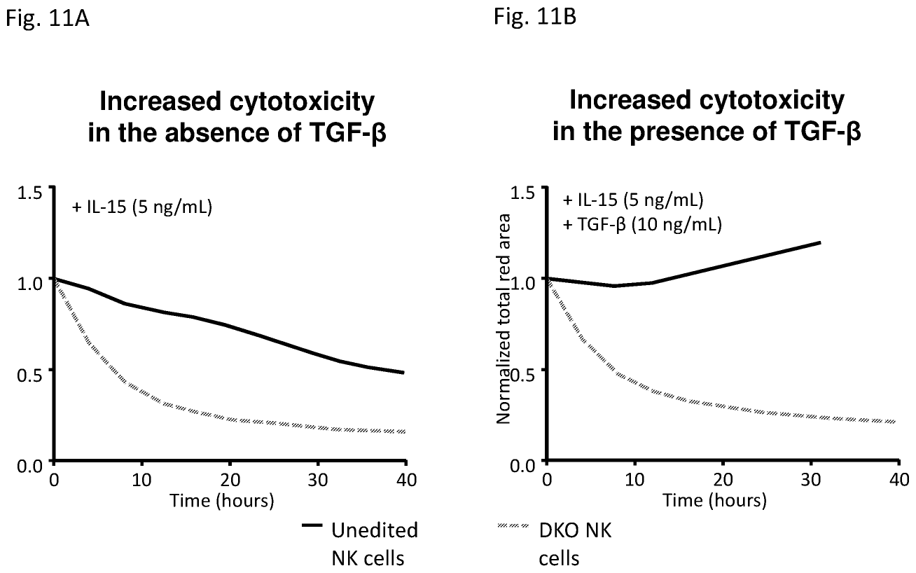

FIGS. 11A and 11B depict that CISH/TGFBR2 double knockout NK cells exhibited enhanced anti-tumor activity against Nalm6 tumor cells in the presence of TGF-β compared to control unedited NK cells. CISH/TGFBR2 DKO NK cells, or unedited control NK cells, were co-cultured with Nalm6 tumor cells at a 20:1 effector tumor ratio in the presence of 5 ng/mL IL-15, without and with the addition of 10 ng/mL TGF-β. Increased cytotoxicity was observed in all conditions while a greater increase was observed when TGF-β was added in the cell culture.

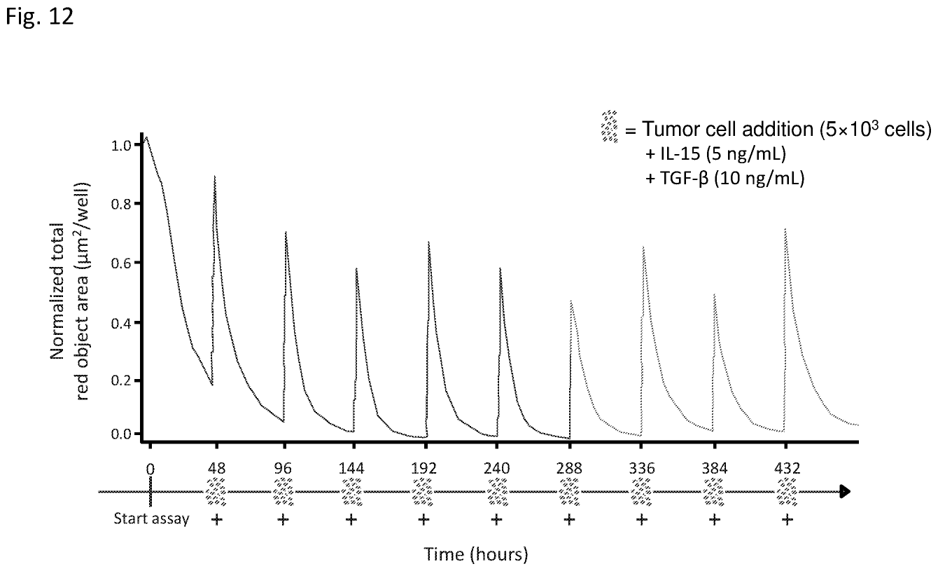

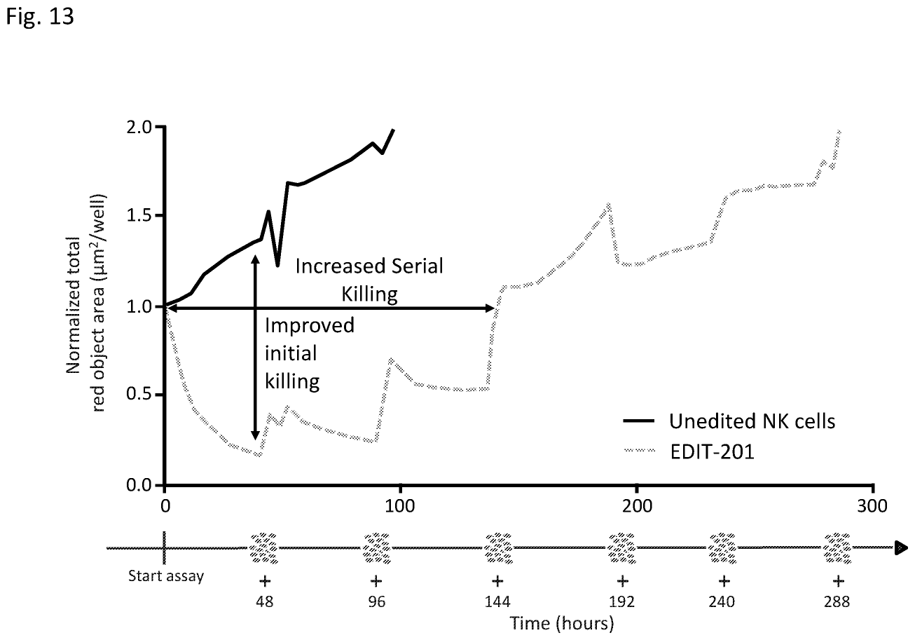

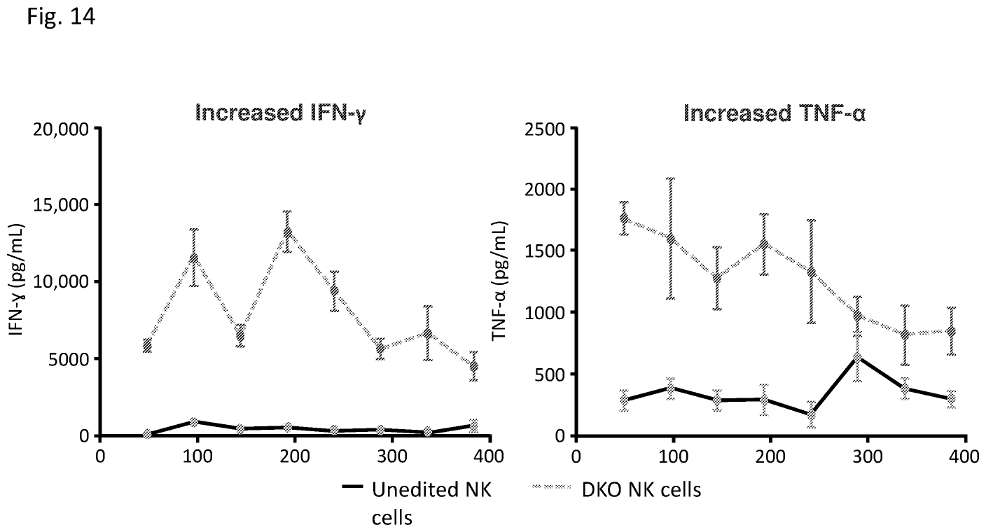

In addition, as shown in FIG. 12 and FIG. 13, CISH/TGFBR2 DKO NK cells continually killed Nalm6 tumor cells for more than 8 days in an in vitro serial killing assay, whereas the unedited NK cells had limited serial killing effect. Nalm6 tumor cells (5×10³ cells) were added to the NK cells every 48 hours in the presence of 5 ng/mL IL-15 and 10 ng/mL TGF-β in this assay. Supernantat from this assay were harvested every 48 hours, and CISH/TGFBR2 DKO NK cells were shown to produce higher levels of IFN-γ and TNF-α versus unedited NK cells over the duration of the assay (FIG. 14), suggesting that CISH/TGFBR2 DKO NK cells can continue to produce these inflammatory cytokines even after serial killing.

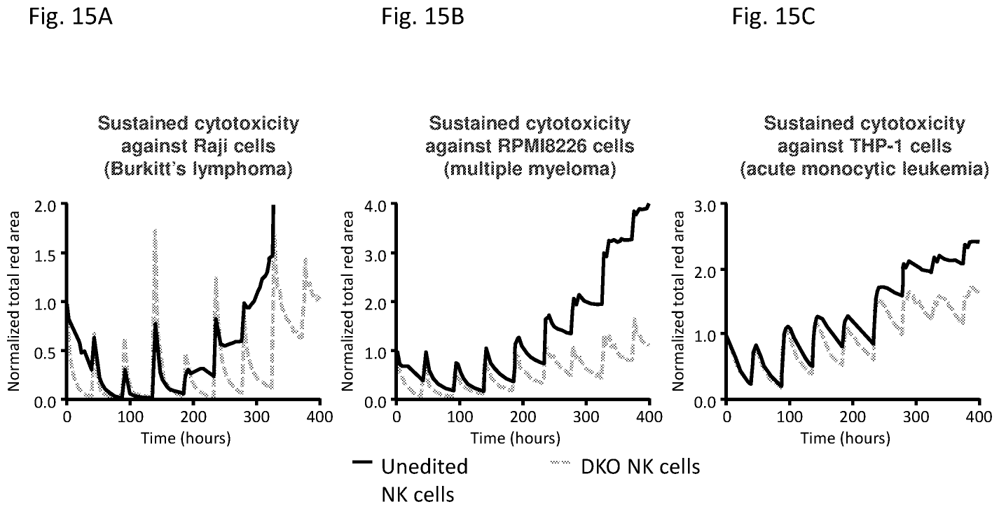

Other hematologic tumor cell lines, such as Raji (Burkitt's lymphoma), RPM18226 (multiple myeloma) and THP-1 (acute monocytic leukemia) cells, were also tested in the serial killing assay. As shown in FIGS. 15A-15C, CISH/TGFBR2 DKO NK cells demonstrated sustained serial killing activity against each of these tumor cell lines in the presence of TGF-β, and the CISH/TGFBR2 DKO NK cells continually killed the cells of each of these tumor cell lines for more than 8 days.

These data suggest that CISH/TGFBR2 DKO NK cells are very effective at targeting multiple types of tumors.

1. A method of inducing antibody-dependent cell-mediated cytotoxicity (ADCC) of a cancer cell, the method comprising contacting the cancer cell with a modified natural killer (NK) cell and an antibody, or an antigen-binding portion thereof, wherein the modified NK cell exhibits a loss of function of transforming growth factor beta receptor 2 (TGFβR2) and cytokine inducible SH2 containing protein (CISH), thereby inducing ADCC of the cancer cell.

2. The method of claim 1, wherein the contacting is in a subject.

3. A method of treating cancer in a subject, the method comprising administering to a subject a modified natural killer (NK) cell and an antibody, or an antigen-binding portion thereof, wherein the modified NK cell exhibits a loss of function of transforming growth factor beta receptor 2 (TGFβR2) and cytokine inducible SH2 containing protein (CISH), wherein the administering induces ADCC of a cancer cell in the subject, thereby treating the cancer in the subject.

4. The method of claim 3, wherein the administering (a) increases ADCC by at least about 20%, at least about 25%, at least about 50%, at least about 75%, at least about 100%, at least about 2-fold, at least about 5-fold or at least about 10-fold as compared to ADCC of a cancer cell using an unmodified NK cell and the antibody: (b) decreases tumor volume in the subject by at least about 10%, at least about 20%, at least about 30%, at least about 40%, at least about 50%, at least about 60%, at least about 70%, at least about 80%, or at least about 90% by about 10 days or about 20 days after administering, and optionally wherein the administering decreases tumor volume in the subject for at least about 10 days, at least about 20 days, at least about 30 days, or at least about 40 days after the administering; and/or (c) increases the survival time of the subject.

5-8. (canceled)

9. The method of claim 1, wherein the contacting is in vitro.

10. The method of claim 9, wherein the modified NK cell comprises (a) an increase in level of TNFα by at least about two fold as compared to a control level expression of TNFα, optionally wherein the control level of TNFα is a level of TNFα produced by an unmodified NK cell under the same conditions: (b) an increase in level of IFNα by at least about two fold as compared to a control level expression of IFNγ, optionally wherein the control level of IFNγ is a level of IFNγ produced by an unmodified NK cell under the same conditions: (c) an increase in level of a cytolytic granule by at least about two fold as compared to a control level expression of the cytolytic granule, optionally wherein the cytolytic granule is selected from the group consisting of GZMB, GZMA and GZMH, optionally wherein the control level of cytolytic granule is a level of cytolytic granule produced by an unmodified NK cell under the same conditions: (d) an increase in production rate of a cytolytic granule by at least about two fold as compared to a control production rate of the cytolytic granule, wherein the cytolytic granule is selected from the group consisting of GZMB, GZMA and GZMH, optionally wherein the control production rate of cytolytic granule is a production rate of cytolytic granule by an unmodified NK cell under the same conditions: (e) an increase in level of CD107a by at least about two fold as compared to a control level expression of CD107a, optionally wherein the control level of CD107a is a level of CD107a in an unmodified NK cell under the same conditions: (f) a decrease in normalized total integrated red object intensity in a tumor spheroid assay by at least about 20% as compared to a control level of normalized total integrated red object intensity, wherein the control level of normalized total integrated red object intensity is a level of normalized total integrated red object intensity produced using an unmodified NK cell under the same conditions: (g) an increase in cytotoxicity activity under a nutrient-depriving condition by at least about 20% as compared to a control level of cytotoxicity activity, optionally wherein the control level of cytotoxicity activity is a cytotoxicity level of an unmodified NK cell under the same conditions; and/or (h) an increase in spare respiratory capacity by at least 20% as compared to a control level of spare respiratory capacity, optionally wherein the control level of spare respiratory capacity is a level of spare respiratory capacity of an unmodified NK cell under the same conditions.

11-25. (canceled)

26. The method of claim 3, wherein the antibody, or antigen-binding portion thereof, binds an antigen on the cancer cell.

27. The method of claim 26, wherein the antigen on the cancer cell is epidermal growth factor receptor (EGFR), HER2, CD20, PD-L1, PD-1 (PEMBRO and NIVO), CTLA-4 (IPI), CD73, or TIGIT.

28. The method of claim 27, wherein the antibody is cetuximab,
rituximab, or trastuzumab, or an antigen-binding portion thereof.

29. The method of claim 3, wherein the modified NK cell is administered concurrently with the antibody, or the antigen-binding portion thereof.

30. The method of claim 3, wherein the antibody, or antigen-binding portion thereof, is administered prior to the modified NK cell, or wherein the modified NK cell is administered prior to the antibody, or the antigen-binding portion thereof.

31. The method of claim 3, wherein the cancer cell is a head and neck cancer cell, breast cancer cell, colorectal cancer cell, gastric cancer cell, renal cell carcinoma (RCC) cell, non-small cell lung cancer (NSCLC) cell, solid tumor cell, bladder cancer cell, hepatocellular carcinoma cell, prostate cancer cell, ovarian/uterine cancer cell, pancreatic cancer cell, mesothelioma cell, melanoma cell, glioblastoma cell, cervical cancer cell, oral cavity cancer cell, cancer of the pharynx, thyroid cancer cell, gallbladder cancer cell, soft tissue sarcoma, or a hematological cancer cell.

32. The method of claim 1, wherein the antibody, or antigen-binding portion thereof, binds an antigen on the cancer cell.

33. The method of claim 32, wherein the antigen on the cancer cell is epidermal growth factor receptor (EGFR), HER2, CD20, PD-L1, PD-1 (PEMBRO and NIVO), CTLA-4 (IPI), CD73, or TIGIT.

34. The method of claim 33, wherein the antibody is cetuximab, rituximab,
or trastuzumab, or an antigen-binding portion thereof.

35. The method of claim 1, wherein the cancer cell is a head and neck cancer cell, breast cancer cell, colorectal cancer cell, gastric cancer cell, renal cell carcinoma (RCC) cell, non-small cell lung cancer (NSCLC) cell, solid tumor cell, bladder cancer cell, hepatocellular carcinoma cell, prostate cancer cell, ovarian/uterine cancer cell, pancreatic cancer cell, mesothelioma cell, melanoma cell, glioblastoma cell, cervical cancer cell, oral cavity cancer cell, cancer of the pharynx, thyroid cancer cell, gallbladder cancer cell, soft tissue sarcoma, or a hematological cancer cell.
